SEQUENCE LISTING (164 sequences)

SEQ ID NO: 1 - protein, 13 aa, synthetic construct
  REGION 1..13: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 8: note = Xaa is 4-methoxy-L-phenylalanine (Yme)
  MOD_RES 9: note = Xaa is 2-naphthyl-L-alanine (Nal)
  MOD_RES 10: note = Xaa is 2-aminoisobutyric acid (Aib)
  MOD_RES 13: note = AMIDATION
XCNTWQCXXXENN

SEQ ID NO: 2 - protein, 13 aa, synthetic construct
  REGION 1..13: note = Artificial Peptide
  MOD_RES 1: note = Xaa is 6-aminohexanoic acid (Ahx)
  REGION 1..11: note = MISC_FEATURE - lactam bridge between Ahx at position X1 and Glu at position X11
  DISULFID 2..7: note = disulfide bridge between Cys at position X2 and Cys at position X7
  MOD_RES 8: note = Xaa is 4-methoxy-L-phenylalanine (Yme)
  MOD_RES 9: note = Xaa is 2-naphthyl-L-alanine (Nal)
  MOD_RES 10: note = Xaa is 2-aminoisobutyric acid (Aib)
  MOD_RES 13: note = AMIDATION
XCNTWQCXXXENN

SEQ ID NO: 3 - protein, 13 aa, synthetic construct
  REGION 1..13: note = Artificial Peptide
  MOD_RES 1: note = Xaa is 6-aminohexanoic acid (Ahx)
  REGION 1..11: note = MISC_FEATURE - lactam bridge between Ahx at position X1 and Asp at position X11
  DISULFID 2..7: note = disulfide bridge between Cys at position X2 and Cys at position X7
  MOD_RES 8: note = Xaa is 4-methoxy-L-phenylalanine (Yme)
  MOD_RES 9: note = Xaa is 2-naphthyl-L-alanine (Nal)
  MOD_RES 10: note = Xaa is 2-aminoisobutyric acid (Aib)
  MOD_RES 13: note = AMIDATION
XCNTWQCXXXDNN

SEQ ID NO: 4 - protein, 13 aa, synthetic construct
  REGION 1..13: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 8: note = Xaa is 4-methoxy-L-phenylalanine (Yme)
  MOD_RES 9: note = Xaa is 2-naphthyl-L-alanine (Nal)
  MOD_RES 10: note = Xaa is 2-aminoisobutyric acid (Aib)
  MOD_RES 13: note = AMIDATION
XCNTWQCXXXENG

SEQ ID NO: 5 - protein, 13 aa, synthetic construct
  REGION 1..13: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 8: note = Xaa is 4-aminoethoxy-L-phenylalanine (Yae)
  MOD_RES 9: note = Xaa is 2-naphthyl-L-alanine (Nal)
  MOD_RES 10: note = Xaa is 2-aminoisobutyric acid (Aib)
  MOD_RES 13: note = AMIDATION
XCNTWQCXXXENN

SEQ ID NO: 6 - protein, 13 aa, synthetic construct
  REGION 1..13: note = Artificial Peptide
  MOD_RES 1: note = Xaa is 6-aminohexanoic acid (Ahx)
  REGION 1..11: note = MISC_FEATURE - lactam bridge between Ahx at position X1 and Asp at position X11
  DISULFID 2..7: note = disulfide bridge between Cys at position X2 and Cys at position X7
  MOD_RES 3: note = Xaa is N-methyl-L-asparagine (Nmn)
  MOD_RES 8: note = Xaa is 4-methoxy-L-phenylalanine (Yme)
  MOD_RES 9: note = Xaa is 2-naphthyl-L-alanine (Nal)
  MOD_RES 10: note = Xaa is 2-aminoisobutyric acid (Aib)
  MOD_RES 13: note = AMIDATION
XCXTWQCXXXDNN

SEQ ID NO: 7 - protein, 13 aa, synthetic construct
  REGION 1..13: note = Artificial Peptide
  MOD_RES 1: note = ACETYLATION
  REGION 1..11: note = MISC_FEATURE - lactam bridge between Lys at position X1 and Asp at position X11
  DISULFID 2..7: note = disulfide bridge between Cys at position X2 and Cys at position X7
  MOD_RES 8: note = Xaa is 4-methoxy-L-phenylalanine (Yme)
  MOD_RES 9: note = Xaa is 2-naphthyl-L-alanine (Nal)
  MOD_RES 10: note = Xaa is 2-aminoisobutyric acid (Aib)
  MOD_RES 13: note = AMIDATION
KCNTWQCXXXDNN

SEQ ID NO: 8 - protein, 13 aa, synthetic construct
  REGION 1..13: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is 2-aminoisobutyric acid (Aib)
  MOD_RES 13: note = AMIDATION
XCNTWQCXXXENN

SEQ ID NO: 9 - protein, 13 aa, synthetic construct
  REGION 1..13: note = Artificial Peptide
  MOD_RES 1: note = Xaa is 6-aminohexanoic acid (Ahx)
  REGION 1..11: note = MISC_FEATURE - lactam bridge between Ahx at position X1 and Asp at position X11
  DISULFID 2..7: note = disulfide bridge between Cys at position X2 and Cys at position X7
  MOD_RES 8: note = Xaa is 4-aminoethoxy-L-phenylalanine (Yae)
  MOD_RES 9: note = Xaa is 2-naphthyl-L-alanine (Nal)
  MOD_RES 10: note = Xaa is 2-aminoisobutyric acid (Aib)
  MOD_RES 13: note = AMIDATION
XCNTWQCXXXDNG

SEQ ID NO: 10 - protein, 13 aa, synthetic construct
  REGION 1..13: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is alpha-methyl-L-leucine (Mle)
  MOD_RES 13: note = Xaa is D-leucine (D-Leu)
  MOD_RES 13: note = AMIDATION
XCNTWQCXXXENX

SEQ ID NO: 11 - protein, 13 aa, synthetic construct
  REGION 1..13: note = Artificial Peptide
  MOD_RES 1: note = Xaa is desamino-aspartamic acid (Dad)
  REGION 1..11: note = MISC_FEATURE - lactam bridge between Dad at position X1 and Lys at position X11
  DISULFID 2..7: note = disulfide bridge between Cys at position X2 and Cys at position X7
  MOD_RES 8: note = Xaa is 4-methoxy-L-phenylalanine (Yme)
  MOD_RES 9: note = Xaa is 2-naphthyl-L-alanine (Nal)
  MOD_RES 10: note = Xaa is 2-aminoisobutyric acid (Aib)
  MOD_RES 13: note = AMIDATION
XCNTWQCXXXKNN

SEQ ID NO: 12 - protein, 12 aa, synthetic construct
  REGION 1..12: note = Artificial Peptide
  MOD_RES 1: note = Xaa is 6-(methylamino)pentanoic acid (Nmapt)
  REGION 1..10: note = MISC_FEATURE - lactam bridge between Nmapt at position X1 and Glu at position X10
  DISULFID 2..6: note = disulfide bridge between Cys at position X2 and Cys at position X6
  REGION 2..3: note = MISC_FEATURE - bond between Cys at position X2 and epsilon amino group of adjacent acetylated Lys
  MOD_RES 3: note = ACETYLATION
  MOD_RES 7: note = Xaa is 4-aminoethoxy-L-phenylalanine (Yae)
  MOD_RES 8: note = Xaa is 2-naphthyl-L-alanine (Nal)
  MOD_RES 9: note = Xaa is 2-aminoisobutyric acid (Aib)
  MOD_RES 12: note = AMIDATION
XCKWQCXXXENN

SEQ ID NO: 13 - protein, 12 aa, synthetic construct
  REGION 1..12: note = Artificial Peptide
  MOD_RES 1: note = Xaa is 6-(methylamino)pentanoic acid (Nmapt)
  REGION 1..10: note = MISC_FEATURE - lactam bridge between Nmapt at position X1 and Glu at position X10
  DISULFID 2..6: note = disulfide bridge between Cys at position X2 and Cys at position X6
  REGION 2..3: note = MISC_FEATURE - bond between Cys at position X2 and epsilon amino group of adjacent acetylated Lys
  MOD_RES 3: note = ACETYLATION
  MOD_RES 7: note = Xaa is 4-aminoethoxy-L-phenylalanine (Yae)
  MOD_RES 8: note = Xaa is 2-naphthyl-L-alanine (Nal)
  MOD_RES 9: note = Xaa is 4-amino-tetrahydropyran-4-carboxylic acid (Thp)
  MOD_RES 12: note = AMIDATION
XCKWQCXXXENN

SEQ ID NO: 14 - protein, 12 aa, synthetic construct
  REGION 1..12: note = Artificial Peptide
  MOD_RES 1: note = Xaa is 6-(methylamino)pentanoic acid (Nmapt)
  REGION 1..10: note = MISC_FEATURE - lactam bridge between Nmapt at position X1 and Glu at position X10
  DISULFID 2..6: note = disulfide bridge between Cys at position X2 and Cys at position X6
  REGION 2..3: note = MISC_FEATURE - bond between Cys at position X2 and epsilon amino group of adjacent acetylated Lys
  MOD_RES 3: note = ACETYLATION
  MOD_RES 7: note = Xaa is 4-aminoethoxy-L-phenylalanine (Yae)
  MOD_RES 8: note = Xaa is 2-naphthyl-L-alanine (Nal)
  MOD_RES 9: note = Xaa is alpha-methyl-L-leucine (Mle)
  MOD_RES 12: note = AMIDATION
XCKWQCXXXENN

SEQ ID NO: 15 - protein, 12 aa, synthetic construct
  REGION 1..12: note = Artificial Peptide
  MOD_RES 1: note = Xaa is 6-(methylamino)pentanoic acid (Nmapt)
  REGION 1..10: note = MISC_FEATURE - lactam bridge between Nmapt at position X1 and Glu at position X10
  DISULFID 2..6: note = disulfide bridge between Cys at position X2 and Cys at position X6
  REGION 2..3: note = MISC_FEATURE - bond between Cys at position X2 and epsilon amino group of adjacent acetylated Lys
  MOD_RES 3: note = ACETYLATION
  MOD_RES 7: note = Xaa is 4-aminoethoxy-L-phenylalanine (Yae)
  MOD_RES 8: note = Xaa is 2-naphthyl-L-alanine (Nal)
  MOD_RES 9: note = Xaa is 2-aminoisobutyric acid (Aib)
  MOD_RES 12: note = AMIDATION
XCKWQCXXXENG

SEQ ID NO: 16 - protein, 12 aa, synthetic construct
  REGION 1..12: note = Artificial Peptide
  MOD_RES 1: note = Xaa is 6-(methylamino)pentanoic acid (Nmapt)
  REGION 1..10: note = MISC_FEATURE - lactam bridge between Nmapt at position X1 and Glu at position X10
  DISULFID 2..6: note = disulfide bridge between Cys at position X2 and Cys at position X6
  REGION 2..3: note = MISC_FEATURE - bond between Cys at position X2 and epsilon amino group of adjacent acetylated Lys
  MOD_RES 3: note = ACETYLATION
  MOD_RES 7: note = Xaa is 4-aminoethoxy-L-phenylalanine (Yae)
  MOD_RES 8: note = Xaa is 2-naphthyl-L-alanine (Nal)
  MOD_RES 9: note = Xaa is 4-amino-tetrahydropyran-4-carboxylic acid (Thp)
  MOD_RES 12: note = AMIDATION
XCKWQCXXXENG

SEQ ID NO: 17 - protein, 12 aa, synthetic construct
  REGION 1..12: note = Artificial Peptide
  MOD_RES 1: note = Xaa is 6-(methylamino)pentanoic acid (Nmapt)
  REGION 1..10: note = MISC_FEATURE - lactam bridge between Nmapt at position X1 and Glu at position X10
  DISULFID 2..6: note = disulfide bridge between Cys at position X2 and Cys at position X6
  REGION 2..3: note = MISC_FEATURE - bond between Cys at position X2 and epsilon amino group of adjacent acetylated Lys
  MOD_RES 3: note = ACETYLATION
  MOD_RES 7: note = Xaa is 4-aminoethoxy-L-phenylalanine (Yae)
  MOD_RES 8: note = Xaa is 2-naphthyl-L-alanine (Nal)
  MOD_RES 9: note = Xaa is 4-amino-tetrahydropyran-4-carboxylic acid (Thp)
  MOD_RES 12: note = AMIDATION
XCKWQCXXXEAG

SEQ ID NO: 18 - protein, 12 aa, synthetic construct
  REGION 1..12: note = Artificial Peptide
  MOD_RES 1: note = Xaa is 6-(methylamino)pentanoic acid (Nmapt)
  REGION 1..10: note = MISC_FEATURE - lactam bridge between Nmapt at position X1 and Glu at position X10
  DISULFID 2..6: note = disulfide bridge between Cys at position X2 and Cys at position X6
  REGION 2..3: note = MISC_FEATURE - bond between Cys at position X2 and epsilon amino group of adjacent acetylated Lys
  MOD_RES 3: note = ACETYLATION
  MOD_RES 7: note = Xaa is 4-aminoethoxy-L-phenylalanine (Yae)
  MOD_RES 8: note = Xaa is 2-naphthyl-L-alanine (Nal)
  MOD_RES 9: note = Xaa is alpha-methyl-L-leucine (Mle)
  MOD_RES 12: note = AMIDATION
XCKWQCXXXEAG

SEQ ID NO: 19 - protein, 12 aa, synthetic construct
  REGION 1..12: note = Artificial Peptide
  MOD_RES 1: note = Xaa is 6-(methylamino)pentanoic acid (Nmapt)
  REGION 1..10: note = MISC_FEATURE - lactam bridge between Nmapt at position X1 and Glu at position X10
  DISULFID 2..6: note = disulfide bridge between Cys at position X2 and Cys at position X6
  REGION 2..3: note = MISC_FEATURE - bond between Cys at position X2 and epsilon amino group of adjacent acetylated Lys
  MOD_RES 3: note = ACETYLATION
  MOD_RES 7: note = Xaa is 4-aminoethoxy-L-phenylalanine (Yae)
  MOD_RES 8: note = Xaa is 2-naphthyl-L-alanine (Nal)
  MOD_RES 9: note = Xaa is 2-aminoisobutyric acid (Aib)
  MOD_RES 12: note = Xaa is D-leucine (D-Leu)
  MOD_RES 12: note = AMIDATION
XCKWQCXXXEAX

SEQ ID NO: 20 - protein, 13 aa, synthetic construct
  REGION 1..13: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 5: note = Xaa is 7-Methyl-L-tryptophan (Wim)
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is 2-aminoisobutyric acid (Aib)
  MOD_RES 13: note = AMIDATION
XCNTXKCXXXENH

SEQ ID NO: 21 - protein, 13 aa, synthetic construct
  REGION 1..13: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 5: note = Xaa is 7-methyl-L-tryptophan (Wim)
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is 4-amino-tetrahydropyran-4-carboxylic acid (Thp)
  MOD_RES 13: note = AMIDATION
XCNTXKCXXXENH

SEQ ID NO: 22 - protein, 13 aa, synthetic construct
  REGION 1..13: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 5: note = Xaa is 7-methyl-L-tryptophan (Wim)
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is alpha-methyl-L-leucine (Mle)
  MOD_RES 13: note = AMIDATION
XCNTXKCXXXENH

SEQ ID NO: 23 - protein, 13 aa, synthetic construct
  REGION 1..13: note = Artificial Peptide
  MOD_RES 1: note = Xaa is desamino-aspartamic acid (Dad)
  REGION 1..11: note = MISC_FEATURE - lactam bridge between Dad at position X1 and Lys at position X11
  DISULFID 2..7: note = disulfide bridge between Cys at position X2 and Cys at position X7
  MOD_RES 5: note = Xaa is 7-methyl-L-tryptophan (Wim)
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is alpha-methyl-L-leucine (Mle)
  MOD_RES 13: note = AMIDATION
XCNTXKCXXXKNH

SEQ ID NO: 24 - protein, 14 aa, synthetic construct
  REGION 1..14: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 5: note = Xaa is 7-methyl-L-tryptophan (Wim)
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is 4-amino-tetrahydropyran-4-carboxylic acid (Thp)
  MOD_RES 13: note = Xaa is L-3-pyridyl-alanine (Pal)
  MOD_RES 14: note = Xaa is sarcosine (Sar)
  MOD_RES 14: note = AMIDATION
XCNTXLCXXXENXX

SEQ ID NO: 25 - protein, 13 aa, synthetic construct
  REGION 1..13: note = Artificial Peptide
  MOD_RES 1: note = Xaa is desamino-aspartamic acid (Dad)
  REGION 1..11: note = MISC_FEATURE - lactam bridge between Dad at position X1 and Kme at position X11
  DISULFID 2..7: note = disulfide bridge between Cys at position X2 and Cys at position X7
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is 2-aminoisobutyric acid (Aib)
  MOD_RES 11: note = Xaa is (2S)-2-amino-6-(methylamino)hexanoic acid (epsilon-N-methyl-lysine) (Kme)
  MOD_RES 13: note = AMIDATION
XCNTWQCXXXXNN

SEQ ID NO: 26 - protein, 13 aa, synthetic construct
  REGION 1..13: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is 4-amino-tetrahydropyran-4-carboxylic acid (Thp)
  MOD_RES 13: note = AMIDATION
XCNTWQCXXXENN

SEQ ID NO: 27 - protein, 13 aa, synthetic construct
  REGION 1..13: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is alpha-methyl-L-leucine (Mle)
  MOD_RES 13: note = AMIDATION
XCNTWQCXXXENN

SEQ ID NO: 28 - protein, 12 aa, synthetic construct
  REGION 1..12: note = Artificial Peptide
  MOD_RES 1: note = Xaa is 5-aminopentanoic acid (Apt)
  METAL 1..10: note = lactam bridge between Apt at position X1 and Glu at position X10
  DISULFID 2..6: note = disulfide bridge between Cys at position X2 and Cys at position X6
  REGION 2..3: note = MISC_FEATURE - bond between Cys at position X2 and epsilon amino group of adjacent acetylated Lys
  MOD_RES 3: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is alpha-methyl-L-leucine (Mle)
  MOD_RES 12: note = AMIDATION
XCKWQCXXXENN

SEQ ID NO: 29 - protein, 12 aa, synthetic construct
  REGION 1..12: note = Artificial Peptide
  MOD_RES 1: note = Xaa is 5-aminopentanoic acid (Apt)
  REGION 1..10: note = MISC_FEATURE - lactam bridge between Apt at position X1 and Glu at position X10
  DISULFID 2..6: note = disulfide bridge between Cys at position X2 and Cys at position X6
  REGION 2..3: note = MISC_FEATURE - bond between Cys at position X2 and epsilon amino group of adjacent acetylated Lys
  MOD_RES 3: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is 2-aminoisobutyric acid (Aib)
  MOD_RES 12: note = AMIDATION
XCKWQCXXXENG

SEQ ID NO: 30 - protein, 12 aa, synthetic construct
  REGION 1..12: note = Artificial Peptide
  MOD_RES 1: note = Xaa is 5-aminopentanoic acid (Apt)
  REGION 1..10: note = MISC_FEATURE - lactam bridge between Apt at position X1 and Glu at position X10
  DISULFID 2..6: note = disulfide bridge between Cys at position X2 and Cys at position X6
  REGION 2..3: note = MISC_FEATURE - bond between Cys at position X2 and epsilon amino group of adjacent acetylated Lys
  MOD_RES 3: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is 4-amino-tetrahydropyran-4-carboxylic acid (Thp)
  MOD_RES 12: note = AMIDATION
XCKWQCXXXENG

SEQ ID NO: 31 - protein, 12 aa, synthetic construct
  REGION 1..12: note = Artificial Peptide
  MOD_RES 1: note = Xaa is 5-aminopentanoic acid (Apt)
  REGION 1..10: note = MISC_FEATURE - lactam bridge between Apt at position X1 and Glu at position X10
  DISULFID 2..6: note = disulfide bridge between Cys at position X2 and Cys at position X6
  REGION 2..3: note = MISC_FEATURE - bond between Cys at position X2 and epsilon amino group of adjacent acetylated Lys
  MOD_RES 3: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is alpha-methyl-L-leucine (Mle)
  MOD_RES 12: note = AMIDATION
XCKWQCXXXENG

SEQ ID NO: 32 - protein, 12 aa, synthetic construct
  REGION 1..12: note = Artificial Peptide
  MOD_RES 1: note = Xaa is 5-aminopentanoic acid (Apt)
  REGION 1..10: note = MISC_FEATURE - lactam bridge between Apt at position X1 and Glu at position X10
  DISULFID 2..6: note = disulfide bridge between Cys at position X2 and Cys at position X6
  REGION 2..3: note = MISC_FEATURE - bond between Cys at position X2 and epsilon amino group of adjacent acetylated Lys
  MOD_RES 3: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is 2-aminoisobutyric acid (Aib)
  MOD_RES 12: note = AMIDATION
XCKWQCXXXEAG

SEQ ID NO: 33 - protein, 12 aa, synthetic construct
  REGION 1..12: note = Artificial Peptide
  MOD_RES 1: note = Xaa is 5-aminopentanoic acid (Apt)
  REGION 1..10: note = MISC_FEATURE - lactam bridge between Apt at position X1 and Glu at position X10
  DISULFID 2..6: note = disulfide bridge between Cys at position X2 and Cys at position X6
  REGION 2..3: note = MISC_FEATURE - bond between Cys at position X2 and epsilon amino group of adjacent acetylated Lys
  MOD_RES 3: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is 4-amino-tetrahydropyran-4-carboxylic acid (Thp)
  MOD_RES 12: note = Xaa is D-leucine (D-Leu)
  MOD_RES 12: note = AMIDATION
XCKWQCXXXEAX

SEQ ID NO: 34 - protein, 12 aa, synthetic construct
  REGION 1..12: note = Artificial Peptide
  MOD_RES 1: note = Xaa is 6-(methylamino)pentanoic acid (Nmapt)
  REGION 1..10: note = MISC_FEATURE - lactam bridge between Apt at position X1 and Glu at position X10
  DISULFID 2..6: note = disulfide bridge between Cys at position X2 and Cys at position X6
  REGION 2..3: note = MISC_FEATURE - bond between Cys at position X2 and epsilon amino group of adjacent acetylated Lys
  MOD_RES 3: note = ACETYLATION
  MOD_RES 7: note = Xaa is 4-aminoethoxy-L-phenylalanine (Yae)
  MOD_RES 8: note = Xaa is 2-naphthyl-L-alanine (Nal)
  MOD_RES 9: note = Xaa is 4-amino-tetrahydropyran-4-carboxylic acid (Thp)
  MOD_RES 12: note = Xaa is D-leucine (D-Leu)
  MOD_RES 12: note = AMIDATION
XCKWQCXXXEAX

SEQ ID NO: 35 - protein, 14 aa, synthetic construct
  REGION 1..14: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 5: note = Xaa is 7-methyl-L-tryptophan (Wim)
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is 4-amino-tetrahydropyran-4-carboxylic acid (Thp)
  MOD_RES 13: note = Xaa is L-3-pyridyl-alanine (Pal)
  MOD_RES 14: note = Xaa is sarcosine (Sar)
  MOD_RES 14: note = AMIDATION
XCNTXKCXXXENXX

SEQ ID NO: 36 - protein, 13 aa, synthetic construct
  REGION 1..13: note = Artificial Peptide
  MOD_RES 1: note = Xaa is 5-aminopentanoic acid (Apt)
  REGION 1..10: note = MISC_FEATURE - lactam bridge between Apt at position X1 and Glu at position X10
  DISULFID 2..6: note = disulfide bridge between Cys at position X2 and Cys at position X6
  MOD_RES 3: note = Xaa is (2S)-6-amino-2-(dimethylamino)hexanoic acid (Ndmk)
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is (2S)-2-amino-6-(dimethylamino)-2-methyl-hexanoic acid (Mkdm)
  MOD_RES 12: note = Xaa is L-3-pyridyl-alanine (Pal)
  MOD_RES 13: note = Xaa is sarcosine (Sar)
  MOD_RES 13: note = AMIDATION
XCXWKCXXXENXX

SEQ ID NO: 37 - protein, 14 aa, synthetic construct
  REGION 1..14: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is 2-aminoisobutyric acid (Aib)
  MOD_RES 12: note = Xaa is L-3-pyridyl-alanine (Pal)
  MOD_RES 13: note = AMIDATION
XCNTWKCXXXENXI

SEQ ID NO: 38 - protein, 14 aa, synthetic construct
  REGION 1..14: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is 2-aminoisobutyric acid (Aib)
  MOD_RES 13: note = AMIDATION
XCNTWKCXXXENNI

SEQ ID NO: 39 - protein, 12 aa, synthetic construct
  REGION 1..12: note = Artificial Peptide
  MOD_RES 1: note = Xaa is 6-aminohexanoic acid (Ahx)
  REGION 1..10: note = MISC_FEATURE - lactam bridge between Ahx at position X1 and Asp at position X10
  REGION 2..6: note = MISC_FEATURE - ester bridge between Glu at position X2 and Thr at position X6
  MOD_RES 3: note = Xaa is 5-aminopentanoic acid (Apt)
  MOD_RES 7: note = Xaa is 4-methoxy-L-phenylalanine (Yme)
  MOD_RES 8: note = Xaa is 2-naphthyl-L-alanine (Nal)
  MOD_RES 9: note = Xaa is 2-aminoisobutyric acid (Aib)
  MOD_RES 12: note = AMIDATION
XEXWQTXXXDNN

SEQ ID NO: 40 - protein, 13 aa, synthetic construct
  REGION 1..13: note = Artificial Peptide
  MOD_RES 1: note = Xaa is 6-(methylamino)hexanoic acid (Nmha)
  REGION 1..11: note = MISC_FEATURE - lactam bridge between Nmha at position X1 and Asp at position X11
  DISULFID 2..7: note = disulfide bridge between Cys at position X2 and Cys at position X7
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is alpha-methyl-L-leucine (Mle)
  MOD_RES 13: note = Xaa is D-leucine (D-Leu)
  MOD_RES 13: note = AMIDATION
XCNTWQCXXXDNX

SEQ ID NO: 41 - protein, 13 aa, synthetic construct
  REGION 1..13: note = Artificial Peptide
  MOD_RES 1: note = Xaa is 6-(methylamino)pentanoic acid (Nmapt)
  REGION 1..11: note = MISC_FEATURE - lactam bridge between Nmapt at position X1 and Glu at position X11
  DISULFID 2..7: note = disulfide bridge between Cys at position X2 and Cys at position X7
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is alpha-methyl-L-leucine (Mle)
  MOD_RES 13: note = AMIDATION
XCNTWQCXXXENN

SEQ ID NO: 42 - protein, 13 aa, synthetic construct
  REGION 1..13: note = Artificial Peptide
  MOD_RES 1: note = Xaa is 6-(methylamino)pentanoic acid (Nmapt)
  REGION 1..11: note = MISC_FEATURE - lactam bridge between Nmapt at position X1 and Glu at position X11
  DISULFID 2..7: note = disulfide bridge between Cys at position X2 and Cys at position X7
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is alpha-methyl-L-leucine (Mle)
  MOD_RES 13: note = AMIDATION
XCNTWQCXXXENN

SEQ ID NO: 43 - protein, 13 aa, synthetic construct
  REGION 1..13: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 5: note = Xaa is 7-methyl-L-tryptophan (Wim)
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is 2-aminoisobutyric acid (Aib)
  MOD_RES 13: note = AMIDATION
XCNTXQCXXXESH

SEQ ID NO: 44 - protein, 12 aa, synthetic construct
  REGION 1..12: note = Artificial Peptide
  MOD_RES 1: note = Xaa is 6-(methylamino)pentanoic acid (Nmapt)
  REGION 1..10: note = MISC_FEATURE - lactam bridge between Nmapt at position X1 and Glu at position X10
  DISULFID 2..6: note = disulfide bridge between Cys at position X2 and Cys at position X6
  REGION 2..3: note = MISC_FEATURE - bond between Cys at position X2 and epsilon amino group of adjacent acetylated Lys
  MOD_RES 3: note = ACETYLATION
  MOD_RES 7: note = Xaa is 4-aminoethoxy-L-phenylalanine (Yae)
  MOD_RES 8: note = Xaa is 2-naphthyl-L-alanine (Nal)
  MOD_RES 9: note = Xaa is alpha-methyl-L-leucine (Mle)
  MOD_RES 12: note = Xaa is D-leucine (D-Leu)
  MOD_RES 12: note = AMIDATION
XCKWQCXXXEAX

SEQ ID NO: 45 - protein, 14 aa, synthetic construct
  REGION 1..14: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 5: note = Xaa is 7-methyl-L-tryptophan (Wim)
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is 2-aminoisobutyric acid (Aib)
  MOD_RES 14: note = Xaa is sarcosine (Sar)
  MOD_RES 14: note = AMIDATION
XCNTXKCXXXENHX

SEQ ID NO: 46 - protein, 14 aa, synthetic construct
  REGION 1..14: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 5: note = Xaa is 7-methyl-L-tryptophan (Wim)
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is 4-amino-tetrahydropyran-4-carboxylic acid (Thp)
  MOD_RES 14: note = Xaa is sarcosine (Sar)
  MOD_RES 14: note = AMIDATION
XCNTXKCXXXENHX

SEQ ID NO: 47 - protein, 14 aa, synthetic construct
  REGION 1..14: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 5: note = Xaa is 7-methyl-L-tryptophan (Wim)
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is alpha-methyl-L-leucine (Mle)
  MOD_RES 14: note = Xaa is sarcosine (Sar)
  MOD_RES 14: note = AMIDATION
XCNTXKCXXXENHX

SEQ ID NO: 48 - protein, 14 aa, synthetic construct
  REGION 1..14: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 5: note = Xaa is 7-methyl-L-tryptophan (Wim)
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is 2-aminoisobutyric acid (Aib)
  MOD_RES 14: note = Xaa is beta-alanine (Bal)
  MOD_RES 14: note = AMIDATION
XCNTXKCXXXENHX

SEQ ID NO: 49 - protein, 14 aa, synthetic construct
  REGION 1..14: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 5: note = Xaa is 7-methyl-L-tryptophan (Wim)
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is 4-amino-tetrahydropyran-4-carboxylic acid (Thp)
  MOD_RES 14: note = Xaa is beta-alanine (Bal)
  MOD_RES 14: note = AMIDATION
XCNTXKCXXXENHX

SEQ ID NO: 50 - protein, 14 aa, synthetic construct
  REGION 1..14: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 5: note = Xaa is 7-methyl-L-tryptophan (Wim)
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is alpha-methyl-L-leucine (Mle)
  MOD_RES 14: note = Xaa is beta-alanine (Bal)
  MOD_RES 14: note = AMIDATION
XCNTXKCXXXENHX

SEQ ID NO: 51 - protein, 13 aa, synthetic construct
  REGION 1..13: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 5: note = Xaa is 7-methyl-L-tryptophan (Wim)
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is 4-amino-tetrahydropyran-4-carboxylic acid (Thp)
  MOD_RES 13: note = Xaa is L-3-pyridyl-alanine (Pal)
  MOD_RES 13: note = AMIDATION
XCNTXKCXXXENX

SEQ ID NO: 52 - protein, 13 aa, synthetic construct
  REGION 1..13: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 5: note = Xaa is 7-methyl-L-tryptophan (Wim)
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is 2-aminoisobutyric acid (Aib)
  MOD_RES 13: note = AMIDATION
XCNTXQCXXXENH

SEQ ID NO: 53 - protein, 12 aa, synthetic construct
  REGION 1..12: note = Artificial Peptide
  MOD_RES 1: note = Xaa is 6-aminohexanoic acid (Ahx)
  REGION 1..10: note = MISC_FEATURE - lactam bridge between Ahx at position X1 and Asp at position X10
  DISULFID 2..6: note = disulfide bridge between Cys at position X2 and Cys at position X6
  MOD_RES 3: note = Xaa is 5-aminopentanoic acid (Apt)
  MOD_RES 7: note = Xaa is 4-aminoethoxy-L-phenylalanine (Yae)
  MOD_RES 8: note = Xaa is 2-naphthyl-L-alanine (Nal)
  MOD_RES 9: note = Xaa is 2-aminoisobutyric acid (Aib)
  MOD_RES 12: note = AMIDATION
XCXWQCXXXDAG

SEQ ID NO: 54 - protein, 13 aa, synthetic construct
  REGION 1..13: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 5: note = Xaa is 7-methyl-L-tryptophan (Wim)
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is (2S)-2-amino-6-(dimethylamino)-2-methyl-hexanoic acid (Mkdm)
  MOD_RES 13: note = Xaa is D-leucine (D-Leu)
  MOD_RES 13: note = AMIDATION
XCNTXKCXXXENX

SEQ ID NO: 55 - protein, 14 aa, synthetic construct
  REGION 1..14: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 5: note = Xaa is 7-methyl-L-tryptophan (Wim)
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is 4-amino-tetrahydropyran-4-carboxylic acid (Thp)
  MOD_RES 14: note = Xaa is (2S)-2-methylpyrrolidine-2-carboxylic acid (alpha-Methyl-L-proline) (Mep)
  MOD_RES 14: note = AMIDATION
XCNTXKCXXXENHX

SEQ ID NO: 56 - protein, 14 aa, synthetic construct
  REGION 1..14: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 5: note = Xaa is 7-methyl-L-tryptophan (Wim)
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is 4-amino-tetrahydropyran-4-carboxylic acid (Thp)
  MOD_RES 13: note = Xaa is L-3-pyridyl-alanine (Pal)
  MOD_RES 14: note = Xaa is sarcosine (Sar)
  MOD_RES 14: note = AMIDATION
XCNTXKCXXXESXX

SEQ ID NO: 57 - protein, 14 aa, synthetic construct
  REGION 1..14: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is 2-aminoisobutyric acid (Aib)
  MOD_RES 14: note = AMIDATION
XCNTWQCXXXENNI

SEQ ID NO: 58 - protein, 14 aa, synthetic construct
  REGION 1..14: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is (2S)-2-amino-6-(dimethylamino)-2-methyl-hexanoic acid (Mkdm)
  MOD_RES 13: note = Xaa is L-3-pyridyl-alanine (Pal)
  MOD_RES 14: note = Xaa is sarcosine (Sar)
  MOD_RES 14: note = AMIDATION
XCNTWKCXXXENXX

SEQ ID NO: 59 - protein, 13 aa, synthetic construct
  REGION 1..13: note = Artificial Peptide
  MOD_RES 1: note = Xaa is 5-aminopentanoic acid (Apt)
  REGION 1..10: note = MISC_FEATURE - lactam bridge between Apt at position X1 and Glu at position X10
  DISULFID 2..6: note = disulfide bridge between Cys at position X2 and Cys at position X6
  REGION 2..3: note = MISC_FEATURE - bond between Cys at position X2 and epsilon amino group of adjacent acetylated Lys
  MOD_RES 3: note = ACETYLATION
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is (2S)-2-amino-6-(dimethylamino)-2-methyl-hexanoic acid (Mkdm)
  MOD_RES 12: note = Xaa is L-3-pyridyl-alanine (Pal)
  MOD_RES 13: note = Xaa is sarcosine (Sar)
  MOD_RES 13: note = AMIDATION
XCKWKCXXXENXX

SEQ ID NO: 60 - protein, 12 aa, synthetic construct
  REGION 1..12: note = Artificial Peptide
  MOD_RES 1: note = Xaa is N-methyl-5-aminopentanoic acid (Nmapt)
  REGION 1..10: note = MISC_FEATURE - lactam bridge between Nmapt at position X1 and Glu at position X10
  DISULFID 2..6: note = disulfide bridge between Cys at position X2 and Cys at position X6
  REGION 2..3: note = MISC_FEATURE - bond between Cys at position X2 and epsilon amino group of adjacent acetylated Lys
  MOD_RES 3: note = ACETYLATION
  MOD_RES 4: note = Xaa is 7-methyl-L-tryptophan (Wim)
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is 2-aminoisobutyric acid (Aib)
  MOD_RES 12: note = AMIDATION
XCKXKCXXXENH

SEQ ID NO: 61 - protein, 13 aa, synthetic construct
  REGION 1..13: note = Artificial Peptide
  MOD_RES 1: note = Xaa is 6-(methylamino)pentanoic acid (Nmapt)
  REGION 1..10: note = MISC_FEATURE - lactam bridge between Nmapt at position X1 and Glu at position X10
  DISULFID 2..6: note = disulfide bridge between Cys at position X2 and Cys at position X6
  MOD_RES 3: note = Xaa is (2S)-6-amino-2-(dimethylamino)hexanoic acid (Ndmk)
  MOD_RES 4: note = Xaa is 7-methyl-L-tryptophan (Wim)
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is (2S)-2-amino-6-(dimethylamino)-2-methyl-hexanoic acid (Mkdm)
  MOD_RES 12: note = Xaa is L-3-pyridyl-alanine (Pal)
  MOD_RES 13: note = Xaa is sarcosine (Sar)
  MOD_RES 13: note = AMIDATION
XCXXKCXXXENXX

SEQ ID NO: 62 - protein, 14 aa, synthetic construct
  REGION 1..14: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 5: note = Xaa is 7-methyl-L-tryptophan (Wim)
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is 2-aminoisobutyric acid (Aib)
  MOD_RES 13: note = Xaa is L-3-pyridyl-alanine (Pal)
  MOD_RES 14: note = Xaa is sarcosine (Sar)
  MOD_RES 14: note = AMIDATION
XCNTXQCXXXENXX

SEQ ID NO: 63 - protein, 14 aa, synthetic construct
  REGION 1..14: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 5: note = Xaa is 7-methyl-L-tryptophan (Wim)
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is alpha-methyl-L-leucine (Mle)
  MOD_RES 13: note = Xaa is L-3-pyridyl-alanine (Pal)
  MOD_RES 14: note = Xaa is sarcosine (Sar)
  MOD_RES 14: note = AMIDATION
XCNTXQCXXXENXX

SEQ ID NO: 64 - protein, 14 aa, synthetic construct
  REGION 1..14: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 5: note = Xaa is 7-methyl-L-tryptophan (Wim)
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is 2-aminoisobutyric acid (Aib)
  MOD_RES 13: note = Xaa is L-3-pyridyl-alanine (Pal)
  MOD_RES 14: note = Xaa is sarcosine (Sar)
  MOD_RES 14: note = AMIDATION
XCNTXKCXXXENXX

SEQ ID NO: 65 - protein, 14 aa, synthetic construct
  REGION 1..14: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 5: note = Xaa is 7-methyl-L-tryptophan (Wim)
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is alpha-methyl-L-leucine (Mle)
  MOD_RES 13: note = Xaa is L-3-pyridyl-alanine (Pal)
  MOD_RES 14: note = Xaa is sarcosine (Sar)
  MOD_RES 14: note = AMIDATION
XCNTXKCXXXENXX

SEQ ID NO: 66 - protein, 13 aa, synthetic construct
  REGION 1..13: note = Artificial Peptide
  MOD_RES 1: note = Xaa is 5-aminopentanoic acid (Apt)
  REGION 1..10: note = MISC_FEATURE - lactam bridge between Apt at position X1 and Glu at position X10
  DISULFID 2..6: note = disulfide bridge between Cys at position X2 and Cys at position X6
  REGION 2..3: note = MISC_FEATURE - bond between Cys at position X2 and epsilon amino group of adjacent acetylated Lys
  MOD_RES 3: note = ACETYLATION
  MOD_RES 4: note = Xaa is 7-methyl-L-tryptophan (Wim)
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is 4-amino-tetrahydropyran-4-carboxylic acid (Thp)
  MOD_RES 12: note = Xaa is L-3-pyridyl-alanine (Pal)
  MOD_RES 13: note = Xaa is sarcosine (Sar)
  MOD_RES 13: note = AMIDATION
XCKXKCXXXENXX

SEQ ID NO: 67 - protein, 13 aa, synthetic construct
  REGION 1..13: note = Artificial Peptide
  MOD_RES 1: note = Xaa is 5-aminopentanoic acid (Apt)
  REGION 1..10: note = MISC_FEATURE - lactam bridge between Apt at position X1 and Glu at position X10
  DISULFID 2..6: note = disulfide bridge between Cys at position X2 and Cys at position X6
  REGION 2..3: note = MISC_FEATURE - bond between Cys at position X2 and epsilon amino group of adjacent acetylated Lys
  MOD_RES 3: note = ACETYLATION
  MOD_RES 4: note = Xaa is 7-methyl-L-tryptophan (Wim)
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is alpha-methyl-L-lysine substituted with palmitic acid (Mly[Palm])
  MOD_RES 12: note = Xaa is L-3-pyridyl-alanine (Pal)
  MOD_RES 13: note = Xaa is sarcosine (Sar)
  MOD_RES 13: note = AMIDATION
XCKXKCXXXENXX

SEQ ID NO: 68 - protein, 13 aa, synthetic construct
  REGION 1..13: note = Artificial Peptide
  MOD_RES 1: note = Xaa is desamino-aspartamic acid (Dad)
  REGION 1..10: note = MISC_FEATURE - lactam bridge between Dad at position X1 and Kme at position X10
  DISULFID 2..6: note = disulfide bridge between Cys at position X2 and Cys at position X6
  REGION 2..3: note = MISC_FEATURE - bond between Cys at position X2 and epsilon amino group of adjacent acetylated Lys
  MOD_RES 3: note = ACETYLATION
  MOD_RES 4: note = Xaa is 7-methyl-L-tryptophan (Wim)
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is alpha-methyl-L-leucine (Mle)
  MOD_RES 10: note = Xaa is (2S)-2-amino-6-(methylamino)hexanoic acid (epsilon-N-methyl-lysine) (Kme)
  MOD_RES 12: note = Xaa is L-3-pyridyl-alanine (Pal)
  MOD_RES 13: note = Xaa is sarcosine (Sar)
  MOD_RES 13: note = AMIDATION
XCKXLCXXXXNXX

SEQ ID NO: 69 - protein, 13 aa, synthetic construct
  REGION 1..13: note = Artificial Peptide
  MOD_RES 1: note = Xaa is desamino-aspartamic acid (Dad)
  REGION 1..10: note = MISC_FEATURE - lactam bridge between Dad at position X1 and Kme at position X10
  DISULFID 2..6: note = disulfide bridge between Nmc at position X2 and Cys at position X6
  MOD_RES 2: note = Xaa is N-methyl-L-cysteine (Nmc)
  REGION 2..3: note = MISC_FEATURE - bond between Nmc at position X2 and epsilon amino group of adjacent acetylated Lys
  MOD_RES 4: note = Xaa is 7-methyl-L-tryptophan (Wim)
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is alpha-methyl-L-leucine (Mle)
  MOD_RES 10: note = Xaa is (2S)-2-amino-6-(methylamino)hexanoic acid (epsilon-N-methyl-lysine) (Kme)
  MOD_RES 12: note = Xaa is L-3-pyridyl-alanine (Pal)
  MOD_RES 13: note = Xaa is sarcosine (Sar)
  MOD_RES 13: note = AMIDATION
XXKXLCXXXXNXX

SEQ ID NO: 70 - protein, 13 aa, synthetic construct
  REGION 1..13: note = Artificial Peptide
  MOD_RES 1: note = Xaa is desamino-aspartamic acid (Dad)
  REGION 1..10: note = MISC_FEATURE - lactam bridge between Dad at position X1 and Kme at position X10
  DISULFID 2..6: note = disulfide bridge between Nmc at position X2 and Cys at position X6
  MOD_RES 2: note = Xaa is N-methyl-L-cysteine (Nmc)
  REGION 2..3: note = MISC_FEATURE - bond between Nmc at position X2 and epsilon amino group of adjacent acetylated Lys
  MOD_RES 3: note = ACETYLATION
  MOD_RES 4: note = Xaa is 7-methyl-L-tryptophan (Wim)
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is alpha-methyl-L-leucine (Mle)
  MOD_RES 10: note = Xaa is (2S)-2-amino-6-(methylamino)hexanoic acid (epsilon-N-methyl-lysine) (Kme)
  MOD_RES 12: note = Xaa is L-3-pyridyl-alanine (Pal)
  MOD_RES 13: note = Xaa is sarcosine (Sar)
  MOD_RES 13: note = AMIDATION
XXKXLCXXXXAXX

SEQ ID NO: 71 - protein, 14 aa, synthetic construct
  REGION 1..14: note = Artificial Peptide
  MOD_RES 1: note = Xaa is desamino-aspartamic acid (Dad)
  REGION 1..11: note = MISC_FEATURE - lactam bridge between Dad at position X1 and Lys at position X11
  DISULFID 2..7: note = disulfide bridge between Cys at position X2 and Cys at position X7
  MOD_RES 5: note = Xaa is 7-methyl-L-tryptophan (Wim)
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is 2-aminoisobutyric acid (Aib)
  MOD_RES 13: note = Xaa is L-3-pyridyl-alanine (Pal)
  MOD_RES 14: note = AMIDATION
  MOD_RES 14: note = Lys is modified at epsilon-NH2 with 18-oxo-18-[[(1S )-1-carboxy-4-[2-[2-[2-[2-[2-(carboxymethoxy)ethox y]ethylamino]-2-oxo-ethoxy]ethoxy]ethylamino]-4-oxo-butyl] amino]o ctadecanoic acid (Sema)
XCNTXQCXXXKNXK

SEQ ID NO: 72 - protein, 13 aa, synthetic construct
  REGION 1..13: note = Artificial Peptide
  MOD_RES 1: note = Xaa is 5-aminopentanoic acid (Apt)
  REGION 1..10: note = MISC_FEATURE - lactam bridge between Apt at position X1 and Glu at position X10
  DISULFID 2..6: note = disulfide bridge between Cys at position X2 and Cys at position X6
  REGION 2..3: note = MISC_FEATURE - bond between Cys at position X2 and epsilon amino group of adjacent acetylated Lys
  MOD_RES 3: note = ACETYLATION
  MOD_RES 4: note = Xaa is 7-methyl-L-tryptophan (Wim)
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is 2-aminoisobutyric acid (Aib)
  MOD_RES 12: note = Xaa is L-3-pyridyl-alanine (Pal)
  MOD_RES 13: note = Xaa is sarcosine (Sar)
  MOD_RES 13: note = AMIDATION
XCKXKCXXXENXX

SEQ ID NO: 73 - protein, 13 aa, synthetic construct
  REGION 1..13: note = Artificial Peptide
  MOD_RES 1: note = Xaa is 5-aminopentanoic acid (Apt)
  REGION 1..10: note = MISC_FEATURE - lactam bridge between Apt at position X1 and Glu at position X10
  DISULFID 2..6: note = disulfide bridge between Cys at position X2 and Cys at position X6
  REGION 2..3: note = MISC_FEATURE - bond between Cys at position X2 and epsilon amino group of adjacent acetylated Lys
  MOD_RES 3: note = ACETYLATION
  MOD_RES 4: note = Xaa is 7-methyl-L-tryptophan (Wim)
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is alpha-methyl-L-leucine (Mle)
  MOD_RES 12: note = Xaa is L-3-pyridyl-alanine (Pal)
  MOD_RES 13: note = Xaa is sarcosine (Sar)
  MOD_RES 13: note = AMIDATION
XCKXKCXXXENXX

SEQ ID NO: 74 - protein, 13 aa, synthetic construct
  REGION 1..13: note = Artificial Peptide
  MOD_RES 1: note = Xaa is desamino-aspartamic acid (Dad)
  REGION 1..10: note = MISC_FEATURE - lactam bridge between Dad at position X1 and Kme at position X10
  MOD_RES 2: note = Xaa is N-methyl-L-cysteine (Nmc)
  DISULFID 2..6: note = disulfide bridge between Nmc at position X2 and Cys at position X6
  REGION 2..3: note = MISC_FEATURE - bond between Nmc at position X2 and epsilon amino group of adjacent acetylated Lys
  MOD_RES 3: note = ACETYLATION
  MOD_RES 4: note = Xaa is 7-methyl-L-tryptophan (Wim)
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is 4-amino-tetrahydropyran-4-carboxylic acid (Thp)
  MOD_RES 10: note = Xaa is (2S)-2-amino-6-(methylamino)hexanoic acid (epsilon-N-methyl-lysine) (Kme)
  MOD_RES 12: note = Xaa is L-3-pyridyl-alanine (Pal)
  MOD_RES 13: note = Xaa is sarcosine (Sar)
  MOD_RES 13: note = AMIDATION
XXKXKCXXXXNXX

SEQ ID NO: 75 - protein, 13 aa, synthetic construct
  REGION 1..13: note = Artificial Peptide
  MOD_RES 1: note = Xaa is 6-aminohexanoic acid (Ahx)
  REGION 1..11: note = MISC_FEATURE - lactam bridge between Ahx at position X1 and Asp at position X11
  DISULFID 2..7: note = disulfide bridge between Cys at position X2 and Cys at position X7
  MOD_RES 8: note = Xaa is 4-aminoethoxy-L-phenylalanine (Yae)
  MOD_RES 9: note = Xaa is 2-naphthyl-L-alanine (Nal)
  MOD_RES 10: note = Xaa is 2-aminoisobutyric acid (Aib)
  MOD_RES 13: note = Lys is modified at epsilon-NH2 with 18-oxo-18-[[(1S )-1-carboxy-4-[2-[2-[2-[2-[2-(carboxymethoxy)ethox y]ethylamino]-2-oxo-ethoxy]ethoxy]ethylamino]-4-oxo-butyl] amino]o ctadecanoic acid (Sema)
  MOD_RES 13: note = AMIDATION
XCNTWQCXXXDNK

SEQ ID NO: 76 - protein, 13 aa, synthetic construct
  REGION 1..13: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  MOD_RES 2: note = Xaa is L-2-aminobutyric acid (Abu)
  REGION 2..7: note = MISC_FEATURE - bridge between Abu at position X2 and Cys at position X7
  MOD_RES 5: note = Xaa is 7-methyl-L-tryptophan (Wim)
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is 4-amino-tetrahydropyran-4-carboxylic acid (Thp)
  MOD_RES 13: note = Xaa is L-3-pyridyl-alanine (Pal)
  MOD_RES 13: note = AMIDATION
XXNTXKCXXXENX

SEQ ID NO: 77 - protein, 14 aa, synthetic construct
  REGION 1..14: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  MOD_RES 2: note = Xaa is L-2-aminobutyric acid (Abu)
  REGION 2..7: note = MISC_FEATURE - bridge between Abu at position X2 and Cys at position X7
  MOD_RES 5: note = Xaa is 7-methyl-L-tryptophan (Wim)
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is 4-amino-tetrahydropyran-4-carboxylic acid (Thp)
  MOD_RES 13: note = Xaa is L-3-pyridyl-alanine (Pal)
  MOD_RES 14: note = Xaa is sarcosine (Sar)
  MOD_RES 14: note = AMIDATION
XXNTXKCXXXENXX

SEQ ID NO: 78 - protein, 14 aa, synthetic construct
  REGION 1..14: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  MOD_RES 2: note = Xaa is L-2-aminobutyric acid (Abu)
  REGION 2..7: note = MISC_FEATURE - bridge between Abu at position X2 and Cys at position X7
  MOD_RES 5: note = Xaa is 1-methyl-L-tryptophan (Wme)
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is 4-amino-tetrahydropyran-4-carboxylic acid (Thp)
  MOD_RES 13: note = Xaa is L-3-pyridyl-alanine (Pal)
  MOD_RES 14: note = Xaa is sarcosine (Sar)
  MOD_RES 14: note = AMIDATION
XXNTXLCXXXENXX

SEQ ID NO: 79 - protein, 14 aa, synthetic construct
  REGION 1..14: note = Artificial Peptide
  MOD_RES 1: note = Xaa is 5-(methylamino)pentanoic acid (Nmapt)
  REGION 1..11: note = MISC_FEATURE - lactam bridge between Nmapt at position X1 and Glu at position X11
  MOD_RES 2: note = Xaa is L-2-aminobutyric acid (Abu)
  REGION 2..7: note = MISC_FEATURE - bridge between Abu at position X2 and Cys at position X7
  MOD_RES 5: note = Xaa is 1-methyl-L-tryptophan (Wme)
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is 4-amino-tetrahydropyran-4-carboxylic acid (Thp)
  MOD_RES 13: note = Xaa is L-3-pyridyl-alanine (Pal)
  MOD_RES 14: note = Xaa is sarcosine (Sar)
  MOD_RES 14: note = AMIDATION
XXNTXLCXXXENXX

SEQ ID NO: 80 - protein, 14 aa, synthetic construct
  REGION 1..14: note = Artificial Peptide
  MOD_RES 1: note = Xaa is 6-(methylamino)hexanoic acid (Nmha)
  REGION 1..11: note = MISC_FEATURE - lactam bridge between Nmha at position X1 and Glu at position X11
  MOD_RES 2: note = Xaa is L-2-aminobutyric acid (Abu)
  REGION 2..7: note = MISC_FEATURE - bridge between Abu at position X2 and Cys at position X7
  MOD_RES 5: note = Xaa is 1-methyl-L-tryptophan (Wme)
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is 4-amino-tetrahydropyran-4-carboxylic acid (Thp)
  MOD_RES 13: note = Xaa is L-3-pyridyl-alanine (Pal)
  MOD_RES 14: note = Xaa is sarcosine (Sar)
  MOD_RES 14: note = AMIDATION
XXNTXLCXXXENXX

SEQ ID NO: 81 - protein, 14 aa, synthetic construct
  REGION 1..14: note = Artificial Peptide
  MOD_RES 1: note = Xaa is 6-(methylamino)hexanoic acid (Nmha)
  REGION 1..11: note = MISC_FEATURE - lactam bridge between Nmha at position X1 and Asp at position X11
  MOD_RES 2: note = Xaa is L-2-aminobutyric acid (Abu)
  REGION 2..7: note = MISC_FEATURE - bridge between Abu at position X2 and Cys at position X7
  MOD_RES 5: note = Xaa is 1-methyl-L-tryptophan (Wme)
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is 4-amino-tetrahydropyran-4-carboxylic acid (Thp)
  MOD_RES 13: note = Xaa is L-3-pyridyl-alanine (Pal)
  MOD_RES 14: note = Xaa is sarcosine (Sar)
  MOD_RES 14: note = AMIDATION
XXNTXLCXXXDNXX

SEQ ID NO: 82 - protein, 14 aa, synthetic construct
  REGION 1..14: note = Artificial Peptide
  MOD_RES 1: note = Xaa is 5-(methylamino)pentanoic acid (Nmapt)
  REGION 1..11: note = MISC_FEATURE - lactam bridge between Nmapt at position X1 and Asp at position X11
  MOD_RES 2: note = Xaa is L-2-aminobutyric acid (Abu)
  REGION 2..7: note = MISC_FEATURE - bridge between Abu at position X2 and Cys at position X7
  MOD_RES 5: note = Xaa is 1-methyl-L-tryptophan (Wme)
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is 4-amino-tetrahydropyran-4-carboxylic acid (Thp)
  MOD_RES 13: note = Xaa is L-3-pyridyl-alanine (Pal)
  MOD_RES 14: note = Xaa is sarcosine (Sar)
  MOD_RES 14: note = AMIDATION
XXNTXLCXXXDNXX

SEQ ID NO: 83 - protein, 12 aa, synthetic construct
  REGION 1..12: note = Artificial Peptide
  MOD_RES 1: note = Xaa is 5-aminopentanoic acid (Apt)
  REGION 1..10: note = MISC_FEATURE - lactam bridge between Apt at position X1 and Glu at position X10
  DISULFID 2..6: note = disulfide bridge between Cys at position X2 and Cys at position X6
  MOD_RES 3: note = Xaa is 5-aminopentanoic acid (Apt)
  MOD_RES 4: note = Xaa is 1-methyl-L-tryptophan (Wme)
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is alpha-methyl-L-leucine (Mle)
  MOD_RES 12: note = Lys is modified at epsilon-NH2 with 18-oxo-18-[[(1S )-1-carboxy-4-[2-[2-[2-[2-[2-(carboxymethoxy)ethox y]ethylamino]-2-oxo-ethoxy]ethoxy]ethylamino]-4-oxo-butyl] amino]o ctadecanoic acid (Sema)
  MOD_RES 12: note = AMIDATION
XCXXLCXXXENK

SEQ ID NO: 84 - protein, 13 aa, synthetic construct
  REGION 1..13: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 5: note = Xaa is 7-methyl-L-tryptophan (Wim)
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is (2S)-2-amino-6-(dimethylamino)-2-methyl-hexanoic acid (Mkdm)
  MOD_RES 13: note = Xaa is L-5-pyrimidine-alanine (Pyal)
  MOD_RES 13: note = AMIDATION
XCNTXKCXXXENX

SEQ ID NO: 85 - protein, 13 aa, synthetic construct
  REGION 1..13: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 5: note = Xaa is 7-methyl-L-tryptophan (Wim)
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is (2S)-2-amino-6-(dimethylamino)-2-methyl-hexanoic acid (Mkdm)
  MOD_RES 13: note = Xaa is L-3-pyridyl-alanine (Pal)
  MOD_RES 13: note = AMIDATION
XCNTXKCXXXENX

SEQ ID NO: 86 - protein, 14 aa, synthetic construct
  REGION 1..14: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 5: note = Xaa is 7-methyl-L-tryptophan (Wim)
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is (2S)-2-amino-6-(dimethylamino)-2-methyl-hexanoic acid (Mkdm)
  MOD_RES 13: note = Xaa is L-3-pyridyl-alanine (Pal)
  MOD_RES 14: note = Xaa is sarcosine (Sar)
  MOD_RES 14: note = AMIDATION
XCNTXKCXXXENXX

SEQ ID NO: 87 - protein, 14 aa, synthetic construct
  REGION 1..14: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 5: note = Xaa is 7-methyl-L-tryptophan (Wim)
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is 2-aminoisobutyric acid (Aib)
  MOD_RES 13: note = Xaa is L-3-pyridyl-alanine (Pal)
  MOD_RES 14: note = AMIDATION
XCNTXKCXXXENXI

SEQ ID NO: 88 - protein, 12 aa, synthetic construct
  REGION 1..12: note = Artificial Peptide
  MOD_RES 1: note = Xaa is 5-aminopentanoic acid (Apt)
  REGION 1..10: note = MISC_FEATURE - lactam bridge between Apt at position X1 and Glu at position X10
  DISULFID 2..6: note = disulfide bridge between Cys at position X2 and Cys at position X6
  REGION 2..3: note = MISC_FEATURE - bond between Cys at position X2 and epsilon amino group of adjacent acetylated Lys
  MOD_RES 3: note = ACETYLATION
  MOD_RES 4: note = Xaa is 7-methyl-L-tryptophan (Wim)
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is (2S)-2-amino-6-(dimethylamino)-2-methyl-hexanoic acid (Mkdm)
  MOD_RES 12: note = Xaa is L-3-pyridyl-alanine (Pal)
  MOD_RES 12: note = AMIDATION
XCKXKCXXXENX

SEQ ID NO: 89 - protein, 12 aa, synthetic construct
  REGION 1..12: note = Artificial Peptide
  MOD_RES 1: note = Xaa is 5-aminopentanoic acid (Apt)
  REGION 1..10: note = MISC_FEATURE - lactam bridge between Apt at position X1 and Glu at position X10
  REGION 2..3: note = MISC_FEATURE - bond between Cys at position X2 and epsilon amino group of adjacent acetylated Lys
  DISULFID 2..6: note = disulfide bridge between Cys at position X2 and Cys at position X6
  MOD_RES 3: note = ACETYLATION
  MOD_RES 4: note = Xaa is 7-methyl-L-tryptophan (Wim)
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is (2S)-2-amino-6-(dimethylamino)-2-methyl-hexanoic acid (Mkdm)
  MOD_RES 12: note = Xaa is L-3-pyridyl-alanine (Pal)
  MOD_RES 12: note = AMIDATION
XCKXQCXXXENX

SEQ ID NO: 90 - protein, 14 aa, synthetic construct
  REGION 1..14: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is 2-aminoisobutyric acid (Aib)
  MOD_RES 13: note = Xaa is L-3-pyridyl-alanine (Pal)
  MOD_RES 14: note = AMIDATION
XCNTWKCXXXENXI

SEQ ID NO: 91 - protein, 12 aa, synthetic construct
  REGION 1..12: note = Artificial Peptide
  MOD_RES 1: note = Xaa is 5-aminopentanoic acid (Apt)
  REGION 1..10: note = MISC_FEATURE - lactam bridge between Apt at position X1 and Glu at position X10
  REGION 2..3: note = MISC_FEATURE - bond between Cys at position X2 and epsilon amino group of adjacent acetylated Lys
  DISULFID 2..6: note = disulfide bridge between Cys at position X2 and Cys at position X6
  MOD_RES 3: note = ACETYLATION
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is (2S)-2-amino-6-(dimethylamino)-2-methyl-hexanoic acid (Mkdm)
  MOD_RES 12: note = Xaa is L-3-pyridyl-alanine (Pal)
  MOD_RES 12: note = AMIDATION
XCKWKCXXXENX

SEQ ID NO: 92 - protein, 12 aa, synthetic construct
  REGION 1..12: note = Artificial Peptide
  MOD_RES 1: note = Xaa is 5-aminopentanoic acid (Apt)
  REGION 1..10: note = MISC_FEATURE - lactam bridge between Apt at position X1 and Glu at position X10
  REGION 2..3: note = MISC_FEATURE - bond between Cys at position X2 and epsilon amino group of adjacent acetylated Lys
  DISULFID 2..6: note = disulfide bridge between Cys at position X2 and Cys at position X6
  MOD_RES 3: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is (2S)-2-amino-6-(dimethylamino)-2-methyl-hexanoic acid (Mkdm)
  MOD_RES 12: note = Xaa is L-3-pyridyl-alanine (Pal)
  MOD_RES 12: note = AMIDATION
XCKWQCXXXENX

SEQ ID NO: 93 - protein, 12 aa, synthetic construct
  REGION 1..12: note = Artificial Peptide
  MOD_RES 1: note = Xaa is desamino-aspartamic acid (Dad)
  REGION 1..10: note = MISC_FEATURE - lactam bridge between Dad at position X1 and Lys at position X10
  REGION 2..3: note = MISC_FEATURE - bond between Cys at position X2 and epsilon amino group of adjacent acetylated Lys
  DISULFID 2..6: note = disulfide bridge between Cys at position X2 and Cys at position X6
  MOD_RES 3: note = ACETYLATION
  MOD_RES 4: note = Xaa is 7-methyl-L-tryptophan (Wim)
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is 2-aminoisobutyric acid (Aib)
  MOD_RES 12: note = Xaa is L-3-pyridyl-alanine (Pal)
  MOD_RES 12: note = AMIDATION
XCKXQCXXXKNX

SEQ ID NO: 94 - protein, 14 aa, synthetic construct
  REGION 1..14: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 5: note = Xaa is 7-methyl-L-tryptophan (Wim)
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is alpha-methyl-L-lysine (Mly)
  MOD_RES 13: note = Xaa is L-3-pyridyl-alanine (Pal)
  MOD_RES 14: note = Xaa is sarcosine (Sar)
  MOD_RES 14: note = AMIDATION
XCNTXQCXXXENXX

SEQ ID NO: 95 - protein, 14 aa, synthetic construct
  REGION 1..14: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 5: note = Xaa is 7-methyl-L-tryptophan (Wim)
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is alpha-methyl-L-lysine (Mly)
  MOD_RES 13: note = Xaa is L-3-pyridyl-alanine (Pal)
  MOD_RES 14: note = Xaa is sarcosine (Sar)
  MOD_RES 14: note = AMIDATION
XCNTXKCXXXENXX

SEQ ID NO: 96 - protein, 14 aa, synthetic construct
  REGION 1..14: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 5: note = Xaa is 7-methyl-L-tryptophan (Wim)
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is alpha-methyl-L-lysine (Mly)
  MOD_RES 13: note = Xaa is L-3-pyridyl-alanine (Pal)
  MOD_RES 14: note = Lys is modified at epsilon-NH2 with 18-oxo-18-[[(1S )-1-carboxy-4-[2-[2-[2-[2-[2-(carboxymethoxy)ethox y]ethylamino]-2-oxo-ethoxy]ethoxy]ethylamino]-4-oxo-butyl] amino]o ctadecanoic acid (Sema)
  MOD_RES 14: note = AMIDATION
XCNTXKCXXXENXK

SEQ ID NO: 97 - protein, 15 aa, synthetic construct
  REGION 1..15: note = Artificial Peptide
  MOD_RES 1: note = Xaa is 5-aminopentanoic acid (Apt)
  REGION 1..11: note = MISC_FEATURE - lactam bridge between Apt at position X1 and Glu at position X11
  DISULFID 2..7: note = disulfide bridge between Cys at position X2 and Cys at position X7
  MOD_RES 5: note = Xaa is 7-methyl-L-tryptophan (Wim)
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is alpha-methyl-L-lysine (Mly)
  MOD_RES 13: note = Xaa is L-3-pyridyl-alanine (Pal)
  MOD_RES 14: note = Xaa is sarcosine (Sar)
  MOD_RES 15: note = Lys is modified at epsilon-NH2 with 18-oxo-18-[[(1S )-1-carboxy-4-[2-[2-[2-[2-[2-(carboxymethoxy)ethox y]ethylamino]-2-oxo-ethoxy]ethoxy]ethylamino]-4-oxo-butyl] amino]o ctadecanoic acid (Sema)
  MOD_RES 15: note = AMIDATION
XCNTXKCXXXENXXK

SEQ ID NO: 98 - protein, 14 aa, synthetic construct
  REGION 1..14: note = Artificial Peptide
  MOD_RES 1: note = Xaa is desamino-aspartamic acid (Dad)
  REGION 1..11: note = MISC_FEATURE - lactam bridge between Dad at position X1 and Kme at position X11
  DISULFID 2..7: note = disulfide bridge between Nmc at position X2 and Cys at position X7
  MOD_RES 2: note = Xaa is N-methyl-L-cysteine (Nmc)
  MOD_RES 5: note = Xaa is 7-methyl-L-tryptophan (Wim)
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is (2S)-2-amino-6-(dimethylamino)-2-methyl-hexanoic acid (Mkdm)
  MOD_RES 11: note = Xaa is (2S)-2-amino-6-(methylamino)hexanoic acid (epsilon-N-methyl-lysine) (Kme)
  MOD_RES 13: note = Xaa is L-3-pyridyl-alanine (Pal)
  MOD_RES 14: note = Xaa is sarcosine (Sar)
  MOD_RES 14: note = AMIDATION
XXNTXKCXXXXNXX

SEQ ID NO: 99 - protein, 14 aa, synthetic construct
  REGION 1..14: note = Artificial Peptide
  MOD_RES 1: note = Xaa is desamino-aspartamic acid (Dad)
  REGION 1..11: note = MISC_FEATURE - lactam bridge between Dad at position X1 and Lys at position X11
  DISULFID 2..7: note = disulfide bridge between Cys at position X2 and Cys at position X7
  MOD_RES 5: note = Xaa is 7-methyl-L-tryptophan (Wim)
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is alpha-methyl-L-lysine substituted with myristic acid (Mly[Myri])
  MOD_RES 13: note = Xaa is L-3-pyridyl-alanine (Pal)
  MOD_RES 14: note = Xaa is sarcosine (Sar)
  MOD_RES 14: note = AMIDATION
XCNTXKCXXXKNXX

SEQ ID NO: 100 - protein, 13 aa, synthetic construct
  REGION 1..13: note = Artificial Peptide
  MOD_RES 1: note = Xaa is 5-aminopentanoic acid (Apt)
  REGION 1..10: note = MISC_FEATURE - lactam bridge between Apt at position X1 and Glu at position X10
  REGION 2..3: note = MISC_FEATURE - bond between Cys at position X2 and epsilon amino group of adjacent acetylated Lys
  DISULFID 2..6: note = disulfide bridge between Cys at position X2 and Cys at position X6
  MOD_RES 3: note = ACETYLATION
  MOD_RES 4: note = Xaa is 7-methyl-L-tryptophan (Wim)
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is alpha-methyl-L-lysine substituted with myristic acid (Mly[Myri])
  MOD_RES 12: note = Xaa is L-3-pyridyl-alanine (Pal)
  MOD_RES 13: note = Xaa is sarcosine (Sar)
  MOD_RES 13: note = AMIDATION
XCKXKCXXXENXX

SEQ ID NO: 101 - protein, 13 aa, synthetic construct
  REGION 1..13: note = Artificial Peptide
  MOD_RES 1: note = Xaa is desamino-aspartamic acid (Dad)
  REGION 1..10: note = MISC_FEATURE - lactam bridge between Dad at position X1 and Lys at position X10
  REGION 2..3: note = MISC_FEATURE - bond between Cys at position X2 and epsilon amino group of adjacent acetylated Lys
  DISULFID 2..6: note = disulfide bridge between Cys at position X2 and Cys at position X6
  MOD_RES 3: note = ACETYLATION
  MOD_RES 4: note = Xaa is 7-methyl-L-tryptophan (Wim)
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is alpha-methyl-L-lysine substituted with palmitic acid (Mly[Palm])
  MOD_RES 12: note = Xaa is L-3-pyridyl-alanine (Pal)
  MOD_RES 13: note = Xaa is sarcosine (Sar)
  MOD_RES 13: note = AMIDATION
XCKXKCXXXKNXX

SEQ ID NO: 102 - protein, 14 aa, synthetic construct
  REGION 1..14: note = Artificial Peptide
  MOD_RES 1: note = Xaa is desamino-aspartamic acid (Dad)
  REGION 1..11: note = MISC_FEATURE - lactam bridge between Dad at position X1 and Lys at position X11
  DISULFID 2..7: note = disulfide bridge between Cys at position X2 and Cys at position X7
  MOD_RES 5: note = Xaa is 7-methyl-L-tryptophan (Wim)
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is 4-amino-tetrahydropyran-4-carboxylic acid (Thp)
  MOD_RES 13: note = Xaa is L-3-pyridyl-alanine (Pal)
  MOD_RES 14: note = Xaa is sarcosine (Sar)
  MOD_RES 14: note = AMIDATION
XCNTXKCXXXKNXX

SEQ ID NO: 103 - protein, 14 aa, synthetic construct
  REGION 1..14: note = Artificial Peptide
  MOD_RES 1: note = Xaa is desamino-aspartamic acid (Dad)
  REGION 1..11: note = MISC_FEATURE - lactam bridge between Dad at position X1 and Lys at position X11
  DISULFID 2..7: note = disulfide bridge between Cys at position X2 and Cys at position X7
  MOD_RES 5: note = Xaa is 7-methyl-L-tryptophan (Wim)
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is alpha-methyl-L-leucine (Mle)
  MOD_RES 13: note = Xaa is L-3-pyridyl-alanine (Pal)
  MOD_RES 14: note = Xaa is sarcosine (Sar)
  MOD_RES 14: note = AMIDATION
XCNTXKCXXXKNXX

SEQ ID NO: 104 - protein, 13 aa, synthetic construct
  REGION 1..13: note = Artificial Peptide
  MOD_RES 1: note = Xaa is desamino-aspartamic acid (Dad)
  REGION 1..10: note = MISC_FEATURE - lactam bridge between Dad at position X1 and Lys at position X10
  REGION 2..3: note = MISC_FEATURE - bond between Cys at position X2 and epsilon amino group of adjacent acetylated Lys
  DISULFID 2..6: note = disulfide bridge between Cys at position X2 and Cys at position X6
  MOD_RES 3: note = ACETYLATION
  MOD_RES 4: note = Xaa is 7-methyl-L-tryptophan (Wim)
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is 2-aminoisobutyric acid (Aib)
  MOD_RES 12: note = Xaa is L-3-pyridyl-alanine (Pal)
  MOD_RES 13: note = Xaa is sarcosine (Sar)
  MOD_RES 13: note = AMIDATION
XCKXKCXXXKNXX

SEQ ID NO: 105 - protein, 13 aa, synthetic construct
  REGION 1..13: note = Artificial Peptide
  MOD_RES 1: note = Xaa is desamino-aspartamic acid (Dad)
  REGION 1..10: note = MISC_FEATURE - lactam bridge between Dad at position X1 and Lys at position X10
  REGION 2..3: note = MISC_FEATURE - bond between Cys at position X2 and epsilon amino group of adjacent acetylated Lys
  DISULFID 2..6: note = disulfide bridge between Cys at position X2 and Cys at position X6
  MOD_RES 3: note = ACETYLATION
  MOD_RES 4: note = Xaa is 7-methyl-L-tryptophan (Wim)
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is 4-amino-tetrahydropyran-4-carboxylic acid (Thp)
  MOD_RES 12: note = Xaa is L-3-pyridyl-alanine (Pal)
  MOD_RES 13: note = Xaa is sarcosine (Sar)
  MOD_RES 13: note = AMIDATION
XCKXKCXXXKNXX

SEQ ID NO: 106 - protein, 13 aa, synthetic construct
  REGION 1..13: note = Artificial Peptide
  MOD_RES 1: note = Xaa is desamino-aspartamic acid (Dad)
  REGION 1..10: note = MISC_FEATURE - lactam bridge between Dad at position X1 and Lys at position X10
  REGION 2..3: note = MISC_FEATURE - bond between Cys at position X2 and epsilon amino group of adjacent acetylated Lys
  DISULFID 2..6: note = disulfide bridge between Cys at position X2 and Cys at position X6
  MOD_RES 3: note = ACETYLATION
  MOD_RES 4: note = Xaa is 7-methyl-L-tryptophan (Wim)
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is alpha-methyl-L-leucine (Mle)
  MOD_RES 12: note = Xaa is L-3-pyridyl-alanine (Pal)
  MOD_RES 13: note = Xaa is sarcosine (Sar)
  MOD_RES 13: note = AMIDATION
XCKXKCXXXKNXX

SEQ ID NO: 107 - protein, 15 aa, synthetic construct
  REGION 1..15: note = Artificial Peptide
  MOD_RES 1: note = Xaa is desamino-aspartamic acid (Dad)
  REGION 1..11: note = MISC_FEATURE - lactam bridge between Dad at position X1 and Lys at position X11
  DISULFID 2..7: note = disulfide bridge between Cys at position X2 and Cys at position X7
  MOD_RES 5: note = Xaa is 7-methyl-L-tryptophan (Wim)
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is 4-amino-tetrahydropyran-4-carboxylic acid (Thp)
  MOD_RES 13: note = Xaa is L-3-pyridyl-alanine (Pal)
  MOD_RES 14: note = Xaa is sarcosine (Sar)
  MOD_RES 15: note = Lys is modified at epsilon-NH2 with 18-oxo-18-[[(1S )-1-carboxy-4-[2-[2-[2-[2-[2-(carboxymethoxy)ethox y]ethylamino]-2-oxo-ethoxy]ethoxy]ethylamino]-4-oxo-butyl] amino]o ctadecanoic acid (Sema)
  MOD_RES 15: note = AMIDATION
XCNTXKCXXXKNXXK

SEQ ID NO: 108 - protein, 13 aa, synthetic construct
  REGION 1..13: note = Artificial Peptide
  MOD_RES 1: note = Xaa is 5-aminopentanoic acid (Apt)
  REGION 1..10: note = MISC_FEATURE - lactam bridge between Apt at position X1 and Glu at position X10
  REGION 2..3: note = MISC_FEATURE - bond between Cys at position X2 and epsilon amino group of adjacent acetylated Lys
  DISULFID 2..6: note = disulfide bridge between Cys at position X2 and Cys at position X6
  MOD_RES 3: note = ACETYLATION
  MOD_RES 4: note = Xaa is 7-methyl-L-tryptophan (Wim)
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is alpha-methyl-L-lysine substituted with myristic acid (Mly[Myri])
  MOD_RES 12: note = Xaa is L-3-pyridyl-alanine (Pal)
  MOD_RES 13: note = Xaa is sarcosine (Sar)
  MOD_RES 13: note = AMIDATION
XCKXKCXXXENXX

SEQ ID NO: 109 - protein, 13 aa, synthetic construct
  REGION 1..13: note = Artificial Peptide
  MOD_RES 1: note = Xaa is desamino-aspartamic acid (Dad)
  REGION 1..10: note = MISC_FEATURE - lactam bridge between Dad at position X1 and Kme at position X10
  MOD_RES 2: note = Xaa is N-methyl-L-cysteine (Nmc)
  DISULFID 2..6: note = disulfide bridge between Nmc at position X2 and Cys at position X6
  REGION 2..3: note = MISC_FEATURE - bond between Nmc at position X2 and epsilon amino group of adjacent acetylated Lys
  MOD_RES 3: note = ACETYLATION
  MOD_RES 4: note = Xaa is 7-methyl-L-tryptophan (Wim)
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is (2S)-2-amino-6-(dimethylamino)-2-methyl-hexanoic acid (Mkdm)
  MOD_RES 10: note = Xaa is (2S)-2-amino-6-(methylamino)hexanoic acid (epsilon-N-methyl-lysine) (Kme)
  MOD_RES 12: note = Xaa is L-3-pyridyl-alanine (Pal)
  MOD_RES 13: note = Xaa is sarcosine (Sar)
  MOD_RES 13: note = AMIDATION
XXKXKCXXXXNXX

SEQ ID NO: 110 - protein, 13 aa, synthetic construct
  REGION 1..13: note = Artificial Peptide
  MOD_RES 1: note = Xaa is desamino-aspartamic acid (Dad)
  REGION 1..10: note = MISC_FEATURE - lactam bridge between Dad at position X1 and Kme at position X10
  REGION 2..3: note = MISC_FEATURE - bond between Cys at position X2 and epsilon amino group of adjacent acetylated Lys
  MOD_RES 3: note = ACETYLATION
  MOD_RES 4: note = Xaa is 7-methyl-L-tryptophan (Wim)
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is 4-amino-tetrahydropyran-4-carboxylic acid (Thp)
  MOD_RES 10: note = Xaa is (2S)-2-amino-6-(methylamino)hexanoic acid (epsilon-N-methyl-lysine) (Kme)
  MOD_RES 12: note = Xaa is L-3-pyridyl-alanine (Pal)
  MOD_RES 13: note = Xaa is sarcosine (Sar)
  MOD_RES 13: note = AMIDATION
XCKXKCXXXXNXX

SEQ ID NO: 111 - protein, 13 aa, synthetic construct
  REGION 1..13: note = Artificial Peptide
  MOD_RES 1: note = Xaa is desamino-aspartamic acid (Dad)
  REGION 1..10: note = MISC_FEATURE - lactam bridge between Dad at position X1 and Lys at position X10
  REGION 2..3: note = MISC_FEATURE - bond between Abu at position X2 and epsilon amino group of adjacent acetylated Lys
  REGION 2..6: note = MISC_FEATURE - bridge between Abu at position X2 and Cys at position X6
  MOD_RES 2: note = Xaa is L-2-aminobutyric acid (Abu)
  MOD_RES 3: note = ACETYLATION
  MOD_RES 4: note = Xaa is 7-methyl-L-tryptophan (Wim)
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is (2S)-2-amino-6-(dimethylamino)-2-methyl-hexanoic acid (Mkdm)
  MOD_RES 12: note = Xaa is L-3-pyridyl-alanine (Pal)
  MOD_RES 13: note = Xaa is sarcosine (Sar)
  MOD_RES 13: note = AMIDATION
XXKXKCXXXKNXX

SEQ ID NO: 112 - protein, 13 aa, synthetic construct
  REGION 1..13: note = Artificial Peptide
  MOD_RES 1: note = Xaa is 5-aminopentanoic acid (Apt)
  REGION 1..10: note = MISC_FEATURE - lactam bridge between Apt at position X1 and Glu at position X10
  MOD_RES 2: note = Xaa is L-2-aminobutyric acid (Abu)
  REGION 2..3: note = MISC_FEATURE - bond between Abu at position X2 and epsilon amino group of adjacent acetylated Lys
  REGION 2..6: note = MISC_FEATURE - bridge between Abu at position X2 and Cys at position X6
  MOD_RES 3: note = ACETYLATION
  MOD_RES 4: note = Xaa is 7-methyl-L-tryptophan (Wim)
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is (2S)-2-amino-6-(dimethylamino)-2-methyl-hexanoic acid (Mkdm)
  MOD_RES 12: note = Xaa is L-3-pyridyl-alanine (Pal)
  MOD_RES 13: note = Xaa is sarcosine (Sar)
  MOD_RES 13: note = AMIDATION
XXKXKCXXXENXX

SEQ ID NO: 113 - protein, 12 aa, synthetic construct
  REGION 1..12: note = Artificial Peptide
  MOD_RES 1: note = Xaa is desamino-aspartamic acid (Dad)
  REGION 1..10: note = MISC_FEATURE - lactam bridge between Dad at position X1 and Lys at position X10
  REGION 2..3: note = MISC_FEATURE - bond between Cys at position X2 and epsilon amino group of adjacent acetylated Lys
  DISULFID 2..6: note = disulfide bridge between Cys at position X2 and Cys at position X6
  MOD_RES 3: note = ACETYLATION
  MOD_RES 4: note = Xaa is 7-methyl-L-tryptophan (Wim)
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is (2S)-2-amino-6-(dimethylamino)-2-methyl-hexanoic acid (Mkdm)
  MOD_RES 12: note = Xaa is 2-(3-pyridyl)ethanamine (PyEA)
XCKXKCXXXKGX

SEQ ID NO: 114 - protein, 13 aa, synthetic construct
  REGION 1..13: note = Artificial Peptide
  MOD_RES 1: note = Xaa is desamino-aspartamic acid (Dad)
  REGION 1..10: note = MISC_FEATURE - lactam bridge between Dad at position X1 and Kme at position X10
  MOD_RES 2: note = Xaa is N-methyl-L-cysteine (Nmc)
  REGION 2..3: note = MISC_FEATURE - bond between Nmc at position X2 and epsilon amino group of adjacent acetylated Lys
  DISULFID 2..6: note = disulfide bridge between Nmc at position X2 and Cys at position X6
  MOD_RES 3: note = ACETYLATION
  MOD_RES 4: note = Xaa is 7-methyl-L-tryptophan (Wim)
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is 4-amino-tetrahydropyran-4-carboxylic acid (Thp)
  MOD_RES 10: note = Xaa is (2S)-2-amino-6-(methylamino)hexanoic acid (epsilon-N-methyl-lysine) (Kme)
  MOD_RES 12: note = Xaa is L-3-pyridyl-alanine (Pal)
  MOD_RES 13: note = Xaa is sarcosine (Sar)
  MOD_RES 13: note = Sar is modified at C-terminus with N(Et)2
XXKXKCXXXXNXX

SEQ ID NO: 115 - protein, 13 aa, synthetic construct
  REGION 1..13: note = Artificial Peptide
  MOD_RES 1: note = Xaa is 5-aminopentanoic acid (Apt)
  REGION 1..10: note = MISC_FEATURE - lactam bridge between Apt at position X1 and Glu at position X10
  MOD_RES 2: note = Xaa is L-2-aminobutyric acid (Abu)
  REGION 2..3: note = MISC_FEATURE - bond between Abu at position X2 and epsilon amino group of adjacent acetylated Lys
  REGION 2..6: note = MISC_FEATURE - bridge between Abu at position X2 and Cys at position X6
  MOD_RES 3: note = ACETYLATION
  MOD_RES 4: note = Xaa is 7-methyl-L-tryptophan (Wim)
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is 4-amino-tetrahydropyran-4-carboxylic acid (Thp)
  MOD_RES 12: note = Xaa is L-3-pyridyl-alanine (Pal)
  MOD_RES 13: note = Xaa is sarcosine (Sar)
  MOD_RES 13: note = AMIDATION
XXKXKCXXXENXX

SEQ ID NO: 116 - protein, 13 aa, synthetic construct
  REGION 1..13: note = Artificial Peptide
  MOD_RES 1: note = Xaa is 5-aminopentanoic acid (Apt)
  REGION 1..10: note = MISC_FEATURE - lactam bridge between Apt at position X1 and Glu at position X10
  MOD_RES 2: note = Xaa is L-2-aminobutyric acid (Abu)
  REGION 2..3: note = MISC_FEATURE - bond between Abu at position X2 and epsilon amino group of adjacent acetylated Lys
  REGION 2..6: note = MISC_FEATURE - bridge between Abu at position X2 and Cys at position X6
  MOD_RES 3: note = ACETYLATION
  MOD_RES 4: note = Xaa is 7-methyl-L-tryptophan (Wim)
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is 2-aminoisobutyric acid (Aib)
  MOD_RES 12: note = Xaa is L-3-pyridyl-alanine (Pal)
  MOD_RES 13: note = Xaa is sarcosine (Sar)
  MOD_RES 13: note = AMIDATION
XXKXKCXXXENXX

SEQ ID NO: 117 - protein, 13 aa, synthetic construct
  REGION 1..13: note = Artificial Peptide
  MOD_RES 1: note = Xaa is 5-aminopentanoic acid (Apt)
  REGION 1..10: note = MISC_FEATURE - lactam bridge between Apt at position X1 and Glu at position X10
  MOD_RES 2: note = Xaa is L-2-aminobutyric acid (Abu)
  REGION 2..3: note = MISC_FEATURE - bond between Abu at position X2 and epsilon amino group of adjacent acetylated Lys
  REGION 2..6: note = MISC_FEATURE - bridge between Abu at position X2 and Cys at position X6
  MOD_RES 3: note = ACETYLATION
  MOD_RES 4: note = Xaa is 7-methyl-L-tryptophan (Wim)
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is alpha-methyl-L-leucine (Mle)
  MOD_RES 12: note = Xaa is L-3-pyridyl-alanine (Pal)
  MOD_RES 13: note = Xaa is sarcosine (Sar)
  MOD_RES 13: note = AMIDATION
XXKXKCXXXENXX

SEQ ID NO: 118 - protein, 14 aa, synthetic construct
  REGION 1..14: note = Artificial Peptide
  MOD_RES 1: note = Xaa is desamino-aspartamic acid (Dad)
  REGION 1..11: note = MISC_FEATURE - lactam bridge between Dad at position X1 and Lys at position X11
  MOD_RES 2: note = Xaa is L-2-aminobutyric acid (Abu)
  REGION 2..7: note = MISC_FEATURE - bridge between Abu at position X2 and Cys at position X7
  MOD_RES 5: note = Xaa is 7-methyl-L-tryptophan (Wim)
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is 2-aminoisobutyric acid (Aib)
  MOD_RES 13: note = Xaa is L-3-pyridyl-alanine (Pal)
  MOD_RES 14: note = Xaa is sarcosine (Sar)
  MOD_RES 14: note = AMIDATION
XXNTXKCXXXKNXX

SEQ ID NO: 119 - protein, 14 aa, synthetic construct
  REGION 1..14: note = Artificial Peptide
  MOD_RES 1: note = Xaa is desamino-aspartamic acid (Dad)
  REGION 1..11: note = MISC_FEATURE - lactam bridge between Dad at position X1 and Lys at position X11
  MOD_RES 2: note = Xaa is L-2-aminobutyric acid (Abu)
  REGION 2..7: note = MISC_FEATURE - bridge between Abu at position X2 and Cys at position X7
  MOD_RES 5: note = Xaa is 7-methyl-L-tryptophan (Wim)
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is 4-amino-tetrahydropyran-4-carboxylic acid (Thp)
  MOD_RES 13: note = Xaa is L-3-pyridyl-alanine (Pal)
  MOD_RES 14: note = Xaa is sarcosine (Sar)
  MOD_RES 14: note = AMIDATION
XXNTXKCXXXKNXX

SEQ ID NO: 120 - protein, 14 aa, synthetic construct
  REGION 1..14: note = Artificial Peptide
  MOD_RES 1: note = Xaa is desamino-aspartamic acid (Dad)
  REGION 1..11: note = MISC_FEATURE - lactam bridge between Dad at position X1 and Lys at position X11
  MOD_RES 2: note = Xaa is L-2-aminobutyric acid (Abu)
  REGION 2..7: note = MISC_FEATURE - bridge between Abu at position X2 and Cys at position X7
  MOD_RES 5: note = Xaa is 7-methyl-L-tryptophan (Wim)
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is alpha-methyl-L-leucine (Mle)
  MOD_RES 13: note = Xaa is L-3-pyridyl-alanine (Pal)
  MOD_RES 14: note = Xaa is sarcosine (Sar)
  MOD_RES 14: note = AMIDATION
XXNTXKCXXXKNXX

SEQ ID NO: 121 - protein, 13 aa, synthetic construct
  REGION 1..13: note = Artificial Peptide
  MOD_RES 1: note = Xaa is desamino-aspartamic acid (Dad)
  REGION 1..10: note = MISC_FEATURE - lactam bridge between Dad at position X1 and Lys at position X10
  MOD_RES 2: note = Xaa is L-2-aminobutyric acid (Abu)
  REGION 2..3: note = MISC_FEATURE - bond between Abu at position X2 and epsilon amino group of adjacent acetylated Lys
  REGION 2..6: note = MISC_FEATURE - bridge between Abu at position X2 and Cys at position X6
  MOD_RES 3: note = ACETYLATION
  MOD_RES 4: note = Xaa is 7-methyl-L-tryptophan (Wim)
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is 2-aminoisobutyric acid (Aib)
  MOD_RES 12: note = Xaa is L-3-pyridyl-alanine (Pal)
  MOD_RES 13: note = Xaa is sarcosine (Sar)
  MOD_RES 13: note = AMIDATION
XXKXKCXXXKNXX

SEQ ID NO: 122 - protein, 13 aa, synthetic construct
  REGION 1..13: note = Artificial Peptide
  MOD_RES 1: note = Xaa is desamino-aspartamic acid (Dad)
  REGION 1..10: note = MISC_FEATURE - lactam bridge between Dad at position X1 and Lys at position X10
  MOD_RES 2: note = Xaa is L-2-aminobutyric acid (Abu)
  REGION 2..3: note = MISC_FEATURE - bond between Abu at position X2 and epsilon amino group of adjacent acetylated Lys
  REGION 2..6: note = MISC_FEATURE - bridge between Abu at position X2 and Cys at position X6
  MOD_RES 3: note = ACETYLATION
  MOD_RES 4: note = Xaa is 7-methyl-L-tryptophan (Wim)
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is 4-amino-tetrahydropyran-4-carboxylic acid (Thp)
  MOD_RES 12: note = Xaa is L-3-pyridyl-alanine (Pal)
  MOD_RES 13: note = Xaa is sarcosine (Sar)
  MOD_RES 13: note = AMIDATION
XXKXKCXXXKNXX

SEQ ID NO: 123 - protein, 13 aa, synthetic construct
  REGION 1..13: note = Artificial Peptide
  MOD_RES 1: note = Xaa is desamino-aspartamic acid (Dad)
  REGION 1..10: note = MISC_FEATURE - lactam bridge between Dad at position X1 and Lys at position X10
  MOD_RES 2: note = Xaa is L-2-aminobutyric acid (Abu)
  REGION 2..3: note = MISC_FEATURE - bond between Abu at position X2 and epsilon amino group of adjacent acetylated Lys
  REGION 2..6: note = MISC_FEATURE - bridge between Abu at position X2 and Cys at position X6
  MOD_RES 3: note = ACETYLATION
  MOD_RES 4: note = Xaa is 7-methyl-L-tryptophan (Wim)
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is alpha-methyl-L-leucine (Mle)
  MOD_RES 12: note = Xaa is L-3-pyridyl-alanine (Pal)
  MOD_RES 13: note = Xaa is sarcosine (Sar)
  MOD_RES 13: note = AMIDATION
XXKXKCXXXKNXX

SEQ ID NO: 124 - protein, 14 aa, synthetic construct
  REGION 1..14: note = Artificial Peptide
  MOD_RES 1: note = Xaa is desamino-aspartamic acid (Dad)
  REGION 1..11: note = MISC_FEATURE - lactam bridge between Dad at position X1 and Kme at position X11
  MOD_RES 2: note = Xaa is L-2-aminobutyric acid (Abu)
  REGION 2..7: note = MISC_FEATURE - bridge between Abu at position X2 and Cys at position X7
  MOD_RES 5: note = Xaa is 7-methyl-L-tryptophan (Wim)
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is 4-amino-tetrahydropyran-4-carboxylic acid (Thp)
  MOD_RES 11: note = Xaa is (2S)-2-amino-6-(methylamino)hexanoic acid (epsilon-N-methyl-lysine) (Kme)
  MOD_RES 13: note = Xaa is L-3-pyridyl-alanine (Pal)
  MOD_RES 14: note = Xaa is sarcosine (Sar)
  MOD_RES 14: note = AMIDATION
XXNTXKCXXXXNXX

SEQ ID NO: 125 - protein, 13 aa, synthetic construct
  REGION 1..13: note = Artificial Peptide
  MOD_RES 1: note = Xaa is desamino-aspartamic acid (Dad)
  REGION 1..10: note = MISC_FEATURE - lactam bridge between Dad at position X1 and Kme at position X10
  MOD_RES 2: note = Xaa is L-2-aminobutyric acid (Abu)
  REGION 2..3: note = MISC_FEATURE - bond between Abu at position X2 and epsilon amino group of adjacent acetylated Lys
  REGION 2..6: note = MISC_FEATURE - bridge between Abu at position X2 and Cys at position X6
  MOD_RES 3: note = ACETYLATION
  MOD_RES 4: note = Xaa is 7-methyl-L-tryptophan (Wim)
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is (2S)-2-amino-6-(dimethylamino)-2-methyl-hexanoic acid (Mkdm)
  MOD_RES 10: note = Xaa is (2S)-2-amino-6-(methylamino)hexanoic acid (epsilon-N-methyl-lysine) (Kme)
  MOD_RES 12: note = Xaa is L-3-pyridyl-alanine (Pal)
  MOD_RES 13: note = Xaa is sarcosine (Sar)
  MOD_RES 13: note = AMIDATION
XXKXKCXXXXNXX

SEQ ID NO: 126 - protein, 13 aa, synthetic construct
  REGION 1..13: note = Artificial Peptide
  MOD_RES 1: note = Xaa is 5-aminopentanoic acid (Apt)
  REGION 1..10: note = MISC_FEATURE - lactam bridge between Apt at position X1 and Glu at position X10
  REGION 2..3: note = MISC_FEATURE - bond between Cys at position X2 and epsilon amino group of adjacent acetylated Lys
  DISULFID 2..6: note = disulfide bridge between Cys at position X2 and Cys at position X6
  MOD_RES 3: note = ACETYLATION
  MOD_RES 4: note = Xaa is 1-methyl-L-tryptophan (Wme)
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is 4-amino-tetrahydropyran-4-carboxylic acid (Thp)
  MOD_RES 12: note = Xaa is L-3-pyridyl-alanine (Pal)
  MOD_RES 13: note = Xaa is sarcosine (Sar)
  MOD_RES 13: note = AMIDATION
XCKXKCXXXENXX

SEQ ID NO: 127 - protein, 12 aa, synthetic construct
  REGION 1..12: note = Artificial Peptide
  MOD_RES 1: note = Xaa is N-methyl-L-cysteine substituted with butyl (Nmc[Butyl])
  REGION 1..9: note = MISC_FEATURE - bridge between Nmc[Butyl] at position X1 and Kme at position X9
  REGION 1..2: note = MISC_FEATURE - bond between Nmc[Butyl] at position X1 and epsilon amino group of adjacent acetylated Lys
  DISULFID 1..5: note = disulfide bridge between Nmc at position X1 and Cys at position X5
  MOD_RES 2: note = ACETYLATION
  MOD_RES 3: note = Xaa is 7-methyl-L-tryptophan (Wim)
  MOD_RES 4: note = ACETYLATION
  MOD_RES 6: note = Xaa is (2S)-2-amino-3-[4-[2-(dimethylamino)ethoxy]p henyl]propanoic acid (Yde)
  MOD_RES 7: note = Xaa is 2-naphthyl-L-alanine (Nal)
  MOD_RES 8: note = Xaa is 4-amino-tetrahydropyran-4-carboxylic acid (Thp)
  MOD_RES 9: note = Xaa is (2S)-2-amino-6-(methylamino)hexanoic acid (epsilon-N-methyl-lysine) (Kme)
  MOD_RES 11: note = Xaa is L-3-pyridyl-alanine (Pal)
  MOD_RES 12: note = Xaa is sarcosine (Sar)
  MOD_RES 12: note = AMIDATION
XKXKCXXXXNXX

SEQ ID NO: 128 - protein, 11 aa, synthetic construct
  REGION 1..11: note = Artificial Peptide
  MOD_RES 1: note = Xaa is N-methyl-L-cysteine substituted with butyl (Nmc[Butyl])
  REGION 1..9: note = MISC_FEATURE - bridge between Nmc[Butyl] at position X1 and Kme at position X9
  REGION 1..2: note = MISC_FEATURE - bond between Nmc[Butyl] at position X1 and epsilon amino group of adjacent acetylated Lys
  DISULFID 1..5: note = disulfide bridge between Nmc at position X1 and Cys at position X5
  MOD_RES 2: note = ACETYLATION
  MOD_RES 3: note = Xaa is 7-methyl-L-tryptophan (Wim)
  MOD_RES 4: note = ACETYLATION
  MOD_RES 6: note = Xaa is (2S)-2-amino-3-[4-[2-(dimethylamino)ethoxy]p henyl]propanoic acid (Yde)
  MOD_RES 7: note = Xaa is 2-naphthyl-L-alanine (Nal)
  MOD_RES 8: note = Xaa is (2S)-2-amino-6-(dimethylamino)-2-methyl-hexanoic acid (Mkdm)
  MOD_RES 9: note = Xaa is (2S)-2-amino-6-(methylamino)hexanoic acid (epsilon-N-methyl-lysine) (Kme)
  MOD_RES 11: note = Xaa is 2-(3-pyridyl)ethanamine (PyEA)
XKXKCXXXXGX

SEQ ID NO: 129 - protein, 13 aa, synthetic construct
  REGION 1..13: note = Artificial Peptide
  MOD_RES 1: note = Xaa is 5-(methylamino)pentanoic acid (Nmapt)
  REGION 1..10: note = MISC_FEATURE - lactam bridge between Nmapt at position X1 and Glu at position X10
  REGION 2..3: note = MISC_FEATURE - bond between Cys at position X2 and epsilon amino group of adjacent acetylated Lys
  DISULFID 2..6: note = disulfide bridge between Cys at position X2 and Cys at position X6
  MOD_RES 3: note = ACETYLATION
  MOD_RES 4: note = Xaa is 7-methyl-L-tryptophan (Wim)
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is (2S)-2-amino-6-(dimethylamino)-2-methyl-hexanoic acid (Mkdm)
  MOD_RES 12: note = Xaa is L-3-pyridyl-alanine (Pal)
  MOD_RES 13: note = Xaa is sarcosine (Sar)
  MOD_RES 13: note = AMIDATION
XCKXKCXXXENXX

SEQ ID NO: 130 - protein, 13 aa, synthetic construct
  REGION 1..13: note = Artificial Peptide
  MOD_RES 1: note = Xaa is desamino-aspartamic acid (Dad)
  REGION 1..10: note = MISC_FEATURE - lactam bridge between Dad at position X1 and Lys at position X10
  MOD_RES 2: note = Xaa is L-2-aminobutyric acid (Abu)
  REGION 2..3: note = MISC_FEATURE - bond between Abu at position X2 and epsilon amino group of adjacent acetylated Lys
  REGION 2..6: note = MISC_FEATURE - bridge between Abu at position X2 and Cys at position X6
  MOD_RES 3: note = ACETYLATION
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is (2S)-2-amino-6-(dimethylamino)-2-methyl-hexanoic acid (Mkdm)
  MOD_RES 12: note = Xaa is L-3-pyridyl-alanine (Pal)
  MOD_RES 13: note = Xaa is sarcosine (Sar)
  MOD_RES 13: note = AMIDATION
XXKWKCXXXKNXX

SEQ ID NO: 131 - protein, 13 aa, synthetic construct
  REGION 1..13: note = Artificial Peptide
  MOD_RES 1: note = Xaa is 5-aminopentanoic acid (Apt)
  REGION 1..10: note = MISC_FEATURE - lactam bridge between Apt at position X1 and Glu at position X10
  MOD_RES 2: note = Xaa is L-2-aminobutyric acid (Abu)
  REGION 2..3: note = MISC_FEATURE - bond between Abu at position X2 and epsilon amino group of adjacent acetylated Lys
  REGION 2..6: note = MISC_FEATURE - bridge between Abu at position X2 and Cys at position X6
  MOD_RES 3: note = ACETYLATION
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is (2S)-2-amino-6-(dimethylamino)-2-methyl-hexanoic acid (Mkdm)
  MOD_RES 12: note = Xaa is L-3-pyridyl-alanine (Pal)
  MOD_RES 13: note = Xaa is sarcosine (Sar)
  MOD_RES 13: note = AMIDATION
XXKWKCXXXENXX

SEQ ID NO: 132 - protein, 13 aa, synthetic construct
  REGION 1..13: note = Artificial Peptide
  MOD_RES 1: note = Xaa is 5-aminopentanoic acid (Apt)
  REGION 1..10: note = MISC_FEATURE - lactam bridge between Apt at position X1 and Glu at position X10
  MOD_RES 2: note = Xaa is L-2-aminobutyric acid (Abu)
  REGION 2..3: note = MISC_FEATURE - bond between Abu at position X2 and epsilon amino group of adjacent acetylated Lys
  REGION 2..6: note = MISC_FEATURE - bridge between Abu at position X2 and Cys at position X6
  MOD_RES 3: note = ACETYLATION
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is 4-amino-tetrahydropyran-4-carboxylic acid (Thp)
  MOD_RES 12: note = Xaa is L-3-pyridyl-alanine (Pal)
  MOD_RES 13: note = Xaa is sarcosine (Sar)
  MOD_RES 13: note = AMIDATION
XXKWKCXXXENXX

SEQ ID NO: 133 - protein, 13 aa, synthetic construct
  REGION 1..13: note = Artificial Peptide
  MOD_RES 1: note = Xaa is 5-aminopentanoic acid (Apt)
  REGION 1..10: note = MISC_FEATURE - lactam bridge between Apt at position X1 and Glu at position X10
  MOD_RES 2: note = Xaa is L-2-aminobutyric acid (Abu)
  REGION 2..3: note = MISC_FEATURE - bond between Abu at position X2 and epsilon amino group of adjacent acetylated Lys
  REGION 2..6: note = MISC_FEATURE - bridge between Abu at position X2 and Cys at position X6
  MOD_RES 3: note = ACETYLATION
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is 2-aminoisobutyric acid (Aib)
  MOD_RES 12: note = Xaa is L-3-pyridyl-alanine (Pal)
  MOD_RES 13: note = Xaa is sarcosine (Sar)
  MOD_RES 13: note = AMIDATION
XXKWKCXXXENXX

SEQ ID NO: 134 - protein, 13 aa, synthetic construct
  REGION 1..13: note = Artificial Peptide
  MOD_RES 1: note = Xaa is 5-aminopentanoic acid (Apt)
  REGION 1..10: note = MISC_FEATURE - lactam bridge between Apt at position X1 and Glu at position X10
  MOD_RES 2: note = Xaa is L-2-aminobutyric acid (Abu)
  REGION 2..3: note = MISC_FEATURE - bond between Abu at position X2 and epsilon amino group of adjacent acetylated Lys
  REGION 2..6: note = MISC_FEATURE - bridge between Abu at position X2 and Cys at position X6
  MOD_RES 3: note = ACETYLATION
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is alpha-methyl-L-leucine (Mle)
  MOD_RES 12: note = Xaa is L-3-pyridyl-alanine (Pal)
  MOD_RES 13: note = Xaa is sarcosine (Sar)
  MOD_RES 13: note = AMIDATION
XXKWKCXXXENXX

SEQ ID NO: 135 - protein, 14 aa, synthetic construct
  REGION 1..14: note = Artificial Peptide
  MOD_RES 1: note = Xaa is desamino-aspartamic acid (Dad)
  REGION 1..11: note = MISC_FEATURE - lactam bridge between Dad at position X1 and Lys at position X11
  MOD_RES 2: note = Xaa is L-2-aminobutyric acid (Abu)
  REGION 2..7: note = MISC_FEATURE - bridge between Abu at position X2 and Cys at position X7
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is 2-aminoisobutyric acid (Aib)
  MOD_RES 13: note = Xaa is L-3-pyridyl-alanine (Pal)
  MOD_RES 14: note = Xaa is sarcosine (Sar)
  MOD_RES 14: note = AMIDATION
XXNTWKCXXXKNXX

SEQ ID NO: 136 - protein, 14 aa, synthetic construct
  REGION 1..14: note = Artificial Peptide
  MOD_RES 1: note = Xaa is desamino-aspartamic acid (Dad)
  REGION 1..11: note = MISC_FEATURE - lactam bridge between Dad at position X1 and Lys at position X11
  MOD_RES 2: note = Xaa is L-2-aminobutyric acid (Abu)
  REGION 2..7: note = MISC_FEATURE - bridge between Abu at position X2 and Cys at position X7
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is 4-amino-tetrahydropyran-4-carboxylic acid (Thp)
  MOD_RES 13: note = Xaa is L-3-pyridyl-alanine (Pal)
  MOD_RES 14: note = Xaa is sarcosine (Sar)
  MOD_RES 14: note = AMIDATION
XXNTWKCXXXKNXX

SEQ ID NO: 137 - protein, 14 aa, synthetic construct
  REGION 1..14: note = Artificial Peptide
  MOD_RES 1: note = Xaa is desamino-aspartamic acid (Dad)
  REGION 1..11: note = MISC_FEATURE - lactam bridge between Dad at position X1 and Lys at position X11
  MOD_RES 2: note = Xaa is L-2-aminobutyric acid (Abu)
  REGION 2..7: note = MISC_FEATURE - bridge between Abu at position X2 and Cys at position X7
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is alpha-methyl-L-leucine (Mle)
  MOD_RES 13: note = Xaa is L-3-pyridyl-alanine (Pal)
  MOD_RES 14: note = Xaa is sarcosine (Sar)
  MOD_RES 14: note = AMIDATION
XXNTWKCXXXKNXX

SEQ ID NO: 138 - protein, 13 aa, synthetic construct
  REGION 1..13: note = Artificial Peptide
  MOD_RES 1: note = Xaa is desamino-aspartamic acid (Dad)
  REGION 1..10: note = MISC_FEATURE - lactam bridge between Dad at position X1 and Lys at position X10
  MOD_RES 2: note = Xaa is L-2-aminobutyric acid (Abu)
  REGION 2..3: note = MISC_FEATURE - bond between Abu at position X2 and epsilon amino group of adjacent acetylated Lys
  REGION 2..6: note = MISC_FEATURE - bridge between Abu at position X2 and Cys at position X6
  MOD_RES 3: note = ACETYLATION
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is 2-aminoisobutyric acid (Aib)
  MOD_RES 12: note = Xaa is L-3-pyridyl-alanine (Pal)
  MOD_RES 13: note = Xaa is sarcosine (Sar)
  MOD_RES 13: note = AMIDATION
XXKWKCXXXKNXX

SEQ ID NO: 139 - protein, 13 aa, synthetic construct
  REGION 1..13: note = Artificial Peptide
  MOD_RES 1: note = Xaa is desamino-aspartamic acid (Dad)
  REGION 1..10: note = MISC_FEATURE - lactam bridge between Dad at position X1 and Lys at position X10
  MOD_RES 2: note = Xaa is L-2-aminobutyric acid (Abu)
  REGION 2..3: note = MISC_FEATURE - bond between Abu at position X2 and epsilon amino group of adjacent acetylated Lys
  REGION 2..6: note = MISC_FEATURE - bridge between Abu at position X2 and Cys at position X6
  MOD_RES 3: note = ACETYLATION
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is 4-amino-tetrahydropyran-4-carboxylic acid (Thp)
  MOD_RES 12: note = Xaa is L-3-pyridyl-alanine (Pal)
  MOD_RES 13: note = Xaa is sarcosine (Sar)
  MOD_RES 13: note = AMIDATION
XXKWKCXXXKNXX

SEQ ID NO: 140 - protein, 13 aa, synthetic construct
  REGION 1..13: note = Artificial Peptide
  MOD_RES 1: note = Xaa is desamino-aspartamic acid (Dad)
  REGION 1..10: note = MISC_FEATURE - lactam bridge between Dad at position X1 and Lys at position X10
  MOD_RES 2: note = Xaa is L-2-aminobutyric acid (Abu)
  REGION 2..3: note = MISC_FEATURE - bond between Abu at position X2 and epsilon amino group of adjacent acetylated Lys
  REGION 2..6: note = MISC_FEATURE - bridge between Abu at position X2 and Cys at position X6
  MOD_RES 3: note = ACETYLATION
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is alpha-methyl-L-leucine (Mle)
  MOD_RES 12: note = Xaa is L-3-pyridyl-alanine (Pal)
  MOD_RES 13: note = Xaa is sarcosine (Sar)
  MOD_RES 13: note = AMIDATION
XXKWKCXXXKNXX

SEQ ID NO: 141 - protein, 14 aa, synthetic construct
  REGION 1..14: note = Artificial Peptide
  MOD_RES 1: note = Xaa is desamino-aspartamic acid (Dad)
  REGION 1..11: note = MISC_FEATURE - lactam bridge between Dad at position X1 and Kme at position X11
  MOD_RES 2: note = Xaa is L-2-aminobutyric acid (Abu)
  REGION 2..7: note = MISC_FEATURE - bridge between Abu at position X2 and Cys at position X7
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is 4-amino-tetrahydropyran-4-carboxylic acid (Thp)
  MOD_RES 11: note = Xaa is (2S)-2-amino-6-(methylamino)hexanoic acid (epsilon-N-methyl-lysine) (Kme)
  MOD_RES 13: note = Xaa is L-3-pyridyl-alanine (Pal)
  MOD_RES 14: note = Xaa is sarcosine (Sar)
  MOD_RES 14: note = AMIDATION
XXNTWKCXXXXNXX

SEQ ID NO: 142 - protein, 13 aa, synthetic construct
  REGION 1..13: note = Artificial Peptide
  MOD_RES 1: note = Xaa is desamino-aspartamic acid (Dad)
  REGION 1..10: note = MISC_FEATURE - lactam bridge between Dad at position X1 and Kme at position X10
  MOD_RES 2: note = Xaa is L-2-aminobutyric acid (Abu)
  REGION 2..3: note = MISC_FEATURE - bond between Abu at position X2 and epsilon amino group of adjacent acetylated Lys
  REGION 2..6: note = MISC_FEATURE - bridge between Abu at position X2 and Cys at position X6
  MOD_RES 3: note = ACETYLATION
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is (2S)-2-amino-6-(dimethylamino)-2-methyl-hexanoic acid (Mkdm)
  MOD_RES 10: note = Xaa is (2S)-2-amino-6-(methylamino)hexanoic acid (epsilon-N-methyl-lysine) (Kme)
  MOD_RES 12: note = Xaa is L-3-pyridyl-alanine (Pal)
  MOD_RES 13: note = Xaa is sarcosine (Sar)
  MOD_RES 13: note = AMIDATION
XXKWKCXXXXNXX

SEQ ID NO: 143 - protein, 12 aa, synthetic construct
  REGION 1..12: note = Artificial Peptide
  MOD_RES 1: note = Cys is modified at N-terminus with butyric acid (C[Buty])
  REGION 1..2: note = MISC_FEATURE - bond between Cys[Buty] at position X1 and epsilon amino group of adjacent acetylated Lys
  REGION 1..9: note = MISC_FEATURE - bridge between Cys[Buty] at position X1 and Kme at position X9
  DISULFID 1..5: note = disulfide bridge between Cys at position X1 and Cys at position X5
  MOD_RES 2: note = ACETYLATION
  MOD_RES 3: note = Xaa is 7-methyl-L-tryptophan (Wim)
  MOD_RES 4: note = ACETYLATION
  MOD_RES 6: note = Xaa is (2S)-2-amino-3-[4-[2-(dimethylamino)ethoxy]p henyl]propanoic acid (Yde)
  MOD_RES 7: note = Xaa is 2-naphthyl-L-alanine (Nal)
  MOD_RES 8: note = Xaa is (2S)-2-amino-6-(dimethylamino)-2-methyl-hexanoic acid (Mkdm)
  MOD_RES 9: note = Xaa is (2S)-2-amino-6-(methylamino)hexanoic acid (epsilon-N-methyl-lysine) (Kme)
  MOD_RES 11: note = Xaa is L-3-pyridyl-alanine (Pal)
  MOD_RES 12: note = Xaa is sarcosine (Sar)
  MOD_RES 12: note = AMIDATION
CKXKCXXXXNXX

SEQ ID NO: 144 - protein, 11 aa, synthetic construct
  REGION 1..11: note = Artificial Peptide
  MOD_RES 1: note = Cys is modified at N-terminus with butyric acid (C[Buty])
  REGION 1..2: note = MISC_FEATURE - bond between Cys[Buty] at position X1 and epsilon amino group of adjacent acetylated Lys
  REGION 1..9: note = MISC_FEATURE - bridge between Cys[Buty] at position X1 and Kme at position X9
  DISULFID 1..5: note = disulfide bridge between Cys at position X1 and Cys at position X5
  MOD_RES 2: note = ACETYLATION
  MOD_RES 3: note = Xaa is 7-methyl-L-tryptophan (Wim)
  MOD_RES 4: note = ACETYLATION
  MOD_RES 6: note = Xaa is (2S)-2-amino-3-[4-[2-(dimethylamino)ethoxy]p henyl]propanoic acid (Yde)
  MOD_RES 7: note = Xaa is 2-naphthyl-L-alanine (Nal)
  MOD_RES 8: note = Xaa is (2S)-2-amino-6-(dimethylamino)-2-methyl-hexanoic acid (Mkdm)
  MOD_RES 9: note = Xaa is (2S)-2-amino-6-(methylamino)hexanoic acid (epsilon-N-methyl-lysine) (Kme)
  MOD_RES 11: note = Xaa is 2-(3-pyridyl)ethanamine (PyEA)
CKXKCXXXXGX

SEQ ID NO: 145 - protein, 12 aa, synthetic construct
  REGION 1..12: note = Artificial Peptide
  MOD_RES 1: note = Xaa is N-methyl-L-cysteine substituted with butyl (Nmc[Butyl])
  REGION 1..2: note = MISC_FEATURE - bond between Nmc[Butyl] at position X1 and epsilon amino group of adjacent acetylated Lys
  REGION 1..9: note = MISC_FEATURE - bridge between Nmc[Butyl] at position X1 and Kme at position X9
  DISULFID 1..5: note = disulfide bridge between Nmc at position X1 and Cys at position X5
  MOD_RES 2: note = ACETYLATION
  MOD_RES 3: note = Xaa is 7-methyl-L-tryptophan (Wim)
  MOD_RES 4: note = ACETYLATION
  MOD_RES 6: note = Xaa is (2S)-2-amino-3-[4-[2-(dimethylamino)ethoxy]p henyl]propanoic acid (Yde)
  MOD_RES 7: note = Xaa is 2-naphthyl-L-alanine (Nal)
  MOD_RES 8: note = Xaa is (2S)-2-amino-6-(dimethylamino)-2-methyl-hexanoic acid (Mkdm)
  MOD_RES 9: note = Xaa is (2S)-2-amino-6-(methylamino)hexanoic acid (epsilon-N-methyl-lysine) (Kme)
  MOD_RES 11: note = Xaa is L-3-pyridyl-alanine (Pal)
  MOD_RES 12: note = Xaa is sarcosine (Sar)
  MOD_RES 12: note = AMIDATION
XKXKCXXXXNXX

SEQ ID NO: 146 - protein, 11 aa, synthetic construct
  REGION 1..11: note = Artificial Peptide
  MOD_RES 1: note = Xaa is N-methyl-L-cysteine substituted with butyl (Nmc[Butyl])
  REGION 1..2: note = MISC_FEATURE - bond between Nmc[Butyl] at position X1 and epsilon amino group of adjacent acetylated Lys
  REGION 1..9: note = MISC_FEATURE - bridge between Nmc[Butyl] at position X1 and Kme at position X9
  DISULFID 1..5: note = disulfide bridge between Nmc at position X1 and Cys at position X5
  MOD_RES 2: note = ACETYLATION
  MOD_RES 3: note = Xaa is 7-methyl-L-tryptophan (Wim)
  MOD_RES 6: note = Xaa is (2S)-2-amino-3-[4-[2-(dimethylamino)ethoxy]p henyl]propanoic acid (Yde)
  MOD_RES 7: note = Xaa is 2-naphthyl-L-alanine (Nal)
  MOD_RES 8: note = Xaa is (2S)-2-amino-6-(dimethylamino)-2-methyl-hexanoic acid (Mkdm)
  MOD_RES 9: note = Xaa is (2S)-2-amino-6-(methylamino)hexanoic acid (epsilon-N-methyl-lysine) (Kme)
  MOD_RES 11: note = Xaa is 2-(3-pyridyl)ethanamine (PyEA)
XKXQCXXXXGX

SEQ ID NO: 147 - protein, 11 aa, synthetic construct
  REGION 1..11: note = Artificial Peptide
  MOD_RES 1: note = Xaa is N-methyl-L-cysteine substituted with butyl (Nmc[Butyl])
  REGION 1..2: note = MISC_FEATURE - bond between Nmc[Butyl] at position X1 and epsilon amino group of adjacent acetylated Lys
  REGION 1..9: note = MISC_FEATURE - bridge between Nmc[Butyl] at position X1 and Kme at position X9
  DISULFID 1..5: note = disulfide bridge between Nmc at position X1 and Cys at position X5
  MOD_RES 2: note = ACETYLATION
  MOD_RES 3: note = Xaa is 1-methyl-L-tryptophan (Wme)
  MOD_RES 4: note = ACETYLATION
  MOD_RES 6: note = Xaa is (2S)-2-amino-3-[4-[2-(dimethylamino)ethoxy]p henyl]propanoic acid (Yde)
  MOD_RES 7: note = Xaa is 2-naphthyl-L-alanine (Nal)
  MOD_RES 8: note = Xaa is (2S)-2-amino-6-(dimethylamino)-2-methyl-hexanoic acid (Mkdm)
  MOD_RES 9: note = Xaa is (2S)-2-amino-6-(methylamino)hexanoic acid (epsilon-N-methyl-lysine) (Kme)
  MOD_RES 11: note = Xaa is 2-(3-pyridyl)ethanamine (PyEA)
XKXKCXXXXGX

SEQ ID NO: 148 - protein, 11 aa, synthetic construct
  REGION 1..11: note = Artificial Peptide
  MOD_RES 1: note = Xaa is N-methyl-L-cysteine substituted with butyl (Nmc[Butyl])
  REGION 1..2: note = MISC_FEATURE - bond between Nmc[Butyl] at position X1 and epsilon amino group of adjacent acetylated Lys
  REGION 1..9: note = MISC_FEATURE - bridge between Nmc[Butyl] at position X1 and Kme at position X9
  DISULFID 1..5: note = disulfide bridge between Nmc at position X1 and Cys at position X5
  MOD_RES 2: note = ACETYLATION
  MOD_RES 3: note = Xaa is 1-methyl-L-tryptophan (Wme)
  MOD_RES 6: note = Xaa is (2S)-2-amino-3-[4-[2-(dimethylamino)ethoxy]p henyl]propanoic acid (Yde)
  MOD_RES 7: note = Xaa is 2-naphthyl-L-alanine (Nal)
  MOD_RES 8: note = Xaa is (2S)-2-amino-6-(dimethylamino)-2-methyl-hexanoic acid (Mkdm)
  MOD_RES 9: note = Xaa is (2S)-2-amino-6-(methylamino)hexanoic acid (epsilon-N-methyl-lysine) (Kme)
  MOD_RES 11: note = Xaa is 2-(3-pyridyl)ethanamine (PyEA)
XKXLCXXXXGX

SEQ ID NO: 149 - protein, 13 aa, synthetic construct
  REGION 1..13: note = Artificial Peptide
  MOD_RES 1: note = Xaa is 5-aminopentanoic acid (Apt)
  REGION 1..10: note = MISC_FEATURE - lactam bridge between Apt at position X1 and Glu at position X10
  REGION 2..3: note = MISC_FEATURE - bond between Cys at position X2 and epsilon amino group of adjacent acetylated Lys
  DISULFID 2..6: note = disulfide bridge between Cys at position X2 and Cys at position X6
  MOD_RES 3: note = ACETYLATION
  MOD_RES 4: note = Xaa is 7-methyl-L-tryptophan (Wim)
  MOD_RES 5: note = ACETYLATION
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is (2S)-2-amino-6-(dimethylamino)-2-methyl-hexanoic acid (Mkdm)
  MOD_RES 12: note = Xaa is L-3-pyridyl-alanine (Pal)
  MOD_RES 13: note = Xaa is sarcosine (Sar)
  MOD_RES 13: note = AMIDATION
XCKXKCXXXENXX

SEQ ID NO: 150 - protein, 13 aa, synthetic construct
  REGION 1..13: note = Artificial Peptide
  MOD_RES 1: note = Xaa is desamino-aspartamic acid (Dad)
  REGION 1..11: note = MISC_FEATURE - lactam bridge between Dad at position X1 and Lys at position X11
  DISULFID 2..7: note = disulfide bridge between Cys at position X2 and Cys at position X7
  MOD_RES 5: note = Xaa is 7-methyl-L-tryptophan (Wim)
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is (2S)-2-amino-6-(dimethylamino)-2-methyl-hexanoic acid (Mkdm)
  MOD_RES 13: note = Xaa is L-3-pyridyl-alanine (Pal)
  MOD_RES 13: note = AMIDATION
XCNTXLCXXXKNX

SEQ ID NO: 151 - protein, 14 aa, synthetic construct
  REGION 1..14: note = Artificial Peptide
  MOD_RES 1: note = Xaa is desamino-aspartamic acid (Dad)
  REGION 1..11: note = MISC_FEATURE - lactam bridge between Dad at position X1 and Lys at position X11
  DISULFID 2..7: note = disulfide bridge between Cys at position X2 and Cys at position X7
  MOD_RES 5: note = Xaa is 7-methyl-L-tryptophan (Wim)
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is (2S)-2-amino-6-(dimethylamino)-2-methyl-hexanoic acid (Mkdm)
  MOD_RES 13: note = Xaa is L-3-pyridyl-alanine (Pal)
  MOD_RES 14: note = Xaa is sarcosine (Sar)
  MOD_RES 14: note = AMIDATION
XCNTXKCXXXKNXX

SEQ ID NO: 152 - protein, 14 aa, synthetic construct
  REGION 1..14: note = Artificial Peptide
  MOD_RES 1: note = Xaa is desamino-aspartamic acid (Dad)
  REGION 1..11: note = MISC_FEATURE - lactam bridge between Dad at position X1 and Lys at position X11
  DISULFID 2..7: note = disulfide bridge between Cys at position X2 and Cys at position X7
  MOD_RES 5: note = Xaa is 7-methyl-L-tryptophan (Wim)
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is (2S)-2-amino-6-(dimethylamino)-2-methyl-hexanoic acid (Mkdm)
  MOD_RES 13: note = Xaa is L-3-pyridyl-alanine (Pal)
  MOD_RES 14: note = Xaa is sarcosine (Sar)
  MOD_RES 14: note = AMIDATION
XCNTXQCXXXKNXX

SEQ ID NO: 153 - protein, 14 aa, synthetic construct
  REGION 1..14: note = Artificial Peptide
  MOD_RES 1: note = Xaa is desamino-aspartamic acid (Dad)
  REGION 1..11: note = MISC_FEATURE - lactam bridge between Dad at position X1 and Lys at position X11
  DISULFID 2..7: note = disulfide bridge between Cys at position X2 and Cys at position X7
  MOD_RES 5: note = Xaa is 7-methyl-L-tryptophan (Wim)
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is (2S)-2-amino-6-(dimethylamino)-2-methyl-hexanoic acid (Mkdm)
  MOD_RES 13: note = Xaa is L-3-pyridyl-alanine (Pal)
  MOD_RES 14: note = Xaa is sarcosine (Sar)
  MOD_RES 14: note = AMIDATION
XCNTXQCXXXKNXX

SEQ ID NO: 154 - protein, 14 aa, synthetic construct
  REGION 1..14: note = Artificial Peptide
  MOD_RES 1: note = Xaa is desamino-aspartamic acid (Dad)
  REGION 1..11: note = MISC_FEATURE - lactam bridge between Dad at position X1 and Lys at position X11
  DISULFID 2..7: note = disulfide bridge between Cys at position X2 and Cys at position X7
  MOD_RES 5: note = Xaa is 7-methyl-L-tryptophan (Wim)
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is (2S)-2-amino-6-(dimethylamino)-2-methyl-hexanoic acid (Mkdm)
  MOD_RES 13: note = Xaa is L-3-pyridyl-alanine (Pal)
  MOD_RES 14: note = Xaa is sarcosine (Sar)
  MOD_RES 14: note = AMIDATION
XCNTXLCXXXKNXX

SEQ ID NO: 155 - protein, 14 aa, synthetic construct
  REGION 1..14: note = Artificial Peptide
  MOD_RES 1: note = Xaa is desamino-aspartamic acid (Dad)
  REGION 1..11: note = MISC_FEATURE - lactam bridge between Dad at position X1 and Lys at position X11
  DISULFID 2..7: note = disulfide bridge between Cys at position X2 and Cys at position X7
  MOD_RES 5: note = Xaa is 7-methyl-L-tryptophan (Wim)
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is (2S)-2-amino-6-(dimethylamino)-2-methyl-hexanoic acid (Mkdm)
  MOD_RES 13: note = Xaa is L-3-pyridyl-alanine (Pal)
  MOD_RES 14: note = Xaa is sarcosine (Sar)
  MOD_RES 14: note = AMIDATION
XCNTXLCXXXKAXX

SEQ ID NO: 156 - protein, 14 aa, synthetic construct
  REGION 1..14: note = Artificial Peptide
  MOD_RES 1: note = Xaa is desamino-aspartamic acid (Dad)
  REGION 1..11: note = MISC_FEATURE - lactam bridge between Dad at position X1 and Kme at position X11
  DISULFID 2..7: note = disulfide bridge between Cys at position X2 and Cys at position X7
  MOD_RES 5: note = Xaa is 7-methyl-L-tryptophan (Wim)
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is (2S)-2-amino-6-(dimethylamino)-2-methyl-hexanoic acid (Mkdm)
  MOD_RES 11: note = Xaa is (2S)-2-amino-6-(methylamino)hexanoic acid (epsilon-N-methyl-lysine) (Kme)
  MOD_RES 13: note = Xaa is L-3-pyridyl-alanine (Pal)
  MOD_RES 14: note = Xaa is sarcosine (Sar)
  MOD_RES 14: note = AMIDATION
XCNTXKCXXXXNXX

SEQ ID NO: 157 - protein, 14 aa, synthetic construct
  REGION 1..14: note = Artificial Peptide
  MOD_RES 1: note = Xaa is desamino-aspartamic acid (Dad)
  REGION 1..11: note = MISC_FEATURE - lactam bridge between Dad at position X1 and Lys at position X11
  DISULFID 2..7: note = disulfide bridge between Cys at position X2 and Cys at position X7
  MOD_RES 5: note = Xaa is 7-methyl-L-tryptophan (Wim)
  MOD_RES 6: note = ACETYLATION
  MOD_RES 8: note = Xaa is (2S)-2-amino-3-[4-[2-(dimethylamino)ethoxy]p henyl]propanoic acid (Yde)
  MOD_RES 9: note = Xaa is 2-naphthyl-L-alanine (Nal)
  MOD_RES 10: note = Xaa is 2-aminoisobutyric acid (Aib)
  MOD_RES 13: note = Xaa is L-3-pyridyl-alanine (Pal)
  MOD_RES 14: note = Xaa is sarcosine (Sar)
  MOD_RES 14: note = AMIDATION
XCNTXKCXXXKNXX

SEQ ID NO: 158 - protein, 13 aa, synthetic construct
  REGION 1..13: note = Artificial Peptide
  MOD_RES 1: note = Xaa is desamino-aspartamic acid (Dad)
  REGION 1..10: note = MISC_FEATURE - lactam bridge between Dad at position X1 and Lys at position X10
  REGION 2..3: note = MISC_FEATURE - bond between Cys at position X2 and epsilon amino group of adjacent acetylated Lys
  DISULFID 2..6: note = disulfide bridge between Cys at position X2 and Cys at position X6
  MOD_RES 3: note = ACETYLATION
  MOD_RES 4: note = Xaa is 7-methyl-L-tryptophan (Wim)
  MOD_RES 7: note = Xaa is (2S)-2-amino-3-[4-[2-(dimethylamino)ethoxy]p henyl]propanoic acid (Yde)
  MOD_RES 8: note = Xaa is 2-naphthyl-L-alanine (Nal)
  MOD_RES 9: note = Xaa is alpha-methyl-L-leucine (Mle)
  MOD_RES 12: note = Xaa is L-3-pyridyl-alanine (Pal)
  MOD_RES 13: note = Xaa is sarcosine (Sar)
  MOD_RES 13: note = AMIDATION
XCKXLCXXXKNXX

SEQ ID NO: 159 - protein, 13 aa, synthetic construct
  MOD_RES 1: note = X1 is 5-aminopentanoic acid (Apt)
  SITE 1..11: note = lactam bridge between Apt at position X1 and Glu at position X11
  MOD_RES 2: note = X2 is 2-amino-3-methyl-3-sulfanylbutanoic acid (Pen)
  SITE 2..7: note = disulfide bridge between Pen at position X2 and Pen at position X7
  MOD_RES 7: note = X7 is 2-amino-3-methyl-3-sulfanylbutanoic acid (Pen)
  MOD_RES 8: note = X8 is 4-methoxy-L-phenylalanine (Yme)
  MOD_RES 9: note = X9 is 2-naphthyl-L-alanine (Nal)
  MOD_RES 10: note = X10 is 2-aminoisobutyric acid (Aib)
XXNTWQXXXXENN

SEQ ID NO: 160 - protein, 13 aa, synthetic construct
  MOD_RES 9: note = X9 is 2-naphthyl-L-alanine (Nal)
  MOD_RES 8: note = X8 is 4-methoxy-L-phenylalanine (Yme)
  MOD_RES 10: note = X10 is 2-aminoisobutyric acid (Aib)
  MOD_RES 2: note = X2 is 2-amino-3-methyl-3-sulfanylbutanoic acid (Pen)
  MOD_RES 7: note = X7 is 2-amino-3-methyl-3-sulfanylbutanoic acid (Pen)
  MOD_RES 1: note = X1 is 6-aminohexanoic acid (Ahx)
  SITE 1..11: note = lactam bridge between Ahx at position X1 and Glu at position X11
  SITE 2..8: note = disulfide bridge between Pen at position X2 and Pen at position X7
XXNTWQXXXXENN

SEQ ID NO: 161 - protein, 13 aa, synthetic construct
  MOD_RES 9: note = X9 is 2-naphthyl-L-alanine (Nal)
  MOD_RES 10: note = X10 is 4-amino-tetrahydropyran-4-carboxylic acid (Thp)
  MOD_RES 1: note = X1 is 6-aminohexanoic acid (Ahx)
  MOD_RES 2: note = X2 is 2-amino-3-methyl-3-sulfanylbutanoic acid (Pen)
  MOD_RES 7: note = X7 is 2-amino-3-methyl-3-sulfanylbutanoic acid (Pen)
  MOD_RES 8: note = X8 is 4-methoxy-L-phenylalanine (Yme)
  SITE 1..11: note = lactam bridge between Ahx at position X1 and Glu at position X11
  SITE 2..8: note = disulfide bridge between Pen at position X2 and Pen at position X7
XXNTWQXXXXENG

SEQ ID NO: 162 - protein, 13 aa, synthetic construct
  MOD_RES 8: note = X8 is 4-methoxy-L-phenylalanine (Yme)
  MOD_RES 2: note = X2 is 2-amino-3-methyl-3-sulfanylbutanoic acid (Pen)
  MOD_RES 9: note = X9 is 2-naphthyl-L-alanine (Nal)
  MOD_RES 10: note = X10 is alpha-methyl-L-leucine (Mle)
  MOD_RES 1: note = X1 is 6-aminohexanoic acid (Ahx)
  MOD_RES 7: note = X7 is 2-amino-3-methyl-3-sulfanylbutanoic acid (Pen)
  SITE 1..11: note = lactam bridge between Ahx at position X1 and Asp at position X11
  SITE 2..8: note = disulfide bridge between Pen at position X2 and Pen at position X7
XXNTWQXXXXDNN

SEQ ID NO: 163 - protein, 13 aa, synthetic construct
  MOD_RES 2: note = X2 is 2-amino-3-methyl-3-sulfanylbutanoic acid (Pen)
  MOD_RES 9: note = X9 is 2-naphthyl-L-alanine (Nal)
  MOD_RES 10: note = X10 is 4-amino-tetrahydropyran-4-carboxylic acid (Thp)
  MOD_RES 1: note = X1 is 5-aminopentanoic acid (Apt)
  MOD_RES 8: note = X8 is 4-aminoethoxy-L-phenylalanine (Yae)
  MOD_RES 7: note = X7 is 2-amino-3-methyl-3-sulfanylbutanoic acid (Pen)
  SITE 1..11: note = lactam bridge between Apt at position X1 and Glu at position X11
  SITE 2..8: note = disulfide bridge between Pen at position X2 and Pen at position X7
XXNTWQXXXXENN

SEQ ID NO: 164 - protein, 13 aa, synthetic construct
  MOD_RES 1: note = X1 is 6-aminohexanoic acid (Ahx)
  MOD_RES 2: note = X2 is 2-amino-3-methyl-3-sulfanylbutanoic acid (Pen)
  MOD_RES 8: note = X8 is 4-methoxy-L-phenylalanine (Yme)
  MOD_RES 9: note = X9 is 2-naphthyl-L-alanine (Nal)
  MOD_RES 10: note = X10 is 2-aminoisobutyric acid (Aib)
  SITE 1..11: note = lactam bridge between Ahx at position X1 and Asp at position X11
  SITE 2..8: note = disulfide bridge between Pen at position X2 and Cys at position X7
XXNTWQCXXXDNN

(19) United States
(12) Patent Application Publication
(10) Pub. No.: US 2026/0258084 A1
(43) Pub. Date: Sep. 3, 2026

(54) New Peptides as Selective IL-23 Receptor Antagonists

(71) Applicant: Sanofi, Paris (FR)

(72) Inventors: Melissa Besenius, Frankfurt am Main (DE); Thomas Boehme, Frankfurt am Main (DE); Torsten Haack, Frankfurt am Main (DE); Michael Kurz, Frankfurt am Main (DE); Stefan Petry, Frankfurt am Main (DE); Christoph Pöverlein, Frankfurt am Main (DE); Garima Tiwari, Frankfurt am Main (DE); Michael Wagner, Frankfurt am Main (DE)

(73) Assignee: Sanofi, Paris (FR)

(21) Appl. No.: 18/877,821

(22) PCT Filed: Jun. 29, 2023
(86) PCT No.: PCT/EP2023/067928

§ 371 Date: Dec. 20, 2024

(30) Foreign Application Priority Data
Jun. 30, 2022 (EP) 22315131.7

(51) Int. Cl. C07K 7/50; A61K 38/00

(52) U.S. Cl. CPC C07K 7/50; A61K 38/00

(57) ABSTRACT

The present disclosure relates to peptide IL23 receptor antagonists, their pharmaceutical compositions and their medical use, for example in the treatment of inflammatory bowel diseases such as Crohn's disease.

The present disclosure relates to peptide IL-23 receptor antagonists and their medical use, for example in the treatment of inflammatory bowel diseases such as Crohn's disease. Compounds disclosed herein are macrolactam peptides and show high solubility and stability under neutral conditions also in simulated intestinal and gastric fluid. The macrolactam peptides show high in vitro potency at the interleukin 23 receptor (IL-23R) with favourable physico-chemical properties.

### BACKGROUND

There is increasing evidence that interleukin-23 (IL-23) cytokine is playing a crucial role in the pathogenesis of autoimmune inflammation and related diseases and disorders, such as asthma, rheumatoid arthritis, psoriasis, multiple sclerosis and inflammatory bowel diseases (IBDs), e.g., ulcerative colitis and Crohn's disease. The primary role of IL-23R could be revealed by acute and chronic mouse models of IBD and downstream effector cytokines in disease pathogenesis. Th17 cells, y8 T cells, natural killer (NK) cells, dendritic cells, macrophages, and innate lymphoid cells, which are found abundantly in the intestine are adaptive and innate immune cells which express IL-23R. IBD patients have an increased gene expression and IL-23R level at the intestine mucosal surface. There is evidence that IL-23 contributes to this effect by promoting the development of a pathogenic CD4+ T cell population that produces IL-6, IL-17, and tumor necrosis factor (TNF).

Concentration of IL-23 is increased in the intestine, where it is thought to play a key role in regulating the balance between tolerance and immunity through T-cell-dependent and T-cell-independent pathways of intestinal inflammation. This regulation has effects on T-helper 1 (Th1) and Th17-associated cytokines, as well as repressing regulatory T-cell responses in the gut, promoting inflammation. Furthermore, IL-23R polymorphisms have been associated with susceptibility to gut inflammation, further confirming the importance of the IL-23 pathway in intestinal homeostasis.

Psoriasis is a chronic skin disease with a prevalence in the Caucasian population of about 2%-3% has been shown to be mediated by the body's T cell inflammatory response mechanisms. Via the induction of interleukin-17, activation of macrophages and regulation of T memory cells, IL-23 is one of several interleukins implicated as a key player in the pathogenesis of psoriasis by maintaining chronic autoimmune inflammation. Neutralizing antibodies against IL-23 showed IL-23-dependent inhibition of psoriasis development in animal models of psoriasis while high expression of IL-23 and IL-23R has been demonstrated to be increased in tissues of patients with psoriasis.

IL-23 is a part of the IL-12 family of cytokines and is composed of a p19 subunit specific to IL-23 and the p40 subunit of IL-12, which is a cytokine involved in the development of interferon-y (IFN-y)-producing T helper 1 (TH1) cells. Although IL-23 and IL-12 both contain the p40 subunit, they have different phenotypic properties. While animals with deficiency in IL-12 are susceptible to inflammatory autoimmune diseases, IL-23 deficient animals are resistant, most likely due to a reduced number of CD4+ T cells producing IL-6, IL-17, and TNF in the CNS. IL-23 binds to IL-23R, which is a heterodimeric receptor composed of IL-12Rβ1 and IL-23R subunits. Binding of IL-23 to IL-23R activates the Jak-Stat signalling molecules, Jak2, Tyk2, Stat1, Stat3, Stat4, and Stat 5, although Stat4 activation is substantially weaker and different DNA-binding Stat complexes form in response to IL-23 as compared with IL-12. IL-23R associates constitutively with Jak2 and in a ligand-dependent manner with Stat3. In contrast to IL-12, which acts mainly on naive CD4(+) T cells, IL-23 preferentially acts on memory CD4(+) T cells.

Based on the biological importance, inhibition of the IL-23 pathway is an attractive option for treatment of IL-23-related diseases and disorders. A number of antibodies that bind to IL-23 or IL-23R have been identified, including ustekinumab, a humanized antibody that binds IL-23, which has been approved for the treatment of psoriasis. More recently, polypeptide inhibitors that bind to IL-23R and inhibit the binding of IL-23 to IL-23R have been identified (see, e.g., US Patent Application Publication No. US2013/0029907). Clinical trials in Crohn's Disease or psoriasis with ustekinumab and briakinumab (which target the common p40 subunit) and tildrakizumab, guselkumab, brazikumab, and risankizumab (which target the unique p19 subunit of IL-23) highlight the potential of IL-23 signalling blockade in treatment of human inflammatory diseases. While these findings are promising, challenges remain with respect to identifying stable and selective agents that preferentially target the IL-23 pathway in the intestine, which can be used for the treatment of intestinal inflammation, such as intestinal bowel diseases, including Crohn's disease, ulcerative colitis and related disorders.

Irritable bowel disease (IBD) patients with autoimmune inflammation in the intestinal tract can benefit from an oral-to-local treatment where compounds specifically target the IL-23 pathway from the luminal side of the gut. The present disclosure addresses these needs by providing novel peptide compounds that bind IL-23R to inhibit IL-23 binding and signalling and which are suitable for oral administration.

### DESCRIPTION

A problem associated with the use of peptide compounds as an orally administered therapeutic in the treatment of inflammatory diseases and other indications is their limited stability in the intestinal environment. Therefore, peptide sequences can be stabilized by introduction of non-genetically encoded amino acids and/or are cyclized to enhance stability against proteases to allow interaction with a target located in the luminal side of the intestine.

Therefore, in developing new therapeutic molecules, there is a need for variants with improved pharmaceutical properties, in particular increased stability against proteases which are present in the gut and/or increased chemical or physical stability and/or a prolonged half-life in vivo and/or increased potency/efficacy in vivo.

Also, there remains a need for anti-inflammatory therapies that avoid or even alleviate the common systemic side effects of IL-23R based therapies (e.g., defence against infections), thereby achieving a strong anti-inflammatory effect with improved tolerability.

Protagonist's WO2016011208A1, WO2017011820A2, WO2018022937A1, WO2018136646A1, WO2020014646, WO2021007433, WO2021146454, WO2021146458 and WO2021146441 as well as Sayago et al, ACS Med. Chem. Lett. 2018, 9, 912-916 disclose peptide antagonists of the IL-23 receptor for oral administration. WO2022109328 discloses compositions of a cyclic peptide with the sequence Ac-Pen-Asn-Thr-Wim-Lys(Ac)-Pen-Yae-Nal-Thp-Glu-Asn-Pal-Sar-NH₂, wherein a disulfide bond is formed between Pen-Pen. A common structural characteristic of the disclosed compounds is a ring formed by 6 amino acids as ring members.

WO2013063468 discloses cyclic peptides which are modified by long chain hydrocarbon groups resulting in amphiphilic molecules useful as drug delivery system including nucleotide delivery to cells.

WO2015179438 also discloses cyclic peptides of a 2 to 10 membered ring formed by amino acids as inhibitors Rac or Rho in a cell or tissue, wherein at least two amino acids are arginine.

Compounds of this disclosure show high affinity on the IL-23 receptor.

Characteristic structural motifs of the disclosed compounds include a macrolactam ring with 34-40 atoms, which is formed by 9, 10 or 11 amino acids as building blocks as well as a thioether, disulfide or ester bridge between side chains of amino acids forming a second bicyclic structure with 5 or 6 amino acids as building blocks.

Therefore, novel bicyclic peptides are provided having high affinity to the IL-23 receptor. Further provided are medical uses of these bicyclic peptides.

Disclosed herein are compounds of the formula (I):

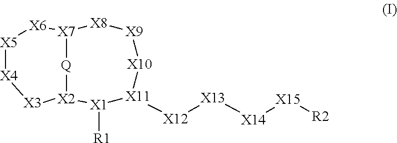

wherein
   X1 is a residue selected from a residue of Ahx, Aoa, Apt, Nmha, Nmapt, Lys, Pipa, Pipb, Pipp, Dab, Dad and Dap, and is linked via an amide bond to X11,
or is a residue selected from a residue of Buty and Butyl, and is linked via an alkyl bond to X11 and when X1 is a residue of Butyl, the residue of Butyl is linked to X2 via an alkyl bond,
   R1 is absent or, if X1 is selected from a residue of Ahx, Aoa, Apt, Lys, Dab and Dap, then R1 is H, C₁-C₄-alkyl or acetyl, and is linked to an amino function of X1,
   X2 is an amino acid residue selected from a residue of Cys, Glu, Asp, Nmc, Nmd, Nme, Nmt, Thr, and Abu, and their corresponding d-forms. In some embodiments, X2 is an amino acid residue selected from a residue of Cys, Glu, Asp, Nmc, Nmd, Nme, Nmt, and Thr, and their corresponding d-forms,
   X7 is an amino acid residue selected from a residue of Cys, Glu, Asp, Thr, and Abu, and their corresponding d-forms,
wherein X2 and X7 are linked to each other via Q, wherein Q is a disulfide bond, a thioether bond or an ester bond,
   X3 is an amino acid residue selected from a residue of Sar, His, Lys, Ala, Ahx, Apt, Arg, Asn, Gab, Gln, Trp, Thr, Ndmk, Nmn, Ntmk, and Lys(αAc),
   X4 is absent, or is an amino acid residue selected from a residue of His, Lys, Ahx, Apt, Arg, Asn, Gab, Gln, Trp, and Thr,
   X5 is an amino acid residue selected from a residue of Trp, Wme, Nak, Wcl, Wim, Wdm, and Wfl,
   X6 is an amino acid residue selected from a residue of Gin, Aib, Glu, Phe, Ile, Iva, Lys, Lys(Ac), Leu, Mle, Mly, Mva, Thr, Val, Trp, and Phe,
   X8 is an amino acid residue selected from a residue of Yme, Tyr, Trp, Yde, and Yae. In some embodiments, X8 is an amino acid residue selected from a residue of Yme, Tyr, Trp, and Yde,
   X9 is an amino acid residue selected from a residue of Nal, Nak, and Trp,
   X10 is an amino acid residue selected from a residue of Aib, Cba, Cit, d-Trp, d-Tza, Lys, Lys(Ac), Leu, Mle, Mly, Gln, Mkdm, and Thp,
   X11 is an amino acid residue selected from a residue of Asp, Bal, Gab, Glu, Kme, Lys, and Orn, and is linked via an amide bond to X1,
   X12 is absent or is an amino acid residue selected from a residue of Asn, Gly, Ser and Ala,
   X13 is absent or is an amino acid residue selected from a residue of Asn, Ala, Bal, Gly, Leu, Ile, Iva, Hol, Hph, Pal, Pyal, PyEA, His, Hme, Val, and their corresponding d-forms,
   X14 is absent or is an amino acid residue selected from a residue of Mep, Sar, Bal, Ile, Dnmy, and Hme,
   X15 is absent or is an amino acid residue of Lys,
   R2 is absent or is —NH₂, —OH or —N(C₂H₅)₂,
and
wherein amide bonds between X1 and X2, X2 and X3, X3 and X4, X4 and X5, X11 and X1 are optionally methylated,
or a salt or solvate thereof.

Compounds of formula (I) inhibit the binding of interleukin-23 to an interleukin-23-receptor.

In a further aspect of any peptide described herein, the compounds may comprise one or more half-life extension moiety and/or one or more linker moiety linked to the peptide.

In a further aspect, compounds of the disclosure may be of formula (I) as detailed thereafter.

In a further aspect, compositions comprising a compound of the formula (I) or (Ia) as described herein, or a salt or solvate thereof, in admixture with a carrier is disclosed.

The disclosure also relates to the use of a compound of the formula (I) or of formula (Ia) as described herein for use as a medicament, for example for the treatment of a condition as described in the specification.

The disclosure also relates to a composition wherein the composition is a pharmaceutically acceptable composition, and the carrier is a pharmaceutically acceptable carrier.

The disclosure also relates to a pharmaceutical composition comprising a compound of the formula (I) or of formula (Ia) as described herein and at least a pharmaceutically acceptable carrier.

The disclosure also relates to a pharmaceutical composition comprising at least one compound of the disclosure, or a physiological acceptable salt or solvate thereof, and at least a pharmaceutically acceptable carrier, for use as a pharmaceutical.

### Definitions

Amino acids. Amino acids are referred to herein by either their name, their commonly known three letter symbols or by the one-letter symbols recommended by the IUPAC-IUB Biochemical Nomenclature Commission. Therefore, the amino acid sequences disclosed herein contain the conventional one letter and three letter codes for naturally occurring amino acids, as well as generally accepted three letter codes for other amino acids, such as Aib for α-aminoisobutyric acid, Orn for ornithine or Ahx for 6-aminohexanoic acid.

The term “amino acid” or “any amino acid” as used here refers to any and all amino acids, including naturally occurring amino acids (e.g., a-amino acids), unnatural amino acids, modified amino acids, and non-natural amino acids. It includes both D- and L-amino acids. Natural amino acids include those found in nature, such as, e.g., the 22 amino acids that combine into peptide chains to form the building-blocks of a vast array of proteins. These are primarily L stereoisomers, although a few D-amino acids occur in bacterial envelopes and some antibiotics.

Unnatural or non-natural. “Unnatural” or “non-natural” amino acids are non-proteinogenic amino acids (i.e., those not naturally encoded or found in the genetic code) that either occur naturally or are chemically synthesized. Over 140 unnatural amino acids are known and thousands of more combinations are possible. Examples of “unnatural” amino acids include β-amino acids (β and β2), homo-amino acids, proline and pyruvic acid derivatives, 3-substituted alanine derivatives, glycine derivatives, ring-substituted phenylalanine and tyrosine derivatives, linear core amino acids, diamino acids, D-amino acids, alpha-methyl amino acids and N-methyl amino acids. Unnatural or non-natural amino acids also include modified amino acids. “Modified” amino acids include amino acids (e.g., natural amino acids) that have been chemically modified to include a group, groups, or chemical moiety not naturally present on the amino acid.

Residue. Within the disclosure, the term “residue” intends to refer to a building block within a peptide compound, which results from the binding of a natural amino acid, or of an unnatural amino acid, or of a compound such as Buty or Butyl.

“To inhibit the binding of interleukin-23 to an Interleukin-23-receptor”. The expression “to inhibit the binding of interleukin-23 to an interleukin-23-receptor” intends to refer to a property of a peptide compound of the disclosure to prevent the binding of the IL-23 receptor by its ligand IL-23. This property is measured as a binding affinity and can be expressed as the “IC₅₀ value”, which is the effective concentration of a compound that induces a half maximal decrease of the binding of IL-2.3 to its receptor in a dose-response experiment. A compound of the disclosure has a binding affinity to the interleukin 23 receptor of 100 nM or below (i.e., IC₅₀≤100 nM).

Furthermore Table 1 shows the codes used for amino acids and further compounds.

TABLE 1
Natural and unnatural amino acids and other compounds

| Abbreviation | Name | Structure |
|---|---|---|
| Abu | L-2-Aminobutyric acid | 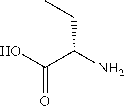 |
| Ac | acetic acid | |
| Aib | 2-aminoisobutyric acid | 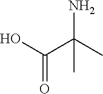 |
| Ahx | 6-aminohexanoic acid | 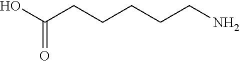 |
| Ala | L-alanine 2-Aminopropanoic acid | 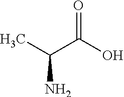 |
| Aoa | 8-aminooctanoic acid | 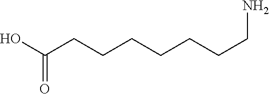 [SMILES: NCCCCCCCC(=O)O] |
| Apt | 5-aminopentanoic acid | 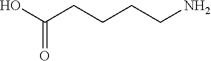 |
| Arn | L-arginine 2-Amino-5-guanidinopentanoic acid | 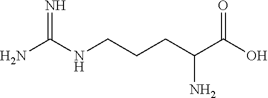 [SMILES: N=C(N)NCCCC(N)C(=O)O] |
| Asn | L-asparagine 2-Amino-3-carbarnoylpropanoic acid | 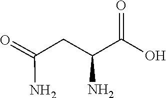 |
| Asp | L-aspartic acid 2-aminobutanedioic acid | 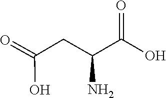 |
| Bal | beta-alanine | 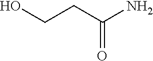 |
| Butyl | butane | 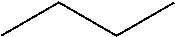 |
| Buty | butyric acid | 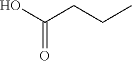 |
| Cba | β-cyclobutyl-L-alanine | 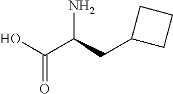 |
| Cit | L-citrulline | 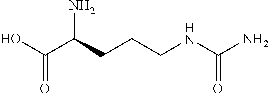 [SMILES: NC(=O)NCCC[C@H](N)C(=O)O] |
| Cys | L-cysteine 2-amino-3-sulfanylpropanoic acid | 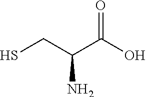 |
| Dab | (2S)-2,4-diaminobutanoic acid | 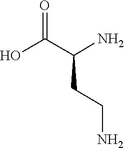 |
| Dad | succinic acid | 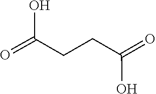 |
| Dap | (2S)-2,3-diamino propionic acid | 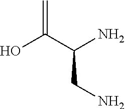 |
| Dnmy | N-methyl-D-tyrosine | 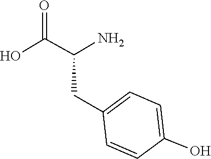 [SMILES: N[C@H](Cc1ccc(O)cc1)C(=O)O] |
| Gab | 4-aminobutyric acid | 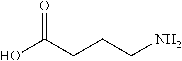 |
| Gln | L-glutamine 2-Amino-4-carbamoylbutanoic acid | 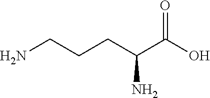 |
| Glu | L-glutamic acid 2-aminopentanedioic acid | 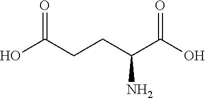 |
| Gly | L-glycine 2-Aminoethanoic acid | 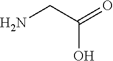 |
| His | L-histidine 2-amino-3-(1H-imidazol-5-yl)propanoic acid | 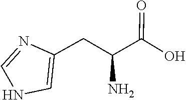 [SMILES: N[C@@H](Cc1c[nH]cn1)C(=O)O] |
| Hme | 3-methyl-L-histidine | 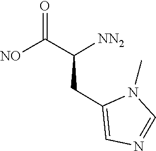 |
| Hol | homo-L-leucine | 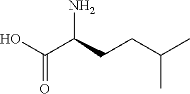 |
| Hph | homo-L-phenylalanine | 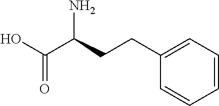 [SMILES: N[C@@H](CCc1ccccc1)C(=O)O] |
| Ile | L-isoleucine 2-amino-3-methylpentanoic acid | 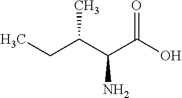 |
| Iva | Isovaline | 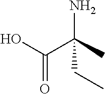 |
| Kme | ε-methyl-L-lysine | 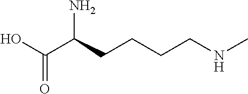 |
| Leu | L-leucine 2-Amino-4-methylpentanoic acid | 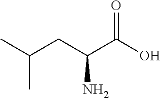 |
| Lys | L-lysine 2,6-diaminohexanoic acid | 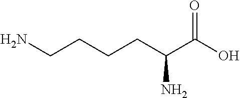 |
| Lys(Ac) K[Ac] | ε-acetyl-L-lysine | 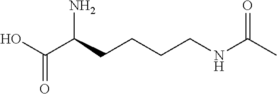 |
| Lys(αAc) K[aAc] | (2S)-2-acetamido-6-amino-hexanoic acid | 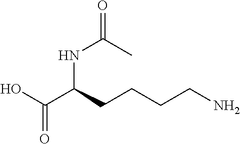 |
| Mep | α-methyl-L-proline | 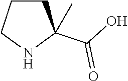 |
| Mkdm | (2S)-2-amino-6-(dimethylamino)-2-methyl- hexanoic acid | 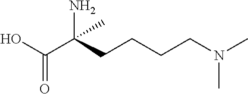 |
| Mle | α-methyl-L-leucine | 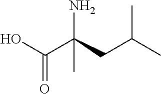 |
| Mly | α-methyl-L-lysine | 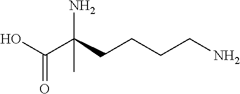 |
| Mva | α-methyl-L-valine | 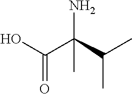 |
| Myri | myristic acid | 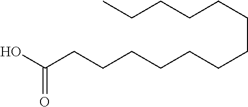 [SMILES: CCCCCCCCCCCCCC(=O)O] |
| Nal | 2-naphthyl-L-alanine | 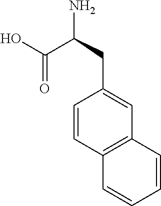 [SMILES: N[C@@H](Cc1ccc2ccccc2c1)C(=O)O] |
| Nak | 1-naphthyl-L-alanine | 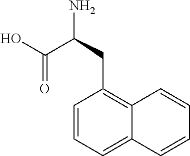 [SMILES: N[C@@H](Cc1cccc2ccccc12)C(=O)O] |
| Ndmk | (2S)-6-amino-2-(dimethylamino)hexanoic acid | 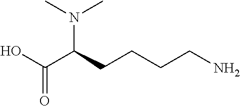 |
| Nmapt | 5-(methylamino)pentanoic acid | 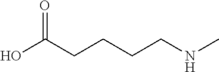 |
| Nmc | N-methyl-L-cysteine | 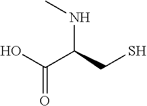 |
| Nmd | N-methyl-L-aspartic acid | 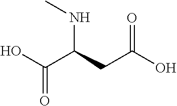 |
| Nme | N-methyl-L-glutamic acid | 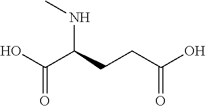 |
| Nmha | 6-(methylamino)hexanoic acid | 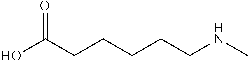 |
| Nmn | N-methyl-L-asparagine | 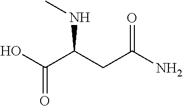 |
| Nmt | N-methyl-L-threonin | 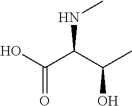 |
| Ntmk | (2S)-2-(dimethylamino)-6- (methylamino)hexanoic acid | 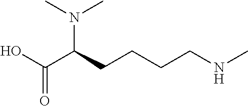 |
| Ornithine | L-Ornithine 2,5-Diaminopentanoic acid | 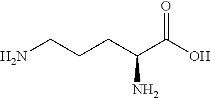 |
| Pal | L-3-pyridyl-alanine | 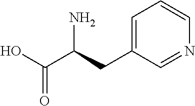 [SMILES: N[C@@H](Cc1cccnc1)C(=O)O] |
| Palm | palmitic acid | |
| Pen | 2-amino-3-methyl-3-sulfanylbutanoic acid | 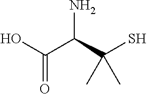 |
| Phe | L-phenylalanine 2-Amino-3-phenylpropanoic acid | 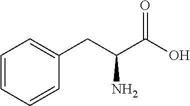 [SMILES: N[C@@H](Cc1ccccc1)C(=O)O] |
| Pipa | 2-(4-piperidyl)acetic acid | 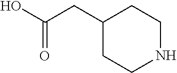 |
| Pipb | 4-(4-piperidyl) butanoic acid | 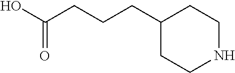 [SMILES: O=C(O)CCCC1CCNCC1] |
| Pipp | 3-(4-piperidyl) propanoic acid | 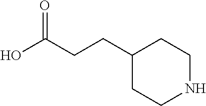 [SMILES: O=C(O)CCC1CCNCC1] |
| Pyal | L-5-pyrimidine-alanine | 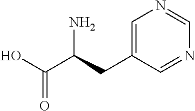 [SMILES: N[C@@H](Cc1cncnc1)C(=O)O] |
| PyEA | 2-(3-pyridyl)ethanamine | 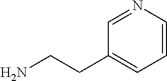 |
| Sar | Sarkosin | 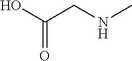 |
| Ser | L-serine 2-Amino-3-hydroxypropanoic acid | 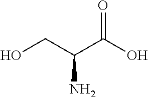 |
| Sema | 18-oxo-18-[(1S)-1-carboxy-4-[2-[2-[2-[2- | |
| | [2-(carboxymethoxy)ethoxy]ethylamino]-2- | |
| | oxo-ethoxy]ethoxy]ethylamino]-4-oxo- | |
| | butyl]amino]octadecanoic acid | |
| Thp | 4-amino-tetrahydropyran-4-carboxylic acid | 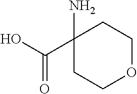 |
| Thr | L-threonine 2-amino-3-hydroxybutanoic acid | 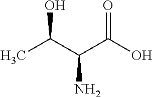 |
| Trp | L-tryptophan 2-amino-3-(1H-indol-3-yl)propanoic acid | 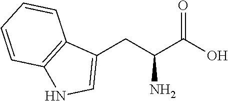 [SMILES: N[C@@H](Cc1c[nH]c2ccccc12)C(=O)O] |
| Tyr | L-tyrosine L-2-Amino-3-(4-hydroxyphenyl)propanoic acid | 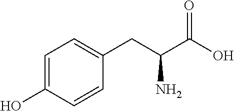 [SMILES: N[C@@H](Cc1ccc(O)cc1)C(=O)O] |
| Tza | β-(4-thiazolyl)-alanine | 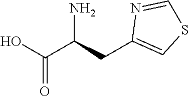 [SMILES: N[C@@H](Cc1cscn1)C(=O)O] |
| Val | L-valine 2-Amino-3-methylbutanoic acid | 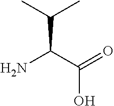 |
| Wcl | 5-chloro-L-tryptophan | 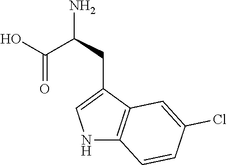 [SMILES: N[C@@H](Cc1c[nH]c2ccc(Cl)cc12)C(=O)O] |
| Wdm | (2S)-2-amino-3-(1,7-dimethylindol-3- yl)propanoic acid | 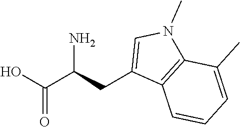 [SMILES: Cc1cccc2c(C[C@H](N)C(=O)O)cn(C)c12] |
| Wfi | 5-fluoro-L-tryptophan | 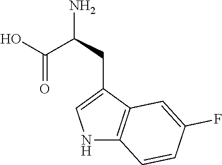 [SMILES: N[C@@H](Cc1c[nH]c2ccc(F)cc12)C(=O)O] |
| Wim | 7-methyl-L-tryptophan | 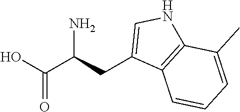 [SMILES: Cc1cccc2c(C[C@H](N)C(=O)O)c[nH]c12] |
| Wme | 1-methyl-L-tryptophan | 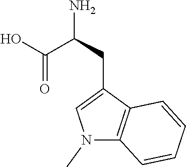 [SMILES: Cn1cc(C[C@H](N)C(=O)O)c2ccccc21] |
| Yae | 4-aminoethoxy-L-phenylalanine | 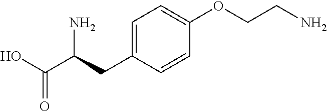 [SMILES: NCCOc1ccc(C[C@H](N)C(=O)O)cc1] |
| Yde | (2S)-2-amino-3-[4-[2- (dimethylamino)ethoxy]phenyl]propanoic acid | 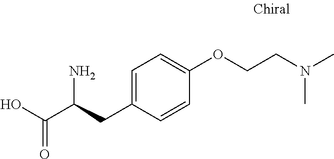 [SMILES: CN(C)CCOc1ccc(C[C@H](N)C(=O)O)cc1] |
| Yme | 4-methoxy-L-phenylalanine | 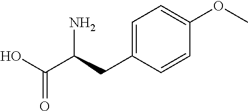 |

### DETAILED DESCRIPTION

### Peptide Compounds

### Compounds of Formula (I)

Disclosed herein are compounds of formula (I):

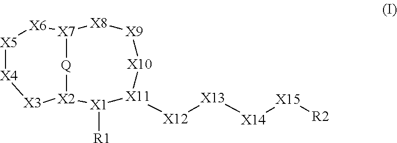

wherein
   X1 is a residue selected from a residue of Ahx, Aoa, Apt, Nmha, Nmapt, Lys, Pipa, Pipb, Pipp, Dab, Dad and Dap, and is linked via an amide bond to X11,
or is a residue selected from a residue of Buty and Butyl, and is linked via an alkyl bond to X11 and when X1 is a residue of Butyl, the residue of Butyl is linked to X2 via an alkyl bond,
   R1 is absent or, if X1 is selected from a residue of Ahx, Aoa, Apt, Lys, Dab and Dap, then R1 is H, C₁-C₄-alkyl or acetyl, and is linked to an amino function of X1,
   X2 is an amino acid residue selected from a residue of Cys, Glu, Asp, Nmc, Nmd, Nme, Nmt, Thr, and Abu, and their corresponding d-forms. In some embodiments, X2 is an amino acid residue selected from a residue of Cys, Glu, Asp, Nmc, Nmd, Nme, Nmt, and Thr, and their corresponding d-forms,
   X7 is an amino acid residue selected from a residue of Cys, Glu, Asp, Thr, and Abu, and their corresponding d-forms,
wherein X2 and X7 are linked to each other via Q, wherein Q is a disulfide bond, a thioether bond or an ester bond,
   X3 is an amino acid residue selected from a residue of Sar, His, Lys, Ala, Ahx, Apt, Arg, Asn, Gab, Gin, Trp, Thr, Ndmk, Nmn, Ntmk, and Lys(αAc),
   X4 is absent, or is an amino acid residue selected from a residue of His, Lys, Ahx, Apt, Arg, Asn, Gab, Gln, Trp, and Thr,
   X5 is an amino acid residue selected from a residue of Trp, Wme, Nak, Wcl, Wim, Wdm, and Wfl,
   X6 is an amino acid residue selected from a residue of Gln, Aib, Glu, Phe, Ile, Iva, Lys, Lys(Ac), Leu, Mle, Mly, Mva, Thr, Val, Trp, and Phe,
   X8 is an amino acid residue selected from a residue of Yme, Tyr, Trp, Yde, and Yae. In some embodiments, X8 is an amino acid residue selected from a residue of Yme, Tyr, Trp, and Yde,
   X9 is an amino acid residue selected from a residue of Nal, Nak, and Trp,
   X10 is an amino acid residue selected from a residue of Aib, Cba, Cit, d-Trp, d-Tza, Lys, Lys(Ac), Leu, Mle, Mly, Gln, Mkdm, and Thp,
   X11 is an amino acid residue selected from a residue of Asp, Bal, Gab, Glu, Kme, Lys, and Orn, and is linked via an amide bond to X1,
   X12 is absent or is an amino acid residue selected from a residue of Asn, Gly, Ser and Ala,
   X13 is absent or is an amino acid residue selected from a residue of Asn, Ala, Bal, Gly, Leu, Ile, Iva, Hol, Hph, Pal, Pyal, PyEA, His, Hme, Val, and their corresponding d-forms,
   X14 is absent or is an amino acid residue selected from a residue of Mep, Sar, Bal, Ile, Dnmy, and Hme,
   X15 is absent or is an amino acid residue of Lys,
   R2 is absent or is —NH₂, —OH or —N(C₂H₅)₂,
and
wherein amide bonds between X1 and X2, X2 and X3, X3 and X4, X4 and X5, X11 and X1 are optionally methylated,
or a salt or solvate thereof.

Compounds of formula (I) inhibit the binding of interleukin-23 to an interleukin-23-receptor.

In some embodiments, a compound of formula (I) may be such that X2 is:
either an amino acid residue selected from an amino acid residue of Cys, Glu, Asp, Nmc, Nmd, Nme, Nmt, Thr and Abu, and their corresponding d-forms, and X8 is an amino acid residue selected from an amino acid residue of Yme, Tyr, Trp, and Yde,
or an amino acid residue selected from an amino acid residue of Cys, Glu, Asp, Nmc, Nmd, Nme, Nmt, and Thr, and their corresponding d-forms, and X8 is an amino acid residue selected from an amino acid residue of Yme, Tyr, Trp, Yde and Yae.

In some embodiments, a compound of formula (I) may be such that when X2 is Abu, or its corresponding d-forms, then X8 is not an amino acid residue of Yae.

In some embodiments, a compound of formula (I) may be such that when X8 is Yae, then X2 is not an amino acid residue of Abu, or its corresponding d-forms.

In a first group of embodiments, a compound of formula (I) is such that:
X1 is a residue of Ahx, Aoa, Apt, Nmha, Nmapt, Lys, Pipa, Pipb, Pipp, Dab, Dad or Dap, and is linked via an amide bond to X11,
or is a residue of Buty or Butyl, and is linked via an alkyl bond to X11 and when X1 is a residue of Butyl, the residue of Butyl is linked to X2 via an alkyl bond,
R1 is absent, or
if X1 is a residue of Ahx, Aoa, Apt, Lys, Dab or Dap, then R1 is H, C₁-C₄-alkyl or acetyl, and is linked to an amino function of X1,
X2 is an amino acid residue of Cys, Glu, Asp, Nmc, Nmd, Nme, Nmt, Thr or Abu, and their corresponding d-forms,
X7 is an amino acid residue of Cys, Glu, Asp, Thr or Abu, and their corresponding d-forms,
wherein X2 and X7 are linked to each other via Q, wherein Q is a disulfide bond, a thioether bond or an ester bond,
X3 is an amino acid residue of Sar, His, Lys, Ala, Ahx, Apt, Arg, Asn, Gab, Gln, Trp, Thr, Ndmk, Nmn, Ntmk or Lys(αAc),
X4 is absent, or is an amino acid residue of His, Lys, Ahx, Apt, Arg, Asn, Gab, Gln, Trp or Thr,
X5 is an amino acid residue of Trp, Wme, Nak, Wcl, Wim, Wdm or Wfl,
X6 is an amino acid residue of Gln, Aib, Glu, Phe, Ile, Iva, Lys, Lys(Ac), Leu, Mle, Mly, Mva, Thr, Val, Trp or Phe,
X8 is an amino acid residue of Yme, Tyr, Trp, or Yde,
X9 is an amino acid residue of Nal, Nak or Trp,
X10 is an amino acid residue of Aib, Cba, Cit, d-Trp, d-Tza, Lys, Lys(Ac), Leu, Mle, Mly, Gln, Mkdm or Thp,
X11 is an amino acid residue of Asp, Bal, Gab, Glu, Kme, Lys or Orn, and is linked via an amide bond to X1,
X12 is absent or is an amino acid residue of Asn, Gly, Ser or Ala,
X13 is absent or is an amino acid residue of Asn, Ala, Bal, Gly, Leu, Ile, Iva, Hol, Hph, Pal, Pyal, PyEA, His, Hme, or Val, and their corresponding d-forms,
X14 is absent or is an amino acid residue of Mep, Sar, Bal, Ile, Dnmy or Hme,
X15 is absent or is an amino acid residue of Lys,
R2 is absent or is —NH₂, —OH or —N(C₂H₅)₂,
and
wherein amide bonds between X1 and X2, X2 and X3, X3 and X4, X4 and
X5, X11 and X1 are optionally methylated, or a salt or solvate thereof.

In such group of embodiments, compounds of formula (I) inhibit the binding of interleukin-23 to an interleukin-23-receptor.

In some embodiments of the first group of embodiments, compounds of formula (I) may be such that:
X1 is a residue selected from a residue of Ahx, Apt, Nmha, Nmapt, Lys and Dad, and is linked via an amide bond to X11,
or is a residue selected from a residue of Buty and Butyl, and is linked via an alkyl bond to X11 and when X1 is a residue of Butyl, the residue of Butyl is linked to X2 via an alkyl bond,
R1 is absent, or
if X1 is a residue selected from a residue of Ahx, Apt and Lys, then R1 is H, C₁-C₄-alkyl or acetyl, and is linked to an amino function of X1,
X2 is an amino acid residue selected from a residue of Cys, Glu, Asp, Nmc, Nmd, Nme, Nmt, Thr and Abu,
X7 is an amino acid residue selected from a residue of Cys, Glu, Asp, Thr and Abu,
wherein X2 and X7 are linked to each other via Q, wherein Q is a disulfide bond, a thioether bond or an ester bond,
X3 is an amino acid residue selected from a residue of Apt, Asn, Ndmk, Nmn and Lys(αAc),
X4 is absent or is an amino acid residue of Thr,
X5 is an amino acid residue selected from a residue of Trp, Wme and Wim,
X6 is an amino acid residue selected from a residue of Gln, Lys(Ac) and Leu,
X8 is an amino acid residue selected from a residue of Yme, Trp, and Yde,
X9 is an amino acid residue of Nal,
X10 is an amino acid residue selected from a residue of Aib, Lys, Lys(Ac), Mle, Mly, Mkdm and Thp,
X11 is an amino acid residue selected from a residue of Asp, Glu, Kme, Lys and Orn, and is linked via an amide bond to X1,
X12 is absent or is an amino acid residue selected from a residue of Asn, Gly, Ser and Ala,
X13 is absent or is an amino acid residue selected from a residue of Asn,
X14 is absent or is an amino acid residue selected from a residue of Mep, Sar, Bal, Ile and Hme,
X15 is absent or is an amino acid residue of Lys,
R2 is absent or is —NH2, —OH or —N(C2H5)2,
or a salt or solvate thereof.

In a second group of embodiments, a compound of formula (I) is such that:
X1 is a residue selected from a residue of Ahx, Aoa, Apt, Nmha, Nmapt, Lys, Pipa, Pipb, Pipp, Dab, Dad and Dap, and is linked via an amide bond to X11,
or is a residue selected from a residue of Buty and Butyl, and is linked via an alkyl bond to X11 and when X1 is a residue of Butyl, the residue of Butyl is linked to X2 via an alkyl bond,
R1 is absent, or
if X1 is a residue selected from a residue of Ahx, Aoa, Apt, Lys, Dab and Dap, then R1 is H, C₁-C₄-alkyl or acetyl, and is linked to an amino function of X1,
X2 is an amino acid residue selected from a residue of Cys, Glu, Asp, Nmc, Nmd, Nme, Nmt, and Thr, and their corresponding d-forms,
X7 is an amino acid residue selected from a residue of Cys, Glu, Asp, Thr and Abu, and their corresponding d-forms,
wherein X2 and X7 are linked to each other via Q, wherein Q is a disulfide bond, a thioether bond or an ester bond,
X3 is an amino acid residue selected from a residue of Sar, His, Lys, Ala, Ahx, Apt, Arg, Asn, Gab, Gln, Trp, Thr, Ndmk, Nmn, Ntmk and Lys(αAc),
X4 is absent, or is an amino acid residue selected from a residue of His, Lys, Ahx, Apt, Arg, Asn, Gab, Gln, Trp and Thr,
X5 is an amino acid residue selected from a residue of Trp, Wme, Nak, Wol, Wim, Wdm and Wil,
X6 is an amino acid residue selected from a residue of Gin, Aib, Glu, Phe, Ile, Iva, Lys, Lys(Ac), Leu, Mle, Mly, Mva, Thr, Val, Trp and Phe,
X8 is an amino acid residue selected from a residue of Yme, Tyr, Trp, Yde and Yae,
X9 is an amino acid residue selected from a residue of Nal, Nak and Trp,
X10 is an amino acid residue selected from a residue of Aib, Cba, Cit, d-Trp, d-Tza, Lys, Lys(Ac), Leu, Mle, Mly, Gin, Mkdm and Thp,
X11 is an amino acid residue selected from a residue of Asp, Bal, Gab, Glu, Kme, Lys and Orn, and is linked via an amide bond to X1,
X12 is absent or is an amino acid residue selected from a residue of Asn, Gly, Ser and Ala,
X13 is absent or is an amino acid residue selected from a residue of Asn, Ala, Bal, Gly, Leu, Ile, Iva, Hol, Hph, Pal, Pyal, PyEA, His, Hme, Val, and their corresponding d-forms,
X14 is absent or is an amino acid residue selected from a residue of Mep, Sar, Bal, Ile, Dnmy and Hme,
X15 is absent or is an amino acid residue of Lys,
R2 is absent or is —NH₂, —OH or —N(C₂H₅)₂,
and
wherein amide bonds between X1 and X2, X2 and X3, X3 and X4, X4 and X5, X11 and X1 are optionally methylated,
or a salt or solvate thereof.

In such group of embodiments, compounds of formula (I) inhibit the binding of interleukin-23 to an interleukin-23-receptor.

In some embodiments of the second group of embodiments, compounds of formula (I) may be such that:
X1 is a residue selected from a residue of Ahx, Apt, Nmha, Nmapt, Lys and Dad, and is linked via an amide bond to X11,
or is a residue selected from a residue of Buty and Butyl, and is linked via an alkyl bond to X11 and when X1 is a residue of Butyl, the residue of Butyl is linked to X2 via an alkyl bond,
R1 is absent, or
if X1 is a residue selected from a residue of Ahx, Apt and Lys, then R1 is H, C₁-C₄-alkyl or acetyl, and is linked to an amino function of X1,
X2 is an amino acid residue selected from a residue of Cys, Glu, Asp, Nmc, Nmd, Nme, Nmt, and Thr,
X7 is an amino acid residue selected from a residue of Cys, Glu, Asp, Thr and Abu,
wherein X2 and X7 are linked to each other via Q, wherein Q is a disulfide bond, a thioether bond or an ester bond,
X3 is an amino acid residue selected from a residue of Apt, Asn, Ndmk, Nmn and Lys(αAc),
X4 is absent or is an amino acid residue of Thr,
X5 is an amino acid residue selected from a residue of Trp, Wme and Wim,
X6 is an amino acid residue selected from a residue of Gln, Lys(Ac) and Leu,
X8 is an amino acid residue selected from a residue of Yme, Trp, Yde and Yae,
X9 is an amino acid residue of Nal,
X10 is an amino acid residue selected from a residue of Aib, Lys, Lys(Ac), Mle, Mly, Mkdm and Thp,
X11 is an amino acid residue selected from a residue of Asp, Glu, Kme, Lys and Orn, and is linked via an amide bond to X1,
X12 is absent or is an amino acid residue selected from a residue of Asn, Gly, Ser and Ala,
X13 is absent or is an amino acid residue selected from a residue of Asn, Ala, Bal, Gly, d-Leu, Ile, Pal, Pyal, PyEA, His and Hme,
X14 is absent or is an amino acid residue selected from a residue of Mep, Sar, Bal, Ile and Hme,
X15 is absent or is an amino acid residue Lys,
R2 is absent or is —NH₂, —OH or —N(C₂H₅)₂,
or a salt or solvate thereof.

Embodiments of compounds of formula (I) are those in which R1 is absent.

Embodiments of compounds of formula (I) are those in which R1 is H.

Embodiments of compounds of formula (I) are those in which R1 is C1-C4-alkyl.

Embodiments of compounds of formula (I) are those in which R1 is acetyl.

Embodiments of compounds of formula (I) are those in which R2 is absent.

Embodiments of compounds of formula (I) are those in which R2 is —NH₂.

Embodiments of compounds of formula (I) are those in which R2 is —OH.

Embodiments of compounds of formula (I) are those in which R2 is —N(C₂H₅)₂.

An embodiment of compounds of formula (I) are those in which:
X1 is a residue selected from a residue of Ahx, Aoa, Apt, Nmha, Nmapt, Lys, Pipa, Pipb, Pipp, Dab, and Dap, and X11 is an amino acid residue selected from a residue of Asp and Glu,
or
X1 is a residue of Dad and X11 is an amino acid residue selected from a residue of Lys and Kme,
or
X1 is a residue selected from a residue of Buty and Butyl and X11 is an amino acid residue of Kme.

Embodiments of compounds of formula (I) are those in which X1 is a residue of Ahx, and X11 is an amino acid residue selected from a residue of Glu or Asp.

Embodiments of compounds of formula (I) are those in which X1 is a residue of Apt, and X11 is an amino acid residue selected from a residue of Glu or Asp.

Embodiments of compounds of formula (I) are those in which X1 is a residue of Apt, and X11 is an amino acid residue of Glu.

Embodiments of compounds of formula (I) are those in which X1 is a residue of Nmapt and X11 is an amino acid residue selected from a residue of Glu or Asp.

Embodiments of compounds of formula (I) are those in which X1 is a residue of Nmapt and X11 is an amino acid residue of Glu.

Embodiments of compounds of formula (I) are those in which X1 is a residue of Nmapt and X11 is an amino acid residue of Asp.

Embodiments of compounds of formula (I) are those in which X1 is a residue of Nmha and X11 is an amino acid residue selected from a residue of Glu or Asp.

Embodiments of compounds of formula (I) are those in which X1 is a residue of Nmha and X11 is an amino acid residue of Glu.

Embodiments of compounds of formula (I) are those in which X1 is a residue of Nmha and X11 is an amino acid residue of Asp.

Embodiments of compounds of formula (I) are those in which X1 is a residue of Lys and X11 is an amino acid residue selected from a residue of Glu or Asp.

Embodiments of compounds of formula (I) are those in which X1 is a residue of Lys and X11 is an amino acid residue of Glu.

Embodiments of compounds of formula (I) are those in which X1 is a residue of Lys and X11 is an amino acid residue of Asp.

Embodiments of compounds of formula (I) are those in which X1 is a residue of Dad and X11 is an amino acid residue selected from a residue of Lys or Kme.

Embodiments of compounds of formula (I) are those in which X1 is a residue of Dad and X11 is an amino acid residue of Lys.

Embodiments of compounds of formula (I) are those in which X1 is a residue of Dad and X11 is an amino acid residue of Kme.

Embodiments of compounds of formula (I) are those in which X1 is a residue of Buty and X11 is an amino acid residue selected from a residue of Lys or Kme.

Embodiments of compounds of formula (I) are those in which X1 is a residue of Buty and X11 is an amino acid residue of Lys.

Embodiments of compounds of formula (I) are those in which X1 is a residue of Buty and X11 is an amino acid residue of Kme.

Embodiments of compounds of formula (I) are those in which X1 is a residue of Butyl and X11 is an amino acid residue selected from a residue of Lys or Kme.

Embodiments of compounds of formula (I) are those in which X1 is a residue of Butyl and X11 is an amino acid residue of Lys.

Embodiments of compounds of formula (I) are those in which X1 is a residue of Butyl and X11 is an amino acid residue of Kme.

In some embodiments of the first group of embodiments, X2 is an amino acid residue selected from a residue of Cys, Glu, Asp, Nmc, Nmd, Nme, Nmt, Thr and Abu, and their corresponding d-forms.

In some embodiments of the second group of embodiments, X2 is an amino acid residue selected from a residue of Cys, Glu, Asp, Nmc, Nmd, Nme, Nmt, and Thr, and their corresponding d-forms.

Embodiments of compounds of formula (I) are those in which X2 is an amino acid residue selected from a residue of Cys and Nmc, and their corresponding d-forms,
X7 is an amino acid residue selected from a residue of Cys and its corresponding d-form,
wherein X2 and X7 are linked to each other via Q, wherein Q is a disulfide bond,
or
X2 is an amino acid residue selected from a residue of Cys, Nmc, Thr, and Abu and their corresponding d-forms,
X7 is an amino acid residue selected from a residue of Cys, Thr, and Abu and their corresponding d-forms,
wherein X2 and X7 are linked to each other via Q, wherein Q is a thioether bond,
or
X2 is an amino acid residue selected from a residue of Glu, Asp, Nmd, Nme, Nmt and Thr and their corresponding d-forms,
X7 is an amino acid residue selected from a residue of Glu, Asp, Thr and their corresponding d-forms,
wherein X2 and X7 are linked to each other via Q, wherein Q is an ester bond.

Embodiments of compounds of formula (I) are those in which X2 is an amino acid residue selected from a residue of Cys and its corresponding d-form,
X7 is an amino acid residue selected from a residue of Cys and its corresponding d-form,
wherein X2 and X7 are linked to each other via Q, wherein Q is a disulfide bond,
or
X2 is an amino acid residue selected from a residue of Cys, Glu, Thr, and Abu and their corresponding d-forms,
X7 is an amino acid residue selected from a residue of Cys, Thr, and Abu and their corresponding d-form,
wherein X2 and X7 are linked to each other via Q, wherein Q is a thioether bond,
or
X2 is an amino acid residue selected from a residue of Glu and Thr and their corresponding d-forms,
X7 is an amino acid residue selected from a residue of Glu, Asp and Thr and their corresponding d-forms,
wherein X2 and X7 are linked to each other via Q, wherein Q is an ester bond.

Embodiments of compounds of formula (I) are those in which X2-Q-X7 is selected from a group of:
Cys-Q-Cys, Nmc-Q-Cys and Cys-Q-Nmc, wherein Q is a disulfide bond, or
Abu-Q-Cys, Cys-Q-Abu, wherein Q is a thioether bond, or
Thr-Q-Asp, Thr-Q-Glu, Glu-Q-Thr, wherein Q is an ester bond.

In some embodiments of the first group of embodiments, compounds of formula (I) are those in which X2-Q-X7 is selected from a group of:
Cys-Q-Cys, Nmc-Q-Cys and Cys-Q-Nmc, wherein Q is a disulfide bond, or
Abu-Q-Cys, Cys-Q-Abu, wherein Q is a thioether bond, or
Thr-Q-Asp, Thr-Q-Glu, Glu-Q-Thr, wherein Q is an ester bond.

In some embodiments of the second group of embodiments, compounds of formula (I) are those in which X2-Q-X7 is selected from a group of:
Cys-Q-Cys, Nmc-Q-Cys and Cys-Q-Nmc, wherein Q is a disulfide bond, or
Cys-Q-Abu, wherein Q is a thioether bond, or
Thr-Q-Asp, Thr-Q-Glu, Glu-Q-Thr, wherein Q is an ester bond.

Embodiments of compounds of formula (I) are those in which X2-Q-X7 is selected from a group of Cys-Q-Cys and Nmc-Q-Cys, wherein Q is a disulfide bond, or Abu-Q-Cys wherein Q is a thioether bond, or Glu-Q-Thr, wherein Q is an ester bond.

Embodiments of compounds of formula (I) are those in which X2-Q-X7 is selected from a group of Cys-Q-Cys and Nmc-Q-Cys, wherein Q is a disulfide bond.

Embodiments of compounds of formula (I) are those in which X2-Q-X7 is Abu-Q-Cys wherein Q is a thioether bond.

Embodiments of compounds of formula (I) are those in which X2-Q-X7 is Glu-Q-Thr, wherein Q is an ester bond.

Embodiments of compounds of formula (I) are those in which X3 is an amino acid residue selected from a residue of Apt, Ndmk, Nmn and Lys(αAc).

Embodiments of compounds of formula (I) are those in which X3 is an amino acid residue of Sar.

Embodiments of compounds of formula (I) are those in which X3 is an amino acid residue of His.

Embodiments of compounds of formula (I) are those in which X3 is an amino acid residue of Lys.

Embodiments of compounds of formula (I) are those in which X3 is an amino acid residue of Ala.

Embodiments of compounds of formula (I) are those in which X3 is an amino acid residue of Ahx.

Embodiments of compounds of formula (I) are those in which X3 is an amino acid residue of Apt.

Embodiments of compounds of formula (I) are those in which X3 is an amino acid residue of Arg.

Embodiments of compounds of formula (I) are those in which X3 is an amino acid residue of Asn.

Embodiments of compounds of formula (I) are those in which X3 is an amino acid residue of Gab.

Embodiments of compounds of formula (I) are those in which X3 is an amino acid residue of Gin.

Embodiments of compounds of formula (I) are those in which X3 is an amino acid residue of Trp.

Embodiments of compounds of formula (I) are those in which X3 is an amino acid residue of Thr.

Embodiments of compounds of formula (I) are those in which X3 is an amino acid residue of Ndmk,

Embodiments of compounds of formula (I) are those in which X3 is an amino acid residue of Nmn.

Embodiments of compounds of formula (I) are those in which X3 is an amino acid residue of Ntmk.

Embodiments of compounds of formula (I) are those in which X3 is an amino acid residue of Lys(αAc).

Embodiments of compounds of formula (I) are those in which X4 is absent.

Embodiments of compounds of formula (I) are those in which X3 is an amino acid residue selected from a residue of Lys, Ahx, Apt, Thr, Ndmk, Nmn, Ntmk and Lys(αAc), and X4 is absent.

Embodiments of compounds of formula (I) are those in which X3 is an amino acid residue selected from a residue of Apt, Ndmk, Nmn and Lys(αAc), and X4 is absent.

Embodiments of compounds of formula (I) are those in which X3 is an amino acid residue selected from a residue of Apt, Ndmk and Lys(αAc), and X4 is absent.

Embodiments of compounds of formula (I) are those in which X3 is an amino acid residue of Lys(αAc), and X4 is absent.

Embodiments of compounds of formula (I) are those in which X4 is an amino acid residue selected from a residue of His, Lys, Arg, Asn, Gab, Gln, Trp and Thr.

Embodiments of compounds of formula (I) are those in which X4 is an amino acid residue selected from a residue of His, Lys, Arg, Asn, Gln, Trp and Thr.

Embodiments of compounds of formula (I) are those in which X4 is an amino acid residue of His.

Embodiments of compounds of formula (I) are those in which X4 is an amino acid residue of Lys.

Embodiments of compounds of formula (I) are those in which X4 is an amino acid residue of Ahx.

Embodiments of compounds of formula (I) are those in which X4 is an amino acid residue of Apt.

Embodiments of compounds of formula (I) are those in which X4 is an amino acid residue of Arg.

Embodiments of compounds of formula (I) are those in which X4 is an amino acid residue of Asn.

Embodiments of compounds of formula (I) are those in which X4 is an amino acid residue of Gab.

Embodiments of compounds of formula (I) are those in which X4 is an amino acid residue of Gin.

Embodiments of compounds of formula (I) are those in which X4 is an amino acid residue of Trp.

Embodiments of compounds of formula (I) are those in which X4 is an amino acid residue of Thr.

Embodiments of compounds of formula (I) are those in which X5 is an amino acid residue selected from a residue of Trp, Wme, Nak, Wcl, Wim, Wdm and Wfl.

Embodiments of compounds of formula (I) are those in which X5 is an amino acid residue selected from a residue of Trp, Wme, Wcl, Wim, Wdm and Wfl.

Embodiments of compounds of formula (I) are those in which X5 is an amino acid residue selected from Trp, and Wim.

Embodiments of compounds of formula (I) are those in which X5 is an amino acid residue of Trp.

Embodiments of compounds of formula (I) are those in which X5 is an amino acid residue of Wme.

Embodiments of compounds of formula (I) are those in which X5 is an amino acid residue of Nak.

Embodiments of compounds of formula (I) are those in which X5 is an amino acid residue of Wcl.

Embodiments of compounds of formula (I) are those in which X5 is an amino acid residue of Wim.

Embodiments of compounds of formula (I) are those in which X5 is an amino acid residue of Wdm.

Embodiments of compounds of formula (I) are those in which X5 is an amino acid residue of Wfl.

Embodiments of compounds of formula (I) are those in which X6 is an amino acid residue selected from a residue of Gin, Aib, Glu, Phe, Ile, Iva, Lys, Lys(Ac), Leu, Mle, Mly, Mva, Thr, Val, Trp and Phe.

Embodiments of compounds of formula (I) are those in which X6 is an amino acid residue selected from a residue of Gln, Aib, Glu, Ile, Iva, Lys, Lys(Ac), Leu, Mle, Mly, Mva, and Val.

Embodiments of compounds of formula (I) are those in which X6 is an amino acid residue selected from a residue of Gln, Lys(Ac) and Leu.

Embodiments of compounds of formula (I) are those in which X6 is an amino acid residue of Gin.

Embodiments of compounds of formula (I) are those in which X6 is an amino acid residue of Aib.

Embodiments of compounds of formula (I) are those in which X6 is an amino acid residue of Glu.

Embodiments of compounds of formula (I) are those in which X6 is an amino acid residue of Phe.

Embodiments of compounds of formula (I) are those in which X6 is an amino acid residue of Ile.

Embodiments of compounds of formula (I) are those in which X6 is an amino acid residue of Iva.

Embodiments of compounds of formula (I) are those in which X6 is an amino acid residue of Lys.

Embodiments of compounds of formula (I) are those in which X6 is an amino acid residue of Lys(Ac).

Embodiments of compounds of formula (I) are those in which X6 is an amino acid residue of Leu.

Embodiments of compounds of formula (I) are those in which X6 is an amino acid residue of Mle.

Embodiments of compounds of formula (I) are those in which X6 is an amino acid residue of Mly.

Embodiments of compounds of formula (I) are those in which X6 is an amino acid residue of Mva.

Embodiments of compounds of formula (I) are those in which X6 is an amino acid residue of Thr.

Embodiments of compounds of formula (I) are those in which X6 is an amino acid residue of Val.

Embodiments of compounds of formula (I) are those in which X6 is an amino acid residue of Trp.

Embodiments of compounds of formula (I) are those in which X6 is an amino acid residue of Phe.

Embodiments of compounds of formula (I) are those in which X8 is an amino acid residue selected from a residue of Yme, Tyr, Trp, Yde and Yae.

In some embodiments of the first group of embodiments, X8 is an amino acid residue selected from a residue of Yme, Tyr, Trp, and Yde

In some embodiments of the second group of embodiments, X8 is an amino acid residue selected from a residue of Yme, Tyr, Trp, Yde, and Yae.

Embodiments of compounds of formula (I) are those in which X8 is an amino acid residue selected from a residue of Yme, Yde and Yae,

Embodiments of compounds of formula (I) are those in which X8 is an amino acid residue of Yme.

Embodiments of compounds of formula (I) are those in which X8 is an amino acid residue of Tyr.

Embodiments of compounds of formula (I) are those in which X8 is an amino acid residue of Trp.

Embodiments of compounds of formula (I) are those in which X8 is an amino acid residue of Yde.

Embodiments of compounds of formula (I) are those in which X8 is an amino acid residue of Yae.

Embodiments of compounds of formula (I) are those in which X9 is an amino acid residue of Nal.

Embodiments of compounds of formula (I) are those in which X9 is an amino acid Nak.

Embodiments of compounds of formula (I) are those in which X9 is an amino acid residue of Trp.

Embodiments of compounds of formula (I) are those in which X10 is an amino acid residue selected from a residue of Aib, Cba, Cit, d-Trp, d-Tza, Lys, Lys(Ac), Leu, Mle, Mly, Gin, Mkdm and Thp.

Embodiments of compounds of formula (I) are those in which X10 is an amino acid residue selected from a residue of Aib, Lys, Lys(Ac), Mle, Mly, Mkdm and Thp.

Embodiments of compounds of formula (I) are those in which X10 is an amino acid residue selected from a residue of Aib, Mle, Mly, Mkdm and Thp.

Embodiments of compounds of formula (I) are those in which X10 is an amino acid residue of Aib.

Embodiments of compounds of formula (I) are those in which X10 is an amino acid residue of Cba.

Embodiments of compounds of formula (I) are those in which X10 is an amino acid residue of Cit.

Embodiments of compounds of formula (I) are those in which X10 is an amino acid residue of d-Trp.

Embodiments of compounds of formula (I) are those in which X10 is an amino acid residue of d-Tza.

Embodiments of compounds of formula (I) are those in which X10 is an amino acid residue of Lys.

Embodiments of compounds of formula (I) are those in which X10 is an amino acid residue of Lys(Ac).

Embodiments of compounds of formula (I) are those in which X10 is an amino acid residue of selected from Leu.

Embodiments of compounds of formula (I) are those in which X10 is an amino acid residue of Mle.

Embodiments of compounds of formula (I) are those in which X10 is an amino acid residue of Mly.

Embodiments of compounds of formula (I) are those in which X10 is an amino acid residue of Gin.

Embodiments of compounds of formula (I) are those in which X10 is an amino acid residue of Mkdm.

Embodiments of compounds of formula (I) are those in which X10 is an amino acid residue of Thp.

Embodiments of compounds of formula (I) are those in which X12 is absent or is an amino acid residue selected from a residue of Asn, Ser and Ala.

Embodiments of compounds of formula (I) are those in which X12 is absent.

Embodiments of compounds of formula (I) are those in which X12 is an amino acid residue of Asn.

Embodiments of compounds of formula (I) are those in which X12 is an amino acid residue of Ser.

Embodiments of compounds of formula (I) are those in which X12 is an amino acid residue of Ala.

Embodiments of compounds of formula (I) are those in which X13 is absent or is an amino acid residue selected from a residue of Asn, Ala, Bal, Gly, Leu, Ile, Iva, Hol, Hph, Pal, Pyal, His, Mhis, Val, and their corresponding d-forms.

Embodiments of compounds of formula (I) are those in which X13 is absent or is an amino acid residue selected from a residue of Asn, Ala, Bal, Gly, Leu, d-Leu, Ile, Iva, Hol, Hph, Pal, Pyal, His, Hme and Val.

Embodiments of compounds of formula (I) are those in which X13 is absent or is an amino acid residue selected from a residue of Asn, Gly, d-Leu, Pal and His.

Embodiments of compounds of formula (I) are those in which X14 is absent or is an amino acid residue selected from a residue of Mep, Sar, Bal, Ile, Dnmy and Hme.

Embodiments of compounds of formula (I) are those in which X14 is absent or is an amino acid residue selected from a residue of Mep, Sar, Bal and Ile.

Embodiments of compounds of formula (I) are those in which X14 is absent.

Embodiments of compounds of formula (I) are those in which X14 is an amino acid residue selected from a residue of Mep, Sar, Bal and Ile.

Embodiments of compounds of formula (I) are those in which X14 is an amino acid residue of Sar.

Embodiments of compounds of formula (I) are those in which X15 is absent or is an amino acid residue of Lys.

Embodiments of compounds of formula (I) are those in which X15 is absent.

Embodiments of compounds of formula (I) are those in which X15 is an amino acid residue of Lys.

In a third group of embodiments, compounds of formula (I) are those in which:
X1 is a residue selected from a residue of Ahx, Apt, Nmha, Nmapt, Lys and Dad, and is linked via an amide bond to X11,
or is a residue selected from a residue of Buty and Butyl, and is linked via an alkyl bond to X11 and when X1 is a residue of Butyl, the residue of Butyl is linked to X2 via an alkyl bond,
R1 is absent, or if X1 is selected from a residue of Ahx, Apt and Lys, then R1 is H, C₁-C₄-alkyl or acetyl, and is linked to and amino function, i
X2 is an amino acid residue selected from a residue of Cys, Glu, Asp, Nmc, Nmd, Nme, Nmt, Thr and Abu,
X7 is an amino acid residue selected from a residue of Cys, Glu, Asp, Thr and Abu,
wherein X2 and X7 are linked to each other via Q, wherein Q is a disulfide bond, a thioether bond or an ester bond,
X3 is an amino acid residue selected from a residue of Apt, Asn, Ndmk, Nmn and Lys(αAc),
X4 is absent, or is an amino acid residue of Thr,
X5 is an amino acid residue selected from a residue of Trp, Wme and Wim,
X6 is an amino acid residue selected from a residue of Gln, Lys(Ac) and Leu,
X8 is an amino acid residue selected from a residue of Yme, Trp, Yde and Yae,
X9 is an amino acid residue of Nal,
X10 is an amino acid residue selected from a residue of Aib, Lys, Lys(Ac), Mle, Mly, Mkdm and Thp,
X11 is an amino acid residue selected from a residue of Asp, Glu, Kme, Lys and Orn and is linked via an amide bond to X1,
X12 is absent or is an amino acid residue selected from a residue of Asn, Gly, Ser and Ala,
X13 is absent or is an amino acid residue selected from a residue of Asn, Ala, Bal, Gly, d-Leu, Ile, Pal, Pyal, PyEA, His and Hme,
X14 is absent or is an amino acid residue selected from a residue of Mep, Sar, Bal, Ile and Hme,
X15 is absent or is an amino acid residue of Lys,
R2 is absent or is —NH₂, —OH or —N(C₂H₅)₂,
or a salt or solvate thereof,
and
wherein the compound of formula (I) inhibits the binding of interleukin-23 to an interleukin-23-receptor.

In the third group of embodiments, compounds of formula (I) may be such that X2 is:
either an amino acid residue selected from an amino acid residue of Cys, Glu, Asp, Nmc, Nmd, Nme, Nmt, Thr and Abu, and X8 is an amino acid residue selected from an amino acid residue of Yme, Trp, and Yde,
or an amino acid residue selected from an amino acid residue of Cys, Glu, Asp, Nmc, Nmd, Nme, Nmt, and Thr, and X8 is an amino acid residue selected from an amino acid residue of Yme, Trp, Yde and Yae.

In a fourth group of embodiments, compounds of formula (I) are those in which:
X1 is a residue selected from a residue of Ahx, Apt, Nmha, Nmapt, Lys and Dad, and is linked via an amide bond to X11,
or is a residue selected from a residue of Buty and Butyl, and is linked via an alkyl bond to X11 and when X1 is a residue of Butyl, the residue of Butyl is linked to X2 via an alkyl bond,
R1 is absent or, if X1 is selected from a residue of Ahx, Apt and Lys, then R1 is H, C₁-C₄-alkyl or acetyl, and is linked to and amino function of X1,
X2 is an amino acid residue selected from a residue of Cys, Glu, Asp, Nmc, Nmd, Nme, Nmt, Thr and Abu,
X7 is an amino acid residue selected from a residue of Cys, Glu, Asp, Thr and Abu,
wherein X2 and X7 are linked to each other via Q, wherein Q is a disulfide bond, a thioether bond or an ester bond,
X3 is an amino acid residue selected from a residue of Asn and Nmn, X4 is an amino acid residue of Thr,
X5 is an amino acid residue selected from a residue of Trp, Wme and Wim,
X6 is an amino acid residue selected from a residue of Gin, Lys(Ac) and Leu,
X8 is an amino acid residue selected from a residue of Yme, Trp, Yde and Yae,
X9 is an amino acid residue of Nal,
X10 is an amino acid residue selected from a residue of Aib, Lys, Lys(Ac), Mle, Mly, Mkdm and Thp,
X11 is an amino acid residue selected from a residue of Asp, Glu, Kme, Lys and Orn and is linked via an amide bond to X1,
X12 is absent or is an amino acid residue selected from a residue of Asn, Gly, Ser and Ala,
X13 is absent or is an amino acid residue selected from a residue of Asn,
X14 is absent or is an amino acid residue selected from a residue of Mep, Sar, Bal, Ile and Hme,
X15 is absent or is an amino acid residue of Lys,
R2 is absent or is —NH₂, —OH or —N(C₂H₅)₂,
or a salt or solvate thereof,
and
wherein the compound of formula (I) inhibits the binding of interleukin-23 to an interleukin-23-receptor.

In the fourth group of embodiments, compounds of formula (I) may be such that X2 is:
either an amino acid residue selected from an amino acid residue of Cys, Glu, Asp, Nmc, Nmd, Nme, Nmt, Thr and Abu, and X8 is an amino acid residue selected from an amino acid residue of Yme, Trp, and Yde,
or an amino acid residue selected from an amino acid residue of Cys, Glu, Asp, Nmc, Nmd, Nme, Nmt, and Thr, and X8 is an amino acid residue selected from an amino acid residue of Yme, Trp, Yde and Yae.

In a fifth group of embodiments, compounds of formula (I) are those in which:
X1 is a residue selected from a residue of Ahx, Apt, Nmha, Nmapt, Lys and Dad, and is linked via an amide bond to X11,
or is a residue selected from a residue of Buty and Butyl, and is linked via an alkyl bond to X11 and when X1 is a residue of Butyl, the residue of Butyl is linked to X2 via an alkyl bond,
R1 is absent or, if X1 is selected from a residue of Ahx, Apt and Lys, then R1 is H, C₁-C₄-alkyl or acetyl, and is linked to an amino function of X1,
X2 is an amino acid residue selected from a residue of Cys, Glu, Asp, Nmc, Nmd, Nme, Nmt, Thr and Abu,
X7 is an amino acid residue selected from a residue of Cys, Glu, Asp, Thr and Abu,
wherein X2 and X7 are linked to each other via Q, wherein Q is a disulfide bond, a thioether bond or an ester bond,
X3 is an amino acid residue selected from a residue of Apt, Ndmk and Lys(αAc),
X4 is absent,
X5 is an amino acid residue selected from a residue of Trp, Wme and Wim,
X6 is an amino acid residue selected from a residue of Gln, Lys(Ac) and Leu,
X8 is an amino acid residue selected from a residue of Yme, Trp, Yde and Yae,
X9 is an amino acid residue of Nal,
X10 is an amino acid residue selected from a residue of Aib, Lys, Lys(Ac), Mle, Mly, Mkdm and Thp,
X11 is an amino acid residue selected from a residue of Asp, Glu, Kme, Lys and Orn and is linked via an amide bond to X1,
X12 is absent or is an amino acid residue selected from a residue of Asn, Gly, Ser and Ala,
X13 is absent or is an amino acid residue selected from a residue of Asn, Ala, Bal, Gly, d-Leu, Ile, Pal, Pyal, PyEA, His and Hme,
X14 is absent or is an amino acid residue selected from a residue of Mep, Sar, Bal, Ile and Hme,
X15 is absent or is an amino acid residue of Lys,
R2 is absent or is —NH₂, —OH or —N(C₂H₅)₂,
or a salt or solvate thereof,
and
wherein the compound of formula (I) inhibits the binding of interleukin-23 to an interleukin-23-receptor.

In the fifth group of embodiments, compounds of formula (I) may be such that X2 is:
either an amino acid residue selected from an amino acid residue of Cys, Glu, Asp, Nmc, Nmd, Nme, Nmt, Thr and Abu, and X8 is an amino acid residue selected from an amino acid residue of Yme, Trp, and Yde,
or an amino acid residue selected from an amino acid residue of Cys, Glu, Asp, Nmc, Nmd, Nme, Nmt, and Thr Abu, and X8 is an amino acid residue selected from an amino acid residue of Yme, Trp, Yde and Yae.

In a sixth group of embodiments, compounds of formula (I) are those in which:
R1 is absent or is H, C₁-C₄-alkyl or acetyl, and is linked to an amino function of X1,
X1 is a residue of Apt, and is linked via an amide bond to X11,
X2 is an amino acid residue of Cys,
X7 is an amino acid residue of Cys,
wherein X2 and X7 are linked to each other via Q, wherein Q is a disulfide bond,
X3 is an amino acid residue selected from a residue of Asn and Lys(αAc),
X4 is absent, or is an amino acid residue of Thr,
X5 is an amino acid residue of Wim,
X6 is an amino acid residue of Lys(Ac),
X8 is an amino acid residue of Yde,
X9 is an amino acid residue of Nal,
X10 is an amino acid residue selected from a residue of Mkdm and Thp,
X11 is an amino acid residue of Glu, and is linked via an amide bond to X1,
X12 is an amino acid residue of Asn,
X13 is an amino acid residue of Pal,
X14 is an amino acid residue of Sar,
X15 is absent,
R2 is —NH₂,
or a salt or solvate thereof,
and
wherein the compound of formula (I) inhibits the binding of interleukin-23 to an interleukin-23-receptor.

In a seventh group of embodiments, compounds of formula (I) are those in which:
R1 is absent or is H, C₁-C₄-alkyl or acetyl, and is linked to an amino function of X1,
X1 is a residue of Apt, and is linked via an amide bond to X11,
X2 is an amino acid residue of Cys,
X7 is an amino acid residue of Cys,
wherein X2 and X7 are linked to each other via Q, wherein Q is a disulfide bond,
X3 is an amino acid residue of Asn,
X4 is an amino acid residue of Thr,
X5 is an amino acid residue of Wim,
X6 is an amino acid residue of Lys(Ac),
X8 is an amino acid residue of Yde,
X9 is an amino acid residue of Nal,
X10 is an amino acid residue selected from a residue of Mkdm and Thp,
X11 is an amino acid residue of Glu, and is linked via an amide bond to X1,
X12 is an amino acid residue of Asn,
X13 is an amino acid residue of Pal,
X14 is an amino acid residue of Sar,
X15 is absent,
R2 is —NH₂,
or a salt or solvate thereof,
and
wherein the compound of formula (I) inhibits the binding of interleukin-23 to an interleukin-23-receptor.

In an eighth group of embodiments, compounds of formula (I) are those in which:
R1 is absent or is H, C₁-C₄-alkyl or acetyl, and is linked to one of the amino functions,
X1 is a residue of Apt, and is linked via an amide bond to X11,
X2 is an amino acid residue of Cys,
X7 is an amino acid residue of Cys,
wherein X2 and X7 are linked to each other via Q, wherein Q is a disulfide bond,
X3 is an amino acid residue of Lys(αAc),
X4 is absent,
X5 is an amino acid residue of Wim,
X6 is an amino acid residue of Lys(Ac),
X8 is an amino acid residue of Yde,
X9 is an amino acid residue of Nal,
X10 is an amino acid residue selected from a residue of Mkdm and Thp,
X11 is an amino acid residue of Glu, and is linked via an amide bond to X1,
X12 is an amino acid residue of Asn,
X13 is an amino acid residue of Pal,
X14 is an amino acid residue of Sar,
X15 is absent,
R2 is —NH₂,
or a salt or solvate thereof,
and
wherein the compound of formula (I) inhibits the binding of interleukin-23 to an interleukin-23-receptor.

In a ninth group of embodiments, compounds of formula (I) are those in which:
R1 is absent or, if X1 is selected from Ahx, Apt and Lys, then R1 is H, C₁-C₄-alkyl or acetyl, and is linked to an amino function of X1,
X1 is a residue selected from a residue of Ahx, Apt, Nmha, Nmapt, Lys and Dad, and is linked via an amide bond to X11,
or is a residue selected from a residue of Buty and Butyl, and is linked via an alkyl bond to X11 and when X1 is a residue of Butyl, the residue of Butyl is linked to X2 via an alkyl bond,
X2 is an amino acid residue selected from a residue of Cys, Nmc and Abu,
X7 is an amino acid residue selected from a residue of Cys, Glu, Asp and Thr,
wherein X2 and X7 are linked to each other via Q, wherein Q is a disulfide bond, a thioether bond or an ester bond,
X3 is an amino acid residue of Lys(αAc),
X4 is absent,
X5 is an amino acid residue selected from a residue of Trp and Wim,
X6 is an amino acid residue of Lys(Ac),
X8 is an amino acid residue of Yde,
X9 is an amino acid residue of Nal,
X10 is an amino acid residue selected from a residue of Mkdm and Thp,
X11 is an amino acid residue selected from a residue of Asp, Glu, Kme and Lys, and is linked via an amide bond to X1,
X12 is an amino acid residue of Gly,
X13 is an amino acid residue of PyEA,
X14 is absent,
X15 is absent,
R2 is absent,
or a salt or solvate thereof,
and
wherein the compound of formula (I) inhibits the binding of interleukin-23 to an interleukin-23-receptor.

In a tenth group of embodiments, compounds of formula (I) are those in which:
X1 is a residue selected from a residue of Ahx, Apt, Nmha, Nmapt, Lys, Dad, and is linked via an amide bond to X11,
R1 is absent or, if X1 is selected from a residue of Ahx, Aoa, Apt, Lys, Dab and Dap, then R1 is H, C₁-C₄-alkyl or acetyl, and is linked to an amino function of X1,
X2-Q-X7 is selected from Cys-Q-Cys and Nmc-Q-Cys, wherein Q is a disulfide bond, or Abu-Q-Cys wherein Q is a thioether bond, or Glu-Q-Thr, wherein Q is an ester bond,
X3 is an amino acid residue of Lys(αAc) and X4 is absent,
or X3 is an amino acid residue of Asn, and X4 is an amino acid residue of Thr,
X5 is an amino acid residue selected from a residue of Trp and Wim,
X6 is an amino acid residue selected from a residue of Gin and Lys(Ac),
X8 is an amino acid residue selected from a residue of Yme, Yde and Yae, X9 is an amino acid residue of Nal,
X10 is an amino acid residue selected from a residue of Aib, Mle, Mly, Mkdm and Thp,
X11 is an amino acid residue selected from a residue of the group of Asp, Glu, Kme and Lys, and is linked via an amide bond to X1,
X12 is an amino acid residue selected from a residue of Asn, Ser and Ala,
X13 is an amino acid residue selected from a residue of Asn, Gly, d-Leu, Pal and His,
X14 is absent or is an amino acid residue selected from a residue of Mep, Sar, Bal and Ile,
X15 is absent,
R2 is —NH₂,
or a salt or solvate thereof,
and
wherein the compound of formula (I) inhibits the binding of interleukin-23 to an interleukin-23-receptor.

In some embodiments of the tenth group of embodiments, compounds of formula (I) may be such that:
X2-Q-X7 is selected from Cys-Q-Cys and Nmc-Q-Cys, wherein Q is a disulfide bond, or Abu-Q-Cys wherein Q is a thioether bond, or Glu-Q-Thr, wherein Q is an ester bond, and X8 is an amino acid residue selected from a residue of Yme, and Yde, or
X7 is selected from Cys-Q-Cys and Nmc-Q-Cys, wherein Q is a disulfide bond, or Glu-Q-Thr, wherein Q is an ester bond, and X8 is an amino acid residue selected from a residue of Yme, Yde and Yae.

In an eleventh group of embodiments, compounds of formula (I) are those in which X2-X3-X4-X5-X6-X7 is selected from:
Cys-Asn-Thr-Trp-Gin-Cys,
Cys-Asn-Thr-Wim-Gin-Cys,
Cys-Asn-Thr-Trp-Lys(Ac)-Cys,
Cys-Asn-Thr-Wim-Lys(Ac)-Cys,
Cys-Nmn-Thr-Trp-Gln-Cys,
Abu-Asn-Thr-Trp-Lys(Ac)-Cys,
Abu-Asn-Thr-Wim-Lys(Ac)-Cys and
Abu-Asn-Thr-Wme-Leu-Cys.

In an twelfth group of embodiments, compounds of formula (I) are those in which X2-X3-X4-X5-X6-X7 is selected from:
Cys-Asn-Thr-Trp-Lys(Ac)-Cys,
Cys-Asn-Thr-Wim-Lys(Ac)-Cys,
Cys-Nmn-Thr-Trp-Gln-Cys,
Abu-Asn-Thr-Trp-Lys(Ac)-Cys and
Abu-Asn-Thr-Wim-Lys(Ac)-Cys.

In a thirteenth group of embodiments, compounds of formula (I) are those in which X2-X3-X4-X5-X6-X7 is selected from:
Cys-Asn-Thr-Trp-Lys(Ac)-Cys and
Cys-Asn-Thr-Wim-Lys(Ac)-Cys.

In a fourteenth group of embodiments, compounds of formula (I) are those in which X2-X3-X5-X6-X7 is selected from:
Cys-Lys(αAc)-Trp-Gln-Cys,
Cys-Lys(αAc)-Wim-Gin-Cys,
Cys-Lys(αAc)-Trp-Lys(Ac)-Cys,
Cys-Lys(αAc)-Wim-Lys(Ac)-Cys,
Cys-Ndmk-Trp-Lys(Ac)-Cys,
Cys-Ndmk-Wim-Lys(Ac)-Cys,
Cys-Lys(αAc)-Wim-Leu-Cys,
Nmc-Lys(αAc)-Wim-Leu-Cys,
Cys-Apt-Trp-Gin-Cys,
Abu-Lys(αAc)-Trp-Lys(Ac)-Cys,
Abu-Lys(αAc)-Wim-Lys(Ac)-Cys, and
Glu-Apt-Trp-Gln-Thr,
and X4 is absent.

In a fifteenth group of embodiments, compounds of formula (I) are those in which X2-X3-X5-X6-X7 is selected from:
Cys-Lys(αAc)-Trp-Gin-Cys,
Cys-Lys(αAc)-Wim-Gin-Cys,
Cys-Lys(αAc)-Trp-Lys(Ac)-Cys,
Cys-Lys(αAc)-Wim-Lys(Ac)-Cys,
Cys-Ndmk-Trp-Lys(Ac)-Cys,
Cys-Ndmk-Wim-Lys(Ac)-Cys,
Cys-Lys(αAc)-Wim-Leu-Cys,
Nmc-Lys(αAc)-Wim-Leu-Cys and
Cys-Apt-Trp-Gin-Cys,
and X4 is absent.

In a sixteenth group of embodiments, compounds of formula (I) are those in which X2-X3-X5-X6-X7 is selected from:
Cys-Lys(αAc)-Trp-Lys(Ac)-Cys,
Cys-Lys(αAc)-Wim-Lys(Ac)-Cys,
Cys-Ndmk-Trp-Lys(Ac)-Cys and
Cys-Ndmk-Wim-Lys(Ac)-Cys,
and X4 is absent.

In a seventeenth group of embodiments, compounds of formula (I) are those in which X8-X9-X10 is X8-Nal-X10, wherein X8 is selected from a residue of Yae, Yde and Yme and X10 is selected from a residue of Aib, Mle, Thp, Mkdm and Mly.

In a eighteenth group of embodiments, compounds of formula (I) are those in which X8-X9-X10 is Yde-Nal-X10, wherein X10 is selected from a residue of Aib, Mle, Thp, Mkdm and Mly.

In a nineteenth group of embodiments, compounds of formula (I) are those in which X8-X9-X10 is selected from the group of:
Yde-Nal-Aib,
Yde-Nal-Mle,
Yde-Nal-Thp,
Yde-Nal-Mkdm,
Yde-Nal-Mly,
Yme-Nal-Aib,
Yae-Nal-Mie,
Yae-Nal-Thp,
Yme-Nal and
Yde-Nal-Aib.

In a twentieth group of embodiments, compounds of formula (I) are those in which X12-X13- is selected from Asn-Asn-, Asn-Gly-, Asn-d-Leu-, Ala-Gly-, Ala-d-Leu-, Asn-His-, Ser-His-, Asn-Pal-, Asn-Pyal- and Gly-PyEA-.

In a twenty-first group of embodiments, compounds of formula (I) are those in which X12-X13- is Gly-PyEA, X14 is absent, X15 is absent, and R2 is absent.

In an twenty-second group of embodiments, compounds of formula (I) are those in which X12-X13- is selected from Asn-Asn-, Asn-Gly-, Asn-d-Leu-, Ala-Gly-, Ala-d-Leu-, Asn-His-, Ser-His-, Asn-Pal- and Asn-Pyal-, X14 is absent, X15 is absent, and R2 is NH₂. absent.

In an twenty-third group of embodiments, compounds of formula (I) are those in which X12-X13-X14- is selected from Asn-Pal-Sar-, Asn-Pal-Ile-, Asn-Asn-Ile-, Asn-His-Sar, Asn-His-Bal-, Asn-His-Mep-, Ser-Pal-Sar- and Ala-Pal-Sar-.

In an twenty-fourth group of embodiments, compounds of formula (I) are those in which X12-X13-X14- is selected from Asn-Pal-Sar-, Asn-Pal-Ile-, Asn-Asn-Ile-, Asn-His-Sar, Asn-His-Bal-, Asn-His-Mep-, Ser-Pal-Sar-, Ala-Pal-Sar-, Asn-Asn-absent-, Asn-Gly-absent-, Asn-d-Leu-absent-, Ala-Gly-absent-, Ala-d-Leu-absent-, Asn-His-absent-, Ser-His-absent-, Asn-Pal-absent-, Asn-Pal-absent-, Asn-absent-absent-, Asn-Pyal-absent- and Gly-PyEA-absent-.

In an twenty-fifth group of embodiments, compounds of formula (I) are those in which X12-X13-X14-X15 is selected from the group of Asn-Asn-absent-absent-, Asn-Gly-absent-absent-, Asn-d-Leu-absent-absent-, Ala-Gly-absent-absent-, Ala-d-Leu-absent-absent-, Asn-His-absent-absent-, Ser-His-absent-absent-, Asn-Pal-absent-absent-, Asn-Pal-Sar-absent-, Asn-Pal-Sar-Lys, Asn-Pal-Ile-absent-, Asn-Asn-Ile-absent-, Asn-His-Sar-absent-, Asn-His-Bal-absent-, Asn-His-Mep-absent, Ser-Pal-Sar-absent-, Ala-Pal-Sar-absent-, Asn-Pal-absent-Lys-, Asn-absent-absent-Lys, Asn-Pyal-absent-absent and Gly-PyEA-absent-absent.

In a twenty-sixth group of embodiments, compounds of formula (I) are those in which:
R1 is H or acetyl, and is linked to an amino function of X1,
X1 is a residue selected from a residue of Ahx, Aoa, Apt, Nmha, Nmapt, Lys, Dab, Dad, Dap and is linked via an amide bond to X11,
X2-X3-X4-X5-X6-X7 is selected from:
Cys-Asn-Thr-Trp-Gln-Cys,
Cys-Asn-Thr-Wim-Gln-Cys,
Cys-Asn-Thr-Trp-Lys(Ac)-Cys,
Cys-Asn-Thr-Wim-Lys(Ac)-Cys,
Cys-Nmn-Thr-Trp-Gln-Cys,
Abu-Asn-Thr-Trp-Lys(Ac)-Cys,
Abu-Asn-Thr-Wim-Lys(Ac)-Cys and
Abu-Asn-Thr-Wme-Leu-Cys,
X2-Q-X7 is selected from a group of Cys-Q-Cys and Nmc-Q-Cys, wherein Q is a disulfide bond, or Abu-Q-Cys wherein Q is a thioether bond, or
Glu-Q-Thr, wherein Q is an ester bond,
X8-X9-X10 are selected from the group of
Yde-Nal-Alb,
Yde-Nal-Mle,
Yde-Nal-Thp,
Yde-Nal-Mkdm,
Yde-Nal-Mly,
Yme-Nal-Aib,
Yae-Nal-Mle,
Yae-Nal-Thp,
Yme-Nal and
Yde-Nal-Aib,
X11 is an amino acid residue selected from a residue of Asp, Glu, Kme and Lys, and is linked via an amide bond to X1,
X12-X13-X14-X15 is selected from the group of Asn-Asn-absent-absent-, Asn-Gly-absent-absent-, Asn-d-Leu-absent-absent-, Ala-Gly-absent-absent-, Ala-d-Leu-absent-absent-, Asn-His-absent-absent-, Ser-His-absent-absent-, Asn-Pal-absent-absent-, Asn-Pal-Sar-absent-, Asn-Pal-Sar-Lys, Asn-Pal-Ile-absent-, Asn-Asn-Ile-absent-, Asn-His-Sar-absent-, Asn-His-Bal-absent-, Asn-His-Mep-absent, Ser-Pal-Sar-absent-, Ala-Pal-Sar-absent-, Asn-Pal-absent-Lys-, Asn-absent-absent-Lys, Asn-Pyal-absent-absent and Gly-PyEA-absent-absent,
R2 is absent or is —NH₂, —OH or —N(C₂H₅)₂,
or a salt or solvate thereof,
and
wherein the compound of formula (I) inhibits the binding of interleukin-23 to an interleukin-23-receptor.

In a twenty-seventh group of embodiments, compounds of formula (I) are those in which:
R1 is H or acetyl, and is linked to an amino function of X1,
X1 is a residue selected from a residue of Ahx, Aoa, Apt, Nmha, Nmapt, Lys, Dab, Dad, Dap and is linked via an amide bond to X11,
X2-X3-X4-X5-X6-X7 is selected from:
Cys-Asn-Thr-Trp-Gln-Cys,
Cys-Asn-Thr-Wim-Gin-Cys,
Cys-Asn-Thr-Trp-Lys(Ac)-Cys,
Cys-Asn-Thr-Wim-Lys(Ac)-Cys,
Cys-Nmn-Thr-Trp-Gln-Cys,
Abu-Asn-Thr-Trp-Lys(Ac)-Cys,
Abu-Asn-Thr-Wim-Lys(Ac)-Cys and
Abu-Asn-Thr-Wme-Leu-Cys,
X2-Q-X7 is selected from a group of Cys-Q-Cys and Nmc-Q-Cys, wherein Q is a disulfide bond, or Abu-Q-Cys wherein Q is a thioether bond, or
Glu-Q-Thr, wherein Q is an ester bond,
X8-X9-X10 are selected from the group of
Yde-Nal-Aib,
Yde-Nal-Mle,
Yde-Nal-Thp,
Yde-Nal-Mkdm,
Yde-Nal-Mly,
Yme-Nal-Aib,
Yae-Nal-Mle,
Yme-Nal and
Yde-Nal-Aib,
X11 is an amino acid residue selected from a residue of Asp, Glu, Kme and Lys, and is linked via an amide bond to X1,
X12-X13-X14-X15 is selected from the group of Asn-Asn-absent-absent-, Asn-Gly-absent-absent-, Asn-d-Leu-absent-absent-, Ala-Gly-absent-absent-, Ala-d-Leu-absent-absent-, Asn-His-absent-absent-, Ser-His-absent-absent-, Asn-Pal-absent-absent-, Asn-Pal-Sar-absent-, Asn-Pal-Sar-Lys, Asn-Pal-Ile-absent-, Asn-Asn-Ile-absent-, Asn-His-Sar-absent-, Asn-His-Bal-absent-, Asn-His-Mep-absent, Ser-Pal-Sar-absent-, Ala-Pal-Sar-absent-, Asn-Pal-absent-Lys-, Asn-absent-absent-Lys, Asn-Pyal-absent-absent and Gly-PyEA-absent-absent,
R2 is absent or is —NH₂, —OH or —N(C₂H₅)₂, or a salt or solvate thereof,
and
wherein the compound of formula (I) inhibits the binding of interleukin-23 to
an interleukin-23-receptor.

In a twenty-eighth group of embodiments, compounds of formula (I) are those in which:
R1 is H or acetyl, and is linked to an amino function of X1,
X1 is a residue selected from a residue of Ahx, Aoa, Apt, Nmha, Nmapt, Lys,
Dab, Dad, Dap and is linked via an amide bond to X11,
X2-X3-X4-X5-X6-X7 is selected from:
Cys-Asn-Thr-Trp-Gin-Cys,
Cys-Asn-Thr-Wim-Gin-Cys,
Cys-Asn-Thr-Trp-Lys(Ac)-Cys,
Cys-Asn-Thr-Wim-Lys(Ac)-Cys,
Cys-Nmn-Thr-Trp-Gin-Cys,
Abu-Asn-Thr-Trp-Lys(Ac)-Cys,
Abu-Asn-Thr-Wme-Leu-Cys,
X2-Q-X7 is selected from a group of Cys-Q-Cys and Nmc-Q-Cys, wherein Q is a disulfide bond, or Abu-Q-Cys wherein Q is a thioether bond, or
Glu-Q-Thr, wherein Q is an ester bond,
X8-X9-X10 are selected from the group of
Yde-Nal-Aib,
Yde-Nal-Mle,
Yde-Nal-Thp,
Yde-Nal-Mkdm,
Yde-Nal-Mly,
Yme-Nal-Aib,
Yae-Nal-Mle,
Yae-Nal-Thp,
Yme-Nal and
Yde-Nal-Aib,
X11 is an amino acid residue selected from a residue of Asp, Glu, Kme and Lys, and is linked via an amide bond to X1,
X12-X13-X14-X15 is selected from the group of Asn-Asn-absent-absent-, Asn-Gly-absent-absent-, Asn-d-Leu-absent-absent-, Ala-Gly-absent-absent-, Ala-d-Leu-absent-absent-, Asn-His-absent-absent-, Ser-His-absent-absent-, Asn-Pal-absent-absent-, Asn-Pal-Sar-absent-, Asn-Pal-Sar-Lys, Asn-Pal-Ile-absent-, Asn-Asn-Ile-absent-, Asn-His-Sar-absent-, Asn-His-Bal-absent-, Asn-His-Mep-absent, Ser-Pal-Sar-absent-, Ala-Pal-Sar-absent-, Asn-Pal-absent-Lys-, Asn-absent-absent-Lys, Asn-Pyal-absent-absent and Gly-PyEA-absent-absent,
R2 is absent or is —NH₂, —OH or —N(C₂H₅)₂,
or a salt or solvate thereof,
and
wherein the compound of formula (I) inhibits the binding of interleukin-23 to an interleukin-23-receptor.

In a twenty-ninth group of embodiments, compounds of formula (I) are those in which:
R1 is H or acetyl, and is linked to an amino function of X1,
X1 is a residue selected from a residue of Ahx, Aoa, Apt, Nmha, Nmapt, Lys, Dab, Dad, Dap and is linked via an amide bond to X11,
X2-X3-X5-X6-X7 is selected from:
Cys-Lys(αAc)-Trp-Gln-Cys,
Cys-Lys(αAc)-Wim-Gin-Cys,
Cys-Lys(αAc)-Trp-Lys(Ac)-Cys,
Cys-Lys(αAc)-Wim-Lys(Ac)-Cys,
Cys-Ndmk-Trp-Lys(Ac)-Cys,
Cys-Ndmk-Wim-Lys(Ac)-Cys,
Cys-Lys(αAc)-Wim-Leu-Cys,
Nmc-Lys(αAc)-Wim-Leu-Cys,
Cys-Apt-Trp-Gln-Cys,
Abu-Lys(αAc)-Trp-Lys(Ac)-Cys,
Abu-Lys(αAc)-Wim-Lys(Ac)-Cys, and
Glu-Apt-Trp-Gln-Thr,
X4 is absent,
X2-Q-X7 is selected from Cys-Q-Cys and Nmc-Q-Cys, wherein Q is a disulfide bond, or Abu-Q-Cys wherein Q is a thioether bond, or
Glu-Q-Thr, wherein Q is an ester bond,
X8-X9-X10 are selected from the group of
Yde-Nal-Aib, Yde-Nal-Mle, Yde-Nal-Thp, Yde-Nal-Mkdm, Yde-Nal-Mly, Yme-Nal-Aib, Yae-Nal-Mle, Yae-Nal-Thp, Yme-Nal and Yde-Nal-Aib,
X11 is an amino acid residue selected from a residue of Asp, Glu, Kme and Lys, and is linked via an amide bond to X1,
X12-X13-X14-X15 is selected from the group of
Asn-Asn-absent-absent-, Asn-Gly-absent-absent-, Asn-d-Leu-absent-absent-, Ala-Gly-absent-absent-, Ala-d-Leu-absent-absent-, Asn-His-absent-absent-, Ser-His-absent-absent-, Asn-Pal-absent-absent-, Asn-Pal-Sar-absent-, Asn-Pal-Sar-Lys, Asn-Pal-Ile-absent-, Asn-Asn-Ile-absent-, Asn-His-Sar-absent-, Asn-His-Bal-absent-, Asn-His-Mep-absent, Ser-Pal-Sar-absent-, Ala-Pal-Sar-absent-, Asn-Pal-absent-Lys-, Asn-absent-absent-Lys, Asn-Pyal-absent-absent and Gly-PyEA-absent-absent,
R2 is absent or is —NH₂, —OH or —N(C₂H₅)₂,
or a salt or solvate thereof,
and
wherein the compound of formula (I) inhibits the binding of interleukin-23 to an interleukin-23-receptor.

In a thirtieth group of embodiments, compounds of formula (I) are those in which:
R1 is H or acetyl, and is linked to an amino function of X1,
X1 is a residue selected from a residue of Ahx, Aoa, Apt, Nmha, Nmapt, Lys, Dab, Dad, Dap and is linked via an amide bond to X11,
X2-X3-X5-X6-X7 is selected from
Cys-Lys(αAc)-Trp-Gln-Cys,
Cys-Lys(αAc)-Wim-Gin-Cys,
Cys-Lys(αAc)-Trp-Lys(Ac)-Cys,
Cys-Lys(αAc)-Wim-Lys(Ac)-Cys,
Cys-Ndmk-Trp-Lys(Ac)-Cys,
Cys-Ndmk-Wim-Lys(Ac)-Cys,
Cys-Lys(αAc)-Wim-Leu-Cys,
Nmc-Lys(αAc)-Wim-Leu-Cys,
Cys-Apt-Trp-Gin-Cys,
Abu-Lys(αAc)-Trp-Lys(Ac)-Cys,
Abu-Lys(αAc)-Wim-Lys(Ac)-Cys, and
Glu-Apt-Trp-Gln-Thr,
X4 is absent,
X2-Q-X7 is selected from Cys-Q-Cys and Nmc-Q-Cys, wherein Q is a disulfide bond, or Abu-Q-Cys wherein Q is a thioether bond, or
Glu-Q-Thr, wherein Q is an ester bond,
X8-X9-X10 are selected from the group of
Yde-Nal-Aib, Yde-Nal-Mle, Yde-Nal-Mkdm, Yde-Nal-Mly, Yme-Nal-Aib, Yae-Nal-Mle, Yae-Nal-Thp, Yme-Nal and Yde-Nal-Aib,
X11 is an amino acid residue selected from a residue of Asp, Glu, Kme and Lys, and is linked via an amide bond to X1,
X12-X13-X14-X15 is selected from the group of
Asn-Asn-absent-absent-, Asn-Gly-absent-absent-, Asn-d-Leu-absent-absent-, Ala-Gly-absent-absent-, Ala-d-Leu-absent-absent-, Asn-His-absent-absent-, Ser-His-absent-absent-, Asn-Pal-absent-absent-, Asn-Pal-Sar-absent-, Asn-Pal-Sar-Lys, Asn-Pal-Ile-absent-, Asn-Asn-Ile-absent-, Asn-His-Sar-absent-, Asn-His-Bal-absent-, Asn-His-Mep-absent, Ser-Pal-Sar-absent-, Ala-Pal-Sar-absent-, Asn-Pal-absent-Lys-, Asn-absent-absent-Lys, Asn-Pyal-absent-absent and Gly-PyEA-absent-absent,
R2 is absent or is —NH₂, —OH or —N(C₂H₅)₂,
or a salt or solvate thereof,
and
wherein the compound of formula (I) inhibits the binding of interleukin-23 to an interleukin-23-receptor.

In a thirty-first group of embodiments, compounds of formula (I) are those in which:
R1 is H or acetyl, and is linked to an amino function of X1,
X1 is a residue selected from a residue of Ahx, Aoa, Apt, Nmha, Nmapt, Lys,
Dab, Dad, Dap and is linked via an amide bond to X11,
X2-X3-X5-X6-X7 is selected from
Cys-Lys(αAc)-Trp-Gin-Cys,
Cys-Lys(αAc)-Wim-Gin-Cys,
Cys-Lys(αAc)-Trp-Lys(Ac)-Cys,
Cys-Lys(αAc)-Wim-Lys(Ac)-Cys,
Cys-Ndmk-Trp-Lys(Ac)-Cys,
Cys-Ndmk-Wim-Lys(Ac)-Cys,
Cys-Lys(αAc)-Wim-Leu-Cys,
Nmc-Lys(αAc)-Wim-Leu-Cys,
Cys-Apt-Trp-Gin-Cys,
Abu-Lys(αAc)-Trp-Lys(Ac)-Cys,
Glu-Apt-Trp-Gln-Thr,
X4 is absent,
X2-Q-X7 is selected from Cys-Q-Cys and Nmc-Q-Cys, wherein Q is a disulfide bond, or Abu-Q-Cys wherein Q is a thioether bond, or
Glu-Q-Thr, wherein Q is an ester bond,
X8-X9-X10 are selected from the group of
Yde-Nal-Aib, Yde-Nal-Mle, Yde-Nal-Thp, Yde-Nal-Mkdm, Yde-Nal-Mly, Yme-Nal-Aib, Yae-Nal-Mle, Yae-Nal-Thp, Yme-Nal and Yde-Nal-Aib,
X11 is an amino acid residue selected from a residue of Asp, Glu, Kme and Lys, and is linked via an amide bond to X1,
X12-X13-X14-X15 is selected from the group of
Asn-Asn-absent-absent-, Asn-Gly-absent-absent-, Asn-d-Leu-absent-absent-, Ala-Gly-absent-absent-, Ala-d-Leu-absent-absent-, Asn-His-absent-absent-, Ser-His-absent-absent-, Asn-Pal-absent-absent-, Asn-Pal-Sar-absent-, Asn-Pal-Sar-Lys, Asn-Pal-Ile-absent-, Asn-Asn-Ile-absent-, Asn-His-Sar-absent-, Asn-His-Bal-absent-, Asn-His-Mep-absent, Ser-Pal-Sar-absent-, Ala-Pal-Sar-absent-, Asn-Pal-absent-Lys-, Asn-absent-absent-Lys, Asn-Pyal-absent-absent and Gly-PyEA-absent-absent,
R2 is absent or is —NH₂, —OH or —N(C₂H₅)₂,
or a salt or solvate thereof,
and
wherein the compound of formula (I) inhibits the binding of interleukin-23 to an interleukin-23-receptor.

### Compounds of Formula (Ia)

In some embodiments, peptide compounds described herein comprise one or more half-life extension moiety and/or more linker moiety linked to the peptide.

Disclosed herein are compounds of formula (Ia)

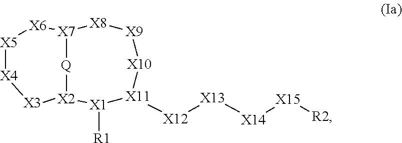

wherein
X1 is a residue selected from a residue of Ahx, Aoa, Apt, Nmha, Nmapt, Lys, Pipa, Pipb, Pipp, Dab, Dad and Dap, and is linked via an amide bond to X11,
or is a residue selected from a residue of Buty and Butyl, and is linked via an alkyl bond to X11 and when X1 is a residue of Butyl, the residue of Butyl is linked to X2 via an alkyl bond,
R1 is absent or, if X1 is selected from a residue of Ahx, Aoa, Apt, Lys, Dab and Dap, then R1 is H, C₁-C₄-alkyl or acetyl, and is linked to an amino function of X1,
X2 is an amino acid residue selected from a residue of Cys, Glu, Asp, Nmc, Nmd, Nme, Nmt, Thr and Abu and their corresponding d-forms,
X7 is an amino acid residue selected from a residue of Cys, Glu, Asp, Thr and Abu and their corresponding d-forms,
wherein X2 and X7 are linked to each other via Q, wherein Q is a disulfide bond, a thioether bond or an ester bond,
X3 is an amino acid residue selected from a residue of Sar, His, Lys, Ala, Ahx, Apt, Arg, Asn, Gab, Gin, Trp, Thr, Ndmk, Nmn, Ntmk and Lys(αAc),
X4 is absent, or is an amino acid residue selected from a residue of His, Lys, Ahx, Apt, Arg, Asn, Gab, Gln, Trp and Thr,
X5 is an amino acid residue selected from a residue of Trp, Wme, Nak, Wcl, Wim, Wdm and Wfl,
X6 is an amino acid residue selected from a residue of Gln, Aib, Glu, Phe, Ile, Iva, Lys, Lys(Ac), Leu, Mle, Mly, Mva, Thr, Val, Trp and Phe,
X8 is an amino acid residue selected from a residue of Yme, Tyr, Trp, Yde and Yae,
X9 is an amino acid residue selected from a residue of Nal, Nak and Trp,
X10 is an amino acid residue selected from a residue of Aib, Cba, Cit, d-Trp, d-Tza, Lys, Lys(Ac), Leu, Mle, Mly, Gin, Mkdm and Thp,
X11 is an amino acid residue selected from a residue of Asp, Bal, Gab, Glu, Kme, Lys and Orn and is linked via an amide bond to X1,
X12 is absent or is an amino acid residue selected from a residue of Asn, Gly, Ser and Ala,
X13 is absent or is an amino acid residue selected from a residue of Asn, Ala, Bal, Gly, Leu, Ile, Iva, Hol, Hph, Pal, Pyal, PyEA, His, Hme, Val, and their corresponding d-forms,
X14 is absent or is an amino acid residue selected from a residue of Mep, Sar, Bal, Ile, Dnmy and Hme,
X15 is absent or is an amino acid residue of Lys,
and wherein
amide bonds between X1 and X2, X2 and X3, X3 and X4, X4 and X5, X11 and X1 are optionally methylated,
R2 is absent or is —NH₂, —OH or —N(C₂H₅)₂,
or a salt or solvate thereof,
and
wherein at least one of X10 and X15
is an amino acid residue having a side chain with an —NH₂ group, wherein the —NH₂ side chain group is functionalized by —C(O)—R5, —C(O)O—R5, —C(O)NH—R5, —S(O)₂—R5 or R5, for example by —C(O)—R5, wherein R5 may be a moiety comprising up to 50 or up to 100 carbon atoms and optionally heteroatoms selected from halogen, N, O, S and P.

In some embodiments, compounds of formula (Ia) inhibit the binding of interleukin-23 to an interleukin-23-receptor.

The embodiments described for R1, X1 to X15, and R2 for formula (I) apply also for compounds of formula (Ia) with at least one of X10 and X15 being functionalized as disclosed herein.

In some embodiments, compounds of formula (Ia) may be such that X2 is:
either an amino acid residue selected from an amino acid residue of Cys, Glu, Asp, Nmc, Nmd, Nme, Nmt, Thr and Abu, and their corresponding d-forms, and X8 is an amino acid residue selected from an amino acid residue of Yme, Tyr, Trp, and Yde,
or an amino acid residue selected from an amino acid residue of Cys, Glu, Asp, Nmc, Nmd, Nme, Nmt, and Thr, and their corresponding d-forms, and X8 is an amino acid residue selected from an amino acid residue of Yme, Tyr, Trp, Yde and Yae.

In some embodiments, compounds of formula (Ia) may be such that when X2 is Abu, or its corresponding d-forms, then X8 is not an amino acid residue of Yae.

In some embodiments, compounds of formula (Ia) may be such that when X8 is Yae, then X2 is not an amino acid residue of Abu, or its corresponding d-forms.

A first group of embodiments of compounds of formula (Ia) may be such that:
X1 is a residue selected from a residue of Ahx, Aoa, Apt, Nmha, Nmapt, Lys, Pipa, Pipb, Pipp, Dab, Dad and Dap, and is linked via an amide bond to X11,
or is a residue selected from a residue of Buty and Butyl, and is linked via an alkyl bond to X11 and when X1 is a residue of Butyl, the residue of Butyl is linked to X2 via an alkyl bond,
R1 is absent, or
if X1 is a residue selected from a residue of Ahx, Aoa, Apt, Lys, Dab and Dap, then R1 is H, C₁-C₄-alkyl or acetyl, and is linked to an amino function of X1,
X2 is an amino acid residue selected from a residue of Cys, Glu, Asp, Nmc, Nmd, Nme, Nmt, Thr and Abu, and their corresponding d-forms,
X7 is an amino acid residue selected from a residue of Cys, Glu, Asp, Thr and Abu, and their corresponding d-forms,
wherein X2 and X7 are linked to each other via Q, wherein Q is a disulfide bond, a thioether bond or an ester bond,
X3 is an amino acid residue selected from a residue of Sar, His, Lys, Ala, Ahx, Apt, Arg, Asn, Gab, Gin, Trp, Thr, Ndmk, Nmn, Ntmk and Lys(αAc),
X4 is absent, or is an amino acid residue selected from a residue of His, Lys, Ahx, Apt, Arg, Asn, Gab, Gln, Trp and Thr,
X5 is an amino acid residue selected from a residue of Trp, Wme, Nak, Wcl, Wim, Wdm and Wfl,
X6 is an amino acid residue selected from a residue of Gln, Aib, Glu, Phe, Ile, Iva, Lys, Lys(Ac), Leu, Mle, Mly, Mva, Thr, Val, Trp and Phe,
X8 is an amino acid residue selected from a residue of Yme, Tyr, Trp, and Yde,
X9 is an amino acid residue selected from a residue of Nal, Nak and Trp,
X10 is an amino acid residue selected from a residue of Aib, Cba, Cit, d-Trp, d-Tza, Lys, Lys(Ac), Leu, Mle, Mly, Gln, Mkdm and Thp,
X11 is an amino acid residue selected from a residue of Asp, Bal, Gab, Glu, Kme, Lys and Orn and is linked via an amide bond to X1,
X12 is absent or is an amino acid residue selected from a residue of Asn, Gly, Ser and Ala,
X13 is absent or is an amino acid residue selected from a residue of Asn, Ala, Bal, Gly, Leu, Ile, Iva, Hol, Hph, Pal, Pyal, PyEA, His, Hme, Val, and their corresponding d-forms,
X14 is absent or is an amino acid residue selected from a residue of Mep, Sar, Bal, Ile, Dnmy and Hme,
X15 is absent or is an amino acid residue of Lys,
R2 is absent or is —NH₂, —OH or —N(C₂H₅)₂,
and
wherein amide bonds between X1 and X2, X2 and X3, X3 and X4, X4 and X5, X11 and X1 are optionally methylated,
and
wherein at least one of X10 and X15 is an amino acid residue having a side chain with an —NH₂ group, wherein the —NH₂ side chain group is functionalized by —C(O)—R5, —C(O)O—R5, —C(O)NH—R5, —S(O)₂—R5 or R5, wherein R5 may be a moiety comprising up to 50 or up to 100 carbon atoms and optionally heteroatoms selected from halogen, N, O, S and P,
or a salt or solvate thereof,
wherein the compound of formula (Ia) inhibits the binding of interleukin-23 to an interleukin-23-receptor.

A second group of embodiments of compounds of formula (Ia) may be such that:
X1 is a residue selected from a residue of Ahx, Aoa, Apt, Nmha, Nmapt, Lys, Pipa, Pipb, Pipp, Dab, Dad and Dap, and is linked via an amide bond to X11,
or is a residue selected from a residue of Buty and Butyl, and is linked via an alkyl bond to X11 and when X1 is a residue of Butyl, the residue of Butyl is linked to X2 via an alkyl bond,
R1 is absent, or
if X1 is a residue selected from a residue of Ahx, Aoa, Apt, Lys, Dab and Dap, then R1 is H, C₁-C₄-alkyl or acetyl, and is linked to an amino function of X1,
X2 is an amino acid residue selected from a residue of Cys, Glu, Asp, Nmc, Nmd, Nme, Nmt, and Thr, and their corresponding d-forms,
X7 is an amino acid residue selected from a residue of Cys, Glu, Asp, Thr and Abu, and their corresponding d-forms,
wherein X2 and X7 are linked to each other via Q, wherein Q is a disulfide bond, a thioether bond or an ester bond,
X3 is an amino acid residue selected from a residue of Sar, His, Lys, Ala, Ahx, Apt, Arg, Asn, Gab, Gin, Trp, Thr, Ndmk, Nmn, Ntmk and Lys(αAc),
X4 is absent or is an amino acid residue selected from a residue of His, Lys, Ahx, Apt, Arg, Asn, Gab, Gln, Trp and Thr,
X5 is an amino acid residue selected from a residue of Trp, Wme, Nak, Wcl, Wim, Wdm and Wfl,
X6 is an amino acid residue selected from a residue of Gin, Aib, Glu, Phe, Ile, Iva, Lys, Lys(Ac), Leu, Mle, Mly, Mva, Thr, Val, Trp and Phe,
X8 is an amino acid residue selected from a residue of Yme, Tyr, Trp, Yde and Yae,
X9 is an amino acid residue selected from a residue of Nal, Nak and Trp,
X10 is an amino acid residue selected from a residue of Aib, Cba, Cit, d-Trp, d-Tza, Lys, Lys(Ac), Leu, Mle, Mly, Gln, Mkdm and Thp,
X11 is an amino acid residue selected from a residue of Asp, Bal, Gab, Glu, Kme, Lys and Orn and is linked via an amide bond to—X1,
X12 is absent or is an amino acid residue selected from a residue of Asn, Gly, Ser and Ala,
X13 is absent or is an amino acid residue selected from a residue of Asn, Ala, Bal, Gly, Leu,
Ile, Iva, Hol, Hph, Pal, Pyal, PyEA, His, Hme, Val, and their corresponding d-forms,
X14 is absent or is an amino acid residue selected from a residue of Mep, Sar, Bal, Ile, Dnmy and Hme,
X15 is absent or is an amino acid residue of Lys,
R2 is absent or is —NH₂, —OH or —N(C₂H₅)₂,
and
wherein amide bonds between X1 and X2, X2 and X3, X3 and X4, X4 and X5, X11 and X1 are optionally methylated, and
wherein at least one of X10 and X15 is an amino acid residue having a side chain with an —NH₂ group, wherein the —NH₂ side chain group is functionalized by —C(O)—R5, —C(O)O—R5, —C(O)NH—R5, —S(O)₂—R5 or R5, wherein R5 may be a moiety comprising up to 50 or up to 100 carbon atoms and optionally heteroatoms selected from halogen, N, O, S and P,
or a salt or solvate thereof,
wherein the compound of formula (Ia) inhibits the binding of interleukin-23 to an interleukin-23-receptor.

In some embodiments, at least one of X10 and X15 is an amino acid residue having a side chain with an —NH₂ group, wherein the —NH₂ side chain group is functionalized by —C(O)—R5, wherein R5 may be a moiety comprising up to 50 or up to 100 carbon atoms and optionally heteroatoms selected from halogen, N, O, S and P.

In one embodiment, X10 or X15 is an amino acid residue with a functionalized-NH₂ side chain group, such as functionalized Lys, Orn, Mly, Dab or Dap, or functionalized Lys or Mly.

An amino acid residue with an —NH₂ side chain group, e.g., Lys, Orn, Mly, Dab or Dap, may be functionalized in that at least one H atom of the —NH₂ side chain group is replaced by —C(O)—R5, —C(O)O—R5, —C(O)NH—R5, —S(O)2-R5 or R5, for example by —C(O)—R5, wherein R5 may be a moiety comprising up to 50 or up to 100 carbon atoms and optionally heteroatoms selected from halogen, N, O, S and P.

In certain embodiments, R5 may comprise a lipophilic moiety, e.g., an acyclic linear or branched saturated hydrocarbon group, wherein R5 for example comprises an acyclic linear or branched (C₄-C₃₀) saturated or unsaturated hydrocarbon group, and/or a cyclic saturated, unsaturated or aromatic group, for example a mono-, bi-, or tricyclic group comprising 4 to 14 carbon atoms and 0, 1, or 2 heteroatoms selected from N, O, and S, e.g., cyclohexyl, phenyl, biphenyl, chromanyl, phenanthrenyl or naphthyl, wherein the acyclic or cyclic group may be unsubstituted or substituted e.g., by halogen, —OH and/or CO₂H.

In some embodiments, R5 may comprise a lipophilic moiety, e.g., an acyclic linear or branched (C₁₂-C₂₂) saturated or unsaturated hydrocarbon group. The lipophilic moiety may be attached to the —NH₂ side chain group by a linker in all stereoisomeric forms, e.g., a linker comprising one or more, e.g., 2, amino acid linker groups such as γ-aminobutyric acid (GABA), ε-aminohexanoic acid (ε-Ahx), γ-Glu and/or β-Ala. In one embodiment the lipophilic moiety is attached to the —NH₂ side chain group by a linker. In another embodiment the lipophilic moiety directly attached to the —NH₂ side chain group. Specific examples of amino acid linker groups are (β-Ala)₁₋₄, (γ-Glu)₁₋₄, (ε-Ahx)₁₋₄, or (GABA)₁₋₄. Embodiments of amino acid linker groups are ß-Ala, γ-Glu, ß-Ala-ß-Ala and γ-Glu-γ-Glu.

Specific examples for —C(O)—R5 groups are listed in the following Table 2, which are selected from the group consisting of (S)-4-Carboxy-4-hexadecanoylamino-butyryl-, (S)-4-Carboxy-4-octadecanoylamino-butyryl-, 4-Hexadecanoylamino-butyryl-, 4-{3-[(R)-2,5,7,8-tetramethyl-2-((4R,8R)-4,8,12-trimethyl-tridecyl)-chroman-6-yloxycarbonyl]-propionylamino}-butyryl-, 4-octadecanoylamino-butyryl-, 4-((Z)-octadec-9-enoylamino)-butyryl-, 6-[(4,4-Diphenyl-cyclohexyloxy)-hydroxy-phosphoryloxy]-hexanoyl-, Hexadecanoyl-, (S)-4-Carboxy-4-(15-carboxy-pentadecanoylamino)-butyryl-, (S)-4-Carboxy-4-{3-[3-((2S,3R,4S,5R)-5-carboxy-2,3,4,5-tetrahydroxy-pentanoylamino)-propionylamino]-propionylamino}-butyryl-, (S)-4-Carboxy-4-{3-[(R)-2,5,7,8-tetramethyl-2-((4R,8R)-4,8,12-trimethyl-tridecyl)-chroman-6-yloxycarbonyl]-propionylamino}-butyryl-, (S)-4-Carboxy-4-((9Z,12Z)-octadeca-9,12-dienoylamino)-butyryl-, (S)-4-Carboxy-4-[6-((2S,3R,4S,5R)-5-carboxy-2,3,4,5-tetrahydroxy-pentanoylamino)-hexanoylamino]-butyryl-, (S)-4-Carboxy-4-((2S,3R,4S,5R)-5-carboxy-2,3,4,5-tetrahydroxy-pentanoylamino)-butyryl-, (S)-4-Carboxy-4-tetradecanoylamino-butyryl-, (S)-4-(11-Benzyloxycarbonyl-undecanoylamino)-4-carboxy-butyryl-, (S)-4-Carboxy-4-[11-((2S,3R,4R,5R)-2,3,4,5,6-pentahydroxy-hexylcarbamoyl)-undecanoylamino]-butyryl-, (S)-4-Carboxy-4-((Z)-octadec-9-enoylamino)-butyryl-, (S)-4-Carboxy-4-(4-dodecyloxy-benzoylamino)-butyryl-, (S)-4-Carboxy-4-henicosanoylamino-butyryl-, (S)-4-Carboxy-4-docosanoylamino-butyryl-, (S)-4-Carboxy-4-((Z)-nonadec-10-enoylamino)-butyryl-, (S)-4-Carboxy-4-(4-decyloxy-benzoylamino)-butyryl-, (S)-4-Carboxy-4-[(4′-octyloxy-biphenyl-4-carbonyl)-amino]-butyryl-, (S)-4-Carboxy-4-(12-phenyl-dodecanoylamino)-butyryl-, (S)-4-Carboxy-4-icosanoylamino-butyryl-, (S)-4-Carboxy-4-((S)-4-carboxy-4-hexadecanoylamino-butyrylamino)-butyryl-, (S)-4-Carboxy-4-((S)-4-carboxy-4-octadecanoylamino-butyrylamino)-butyryl-, 3-(3-Octadecanoylamino-propionylamino)-propionyl-, 3-(3-Hexadecanoylamino-propionylamino)-propionyl-, 3-Hexadecanoylamino-propionyl-, (S)-4-Carboxy-4-[(R)-4-((3R,5S,7R,8R,9R,10S,12S,13R,14R,17R)-3,7,12-trihydroxy-8,10,13-trimethyl-hexadecahydro-cyclopenta[a]phenanthren-17-yl)-pentanoylamino]-butyryl-, (S)-4-Carboxy-4-[(R)-4-((3R,5R,8R,9S,10S,13R,14S,17R)-3-hydroxy-10,13-dimethyl-hexadecahydro-cyclopenta[a]phenanthren-17-yl)-pentanoylamino]-butyryl-, (S)-4-Carboxy-4-((9S,10R)-9,10,16-trihydroxy-hexadecanoylamino)-butyryl-, Tetradecanoyl-, 11-Carboxy-undecanoyl-, 11-Benzyloxycarbonyl-undecanoyl-, (S)-4-Carboxy-4-((S)-4-carboxy-4-tetradecanoylamino-butyrylamino)-butyryl-, 6-[Hydroxy-(naphthalene-2-yloxy)-phosphoryloxy]-hexanoyl-, 6-[Hydroxy-(5-phenyl-pentyloxy)-phosphoryloxy]-hexanoyl-, 4 -(Naphthalene-2-sulfonylamino)-4-oxo-butyryl-, 4-(Biphenyl-4-sulfonylamino)-4-oxo-butyryl-, (S)-4-Carboxy-4-{(S)-4-carboxy-4-[2-(2-{2-[2-(2-(2-[(S)-4-carboxy-4-(17-carboxy-heptadecanoylamino)-butyrylamino]-ethoxy}-ethoxy)-acetylamino]-ethoxy}-ethoxy)-acetylamino]-butyrylamino}-butyryl-, (S)-4-Carboxy-4-[2-(2-{2-[2-(2-{2-[(S)-4-carboxy-4-(17-carboxy-heptadecanoylamino)-butyrylamino]-ethoxy}-ethoxy)-acetylamino]-ethoxy}-ethoxy)-acetylamino]-butyryl-, (S)-4-Carboxy-2-{(S)-4-carboxy-2-[2-(2-{2-[2-(2-{2-[(S)-4-carboxy-4-(17-carboxy-heptadecanoylamino)-butyrylamino]-ethoxy}-ethoxy)-acetylamino]-ethoxy}-ethoxy)-acetylamino]-butyrylamino}-butyryl-, (S)-4-Carboxy-2-[2-(2-(2-[2-(2-{2-[(S)-4-carboxy-4-(17-carboxy-heptadecanoylamino)-butyrylamino]-ethoxy}-ethoxy)-acetylamino]-ethoxy}-ethoxy)-acetylamino]-butyryl-, (S)-4-Carboxy-4-{(S)-4-carboxy-4-[2-(2-{2-[(S)-4-carboxy-4-(17-carboxy-heptadecanoylamino)-butyrylamino]-ethoxy}-ethoxy)-acetylamino]-butyrylamino]-butyryl-, (S)-4-Carboxy-4-[2-(2-{2-[(S)-4-carboxy-4-(17-carboxy-heptadecanoylamino)-butyrylamino]-ethoxy}-ethoxy)-acetylamino]-butyryl-, (S)-4-Carboxy-2-{(S)-4-carboxy-2-[2-(2-{2-[(S)-4-carboxy-4-(17-carboxy-heptadecanoylamino)-butyrylamino]-ethoxy}-ethoxy)-acetylamino]-butyrylamino}-butyryl-, (S)-4-Carboxy-2-[2-(2-{2-[(S)-4-carboxy-4-(17-carboxy-heptadecanoylamino)-butyrylamino]-ethoxy)-ethoxy)-acetylamino]-butyryl-, 2-(2-{2-[2-(2-{2-[(S)-4-Carboxy-4-(17-carboxy-hepta-decanoylamino)-butyrylamino]-ethoxy}-ethoxy)-acetylamino]-ethoxy}-ethoxy)-acetyl-, 2-(2-{2-[(S)-4-Carboxy-4-(17-carboxy-heptadecanoylamino)-butyrylamino]-ethoxy]-ethoxy)-acetyl, (S)-4-Carboxy-4-((S)-4-carboxy-4-{(S)-4-carboxy-4-[(S)-4-carboxy-4-(19-carboxy-nonadecanoylamino)-butyrylamino]-butyrylamino}-butyrylamino)-butyryl, 2-(2-{2-[2-(2-{2-[(S)-4-Carboxy-4-(16-1H-tetrazol-5-yl-hexadecanoylamino)-butyrylamino]-ethoxy}-ethoxy)-acetylamino]-ethoxy}-ethoxy)-acetyl-, 2-(2-{2-[2-(2-{2-[(S)-4-Carboxy-4-(16-carboxy-hexadecanoylamino)-butyrylamino]-ethoxy]-ethoxy)-acetylamino]-ethoxy}-ethoxy)-acetyl-, (S)-4-Carboxy-4-{(S)-4-carboxy-4-[(S)-4-carboxy-4-(17-carboxy-heptadecanoylamino)-butyrylamino]-butyrylamino}-butyryl-, (S)-4-Carboxy-4-((S)-4-carboxy-4-{2-[2-(2-{2-[2-(2-{(S)-4-carboxy-4-[10-(4-carboxy-phenoxy)-decanoylamino]-butyrylamino}-ethoxy)-ethoxy]-acetylamino}-ethoxy)-ethoxy]-acetylamino}-butyryl-, (S)-4-Carboxy-4-{(S)-4-carboxy-4-[2-(2-{2-[2-(2-{2-[(S)-4-carboxy-4-(7-carboxy-heptanoylamino)-butyrylamino]-ethoxy}-ethoxy)-acetylamino]-ethoxy}-ethoxy)-acetylamino]-butyrylamino}-butyryl-, (S)-4-Carboxy-4-{(S)-4-carboxy-4-[2-(2-{2-[2-(2-{2-[(S)-4-carboxy-4-(11-carboxy-undecanoylamino)-butyrylamino]-ethoxy}-ethoxy)-acetylamino]-ethoxy]-ethoxy)-acetylamino]-butyrylamino}-butyryl-, (S)-4-Carboxy-4-{(S)-4-carboxy-4-[2-(2-{2-[2-(2-{2-[(S)-4-carboxy-4-(13-carboxy-tridecanoylamino)-butyrylamino]-ethoxy}-ethoxy)-acetylamino]-ethoxy]-ethoxy)-acetylamino]-butyrylamino}-butyryl-, (S)-4-Carboxy-4-{(S)-4-carboxy-4-[2-(2-{2-[2-(2-{2-[ (S)-4-carboxy-4-(15-carboxy-pentadecanoylamino)-butyrylamino]-ethoxy]-ethoxy)-acetylamino]-ethoxy)-ethoxy)-acetylamino]-butyrylamino}-butyryl-, and (S)-4-Carboxy-4-{(S)-4-carboxy-4-[2-(2-{2-[2-(2-{2-[(S)-4-carboxy-4-(19-carboxy-nonadecanoylamino)-butyrylamino]-ethoxy}-ethoxy)-acetylamino]-ethoxy}-ethoxy)-acetylamino]-butyrylamino}-butyryl-.

Other specific examples for-C(O)—R5 groups are tetradecanoyl, hexadecanoyl and 2-(2-{2-[2-(2-{2-[(S)-4-Carboxy-4-(17-carboxy-heptadecanoylamino)-butyrylamino]-ethoxy}-ethoxy)-acetylamino]-ethoxy}-ethoxy)-acetyl-.

Other specific examples for-C(O)—R5 groups are listed in Table 2 as Myri, Palm and Sema.

Further embodiments are stereoisomers, for example enantiomers of these groups, either S- or R-enantiomers. The term “R” in Table 2 is intended to mean the attachment site of —C(O)—R5 at the peptide back bone, i.e., for example the s-amino group of Lys or Mly.

TABLE 2
—C(O)R5 functionalizing groups

| structure | IUPAC | name |
|---|---|---|
| 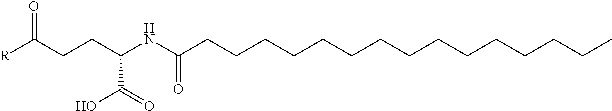 | (S)-4-Carboxy-4-hexadecanoylamino- butyryl- | _{Y}E-x53 |
| 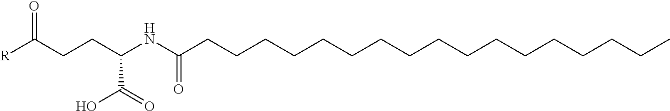 | (S)-4-Carboxy-4-octadecanoylamino- butyryl- | _{Y}E-x70 |
| 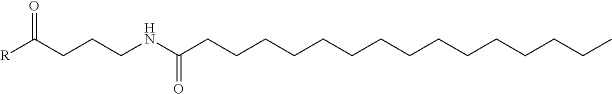 | Hexa ecanoylamino -butyryl- | GABA-x53 |
| 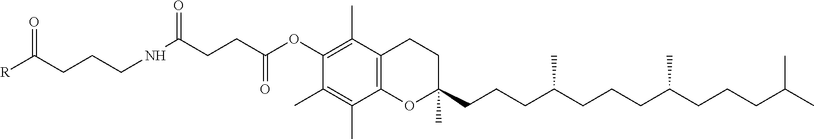 | 4-(3-((R)-2,5,7,8-tetramethyl-2-((4R, BR)- 4,8,12-trimethyl-tridecyl)-chroman-6- yloxycarbonyl]-propionylamino)-butyryl | GABA-x60 |
| 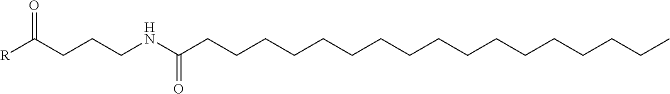 | 4-octadecanoylamino-butyryl- | GABA-x70 |
| 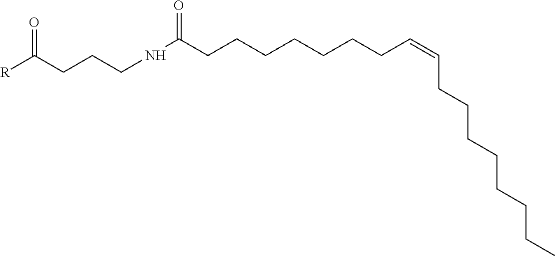 | 4-(Z)-octadec-9-enoylamino)-butyryl- | GABA-x74 |
| 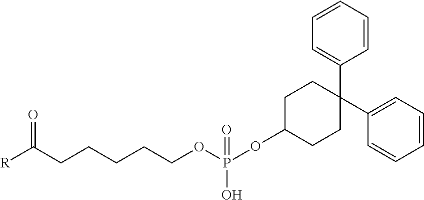 | 6-[(4,4-Diphenyl-cyclohexyloxy)- hydroxy-phosphoryloxy]-hexanoyl- | Phospho1 |
| 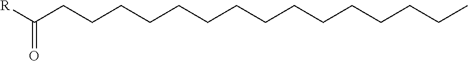 | Hexadecanoyl- | x53 |
| 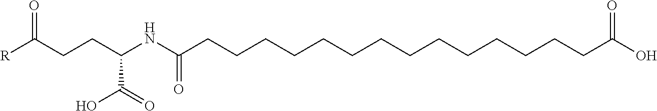 | (S)-4-Carboxy-4-(15-carboxy- pentadecanoylamino)-butyryl- | x52 |
| 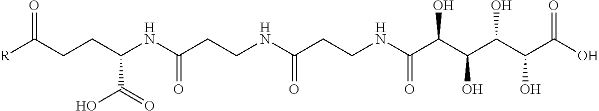 | (S)-4-Carboxy-4-(3-[3-((2S,3R,4S,5R)-5- carboxy-2,3,4,5-tetrahydroxy- pentanoylamino)-propionylamino]- propionylamino)-butyryl | _{Y}E-x59 |
| 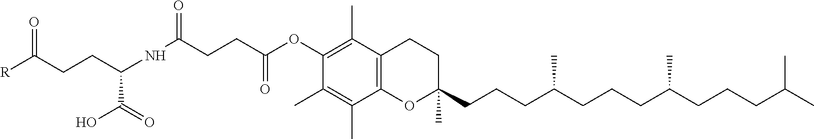 | (S)-4- Carboxy-4-(3-[(R)-2,5,7,8- tetramethyl-2-((4R,8R)-4,8,12-trimethyl- tridecyl)-chroman-6-yloxycarbonyl). [propionylamino)-butyryl- | _{Y}E-x60 |
| 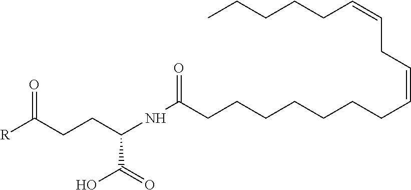 | (S)-4-Carboxy-4-((9Z,12Z)-octadeca-9,12 dienoylamino)-butyryl- | _{Y}E-x61 |
| 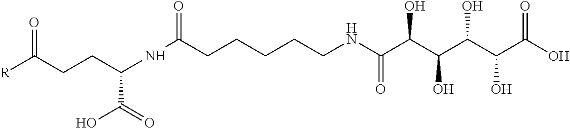 | (S)-4-Carboxy-4-[6-((2S,3R,4S,5R)-5- carboxy-2,3,4,5-tetrahydroxy- pentanoylamino)-hexanoylamino]- butyryl | _{Y}E-x64 |
| 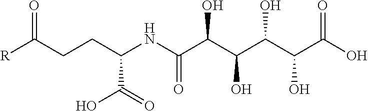 | (S)-4-Carboxy-4-((2S,3R,4S,5R)-5- carboxy-2,3,4,5 tetrahydroxy- pentanoylamino)-butyryl | _{Y}E-x65 |
| 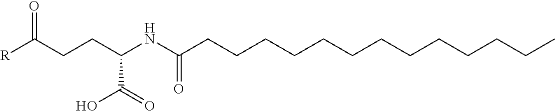 | (S)-4-Carboxy-4-tetradecanoylamino- butyryl- | _{Y}E-x69 |
| 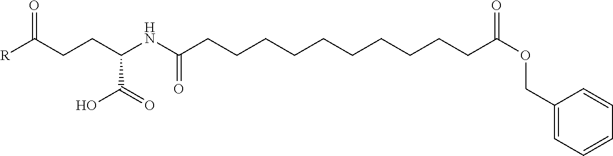 | (S)-4-(11-Benzyloxycarbonyl- undecanoylamino)-4-carboxy-butyryl | _{Y}E-x72 |
| 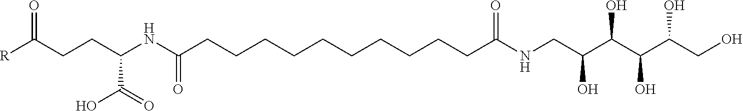 | (S)-4-Carboxy-4-[11-((2S,3R,4R,5R)- 2.3,4,5,6-pentahydroxy-hexylcarbamoyl) undecanoylaminoj-butyryl- | _{Y}E-x73 |
| 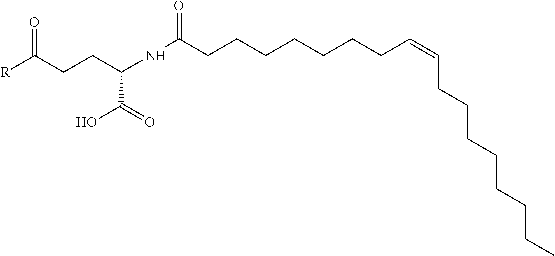 | (S)-4-Carboxy-4-((Z)-octadec-9- enoylamino)-butyryl- | _{Y}E-x74 |
| 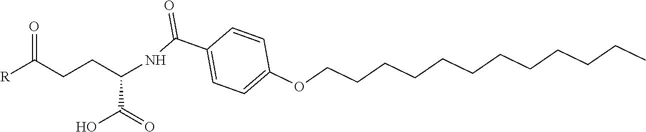 | (S)-4-Carboxy-4-(4-dodecyloxy- benzoylamino)-butyryl- | _{Y}E-x75 |
| 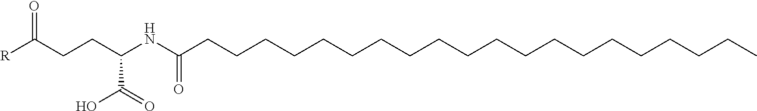 | (S)-4-Carboxy-4-henicosanoylamino- butyryl- | _{Y}E-x76 |
| 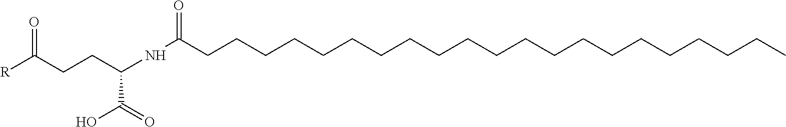 | (S)-4-Carboxy-4-docosanoylamino- butyryl- | _{Y}E-x77 |
| 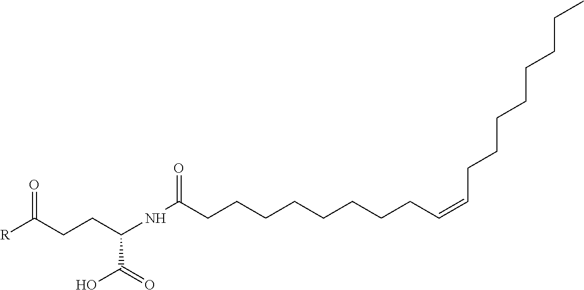 | (S)-4-Carboxy-4-((Z)-nonadec-10- enoylamino)-butyryl- | _{Y}E-x79 |
| 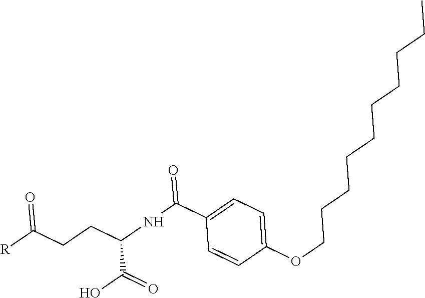 | (S)-4-Carboxy-4-(4-decyloxy- benzoylamino)-butyryl- | _{Y}E-x80 |
| 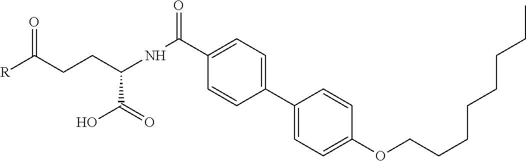 | (S)-4-Carboxy-4-[(4′-octyloxy-biphenyl-4 carbonyl)-amino]-butyryl- | _{Y}E-x81 |
| 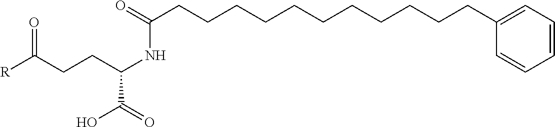 | (S)-4-Carboxy-4-(12-phenyl- dodecanoylamino)-butyryl- | _{Y}E-x81 |
| 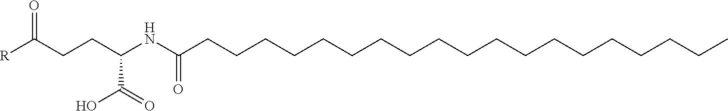 | (S)-4-Carboxy-4-icosanoylamino- butyryl- | _{Y}E-X95 |
| 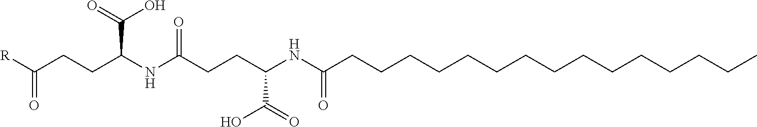 | (S)-4-Carboxy-4-((S)-4-carboxy-4 hexadecanoylamino-butyrylamino)- butyryl- | _{Y}E-_{Y}E-x53 |
| 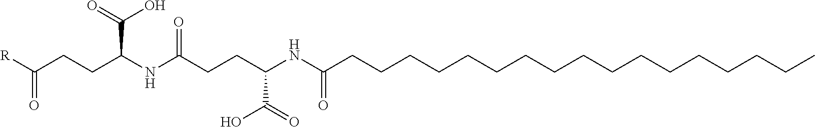 | (S)-4-Carboxy-4-((S)-4-carboxy-4- octadecanoylamino-butyrylamino) butyryl- | _{Y}E-_{Y}E-x70 |
| 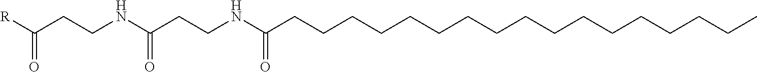 | 3-(3-Octadecanoylamino- propionylamino) propionyk- | β-Ala-β-Ala-x70 |
| 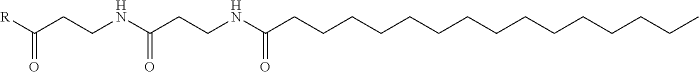 | 3-(3-Hexadecanoylamino- propionylamino)propionyl- | β-Ala-β-Ala-x53 |
| 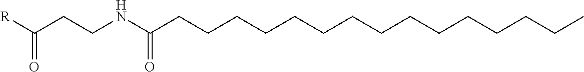 | 3-Hexadecanoylamino-propionyl- | β-Ala-x53 |
| 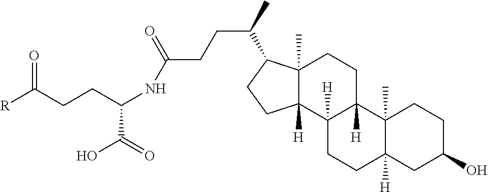 | (S)-4-Carboxy-4-{(R)-4+ (3R.5S.7R,8R,9R,10S,12S,13R,14R,17R) 3,7,12-trihydroxy-8,10,13-trimethyl- hexadecahydro- cyclopenta[a]phenanthren-17-yl)- pentanoylamino]-butyryl- | _{Y}E-x16 |
| 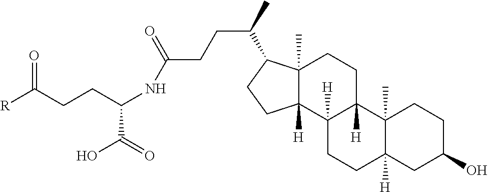 | (S)-4-Carboxy-4[(R)-4- (3R,5R,8R,9S,10S,13R,14S,17R)-3- hydroxy-10,13-dimethyl-hexadecahydro cyclopenta[a]phenanthren-17-yl)- pentanoylamino]-butyryl- | _{Y}E-x19 |
| 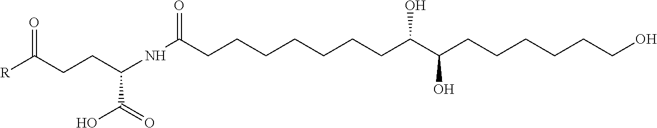 | (S)-4-Carboxy-4-((9S,10R)-9,10,16- trihydroxy-hexadecanoylamino)-butyryl- | _{Y}E-x25 |
| 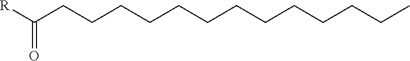 | tetradecanoyl- | x69 |
| 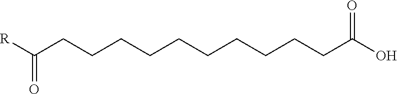 | 11-Carboxy-undecanoyl- | x71 |
| 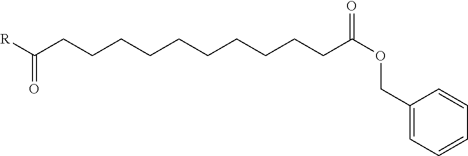 | 11-Benzyloxycarbonyl-undecanoyl | x72 |
| 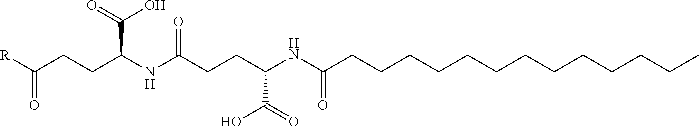 | (S)-4-Carboxy-4-((S)-4-carboxy-4 tetradecanoylamino-butyrylamino)- butyryl- | _{Y}E-_{Y}E-x69 |
| 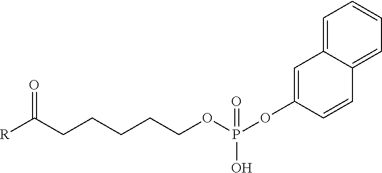 | 6-[Hydroxy-(naphthalen-2-yloxy). phosphoryloxy]-hexanoyl- | Phospho2 |
| 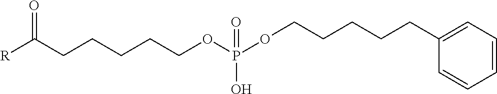 | 6-[Hydroxy-(5-phenyl-pentyloxy)- phosphoryloxyj-hexanoyl- | Phospho3 |
| 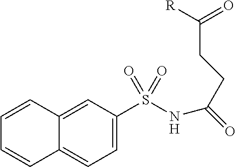 | 4-(Naphthalene-2-sulfonylamino)-4-oxo- butyryl- | Sulfonamid 1 |
| 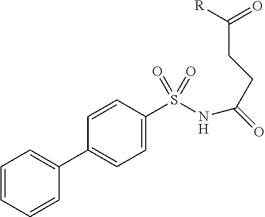 | 4-(Biphenyl-4-sulfonylamino)-4-oxo- butyryl- | Sulfonamid 2 |
| 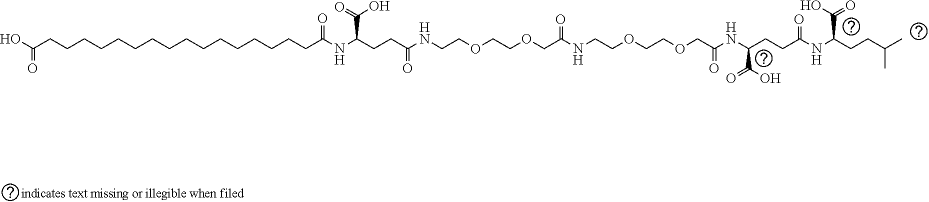 [SMILES: CC(C)CC[C@@H](NC(=O)CC[C@H](NC(=O)COCCOCCNC(=O)COCCOCCNC(=O)CC[C@@H](NC(=O)CCCCCCCCCCCCCCCCC(=O)O)C(=O)O)C(=O)O)C(=O)O] | (S)-4-Carboxy-4-((S)-4-carboxy-4-[2-(2- (2-[2-(2-(2-[(S)-4 carboxy-4-(17-carboxy- heptadecanoylamino)-butyrylamino)- ethoxy)ethoxy)-acetylamino]-ethoxy)- ethoxy)-acetylamino]-butyrylamino)- butyryl- | x100 |
| 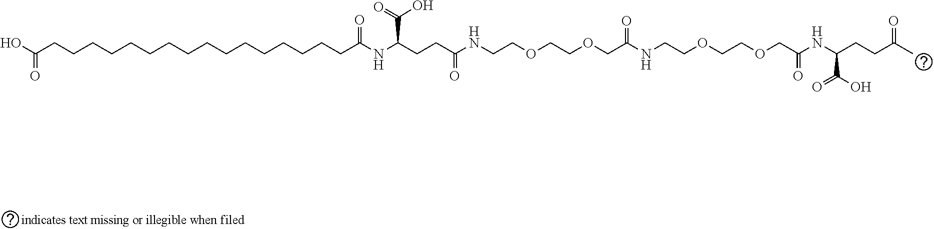 [SMILES: CC(=O)CC[C@H](NC(=O)COCCOCCNC(=O)COCCOCCNC(=O)CC[C@@H](NC(=O)CCCCCCCCCCCCCCCCC(=O)O)C(=O)O)C(=O)O] | (S)-4-Carboxy-4-[2-(2-[2-[2-(2-(2-{(S)-4- carboxy-4-(17-carboxy- heptadecanoylamino)butyrylamino]- ethoxy)-ethoxy)-acetylamino]-ethoxy)- ethoxy)-acetylamino]-butyryl | x101 |
| 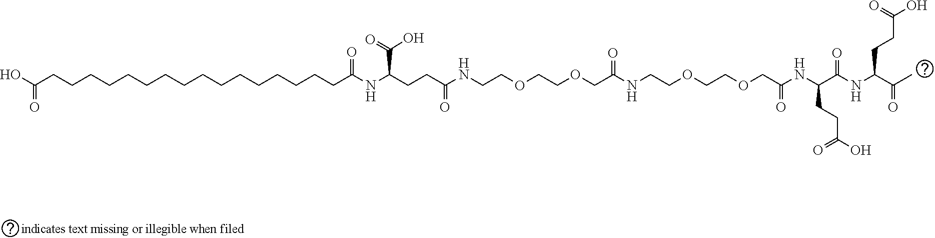 [SMILES: CC(=O)[C@H](CCC(=O)O)NC(=O)[C@@H](CCC(=O)O)NC(=O)COCCOCCNC(=O)COCCOCCNC(=O)CC[C@@H](NC(=O)CCCCCCCCCCCCCCCCC(=O)O)C(=O)O] | (S)-4-Carboxy-2-((S)-4-carboxy-2-[2-(2- (2-[2-(2-{2-(S)-4-carboxy-4-(17-carboxy- heptadecanoylamino)-butyrylamino]- ethoxy)-ethoxy)-acetylamino]-ethoxy) ethoxy)-acetylamino]-butyrylamino}- butyryl | x102 |
| 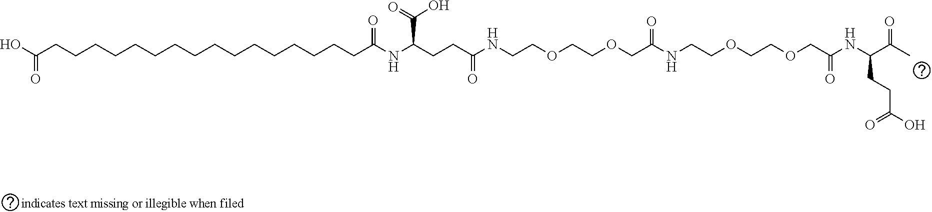 [SMILES: CC(=O)[C@@H](CCC(=O)O)NC(=O)COCCOCCNC(=O)COCCOCCNC(=O)CC[C@@H](NC(=O)CCCCCCCCCCCCCCCCC(=O)O)C(=O)O] | (S)-4-Carboxy-2-[2-(2-(2-[2-(2-(2-[(9)-4- carboxy-4-(17-carboxy- heptadecanoylamino)-butyrylamino)- ethoxy)-ethoxy)-acetylamino]-ethoxy). ethoxy)-acetylamino]-butyryl | x103 |
| 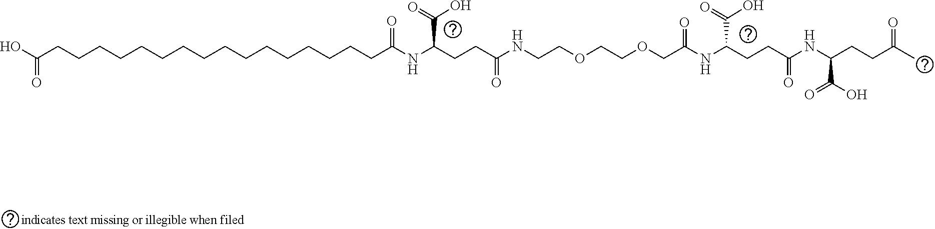 [SMILES: CC(=O)CC[C@H](NC(=O)CC[C@H](NC(=O)COCCOCCNC(=O)CC[C@@H](NC(=O)CCCCCCCCCCCCCCCCC(=O)O)C(=O)O)C(=O)O)C(=O)O] | (S)-4-Carboxy-4-((S)-4-carboxy-4-(2-(2- (2-[(S)-4-carboxy-4-(17-carboxy- heptadecanoylamino)-butyrylamino]- ethoxy]-ethoxy)-acetylamino]- butyrylamino)-butyryl | x104 |
| 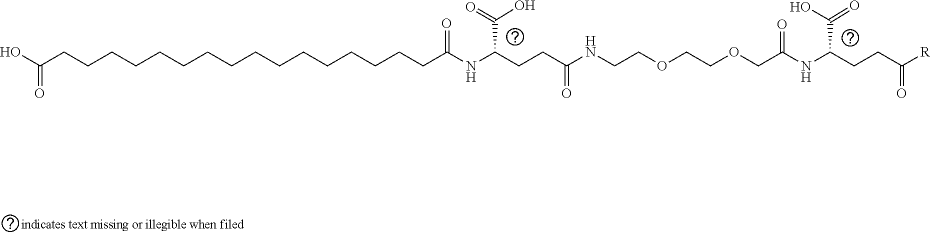 | (S)-4-Carboxy-4-[2-(2-(2-[(S)-4-carboxy- 4-(17-carboxy-heptadecanoylamino)- butyrylamino)-ethoxy)-ethoxy)- acetylamino]-butyryl | x105 |
| 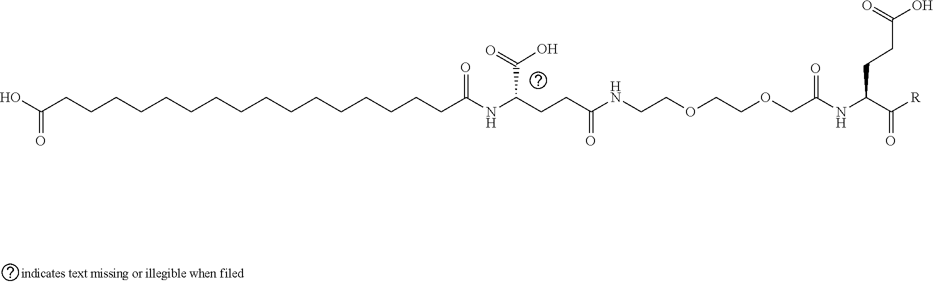 | (S)-4-Carboxy-2-((S)-4-carboxy-2-(2-(2- [2-[(S)-4-carboxy-4-(17-carboxy- heptadecanoylamino)-butyrylamino]- ethoxy)-ethoxy)-acetylamino]- butyrylamino)-butyryl | x106 |
| 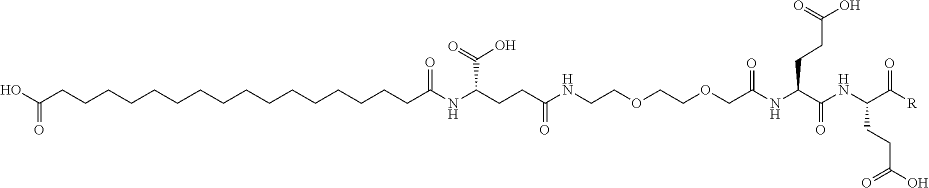 | [(S)-4-Carboxy-2-[2-(2-(2-[(S)-4-carboxy- 4-(17-carboxy-heptadecanoylamino)- butyrylamino]-ethoxy)-ethoxy)- acetylamino]-butyryl | x107 |
| 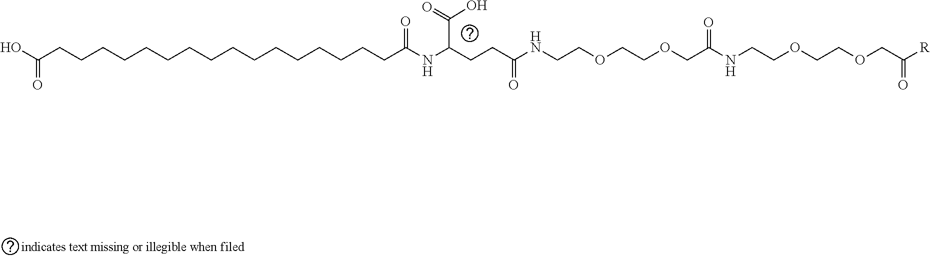 | 2-(2-(2-[2-(2-(2-(S)-4-Carboxy-4-(17- carboxy-heptadecanoylamino)- butyrylamino]-ethoxy)-ethoxy)- acetylamino)-ethoxy)-ethoxy)-acetyl- | x108 |
| 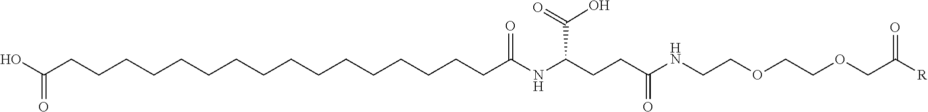 | 2-(2-(2-[(S)-4-Carboxy-4-(17-carboxy- heptadecanoylamino)-butyrylamino]- ethoxy)-ethoxy)-acetyl | x109 |
| 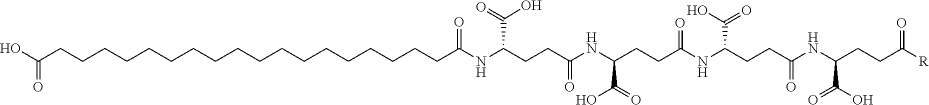 | [(S)-4-Carboxy-4-((S)-4-carboxy-4-((S)-4- carboxy-4-[(S) 4-carboxy-4-(19-carboxy- nonadecanoylamino)-butyrylamino)- butyrylamino)-butyrylamino)-butyryl | x110 |
| 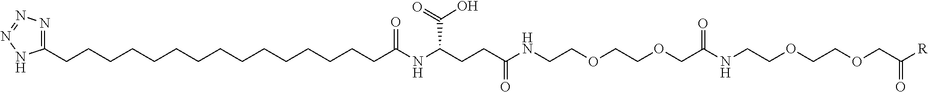 | 2-(2-(2-[2-(2-(2-[(S)-4-Carboxy-4-(16-1H- tetrazol-5-yl-hexadecanoylamino)- butyrylamino]-ethoxy)-ethoxy)- acetylamino]-othoxy}-ethoxy)-acetyl | x111 |
| 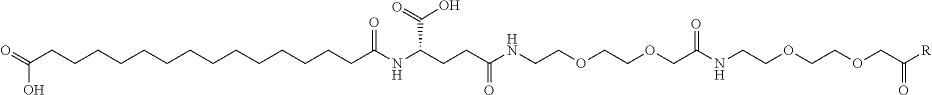 | 2-(2-(2-[2-(2-(2-(S)-4-Carboxy-4-(16- carboxy-hexadecanoylamino)- butyrylamino]-ethoxy)-ethoxy)- acetylamino]-ethoxy)-ethoxy)-acetyl | x112 |
| 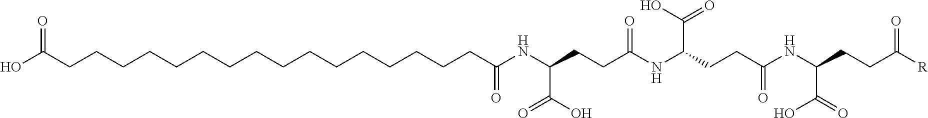 | (S)-4-Carboxy-4-((S)-4-carboxy-4-((S)-4- carboxy-4-(17-carboxy- heptadecanoylamino)-butyrylamino]- butyrylamino)-butyryl | x113 |
| 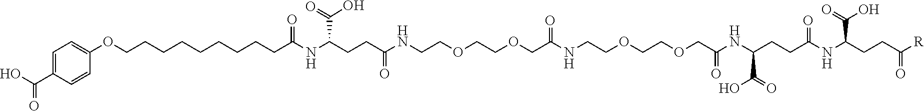 | (S)-4-Carboxy-4-((S)-4-carboxy-4-(2-[2- (2-(2-[2-(2-((S)-4 carboxy-4-[10-(4- carboxy-phenoxy)-decanoylamino)- butyrylamino)-ethoxy)-ethoxy]- acetylamino)-ethoxy)-ethoxy]. acetylamino)-butyryl | x114 |
| 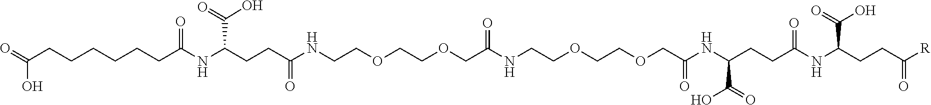 | (S)-4-Carboxy-4-((S)-4-carboxy-4-[2-(2- (2-[2-(2-{2-[(S)-4-carboxy-4-(7-carboxy- [heptanoylamino)-butyrylamino]-ethoxy)- ethoxy)-acetylamino]-ethoxy)-ethoxy)- acetylamino]-butyrylamino)-butyryl | x115 |
| 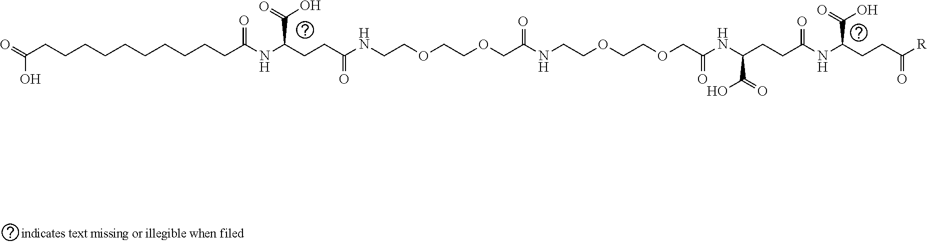 | (S)-4-Carboxy-4-((S)-4-carboxy-4-[2-(2- (2-[2-(2-(2-[(S)-4-carboxy-4-(11-carboxy- undecanoylamino)-butyrylamino)- ethoxy)-ethoxy)-acetylamino]-ethoxy)- ethoxy) acetylamino]-butyrylamino)- butyryl | x116 |
| 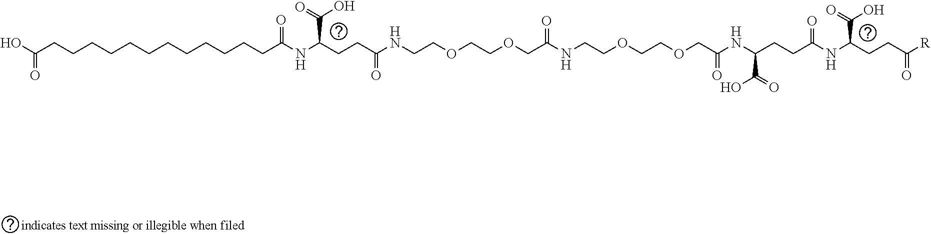 | (S)-4-Carboxy-4-((S)-4-carboxy-4-[2-(2- (2-[2-(2-(2-[(S)-4-carboxy-4-(13-carboxy- tridecanoylamino)-butyrylamino]- ethoxy]-ethoxy)-acetylamino] ethoxy)- ethoxy)-acetylamino]-butyrylamino)- butyryl | x117 |
| 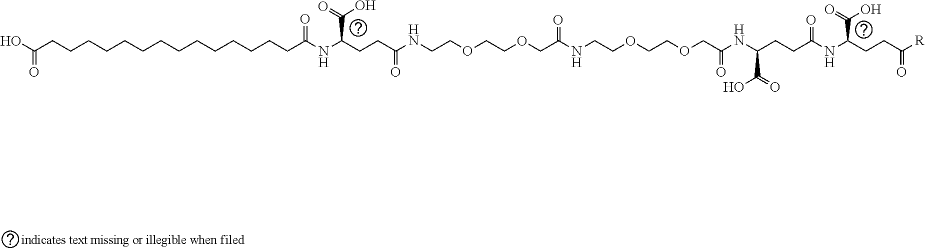 | (S)-4-Carboxy-4-{(S)-4-carboxy -4-[2-(2- (2-[2-(2-(2-[(S)-4-carboxy-4-(15-carboxy- pentadecanoylamino)-butyrylamino]- ethoxy)-ethoxy)-acetylamino]-ethoxy)- ethoxy)-acetylamino]-butyrylamino)- butyryl | x118 |
| 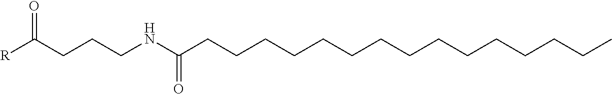 | (S)-4-Carboxy-4-{(S)-4-carboxy-4-[2-(2- (2-[2-(2-(2-[(S)-4-carboxy-4-(19-carboxy- nonadecanoylamino) butyrylamino)- ethoxy)-ethoxy)-acetylamino]-ethoxy)- ethoxy)-acetylamino]-butyrylamino)- butyryl | x119 |

According to one embodiment, R5 is selected from the group consisting of (S)-4-carboxy-4-hexadecanoylamino-butyryl (γE-x53), (S)-4-carboxy-4-octadecanoylamino-butyryl (γE-x70), 4-hexadecanoylamino-butyryl (GABA-x53), 4-{3-[(R)-2,5,7,8-tetramethyl-2-((4R,8R)-4,8,12-trimethyl-tridecyl)-chroman-6-yloxycarbonyl]-propionylamino}-butyryl-(GABA-x60), 4-octadecanoylamino-butyryl (GABA-x70), 4-((Z)-octadec-9-enoylamino)-butyryl (GABA-x74), 6-[(4,4-Diphenyl-cyclohexyloxy)-hydroxy-phosphoryloxy]-hexanoyl (Phospho1), Hexadecanoyl (x53),), Tetradecanoyl (x69), (S)-4-Carboxy-4-(15-carboxy-pentadecanoylamino)-butyryl (x52), (S)-4-Carboxy-4-{3-[3-((2S,3R,4S,5R)-5-carboxy-2,3,4,5-tetrahydroxy-pentanoylamino)-propionylamino]-propionylamino}-butyryl (γE-x59), (S)-4-Carboxy-4-{3-[(R)-2,5,7,8-tetramethyl-2-((4R,8R)-4,8,12-trimethyl-tridecyl)-chroman-6-yloxycarbonyl]-propionylamino}-butyryl (γE-x60), (S)-4-Carboxy-4-((9Z,12Z)-octadeca-9,12-dienoylamino)-butyryl (γE-x61), (S)-4-Carboxy-4-[6-((2S,3R,4S,5R)-5-carboxy-2,3,4,5-tetrahydroxy-pentanoylamino)-hexanoylamino]-butyryl (γE-x64), (S)-4-Carboxy-4-((2S,3R,4S,5R)-5-carboxy-2,3,4,5-tetrahydroxy-pentanoylamino)-butyryl (γE-x65), (S)-4-carboxy-4-tetradecanoylamino-butyryl (γE-x69), (S)-4-(11-Benzyloxycarbonyl-undecanoylamino)-4-carboxy-butyryl (γE-x72), (S)-4-carboxy-4-[11-((2S,3R,4R,5R)-2,3,4,5,6-pentahydroxy-hexylcarbamoyl)-undecanoylamino]-butyryl (γE-x73), (S)-4-Carboxy-4-((Z)-octadec-9-enoylamino)-butyryl (γE-x74), (S)-4-Carboxy-4-(4-dodecyloxy-benzoylamino)-butyryl (γE-x75), (S)-4-Carboxy-4-henicosanoylamino-butyryl (γE-x76), (S)-4-Carboxy-4-docosanoylamino-butyryl (γE-x77), (S)-4-Carboxy-4-((Z)-nonadec-10-enoylamino)-butyryl (γE-x79), (S)-4-Carboxy-4-(4-decyloxy-benzoylamino)-butyryl (γE-x80), (S)-4-Carboxy-4-[(4′-octyloxy-biphenyl-4-carbonyl)-amino]-butyryl (γE-x81), (S)-4-Carboxy-4-(12-phenyl-dodecanoylamino)-butyryl (γE-x82), (S)-4-Carboxy-4-icosanoylamino-butyryl (γE-x95), (S)-4-Carboxy-4-((S)-4-carboxy-4-hexadecanoylamino-butyrylamino)-butyryl (γE-γE-x53), (S)-4-Carboxy-4-((S)-4-carboxy-4-octadecanoylamino-butyrylamino)-butyryl (γE-γE-x70), and 3-(3-Octadecanoylamino-propionylamino)-propionyl (β-Ala-β-Ala-x70).

According to another embodiment, R5 is selected from the group consisting of (S)-4-carboxy-4-octadecanoylamino-butyryl (γE-x70), (S)-4-carboxy-4-hexadecanoylamino-butyryl (γE-x53), and hexadecanoyl (x53).

According to yet another embodiment, R5 is (S)-4-carboxy-4-hexadecanoylamino-butyryl (γE-x53).

In some embodiments, position X10 or X15 represents Lys or Mly. According to some embodiments, Lys or Mly at position X10, Lys at position X15 is functionalized, e.g., with a group —C(O)R5 as described above.

In other embodiments, X15 is absent and X10 is Mly functionalized with —C(O)—R5, —C(O)O—R5, —C(O)NH—R5, —S(O)2-R5 or R5, for example by —C(O)—R5, wherein R5 is as defined above.

In other embodiments, X10 is not functionalized and X15 is Lys functionalized with —C(O)—R5, —C(O)O—R5, —C(O)NH—R5, —S(O)2-R5 or R5, for example by —C(O)—R5, wherein R5 is as defined above.

In other embodiments, X10 is not functionalized, X14 is absent and X15 is Lys functionalized with —C(O)—R5, —C(O)O—R5, —C(O)NH—R5, —S(O)2-R5 or R5, for example by —C(O)—R5, wherein R5 is as defined above.

In some embodiments, X15 is absent and X10 is Mly functionalized with C(O)—R5, wherein R5 is selected from the group consisting of (S)-4-carboxy-4-hexadecanoylamino-butyryl (γE-x53), (S)-4-carboxy-4-octadecanoylamino-butyryl (γE-x70), 4-hexadecanoylamino-butyryl (GABA-x53), 4-{3-[(R)-2,5,7,8-tetramethyl-2-((4R,8R)-4,8,12-trimethyl-tridecyl)-chroman-6-yloxycarbonyl]-propionylamino}-butyryl-(GABA-x60), 4-octadecanoylamino-butyryl (GABA-x70), 4-((Z)-octadec-9-enoylamino)-butyryl (GABA-x74), 6-[(4,4-Diphenyl-cyclohexyloxy)-hydroxy-phosphoryloxy]-hexanoyl (Phospho1), Hexadecanoyl (x53), (S)-4-Carboxy-4-(15-carboxy-pentadecanoylamino)-butyryl (x52), (S)-4-Carboxy-4-{3-[3-((2S,3R,4S,5R)-5-carboxy-2,3,4,5-tetrahydroxy-pentanoylamino)-propionylamino]-propionylamino}-butyryl (γE-x59), (S)-4-Carboxy-4-{3-[(R)-2,5,7,8-tetramethyl-2-((4R,8R)-4,8,12-trimethyl-tridecyl)-chroman-6-yloxycarbonyl]-propionylamino}-butyryl (γE-x60), (S)-4-Carboxy-4-((9Z,12Z)-octadeca-9,12-dienoylamino)-butyryl (γE-x61), (S)-4-Carboxy-4-[6-((2S,3R,4S,5R)-5-carboxy-2,3,4,5-tetrahydroxy-pentanoylamino)-hexanoylamino]-butyryl (γE-x64), (S)-4-Carboxy-4-((2S,3R,4S,5R)-5-carboxy-2,3,4,5-tetrahydroxy-pentanoylamino)-butyryl (γE-x65), (S)-4-carboxy-4-tetradecanoylamino-butyryl (γE-x69), (S)-4-(11-Benzyloxycarbonyl-undecanoylamino)-4-carboxy-butyryl (γE-x72), (S)-4-carboxy-4-[11-((2S,3R,4R,5R)-2,3,4,5,6-pentahydroxy-hexylcarbamoyl)-undecanoylamino]-butyryl (γE-x73), (S)-4-Carboxy-4-((Z)-octadec-9-enoylamino)-butyryl (γE-x74), (S)-4-Carboxy-4-(4-dodecyloxy-benzoylamino)-butyryl (γE-x75), (S)-4-Carboxy-4-henicosanoylamino-butyryl (γE-x76), (S)-4-Carboxy-4-docosanoylamino-butyryl (γE-x77), (S)-4-Carboxy-4-((Z)-nonadec-10-enoylamino)-butyryl (γE-x79), (S)-4-Carboxy-4-(4-decyloxy-benzoylamino)-butyryl (γE-x 80), (S)-4-Carboxy-4-[(4′-octyloxy-biphenyl-4-carbonyl)-amino]-butyryl (γE-x81), (S)-4-Carboxy-4-(12-phenyl-dodecanoylamino)-butyryl (γE-x82), (S)-4-Carboxy-4-icosanoylamino-butyryl (γE-x95), (S)-4-Carboxy-4-((S)-4-carboxy-4-hexadecanoylamino-butyrylamino)-butyryl (γE-γE-x53), (S)-4-Carboxy-4-((S)-4-carboxy-4-octadecanoylamino-butyrylamino)-butyryl (γE-γE-x70), and 3-(3-Octadecanoylamino-propionylamino)-propionyl (β-Ala-β-Ala-x70).

In some embodiments, X10 is not functionalized and X15 is Lys functionalized with C(O)—R5, wherein R5 is selected from the group consisting of (S)-4-carboxy-4-hexadecanoylamino-butyryl (γE-x53), (S)-4-carboxy-4-octadecanoylamino-butyryl (γE-x70), 4-hexadecanoylamino-butyryl (GABA-x53), 4-{3-[(R)-2,5,7,8-tetramethyl-2-((4R,8R)-4,8,12-trimethyl-tridecyl)-chroman-6-yloxycarbonyl]-propionylamino}-butyryl-(GABA-x60), 4-octadecanoylamino-butyryl (GABA-x70), 4-((Z)-octadec-9-enoylamino)-butyryl (GABA-x74), 6-[(4,4-Diphenyl-cyclohexyloxy)-hydroxy-phosphoryloxy]-hexanoyl (Phospho1), Hexadecanoyl (x53), (S)-4-Carboxy-4-(15-carboxy-pentadecanoylamino)-butyryl (x52), (S)-4-Carboxy-4-{3-[3-((2S,3R,4S,5R)-5-carboxy-2,3,4,5-tetrahydroxy-pentanoylamino)-propionylamino]-propionylamino}-butyryl (γE-x59), (S)-4-Carboxy-4-{3-[(R)-2,5,7,8-tetramethyl-2-((4R,8R)-4,8,12-trimethyl-tridecyl)-chroman-6-yloxycarbonyl]-propionylamino}-butyryl (γE-x60), (S)-4-Carboxy-4-((9Z,12Z)-octadeca-9,12-dienoylamino)-butyryl (γE-x61), (S)-4-Carboxy-4-[6-((2S,3R,4S,5R)-5-carboxy-2,3,4,5-tetrahydroxy-pentanoylamino)-hexanoylamino]-butyryl (γE-x64), (S)-4-Carboxy-4-((2S,3R,4S,5R)-5-carboxy-2,3,4,5-tetrahydroxy-pentanoylamino)-butyryl (γE-x65), (S)-4-carboxy-4-tetradecanoylamino-butyryl (γE-x69), (S)-4-(11-Benzyloxycarbonyl-undecanoylamino)-4-carboxy-butyryl (γE-x72), (S)-4-carboxy-4-[11-((2S,3R,4R,5R)-2,3,4,5,6-pentahydroxy-hexylcarbamoyl)-undecanoylamino]-butyryl (γE-x73), (S)-4-Carboxy-4-((Z)-octadec-9-enoylamino)-butyryl (γE-x74), (S)-4-Carboxy-4-(4-dodecyloxy-benzoylamino)-butyryl (γE-x75), (S)-4-Carboxy-4-henicosanoylamino-butyryl (γE-x76), (S)-4-Carboxy-4-docosanoylamino-butyryl (γE-x77), (S)-4-Carboxy-4-((Z)-nonadec-10-enoylamino)-butyryl (γE-x79), (S)-4-Carboxy-4-(4-decyloxy-benzoylamino)-butyryl (γE-x80), (S)-4-Carboxy-4-[(4′-octyloxy-biphenyl-4-carbonyl)-amino]-butyryl (γE-x81), (S)-4-Carboxy-4-(12-phenyl-dodecanoylamino)-butyryl (γE-x82), (S)-4-Carboxy-4-icosanoylamino-butyryl (γE-x95), (S)-4-Carboxy-4-((S)-4-carboxy-4-hexadecanoylamino-butyrylamino)-butyryl (γE-γE-x53), (S)-4-Carboxy-4-((S)-4-carboxy-4-octadecanoylamino-butyrylamino)-butyryl (γE-γE-x70), and 3-(3-Octadecanoylamino-propionylamino)-propionyl (β-Ala-β-Ala-x70).

Specific examples of peptide compounds of formula (I) and formula (Ia) are compounds of SEQ ID NO: 1-158, or a salt or a solvate thereof.

Specific examples of peptide compounds of formula (I) are compounds of SEQ ID NO: 1-66, 68-70, 72-74, 76-82, 84-95, 98, 102-106, 108-158, or a salt or a solvate thereof.

Specific examples of peptide compounds of formula (Ia) are compounds of SEQ ID NO: 67, 71, 75, 83, 96, 97, 99, 100, 101, 107 and 108, or a salt or a solvate thereof.

In some embodiments, compounds of the disclosure are selected from compounds of SEQ ID NO: 1-158, or a salt or a solvate thereof.

In some embodiments, compounds of the disclosure are selected from compounds of SEQ ID NO: 1 to 74, 101 to 106, 115, 116, 122, 126, 127, and 149 to 158, or a salt or a solvate thereof.

In some embodiments, compounds of the disclosure are selected from compounds of SEQ ID NO: 75 to 100, 107 to 114, 117 to 121, 123 to 125, and 128 to 148, or a salt or a solvate thereof.

In some embodiments, compounds of the disclosure are selected from compounds of SEQ ID NO: 1 to 70, 72 to 74, 101 to 106, 115, 116, 122, 126, 127, and 149 to 158, or a salt or a solvate thereof.

In some embodiments, compounds of the disclosure are selected from compounds of SEQ ID NO: 71, 75 to 100, 107 to 114, 117 to 121, 123 to 125, and 128 to 148, or a salt or a solvate thereof.

In some embodiments, compounds of the disclosure are selected from compounds of SEQ ID NO: 1 to 74, 102 to 106, 122, 126, 127, and 149 to 158, or a salt or a solvate thereof.

In some embodiments, compounds of the disclosure are selected from compounds of SEQ ID NO: 75 to 101, 107 to 121, 123 to 125, and 128 to 148, or a salt or a solvate thereof.

In some embodiments, compounds of the disclosure are selected from compounds of SEQ ID NO: 1 to 74, 102 to 106, 122, 126, and 149 to 158, or a salt or a solvate thereof.

In some embodiments, compounds of the disclosure are selected from compounds of SEQ ID NO: 75 to 101, 107 to 121, 123 to 127, and 128 to 148, or a salt or a solvate thereof.

In some embodiments, compounds of the disclosure are selected from compounds of SEQ ID NO: 1 to 38, 40 to 74, 108-106, 126, 127, 149, 151, 152, and 154-158, or a salt or a solvate thereof.

In some embodiments, compounds of the disclosure are compounds of SEQ ID NO: 67, 71, 75, 83, 96, 97, 99, 100, 101, 107 and 108, or a salt or a solvate thereof.

In some embodiments, compounds of the disclosure are compounds of SEQ ID NO: 35, 66, 113, 128, 144, 146, 147, 147, 148, and 149, or a salt or a solvate thereof.

Table 3 shows the structures of compounds of SEQ ID NO: 1 to 158.

TABLE 3
Compounds of SEQ ID NO: 1 to 158

| SEQ ID | |
|---|---|
| NO: | Sequence |
| 1 | Apt(§1)-C(@2)-N-T-W-Q-C(@2)-Yme-Nal-Aib-E(§1)-N—N—NH₂ |
| 2 | Ahx(§1)-C(@2)-N-T-W-Q-C(@2)-Yme-Nal-Aib-E(§1)-N—N—NH₂ |
| 3 | Ahx(§1)-C(@2)-N-T-W-Q-C(@2)-Yme-Nal-Aib-D(§1)-N—N—NH₂ |
| 4 | Apt(§1)-C(@2)-N-T-W-Q-C(@2)-Yme-Nal-Aib-E(§1)-N-G-NH₂ |
| 5 | Apt(§1)-C(@2)-N-T-W-Q-C(@2)-Yae-Nal-Aib-E(§1)-N—N—NH₂ |
| 6 | Ahx(§1)-C(@2)-Nmn-T-W-Q-C(@2)-Yme-Nal-Aib-D(§1)-N—N—NH₂ |
| 7 | K[Ac](§1)-C(@2)-N-T-W-Q-C(@2)-Yme-Nal-Aib-D(§1)-N—N—NH₂ |
| 8 | Apt(§1)-C(@2)-N-T-W-Q-C(@2)-Yde-Nal-Aib-E(§1)-N—N—NH₂ |
| 9 | Ahx(§1)-C(@2)-N-T-W-Q-C(@2)-Yae-Nal-Aib-D(§1)-N-G-NH2 |
| 10 | Apt(§1)-C(@2)-N-T-W-Q-C(@2)-Yde-Nal-Mile-E(§1)-N-(D-Leu)-NH2 |
| 11 | Dad(§1)-C(@2)-N-T-W-Q-C(@2)-Yme-Nal-Aib-K(§1)-N—N—NH2 |
| 12 | Nmapt(§1)-C(§3)(@2)-K[aAc](@3)-W-Q-C(@2)-Yae-Nal-Aib-E(§1)-N—N—NH2 |
| 13 | Nmapt(§1)-C(§3)(@2)-K[aAc](@3)-W-Q-C(@2)-Yae-Nal-Thp-E(§1)-N—N—NH2 |
| 14 | Nmapt(§1)-C(§3)(@2)-K[aAc](@3)-W-Q-C(@2)-Yae-Nal-Mle-E(§1)-N—N—NH2 |
| 15 | Nmapt(§1)-C(§3)(@2)-K[aAc](@3)-W-Q-C(@2)-Yae-Nal-Aib-E(§1)-N-G-NH2 |
| 16 | Nmapt(§1)-C(§3)(@2)-K[aAc](@3)-W-Q-C(@2)-Yae-Nal-Thp-E(§1)-N-G-NH2 |
| 17 | Nmapt(§1)-C(§3)(@2)-K[aAc](@3)-W-Q-C(@2)-Yae-Nal-Thp-E(§1)-A-G-NH2 |
| 18 | Nmapt(§1)-C(§3)(@2)-K[aAc](@3)-W-Q-C(@2)-Yae-Nal-Mle-E(§1)-A-G-NH2 |
| 19 | Nmapt(§1)-C(§3)(@2)-K[aAc](@3)-W-Q-C(@2)-Yae-Nal-Aib-E(§1)-A-(D-Leu)-NH2 |
| 20 | Apt(§1)-C(@2)-N-T-Wim-K[Ac]-C(@2)-Yde-Nal-Aib-E(§1)-N—H—NH2 |
| 21 | Apt(§1)-C(@2)-N-T-Wim-K[Ac]-C(@2)-Yde-Nal-Thp-E(§1)-N—H—NH2 |
| 22 | Apt(§1)-C(@2)-N-T-Wim-K[Ac]-C(@2)-Yde-Nal-Mle-E(§1)-N—H—NH2 |
| 23 | Dad(§1)-C(@2)-N-T-Wim-K[Ac]-C(@2)-Yde-Nal-Mle-K(§1)-N—H—NH2 |
| 24 | Apt(§1)-C(@2)-N-T-Wim-L-C(@2)-Yde-Nal-Thp-E(§1)-N-Pal-Sar-NH2 |
| 25 | Dad(§1)-C(@2)-N-T-W-Q-C(@2)-Yde-Nal-Aib-Kme(§1)-N—N—NH2 |
| 26 | Apt(§1)-C(@2)-N-T-W-Q-C(@2)-Yde-Nal-Thp-E(§1)-N—N—NH2 |
| 27 | Apt(§1)-C(@2)-N-T-W-Q-C(@2)-Yde-Nal-Mle-E(§1)-N—N—NH2 |
| 28 | Apt(§1)-C(§3)(@2)-K[aAc](@3)-W-Q-C(@2)-Yde-Nal-Mle-E(§1)-N—N—NH2 |
| 29 | Apt(§1)-C(§3)(@2)-K[aAc](@3)-W-Q-C(@2)-Yde-Nal-Aib-E(§1)-N-G-NH2 |
| 30 | Apt(§1)-C(§3)(@2)-K[aAc](@3)-W-Q-C(@2)-Yde-Nal-Thp-E(§1)-N-G-NH2 |
| 31 | Apt(§1)-C(§3)(@2)-K[aAc](@3)-W-Q-C(@2)-Yde-Nal-Mle-E(§1)-N-G-NH2 |
| 32 | Apt($1)-C(§3)(@2)-KlaAc](@3)-W-Q-C(@2)-Yde-Nal-Aib-E(§1)-A-G-NH2 |
| 33 | Apt(§1)-C(§3)(@2)- K[aAc](@3)-W-Q-C(@2)-Yde-Nal-Thp-E(§1)-A-(D-Leu)-NH2 |
| 34 | Nmapt(§1)-C(§3)(@2)-K[aAc](@3)-W-Q-C(@2)-Yae-Nal-Thp-E(§1)-A-(D-Leu)-NH2 |
| 35 | Apt(§1)-C(@2)-N-T-Wim-K[Ac]-C(@2)-Yde-Nal-Thp-E(§1)-N-Pal-Sar-NH2 |
| 36 | Apt(§1)-C(@2)-NdmK-W-K[Ac]-C(@2)-Yde-Nal-MKdm-E(§1)-N-Pal-Sar-NH2 |
| 37 | Apt(§1)-C(@2)-N-T-W-K[Ac]-C(@2)-Yde-Nal-Aib-E(§1)-N-Pal-Ile-NH2 |
| 38 | Apt(§1)-C(@2)-N-T-W-K[Ac]-C(@2)-Yde-Nal-Aib-E(§1)-N-N-Ile-NH2 |
| 39 | Ahx(§1)-E(@2)-Apt-W-Q-T(@2)-Yme-Nal-Aib-D(§1)-N—N—NH2 |
| 40 | Nmha(§1)-C(@2)-N-T-W-Q-C(@2)-Yde-Nal-Mle-D(§1)-N-(D-Leu)-NH2 |
| 41 | Nmapt(§1)-C(@2)-N-T-W-Q-C(@2)-Yde-Nal-Mle-E(§1)-N—N—NH2 |
| 42 | Nmapt(§1)-C(@2)-N-T-W-Q-C(@2)-Yde-Nal-Mle-E(§1)-N—N—NH2 |
| 43 | Apt(§1)-C(@2)-N-T-Wim-Q-C(@2)-Yde-Nal-Aib-E(§1)-S—H—NH2 |
| 44 | Nmapt(§1)-C(§3)(@2)-K[aAc](@3)-W-Q-C(@2)-Yae-Nal-Mle-E(§1)-A-(D-Leu)-NH2 |
| 45 | Apt(§1)-C(@2)-N-T-Wim-K[Ac]-C(@2)-Yde-Nal-Aib-E(§1)-N—H-Sar-NH2 |
| 46 | Apt(§1)-C(@2)-N-T-Wim-K[Ac]-C(@2)-Yde-Nal-Thp-E(§1)-N—H-Sar-NH2 |
| 47 | Apt(§1)-C(@2)-N-T-Wim-K[Ac]-C(@2)-Yde-Nal-Mle-E(§1)-N—H-Sar-NH2 |
| 48 | Apt(§1)-C(@2)-N-T-Wim-K[Ac]-C(@2)-Yde-Nal-Aib-E(§1)-N—H-Bal-NH2 |
| 49 | Apt(§1)-C(@2)-N-T-Wim-K[Ac]-C(@2)-Yde-Nal-Thp-E(§1)-N—H-Bal-NH2 |
| 50 | Apt(§1)-C(@2)-N-T-Wim-K[Ac]-C(@2)-Yde-Nal-Mle-E(§1)-N—H-Bal-NH2 |
| 51 | Apt(§1)-C(@2)-N-T-Wim-K[Ac]-C(@2)-Yde-Nal-Thp-E(§1)-N-Pal-NH2 |
| 52 | Apt(§1)-C(@2)-N-T-Wim-Q-C(@2)-Yde-Nal-Aib-E(§1)-N—H-NH2 |
| 53 | Ahx(§1)-C(@2)-Apt-W-Q-C(@2)-Yae-Nal-Aib-D(§1)-A-G-NH2 |
| 54 | Apt(§1)-C(@2)-N-T-Wim-K[Ac]-C(@2)-Yde-Nal-MKdm-E(§1)-N-(D-Leu)-NH2 |
| 55 | Apt(§1)-C(@2)-N-T-Wim-K[Ac]-C(@2)-Yde-Nal-Thp-E(§1)-N—H-Mep-NH2 |
| 56 | Apt(§1)-C(@2)-N-T-Wim-K[Ac]-C(@2)-Yde-Nal-Thp-E(§1)-S-Pal-Sar-NH2 |
| 57 | Apt(§1)-C(@2)-N-T-W-Q-C(@2)-Yde-Nal-Aib-E(§1)-N—N-Ile-NH2 |
| 58 | Apt(§1)-C(@2)-N-T-W-K[Ac]-C(@2)-Yde-Nal-MKdm-E(§1)-N-Pal-Sar-NH2 |
| 59 | Apt(§1)-C(§3)(@2)-K[aAc](@3)-W-K[Ac]-C(@2)-Yde-Nal-MKdm-E(§1)-N-Pal-Sar-NH2 |
| 60 | Nmapt(§1)-C(§3)(@2)-K[aAc](@3)-Wim-K[Ac]-C(@2)-Yde-Nal-Aib-E(§1)-N—H—NH2 |
| 61 | Nmapt(§1)-C(@2)-NdmK-Wim-K[Ac]-C(@2)-Yde-Nal-MKdm-E(§1)-N-Pal-Sar-NH2 |
| 62 | Apt(§1)-C(@2)-N-T-Wim-Q-C(@2)-Yde-Nal-Aib-E(§1)-N-Pal-Sar-NH2 |
| 63 | Apt(§1)-C(@2)-N-T-Wim-Q-C(@2)-Yde-Nal-Mle-E(§1)-N-Pal-Sar-NH2 |
| 64 | Apt(§1)-C(@2)-N-T-Wim-K[Ac]-C(@2)-Yde-Nal-Aib-E(§1)-N-Pal-Sar-NH2 |
| 65 | Apt(§1)-C(@2)-N-T-Wim-K[Ac]-C(@2)-Yde-Nal-Mle-E(§1)-N-Pal-Sar-NH2 |
| 66 | Apt(§1)-C(§3)(@2)-K[aAc](@3)-Wim-K[Ac]-C(@2)-Yde-Nal-Thp-E(§1)-N-Pal-Sar-NH2 |
| 67 | Apt(§1)-C(§3)(@2)-K[aAc](@3)-Wim-K[Ac]-C(@2)-Yde-Nal-Mly[Palm]-E(§1)-N-Pal-Sar- |
| | NH2 |
| 68 | Dad(§1)-C(§3)(@2)-K[aAc](@3)-Wim-L-C(@2)-Yde-Nal-Mle-Kme(§1)-N-Pal-Sar-NH2 |
| 69 | Dad(§1)-Nmc(§3)(@2)-K[aAc](@3)-Wim-L-C(@2)-Yde-Nal-Mle-Kme(§1)-N-Pal-Sar-NH2 |
| 70 | Dad(§1)-Nmc(§3)(@2)-K[aAc](@3)-Wim-L-C(@2)-Yde-Nal-Mie-Kme(§1)-A-Pal-Sar-NH2 |
| 71 | Dad(§1)-C(@2)-N-T-Wim-Q-C(@2)-Yde-Nal-Aib-K(§1)-N-Pal-K[Sema]-NH2 |
| 72 | Apt(§1)-C(§3)(@2)-K[aAc](@3)-Wim-K[Ac]-C(@2)-Yde-Nal-Aib-E(§1)-N-Pal-Sar-NH2 |
| 73 | Apt(§1)-C(§3)(@2)-K[aAc](@3)-Wim-K[Ac]-C(@2)-Yde-Nal-Mle-E(§1)-N-Pal-Sar-NH2 |
| 74 | Dad(§1)-Nmc(§3)(@2)-K[aAc](@3)-Wim-K[Ac]-C(@2)-Yde-Nal-Thp-Kme(§1)-N-Pal-Sar- |
| | NH2 |
| 75 | Ahx(§1)-C(@2)-N-T-W-Q-C(@2)-Yae-Nal-Aib-D(§1)-N-K[Sema]-NH2 |
| 76 | Apt(§1)-Abu(@2)-N-T-Wim-K[Ac]-C(@2)-Yde-Nal-Thp-E(§1)-N-Pal-NH2 |
| 77 | Apt(§1)-Abu(@2)-N-T-Wim-K[Ac]-C(@2)-Yde-Nal-Thp-E(§1)-N-Pal-Sar-NH2 |
| 78 | Apt(§1)-Abu(@2)-N-T-Wme-L-C(@2)-Yde-Nal-Thp-E(§1)-N-Pal-Sar-NH2 |
| 79 | Nmapt(§1)-Abu(@2)-N-T-Wme-L-C(@2)-Yde-Nal-Thp-E(§1)-N-Pal-Sar-NH2 |
| 80 | Nmha(§1)-Abu(@2)-N-T-Wme-L-C(@2)-Yde-Nal-Thp-E(§1)-N-Pal-Sar-NH2 |
| 81 | Nmha(§1)-Abu(@2)-N-T-Wme-L-C(@2)-Yde-Nal-Thp-D(§1)-N-Pal-Sar-NH2 |
| 82 | Nmapt(§1)-Abu(@2)-N-T-Wme-L-C(@2)-Yde-Nal-Thp-D(§1)-N-Pal-Sar-NH2 |
| 83 | Apt(§1)-C(@2)-Apt-Wme-L-C(@2)-Yde-Nal-Mle-E(§1)-N-K[Sema]-NH2 |
| 84 | Apt(§1)-C(@2)-N-T-Wim-K[Ac]-C(@2)-Yde-Nal-MKdm-E(§1)-N-Pyal-NH2 |
| 85 | Apt(§1)-C(@2)-N-T-Wim-K[Ac]-C(@2)-Yde-Nal-MKdm-E(§1)-N-Pal-NH2 |
| 86 | Apt(§1)-C(@2)-N-T-Wim-K[Ac]-C(@2)-Yde-Nal-MKdm-E(§1)-N-Pal-Sar-NH2 |
| 87 | Apt(§1)-C(@2)-N-T-Wim-K[Ac]-C(@2)-Yde-Nal-Aib-E(§1)-N-Pal-Ile-NH2 |
| 88 | Apt(§1)-C(§3)(@2)-K[aAc](@3)-Wim-K[Ac]-C(@2)-Yde-Nal-MKdm-E(§1)-N-Pal-NH2 |
| 89 | Apt(§1)-C(§3)(@2)-K[aAc](@3)-Wim-Q-C(@2)-Yde-Nal-MKdm-E(§1)-N-Pal-NH2 |
| 90 | Apt(§1)-C(@2)-N-T-W-K[Ac]-C(@2)-Yde-Nal-Aib-E(§1)-N-Pal-Ile-NH2 |
| 91 | Apt(§1)-C(§3)(@2)-KlaAc](@3)-W-K[Ac]-C(@2)-Yde-Nal-MKdm-E(§1)-N-Pal-NH2 |
| 92 | Apt(§1)-C(§3)(@2)-K[aAc](@3)-W-Q-C(@2)-Yde-Nal-MKdm-E(§1)-N-Pal-NH2 |
| 93 | Dad(§1)-C(§3)(@2)-K[aAc](@3)-Wim-Q-C(@2)-Yde-Nal-Aib-K(§1)-N-Pal-NH2 |
| 94 | Apt(§1)-C(@2)-N-T-Wim-Q-C(@2)-Yde-Nal-Mly-E(§1)-N-Pal-Sar-NH2 |
| 95 | Apt(§1)-C(@2)-N-T-Wim-K[Ac]-C(@2)-Yde-Nal-Mly-E(§1)-N-Pal-Sar-NH2 |
| 96 | Apt(§1)-C(@2)-N-T-Wim-K[Ac]-C(@2)-Yde-Nal-Mly-E(§1)-N-Pal-K[Sema]-NH2 |
| 97 | Apt(§1)-C(@2)-N-T-Wim-K[Ac]-C(@2)-Yde-Nal-Mly-E(§1)-N-Pal-Sar-K[Sema]-NH2 |
| 98 | Dad(§1)-Nmc(@2)-N-T-Wim-K[Ac]-C(@2)-Yde-Nal-MKdm-Kme(§1)-N-Pal-Sar-NH2 |
| 99 | Dad(§1)-C(@2)-N-T-Wim-K[Ac]-C(@2)-Yde-Nal-Mly(Myri)-K(§1)-N-Pal-Sar-NH2 |
| 100 | Apt(§1)-C(§3)(@2)-K[aAc](@3)-Wim-K[Ac]-C(@2)-Yde-Nal-Mly(Myri)-E(§1)-N-Pal-Sar- |
| | NH2 |
| 101 | Dad(§1)-C(§3)(@2)-K[aAc](@3)-Wim-K[Ac]-C(@2)-Yde-Nal-Mly(Palm)-K(§1)-N-Pal-Sar- |
| | NH2 |
| 102 | Dad(§1)-C(@2)-N-T-Wim-K[Ac]-C(@2)-Yde-Nal-Thp-K(§1)-N-Pal-Sar-NH2 |
| 103 | Dad(§1)-C(@2)-N-T-Wim-K[Ac]-C(@2)-Yde-Nal-Mle-K(§1)-N-Pal-Sar-NH2 |
| 104 | Dad(§1)-C(§3)(@2)-K[aAc](@3)-Wim-K[Ac]-C(@2)-Yde-Nal-Aib-K(§1)-N-Pal-Sar-NH2 |
| 105 | Dad(§1)-C(§3)(@2)-K[aAc](@3)-Wim-K[Ac]-C(@2)-Yde-Nal-Thp-K(§1)-N-Pal-Sar-NH2 |
| 106 | Dad(§1)-C(§3)(@2)-K[aAc](@3)-Wim-K[Ac]-C(@2)-Yde-Nal-Mle-K(§1)-N-Pal-Sar-NH2 |
| 107 | Dad(§1)-C(@2)-N-T-Wim-K[Ac]-C(@2)-Yde-Nal-Thp-K(§1)-N-Pal-Sar-K[Sema]-NH2 |
| 108 | Apt(§1)-C(§3)(@2)-K[aAc](@3)-Wim-K[Ac]-C(@2)-Yde-Nal-Mly(Myri)-E(§1)-N-Pal-Sar- |
| | NH2 |
| 109 | Dad(§1)-Nmc(§3)(@2)-K[aAc](@3)-Wim-K[Ac]-C(@2)-Yde-Nal-MKdm-Kme(§1)-N-Pal- |
| | Sar-NH2 |
| 110 | Dad(§1)-C(§3)(@2)-K[aAc](@3)-Wim-K[Ac]-C(@2)-Yde-Nal-Thp-Kme(§1)-N-Pal-Sar- |
| | NH2 |
| 111 | Dad(§1)-Abu(§3)(@2)-K[aAc](@3)-Wim-K[Ac]-C(@2)-Yde-Nal-MKdm-K(§1)-N-Pal-Sar- |
| | NH2 |
| 112 | Apt(§1)-Abu(§3)(@2)-K[aAc](@3)-Wim-K[Ac]-C(@2)-Yde-Nal-MKdm-E(§1)-N-Pal-Sar- |
| | NH2 |
| 113 | Dad(§1)-C(§3)(@2)-K[aAc](@3)-Wim-K[Ac]-C(@2)-Yde-Nal-MKdm-K(§1)-G-PyEA |
| 114 | Dad(§1)-Nmc(§3)(@2)-K[aAc](@3)-Wim-K[Ac]-C(@2)-Yde-Nal-Thp-Kme(§1)-N-Pal-Sar- |
| | N(Et)2 |
| 115 | Apt(§1)-Abu(§3)(@2)-K[aAc](@3)-Wim-K[Ac]-C(@2)-Yde-Nal-Thp-E(§1)-N-Pal-Sar-NH2 |
| 116 | Apt(§1)-Abu(§3)(@2)-K[aAc](@3)-Wim-K[Ac]-C(@2)-Yde-Nal-Aib-E(§1)-N-Pal-Sar-NH2 |
| 117 | Apt(§1)-Abu(§3)(@2)-K[aAc](@3)-Wim-K[Ac]-C(@2)-Yde-Nal-Mle-E(§1)-N-Pal-Sar-NH2 |
| 118 | Dad(§1)-Abu(@2)-N-T-Wim-K[Ac]-C(@2)-YdeNal-Aib-K(§1)-N-Pal-Sar-NH2 |
| 119 | Dad(§1)-Abu(@2)-N-T-Wir-K[Ac]-C(@2)-Yde-Nal-Thp-K(§1)-N-Pal-Sar-NH2 |
| 120 | Dad(§1)-Abu(@2)-N-T-Wim-K[Ac]-C(@2)-Yde-Nal-Mle-K(§1)-N-Pal-Sar-NH2 |
| 121 | Dad(§1)-Abu(§3)(@2)-K[aAc](@3)-Wim-K[Ac]-C(@2)-Yde-Nal-Aib-K(§1)-N-Pal-Sar-NH2 |
| 122 | Dad(§1)-Abu(§3)(@2)-K[aAc](@3)-Wim-K[Ac]-C(@2)-Yde-Nal-Thp-K(§1)-N-Pal-Sar-NH2 |
| 123 | Dad(§1)-Abu(§3)(@2)-K[aAc](@3)-Wim-K[Ac]-C(@2)-Yde-Nal-Mle-K(§1)-N-Pal-Sar-NH2 |
| 124 | Dad(§1)-Abu(@2)-N-T-Wim-K[Ac]-C(@2)-Yde-Nal-Thp-Kme(§1)-N-Pal-Sar-NH2 |
| 125 | Dad(§1)-Abu(§3)(@2)-K[aAc](@3)-Wim-K[Ac]-C(@2)-Yde-Nal-MKdm-Kme(§1)-N-Pal- |
| | Sar-NH2 |
| 126 | Apt(§1)-C(§3)(@2)-K[aAc](@3)-Wme-K[Ac]-C(@2)-Yde-Nal-ThpE(§1)-N-Pal-Sar-NH2 |
| 127 | Nmc(Butyl§1)(§3)(@2)-K[aAc](@3)-Wim-K[Ac]-C(@2)-Yde-Nal-Thp-Kme(§1)-N-Pal-Sar- |
| | NH2 |
| 128 | Nmc(Butyl§1)(§3)(@2)-K[aAc](@3)-Wim-K[Ac]-C(@2)-Yde-Nal-MKdm-Kme(§1)-G-PyEA |
| 129 | Nmapt(§1)-C(§3)(@2)-K[aAc](@3)-Wim-K[Ac]-C(@2)-Yde-Nal-MKdm-E(§1)-N-Pal-Sar- |
| | NH2 |
| 130 | Dad(§1)-Abu(§3)(@2)-K[aAc](@3)-W-K[Ac]-C(@2)-Yde-Nal-MKdm-K(§1)-N-Pal-Sar-NH2 |
| 131 | Apt(§1)-Abu(§3)(@2)-K[aAc](@3)-W-K[Ac]-C(@2)-Yde-Nal-MKdm-E(§1)-N-Pal-Sar-NH2 |
| 132 | Apt(§1)-Abu(§3)(@2)-K[aAc](@3)-W-K[Ac]-C(@2)-Yde-Nal-Thp-E(§1)-N-Pal-Sar-NH2 |
| 133 | Apt(§1)-Abu(§3)(@2)-K[aAc](@3)-W-K[Ac]-C(@2)-Yde-Nal-Aib-E(§1)-N-Pal-Sar-NH2 |
| 134 | Apt(§1)-Abu(§3)(@2)-K[aAc](@3)-W-K[Ac]-C(@2)-Yde-Nal-Mle-E(§1)-N-Pal-Sar-NH2 |
| 135 | Dad(§1)-Abu(@2)-N-T-W-K[Ac]-C(@2)-Yde-Nal-Aib-K(§1)-N-Pal-Sar-NH2 |
| 136 | Dad(§1)-Abu(@2)-N-T-W-K[Ac]-C(@2)-Yde-Nal-Thp-K(§1)-N-Pal-Sar-NH2 |
| 137 | Dad(§1)-Abu(@2)-N-T-W-K[Ac]-C(@2)-Yde-Nal-Mle-K(§1)-N-Pal-Sar-NH2 |
| 138 | Dad(§1)-Abu(§3)(@2)-K[aAc](@3)-W-K[Ac]-C(@2)-Yde-Nal-Alb-K(§1)-N-Pal-Sar-NH2 |
| 139 | Dad(§1)-Abu(§3)(@2)-K[aAc](@3)-W-K[Ac]-C(@2)-Yde-Nal-Thp-K(§1)-N-Pal-Sar-NH2 |
| 140 | Dad(§1)-Abu(§3)(@2)-K[aAc](@3)-W-K[Ac]-C(@2)-Yde-Nal-Mle-K(§1)-N-Pal-Sar-NH2 |
| 141 | Dad(§1)-Abu(@2)-N-T-W-K[Ac]-C(@2)-Yde-Nal-Thp-Kme(§1)-N-Pal-Sar-NH2 |
| 142 | Dad(§1)-Abu(§3)(@2)-K[aAc](@3)-W-K[Ac]-C(@2)-Yde-Nal-MKdm-Kme(§1)-N-Pal-Sar- |
| | NH2 |
| 143 | C(Buty§1)(§3)(@2)-K[aAc](@3)-Wim-K[Ac]-C(@2)-Yde-Nal-MKdm-Kme(§1)-N-Pal-Sar- |
| | NH2 |
| 144 | C(Buty§1)(§3)(@2)-K[aAc](@3)-Wim-K[Ac]-C(@2)-Yde-Nal-MKdm-Kme(§1)-G-PyEA |
| 145 | Nmc(Butyl§1)(§3)(@2)-K[aAc](@3)-Wim-K[Ac]-C(@2)-Yde-Nal-MKdm-Kme(§1)-N-Pal- |
| | Sar-NH2 |
| 146 | Nmc(Butyl§1)(§3)(@2)-K[aAc](@3)-Wim-Q-C(@2)-Yde-Nal-MKdm-Kme(§1)-G-PyEA |
| 147 | Nmc(Butyl§1)(§3)(@2)-K[aAc](@3)-Wme-K[Ac]-C(@2)-Yde-Nal-MKdm-Kme(§1)-G-PyEA |
| 148 | Nmc(Butyl§1)(§3)(@2)-K[aAc](@3)-Wme-L-C(@2)-Yde-Nal-MKdm-Kme(§1)-G-PyEA |
| 149 | Apt(§1)-C(§3)(@2)-K[aAc](@3)-Wim-K[Ac]-C(@2)-Yde-Nal-MKdm-E(§1)-N-Pal-Sar-NH2 |
| 150 | Dad(§1)-C(@2)-N-T-Wim-L-C(@2)-Yde-Nal-MKdm-K(§1)-N-Pal-NH2 |
| 151 | Dad(§1)-C(@2)-N-T-Wim-K[Ac]-C(@2)-Yde-Nal-MKdm-K(§1)-N-Pal-Sar-NH2 |
| 152 | Dad(§1)-C(@2)-N-T-Wim-Q-C(@2)-Yde-Nal-MKdm-K(§1)-N-Pal-Sar-NH2 |
| 153 | Dad(§1)-C(@2)-N-T-Wim-Q-C(@2)-Yde-Nal-MKdm-K(§1)-N-Pal-Sar-NH2 |
| 154 | Dad(§1)-C(@2)-N-T-Wim-L-C(@2)-Yde-Nal-MKdm-K(§1)-N-Pal-Sar-NH2 |
| 155 | Dad(§1)-C(@2)-N-T-Wim-L-C(@2)-Yde-Nal-MKdm-K(§1)-A-Pal-Sar-NH2 |
| 156 | Dad(§1)-C(@2)-N-T-Wim-K[Ac]-C(@2)-Yde-Nal-MKdm-Kme(§1)-N-Pal-Sar-NH2 |
| 157 | Dad(§1)-C(@2)-N-T-Wim-K[Ac]-C(@2)-Yde-Nal-Aib-K(§1)-N-Pal-Sar-NH2 |
| 158 | Dad(§1)-C(§3)(@2)-K[aAc](@3)-Wim-L-C(@2)-Yde-Nal-Mle-K(§1)-N-Pal-Sar-NH2 |

(@2) indicate the formation of an ester, a thioether or a disulfide bridge between amino acid residues.

(§ 1) indicate the formation of a lactam bridge between the between amino acid residues.

C (§ 3), Nmc (§ 3) or Abu (§ 3) and K[aAc] (@3) indicate the formation of a peptide bond between the epsilon-NH2 group of K[aAc] and the C-terminus of C, Nmc or Abu.

[Ac] indicates acetylation in R1 of the X1 amino acid residue or the acetylation of a side chain of an amino acid.

Compounds disclosed herein have a binding affinity to the interleukin 23 receptor determined using the method described in the Examples (i.e., Human ELISA IL23/IL23R) of 100 nM or below (i.e., IC₅₀≤100 nM), or 50 nM or below (i.e., IC₅₀≤50.0 nM), or 10 nM or below (i.e., IC₅₀≤10.0 nM), and even of 5 nM or below (i.e., IC₅₀≤5.0 nM).

The term “activity” as used herein refers to the capability of a compound to inhibit the human interleukin-23 receptor. For example, the term “activity” as used herein refers to the capability of a compound to stimulate intracellular STAT3 phosphorylation. Sometimes, reference may also be made to the term “potency” or “in vitro potency” instead of “activity”. Accordingly, “potency” is a measure for the ability of a compound to inhibit the interleukin-23 receptors for in a cell-based assay. Numerically, it is expressed as the “IC₅₀ value”, which is the effective concentration of a compound that inhibits a half maximal increase of response (e.g., formation of phosphorylation of STAT3) in a dose-response experiment.

Compounds disclosed herein have interleukin-23 receptor affinity. This term refers to the ability to bind to the interleukin-23 receptor and inhibit a signal transduction pathway resulting in Th17 cell activation or other physiological effects as is known in the art. For example, compounds disclosed herein can be tested for interleukin-23 receptor affinity or activity using the assays described in Methods and results shown in Table 10 herein.

In a further embodiment, compounds disclosed herein are sufficiently chemical stable in solution at different pH.

Compounds disclosed herein have a chemical stability in solution at pH 1.2 of less than 30% relative purity loss over 24 h at 37° C., or less than 25%.

Compounds disclosed herein have a chemical stability in solution at pH 6.5 of less than 5% relative purity loss over 24 h at 37° C., or less than 2%.

Compounds disclosed herein have a chemical stability in solution at pH 7.4 of less than 5% relative purity loss over 24 h at 37° C., or less than 3%.

In a further embodiment, compounds disclosed herein are sufficiently stable in the intestinal environment to execute a pharmacological effect.

### Peptide Synthesis

The skilled person is aware of a variety of different methods to prepare peptides. These methods include but are not limited to synthetic approaches and recombinant gene expression. Thus, one way of preparing peptides is the synthesis in solution or on a solid support and subsequent isolation and purification.

A way to prepare the compounds disclosed herein is solid phase synthesis on a suitable resin. Solid phase peptide synthesis is a well-established methodology (see for example: Stewart and Young, Solid Phase Peptide Synthesis, Pierce Chemical Co., Rockford, Ill., 1984; E. Atherton and R. C. Sheppard, Solid Phase Peptide Synthesis. A Practical Approach, Oxford-IRL Press, New York, 1989). Solid phase synthesis is initiated by attaching an N-terminally protected amino acid with its carboxy terminus to an inert solid support carrying a cleavable linker. This solid support can be any polymer that allows coupling of the initial amino acid, e.g., a trityl resin, a chlorotrityl resin, a Wang resin or a Rink resin in which the linkage of the carboxy group (or carboxamide for Rink resin) to the resin is sensitive to acid (when Fmoc strategy is used). The polymer support must be stable under the conditions used to deprotect the α-amino group during the peptide synthesis.

After the N-terminally protected first amino acid has been coupled to the solid support, the α-amino protecting group of this amino acid is removed. The remaining protected amino acids are then coupled one after the other or with a preformed dipeptide, tripeptide or tetrapeptide or with an amino acid building block with a modified sidechain as e.g., N-alpha-(9-fluorenylmethyloxycarbonyl)-N-epsilon-(N-alpha′-octadecandionyl-t-butyl ester-L-glutamic-acid alpha′-t-butyl ester-2-[2-[2-[2-[2-(2-aminoethoxy)ethoxy]acetylamino]ethoxy]ethoxy]acetyl)-L-lysine, (2S)-2-(9H-fluoren-9-ylmethoxycarbonylamino)-2-methyl-6-(tetradecanoylamino) hexanoic acid or (2S)-2-(9H-fluoren-9-ylmethoxycarbonylamino)-6-(hexadecanoylamino)-2-methyl-hexanoic acid, in the order represented by the peptide sequence using appropriate amide coupling reagents, for example BOP, HBTU, HATU or DIC (N,N′-diisopropylcarbodiimide)/HOBt (1-hydroxybenzotriazole), wherein BOP, HBTU and HATU are used with tertiary amine bases. Alternatively, the liberated N-terminus can be functionalized with groups other than amino acids, for example carboxylic acids.

Usually, reactive side-chain groups of the amino acids are protected with suitable blocking groups. These protecting groups are removed after the desired peptides have been assembled. They are removed concomitantly with the cleavage of the desired product from the resin under the same conditions. Protecting groups and the procedures to introduce protecting groups can be found in Protective Groups in Organic Synthesis, 3d ed., Greene, T. W. and Wuts, P. G. M., Wiley & Sons (New York: 1999).

In some cases, it might be desirable to have side chain protecting groups that can selectively be removed while other side chain protecting groups remain intact. In this case the liberated functionality can be selectively functionalized. For example, a lysine may be protected with an ivDde ([1-(4,4-dimethyl-2,6-dioxocyclohex-1-ylidene)-3-methylbutyl) protecting group (S. R. Chhabra et al., Tetrahedron Lett. 39, (1998), 1603) which is labile to a very nucleophilic base, for example 4% hydrazine in DMF (dimethyl formamide). Thus, if the N-terminal amino group and all side-chain functionalities are protected with acid labile protecting groups, the ivDde group can be selectively removed using 4% hydrazine in DMF and the corresponding free amino group can then be further modified, e.g., by acylation. The lysine can alternatively be coupled to a protected amino acid and the amino group of this amino acid can then be deprotected resulting in another free amino group which can be acylated or attached to further amino acids.

Finally, the peptide is cleaved from the resin. This can be achieved by using King's cocktail (D. S. King, C. G. Fields, G. B. Fields, Int. J. Peptide Protein Res. 36, 1990, 255-266) similar cleavage cocktails known to the person skilled in the art. For example, EDT can be replaced by DODT or a mixture of TIS, water and TFA can be used. The raw material can then be purified by chromatography, e.g., preparative RP-HPLC, if necessary.

All starting materials such as amino acids and chemicals have been ordered from vendors. For example (2S)-6-(tert-butoxycarbonylamino)-2-(9H-fluoren-9-ylmethoxycarbonylamino) hexanoic acid was purchased from Fisher Scientific (eKat). Other suppliers are Bachem AG, Hauptstrasse 144, 4416 Bubendorf, Switzerland; Iris Biotech GmbH, Adalbert-Zoellner-Str. 1, 95615 Marktredwitz; Gyros Protein Technologies, Uppsala Science Park, Dag Hammarskjölds väg 54, SE-751 83 Uppsala, Sweden; Thermo Fisher Scientific, 168 Third Avenue, Waltham, MA USA 02451

### Cyclization/Lactam Formation

Methods for lactam bond formation are known to the person skilled in the art. Exemplary methods are given in the examples. The amino acid residue homoSerine (hSer) or homoSerine (Br) in its unbonded form may take the form of 2-aminobutyric acid (Abu) when participating in an intramolecular bond according to the present disclosure.

Synthesis of Sema is described in WO2011080102, which is incorporated by reference.

### Potency

As used herein, the term “potency” or “in vitro potency” is a measure for the ability of a compound to inactivate the receptor for interleukin-23 in a cell-based assay. Numerically, it is expressed as the “IC₅₀ value”, which is the effective concentration of a compound that induces a half maximal decrease of response (e.g., status of intracellular STAT3) in a dose-response experiment.

### Therapeutic Uses

The term “disease or disorder” refers to any pathological or unhealthy state, in particular diseases related to the immune system such as Colitis ulcerosa, Crohn's disease and Psoriasis.

By “treat” or “treating” is meant to administer a compound or composition or a combination of compounds or compositions to a subject in order to eliminate a disease or disorder; arrest or slow a disease or disorder in a subject; inhibit or slow the development of a new disease or disorder in a subject; decrease the frequency or severity of symptoms and/or recurrences in a subject who currently has or who previously has had a disease or disorder; and/or prolong, i.e., increase, the lifespan of the subject. For example, the term “treating/treatment of a disease or disorder” includes curing, shortening the duration, ameliorating, slowing down or inhibiting progression or worsening of a disease or disorder or the symptoms thereof.

By “prevent” or “preventing” is meant to administer a compound or composition or a combination of compounds or compositions to a subject in order to inhibit or delay the onset of a disease or disorder in a subject.

The term “subject” means according to the disclosure a subject for treatment, in particular a diseased subject (also referred to as “patient”), including human beings, non-human primates or other animals, in particular mammals, such as cows, horses, pigs, sheep, goats, dogs, cats, rabbits or rodents, such as mice, rats, guinea pigs and hamsters. In one embodiment, the subject/patient is a human being.

In certain embodiments, the present disclosure includes methods of inhibiting IL-23 signalling by a cell, comprising contacting the IL-23 with a peptide compound disclosed herein. In certain embodiments, the cell is a mammalian cell. In particular embodiments, the method is performed in vitro or in vivo. In some embodiments, the inhibition of IL-23 signalling may be determined by measuring changes in phospho-STAT3 levels in the cell.

In some embodiments, the inhibition of IL-23 binding to IL-23R occurs in particular organs or tissues of the subject, e.g., the stomach, small intestine, large intestine/colon, intestinal mucosa, lamina propria, Peyer's Patches, mesenteric lymph nodes, or lymphatic ducts.

Further provided is the use of compounds of formula (I) or (Ia) of the disclosure, or a composition comprising a compound of formula (I) or (Ia) of the disclosure, in the preparation of a pharmaceutical composition, for example for treating an autoimmune disease or an inflammatory disease.

The autoimmune or inflammatory disease may for example be inflammatory bowel disease, such as Crohn's disease and ulcerative colitis, psoriasis, psoriatic arthritis and hidradenitis suppurativa.

In certain embodiments, the disease or disorder is autoimmune inflammation and related diseases and disorders, such as multiple sclerosis, asthma, rheumatoid arthritis, inflammatory bowel diseases (IBDs), juvenile IBD, adolescent IBD, Crohn's disease, sarcoidosis, Systemic Lupus Erythematosus, ankylosing spondylitis (axial spondylarthrites), psoriatic arthritis, or psoriasis. In some embodiments, the disease or disorder is psoriasis (e.g., plaque psoriasis, guttate psoriasis, inverse psoriasis, pustular psoriasis, Palmo-Plantar Pustulosis, psoriasis vulgaris, or erythrodermic psoriasis), atopic dermatitis, acne ectopica, ulcerative colitis, Crohn's disease, Celiac disease (nontropical Sprue), enteropathy associated with seronegative arthropathies, microscopic colitis, collagenous colitis, eosinophilic gastroenteritis/esophagitis, colitis associated with radio- or chemo-therapy, colitis associated with disorders of innate immunity as in leukocyte adhesion deficiency-1, chronic granulomatous disease, glycogen storage disease type Ib, Hermansky-Pudlak syndrome, Chediak-Higashi syndrome, Wiskott-Aldrich Syndrome, pouchitis resulting after proctocolectomy and ileoanal anastomosis, gastrointestinal cancer, pancreatitis, insulindependent diabetes mellitus, mastitis, cholecystitis, cholangitis, primary biliary cirrhosis, viral-associated enteropathy, pericholangitis, chronic bronchitis, chronic sinusitis, asthma, uveitis, or graft versus host disease.

In a further related embodiment, the present disclosure includes a method of inhibiting gastro-intestinal (GI) inflammation and/or neutrophil infiltration to the GI, comprising providing to a subject in need thereof a peptide compound disclosed herein.

### Pharmaceutical Compositions

The term “pharmaceutical composition” indicates a mixture containing ingredients that are compatible when mixed and which may be administered. A pharmaceutical composition may include one or more medicinal drugs. Additionally, the pharmaceutical composition may include carriers, buffers, acidifying agents, alkalizing agents, solvents, adjuvants, tonicity adjusters, emollients, expanders, preservatives, physical and chemical stabilizers e.g., surfactants, antioxidants and other components, whether these are considered active or inactive ingredients. Examples of preservatives are benzylic alcohol or phenolic compounds like phenol or m-cresol.

A pharmaceutical composition may include one or more compounds of the disclosure and at least one pharmaceutically acceptable carrier.

A “pharmaceutically acceptable carrier” is a carrier which is physiologically acceptable (e.g., physiologically acceptable pH) while retaining the therapeutic properties of the substance with which it is administered. A pharmaceutically acceptable carrier, diluent or excipient refers to a non-toxic solid, semi-solid or liquid filler, diluent, encapsulating material or Formulation auxiliary of any type.

Standard acceptable pharmaceutical carriers and their formulations are known to one skilled in the art and described, for example, in Remington: The Science and Practice of Pharmacy, (20th ed.) ed. A. R. Gennaro A. R., 2000, Lippencott Williams & Wilkins and in R. C. Rowe et al. (Ed), Handbook of Pharmaceutical excipients, PhP, May 2013 update. One exemplary pharmaceutically acceptable carrier is physiological saline solution.

The term “pharmaceutically acceptable salt” means salts of the compounds disclosed herein which are safe and effective for use in mammals, e.g., acetate salts, chloride salts or sodium salts.

The term “solvate” means complexes of the compounds disclosed herein or salts thereof with solvent molecules, e.g., organic solvent molecules and/or water.

Acceptable pharmaceutical carriers or diluents include those used in formulations suitable for oral, rectal, nasal or parenteral (including subcutaneous, intramuscular, intravenous, intradermal, and transdermal) administration.

In certain embodiments, the compositions are administered orally, parenterally, intracisternally, intravaginally, intraperitoneally, intrarectally, topically (as by powders, ointments, drops, suppository, or transdermal patch), by inhalation (such as intranasal spray), ocularly (such as intraocularly) or buccally. The term “parenteral” as used herein refers to modes of administration which include intravenous, intramuscular, intraperitoneal, intrasternal, subcutaneous, intradermal and intraarticular injection and infusion. Accordingly, in certain embodiments, the compositions are Formulated for delivery by any of these routes of administration.

In certain embodiments, pharmaceutical compositions for parenteral injection comprise pharmaceutically acceptable sterile aqueous or nonaqueous solutions, dispersions, suspensions or emulsions, or sterile powders, for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, βcyclodextrin, vegetable oils (such as olive oil), and injectable organic esters such as ethyl oleate. Proper fluidity may be maintained, for example, by the use of coating materials such as lecithin, by the maintenance of the required particle size in the case of dispersions, and by the use of surfactants. These compositions may also contain adjuvants such as preservative, wetting agents, emulsifying agents, and dispersing agents. Prolonged absorption of an injectable pharmaceutical form may be brought about by the inclusion of agents which delay absorption, such as aluminium monostearate and gelatine.

In some embodiments, a compound of the disclosure, or the pharmaceutical composition comprising a compound of the disclosure, is suspended in a sustained-release matrix. A sustained-release matrix, as used herein, is a matrix made of materials, usually polymers, which are degradable by enzymatic or acid-base hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid) polyanhydrides, poly(ortho) esters, polypeptides, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. One embodiment of a biodegradable matrix is a matrix of one of either polylactide, polyglycolide, or polylactide co-glycolide (co-polymers of lactic acid and glycolic acid).

Injectable depot forms include those made by forming microencapsule matrices of the peptide compound in one or more biodegradable polymers such as polylactide-polyglycolide, poly(orthoesters), poly(anhydrides), and (poly)glycols, such as PEG. Depending upon the ratio of peptide to polymer and the nature of the particular polymer employed, the rate of release of the peptide compound can be controlled. Depot injectable Formulations are also prepared by entrapping the peptide compound in liposomes or microemulsions compatible with body tissues.

The injectable formulations may be sterilized, for example, by filtration through a bacterial-retaining filter, or by incorporating sterilizing agents in the form of sterile solid compositions which can be dissolved or dispersed in sterile water or other sterile injectable medium just prior to use.

Topical administration includes administration to the skin or mucosa, including surfaces of the lung and eye. Compositions for topical lung administration, including those for inhalation and intranasal, may involve solutions and suspensions in aqueous and non-aqueous formulations and can be prepared as a dry powder which may be pressurized or non-pressurized. In non-pressurized powder compositions, the active ingredient may be finely divided form may be used in admixture with a larger-sized pharmaceutically acceptable inert carrier comprising particles having a size, for example, of up to 100 micrometers in diameter. Suitable inert carriers include sugars such as lactose.

Alternatively, the composition may be pressurized and contain a compressed gas, such as nitrogen or a liquefied gas propellant. The liquefied propellant medium and indeed the total composition may bey such that the active ingredient does not dissolve therein to any substantial extent. The pressurized composition may also contain a surface active agent, such as a liquid or solid non-ionic surface active agent or may be a solid anionic surface active agent. In some embodiments, one may use the solid anionic surface-active agent in the form of a sodium salt.

A further form of topical administration is to the eye. A peptide compound of the disclosure may be delivered in a pharmaceutically acceptable ophthalmic vehicle, such that the peptide compound is maintained in contact with the ocular surface for a sufficient time period to allow the peptide compound to penetrate the corneal and internal regions of the eye, as for example the anterior chamber, posterior chamber, vitreous body, aqueous humor, vitreous humor, cornea, iris/ciliary, lens, choroid/retina and sclera. The pharmaceutically acceptable ophthalmic vehicle may, for example, be an ointment, vegetable oil or an encapsulating material. Alternatively, the peptide compounds disclosed herein may be injected directly into the vitreous and aqueous humour.

Compositions for rectal or vaginal administration include suppositories which may be prepared by mixing the peptides of this disclosure with suitable non-irritating excipients or carriers such as cocoa butter, polyethylene glycol or a suppository wax, which are solid at room temperature but liquid at body temperature and, therefore, melt in the rectum or vaginal cavity and release the active compound.

Peptide compounds disclosed herein may also be administered in liposomes or other lipid-based carriers. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to a peptide compound disclosed herein, stabilizers, preservatives, excipients, and the like. In certain embodiments, the lipids comprise phospholipids, including the phosphatidyl cholines (lecithins) and serines, both natural and synthetic. Methods to form liposomes are known in the art.

Pharmaceutical compositions to be used in the disclosure suitable for parenteral administration may comprise sterile aqueous solutions and/or suspensions of the peptide compounds made isotonic with the blood of the recipient, generally using sodium chloride, glycerine, glucose, mannitol, sorbitol, and the like.

### Oral Administration

One example of way of administration of a compound disclosed herein or a pharmaceutical composition thereof is oral administration.

The disclosure provides a pharmaceutical composition for oral delivery. Compositions and peptides of the instant disclosure may be prepared for oral administration according to any of the methods, techniques, and/or delivery vehicles described herein. Further, one having skill in the art will appreciate that the peptides of the instant disclosure may be modified or integrated into a system or delivery vehicle that is not disclosed herein, yet is well known in the art and compatible for use in oral delivery of peptides.

In certain embodiments, formulations for oral administration may comprise adjuvants (e.g., resorcinols and/or nonionic surfactants such as polyoxyethylene oleyl ether and n-hexadecylpolyethylene ether) to artificially increase the permeability of the intestinal walls, and/or enzymatic inhibitors (e.g., pancreatic trypsin inhibitors, diisopropylfluorophosphate (DFF) or trasylol) to inhibit enzymatic degradation. In certain embodiments, the peptide compound of a solid-type dosage form for oral administration can be mixed with at least one additive, such as sucrose, lactose, cellulose, mannitol, trehalose, raffinose, maltitol, dextran, starches, agar, alginates, chitins, chitosans, pectins, gum tragacanth, gum arabic, gelatine, collagen, casein, albumin, synthetic or semisynthetic polymer, or glyceride. These dosage forms can also contain other type(s) of additives, e.g., inactive diluting agent, lubricant such as magnesium stearate, paraben, preserving agent such as sorbic acid, ascorbic acid, alpha-tocopherol, antioxidants such as cysteine, disintegrators, binders, thickeners, buffering agents, pH adjusting agents, sweetening agents, flavoring agents, or perfuming agents.

In some embodiments, oral dosage forms or unit doses compatible for use with the peptide compounds disclosed herein may include a mixture of peptide compound and non-drug components or excipients, as well as other non-reusable materials that may be considered either as an ingredient or packaging. Oral compositions may include at least one of a liquid, a solid, and a semi-solid dosage form. In some embodiments, an oral dosage form is provided comprising an effective amount of peptide compound, wherein the dosage form comprises at least one of a pill, a tablet, a capsule, a gel, a paste, a drink, a syrup, ointment, and suppository. In some instances, an oral dosage form is provided that is designed and configured to achieve delayed release of the peptide compound in the subject's small intestine and/or colon.

In one embodiment, an oral pharmaceutical composition comprising a peptide compound disclosed herein comprises an enteric coating that is designed to delay release of the peptide compound in the small intestine. In at least some embodiments, a pharmaceutical composition is provided which comprises a peptide compound disclosed herein and a protease inhibitor, such as aprotinin, in a delayed release pharmaceutical Formulation. In some instances, pharmaceutical compositions of the instant disclosure comprise an enteric coat that is soluble in gastric juice at a pH of about 5.0 or higher. In at least one embodiment, a pharmaceutical composition is provided comprising an enteric coating comprising a polymer having dissociable carboxylic groups, such as derivatives of cellulose, including hydroxypropylmethyl cellulose phthalate, cellulose acetate phthalate and cellulose acetate trimellitate and similar derivatives of cellulose and other carbohydrate polymers.

In one embodiment, a pharmaceutical composition comprising a peptide compound disclosed herein is provided in an enteric coating, the enteric coating being designed to protect and release the pharmaceutical composition in a controlled manner within the subject's lower gastrointestinal system, and to avoid systemic side effects. In addition to enteric coatings, the peptide compounds of the instant disclosure may be encapsulated, coated, engaged or otherwise associated within any compatible oral drug delivery system or component. For example, in some embodiments a peptide compound disclosed herein is provided in a lipid carrier system comprising at least one of polymeric hydrogels, nanoparticles, microspheres, micelles, and other lipid systems.

To overcome peptide degradation in the small intestine, some embodiments disclosed herein comprise a hydrogel polymer carrier system in which a peptide compound disclosed herein is contained, whereby the hydrogel polymer protects the peptide compound from proteolysis in the small intestine and/or colon. The peptide compounds disclosed herein may further be Formulated for compatible use with a carrier system that is designed to increase the dissolution kinetics and enhance intestinal absorption of the peptide. These methods include the use of liposomes, micelles and nanoparticles to increase GI tract permeation of peptides.

Various bio-responsive systems may also be combined with one or more peptide compound disclosed herein to provide a pharmaceutical agent for oral delivery. In some embodiments, a peptide of the instant disclosure is used in combination with a bioresponsive system, such as hydrogels and mucoadhesive polymers with hydrogen bonding groups (e.g., PEG, poly(methacrylic) acid [PMAA], cellulose, Eudragit®, chitosan and alginate) to provide a therapeutic agent for oral administration. Other embodiments include a method for optimizing or prolonging drug residence time for a peptide compound disclosed herein, wherein the surface of the peptide compound surface is modified to comprise mucoadhesive properties through hydrogen bonds, polymers with linked mucins or/and hydrophobic interactions. These modified peptide molecules may demonstrate increase drug residence time within the subject, in accordance with a desired feature of the disclosure. Moreover, targeted mucoadhesive systems may specifically bind to receptors at the enterocytes and M-cell surfaces, thereby further increasing the uptake of particles containing the peptide compound.

### Permeation Enhancer

Other embodiments comprise a method for oral delivery of a peptide compound disclosed herein, wherein the peptide compound is provided to a subject in combination with permeation enhancers that promote the transport of the peptides across the intestinal mucosa by increasing paracellular or transcellular permeation. Various permeation enhancers and methods for the oral delivery of therapeutic agents is described in Bray den, D. J., Mrsny, R. J., 2011. Oral peptide delivery: prioritizing the leading technologies. Ther. Delivery 2 (12), 1567-1573.

In certain embodiments, pharmaceutical compositions and Formulations disclosed herein comprises a peptide compound disclosed herein and one or more permeation enhancer. Examples of absorption enhancers may include Bile salts, fatty acids, surfactants (anionic, cationic, and non-ionic) chelators, Zonular OT, esters, cyclodextrin, dextran sulfate, azone, crown ethers, EDTA, sucrose esters, and phosphotidyl choline, for example. Although absorption enhancers are not typically carriers by themselves, they are also widely associated with other carriers to improve oral bioavailability by transporting of peptides and proteins across the intestinal mucosa. Such substances can be added to the formulation as excipients or incorporated to form non specific interactions with the intended peptide compound.

Dietary components and or other naturally occurring substances affirmed as enhancing tight junction permeation and as Generally Recognized As Safe (GRAS) include, e.g., as glycerides, acylcarnitines, bile salts, and medium chain fatty acids. Sodium salts of medium chain fatty acids (MCFAS) were also suggested to be permeation enhancers. The most extensively studied MCFAS is sodium caprate, a salt of capric acid, which comprises 2-3% of the fatty acids in the milk fat fraction. To date, sodium caprate is mainly used as an excipient in a suppository Formulation (Doktacillin™) for improving rectal ampicillin absorption. The permeation properties of another dietary MCFAS, sodium caprylate (8-carbon), were shown in vitro to be lower when compared to sodium caprate. Sodium caprylate and a peptide drug were Formulated in an admixture with other excipients in oil to generate an oily suspension (OS) that enhanced permeability (Tuvia, S. et al., Pharmaceutical Research, Vol. 31, No. 8, pp. 2010-2021 (2014).

For example, in one embodiment, a permeation enhancer is combined with a peptide compound, wherein the permeation enhancer comprises at least one of a medium-chain fatty acid, a long-chain fatty acid, a bile salt, an amphiphilic surfactant, and a chelating agent. In certain embodiments, medium-chain fatty acid salts promote absorption by increasing paracellular permeability of the intestinal epithelium. In one embodiment, a permeation enhancer comprising sodium N-[hydroxybenzoyl)amino] caprylate is used to form a weak noncovalent association with the peptide of the instant disclosure, wherein the permeation enhancer favours membrane transport and further dissociation once reaching the blood circulation. In another embodiment, a peptide compound disclosed herein is linked to oligoarginine, thereby increasing cellular penetration of the peptide into various cell types. Further, in at least one embodiment a noncovalent bond is provided between a peptide compound disclosed herein and a permeation enhancer selected from the group consisting of a cyclodextrin (CD) and a dendrimers, wherein the permeation enhancer reduces peptide aggregation and increasing stability and solubility for the peptide compound molecule.

In certain embodiments, a pharmaceutical composition or formulation comprises a peptide compound disclosed herein and a transient permeability enhancer (TPE). Permeation enhancers and TPEs may be used to increase orally bioavailability or the peptide compound. One example of a TPE that may be used is an oily suspension Formulation that disperses a powder containing sodium caprylate and a therapeutic agent (Tuvia, S. et al, Pharmaceutical Research, Vol. 31, No. 8, pp. 2010-2021 (2014).

In certain embodiments, pharmaceutical composition and formulations may include a peptide compound disclosed herein and one or more absorption enhancers, enzyme inhibitors, or mucosa adhesive polymers.

In some embodiments, peptide compounds disclosed herein are formulated in a formulation vehicle, such as, e.g., emulsions, liposomes, microsphere or nanoparticles.

Other embodiments disclosed herein provide a method for treating a subject with a peptide compound disclosed herein having an increased half-life. In one aspect, the present disclosure provides a peptide compound having a half-life of at least several hours to one day in vitro or in vivo (e.g., when administered to a human subject) sufficient for daily (q.d.) or twice daily (b.i.d.) dosing of a therapeutically effective amount. In another embodiment, the peptide compound has a half-life of three days or longer sufficient for weekly (q.w.) dosing of a therapeutically effective amount. Further, in another embodiment, the peptide compound has a half-life of eight days or longer sufficient for bi-weekly (b.i.w.) or monthly dosing of a therapeutically effective amount. In another embodiment, the peptide compound is derivatized or modified such that is has a longer half-life as compared to the underivatized or unmodified peptide compound. In another embodiment, the peptide compound contains one or more chemical modifications to increase serum half-life.

When used in at least one of the treatments or delivery systems described herein, a peptide compound disclosed herein may be employed in pure form or, where such forms exist, in pharmaceutically acceptable salt form.

The total daily usage of the peptide compounds and compositions disclosed herein can be decided by the attending physician within the scope of sound medical judgment. The specific therapeutically effective dose level for any particular subject will depend upon a variety of factors including: a) the disorder being treated and the severity of the disorder; b) activity of the specific compound employed; c) the specific composition employed, the age, body weight, general health, sex and diet of the patient; d) the time of administration, route of administration, and rate of excretion of the specific peptide compound employed; e) the duration of the treatment; f drugs used in combination or coincidental with the specific peptide compound employed, and like factors well known in the medical arts.

### Dosing

The term “therapeutically effective amount” of a compound refers to a nontoxic but sufficient amount of the compound to provide the desired effect. The amount of a compound of the formula (I) or (Ia) necessary to achieve the desired biological effect depends on a number of factors, for example the specific compound chosen, the intended use, the mode of administration and the clinical condition of the patient. An appropriate “effective” amount in any individual case may be determined by one of ordinary skill in the art using routine experimentation. For example, the “therapeutically effective amount” of a compound of formula (I) or (Ia) is about 0.01 to 50 mg/dose, or 0.02 to 1 mg/dose or for example, from 0.0001 to 300 mg/kg body weight daily or 1 to 300 mg/kg body weight daily.

In case of parenteral application, it could be favorable for the corresponding formulations to include at least one antimicrobial preservative in order to inhibit the growth of microbes and bacteria between administrations.

### Administration Unit, Package, Pen Device and Administration

The compound(s) disclosed herein can be prepared for use in suitable pharmaceutical compositions. The suitable pharmaceutical compositions may be in the form of one or more administration units.

The compositions may be prepared by any suitable pharmaceutical method which includes a step in which the compound(s) disclosed herein and the carrier (which may consist of one or more additional ingredients) are brought into contact.

The administration units may be for example capsules, tablets, dragées, granules sachets, drops, solutions, suspensions, lyophilizates and powders, each of which contains a defined amount of the compound(s) disclosed herein.

Each of the above-mentioned administration units of the compound(s) disclosed herein or pharmaceutical compositions disclosed herein (administration units) may be provided in a package for easy transport and storage. The administration units are packaged in standard single or multi-dosage packaging, their form, material and shape depending on the type of units prepared.

In some embodiments, the present disclosure provides kits that comprise a compound of formula (I) or (Ia), in any of its stereoisomeric forms, or a physiologically acceptable salt or solvate thereof, and a set of instructions relating to the use of the compound for the methods described herein. In some embodiments, the kit further comprises one or more inert carriers and/or diluents. In some embodiments, the kit further comprises one or more other pharmacologically active compounds, such as those described herein.

In certain embodiments administration units may be provided together with a device for application, for example together with a syringe, an injection pen or an autoinjector. Such devices may be provided separate from a pharmaceutical composition or prefilled with the pharmaceutical composition.

A “pen-type injection device”, often briefly referred to as “injection pen”, is typically an injection device having an elongated shape that resembles to a fountain pen for writing. Although such pens usually have a tubular cross-section, they could easily have a different cross-section such as triangular, rectangular or square or any variation around these geometries. Generally, pen-type injection devices comprise three primary elements: a cartridge section that includes a cartridge often contained within a housing or holder; a needle assembly connected to one end of the cartridge section; and a dosing section connected to the other end of the cartridge section. The cartridge, often also referred to as “ampoule”, typically includes a reservoir that is filled with a medication, a movable rubber type bung or stopper located at one end of the cartridge reservoir, and a top having a pierceable rubber seal located at the other, often necked-down, end. A crimped annular metal band is typically used to hold the rubber seal in place. While the cartridge housing may be typically made of plastic, cartridge reservoirs have historically been made of glass.

### Combination Therapy

In some embodiments, methods disclosed herein comprise providing a peptide compound disclosed herein (i.e., a first therapeutic agent) to a subject in need thereof in combination with a second therapeutic agent. In certain embodiments, the second therapeutic agent is provided to the subject before and/or simultaneously with and/or after the peptide compound is administered to the subject. In some embodiments, the second therapeutic agent is an anti-inflammatory agent. In certain embodiments, the second therapeutic agent is a non-steroidal anti-inflammatory drug, steroid, or immune modulating agent.

In another embodiment, the method comprises administering to the subject a third therapeutic agent. In certain embodiments, the second therapeutic agent is an antibody that binds IL-23 or IL-23R.

The active ingredient combinations can be used especially for a synergistic improvement in action. They can be applied either by separate administration of the active ingredients to the patient or in the form of combination products in which a plurality of active ingredients is present in one pharmaceutical preparation. The amount of the compound disclosed herein, and the other pharmaceutically active ingredient(s) and the relative timings of administration will be selected in order to achieve the desired combined therapeutic effect. The administration of the combination may be concomitantly in: (1) a unitary pharmaceutical composition including all pharmaceutically active ingredients; or (2) separate pharmaceutical compositions each including at least one of the pharmaceutically active ingredients. Alternatively, the combination may be administered separately in a sequential manner wherein one treatment agent is administered first, and the other treatment agent is administered second, or vice versa. When the active ingredients are administered by separate administration of the active ingredients, this can be done simultaneously or successively.

Other active substances which are suitable for such combinations include for example those which potentiate the therapeutic effect of one or more active substances with respect to one of the indications mentioned and/or which allow the dosage of one or more active substances to be reduced.

In another aspect, this disclosure relates to the use of a compound according to the disclosure or a physiologically acceptable salt thereof combined with at least one of the active substances described above as a combination partner, for preparing a medicament which is suitable for the treatment or prevention of diseases or conditions which can be affected by binding to the receptor interleukin-23 and by modulating the activity. This is for example a disease in the context of the immune system, for example one of the diseases or conditions listed above, for example irritable bowel syndrome, Crohn's disease, psoriasis and Colitis ulcerosa or complications thereof.

The use of the compounds according to the disclosure, or a physiologically acceptable salt thereof, in combination with one or more active substances may take place simultaneously, separately or sequentially.

The use of the compound according to the disclosure, or a physiologically acceptable salt thereof, in combination with another active substance may take place simultaneously or at staggered times, but for example within a short space of time. If they are administered simultaneously, the two active substances are given to the patient together.

Consequently, in another aspect, this disclosure relates to a medicament which comprises a compound according to the disclosure or a physiologically acceptable salt of such a compound and at least one of the active substances described above as combination partners, optionally together with one or more inert carriers and/or diluents.

The compound according to the disclosure, or physiologically acceptable salt or solvate thereof, and the additional active substance to be combined therewith may both be present together in one formulation, for example a tablet, capsule or solution, or separately in two identical or different formulations, for example as so-called kit-of-parts.

Another subject disclosed herein are processes for the preparation of the compounds of formula (I) or (Ia) and their salts and solvates, by which the compounds are obtainable, and which are exemplified in the following.

### EXAMPLES

### Methods

Abbreviations employed are as follows:
AA amino acid
ACN acetonitrile
Aib alpha-amino-isobutyric acid
Boc tert-butyloxycarbonyl
tBu tertiary butyl
CV Column volume
DCM dichloromethane
DIC N,N′-diisopropylcarbodiimide
DIPEA N,N-diisopropylethylamine
DMF dimethyl formamide
DODT 3,6-dioxa-1,8-octanedithiol
DPBS Dulbecco's phosphate-buffered saline
EDC 1-Ethyl-3-(3-dimethylaminopropyl) carbodiimide
EDT ethanedithiol
Fmoc fluorenylmethyloxycarbonyl
g gram
HATU O-(7-azabenzotriazol-1-yl)-N,N,N′,N′-tetramethyluronium hexafluorophosphate
HBTU 2-(1H-benzotriazol-1-yl)-1,1,3,3-tetramethyl-uronium hexafluorophosphate
HOAt 1-hydroxy-7-azabenzotriazole
HOBt 1-hydroxybenzotriazole
HPLC High Performance Liquid Chromatography
LC/MS Liquid Chromatography/Mass Spectrometry
mM millimolar
Mmt monomethoxy-trityl
n.a. not available
n.d. not determined
nM nanomolar
PBS phosphate buffered saline
Pfp 2,2,4,6,7-pentamethyldihydrobenzofuran-5-sulfonyl
PyBOP benzotriazol-1-yloxytripyrrolidinophosphonium hexafluorophosphate
tBu t-butyl
TFA trifluoroacetic acid
Trt trityl/triphenymethyl
TSTU N,N,N′,N′-Tetramethyl-O—(N-succinimidyl)uroniumtetrafluorborat
UPLC Ultra Performance Liquid Chromatography
UV ultraviolet

### Materials

Different Rink-Amide resins (e.g., 4-(2′,4′-Dimethoxyphenyl-Fmoc-aminomethyl)-phenoxyacetamido-norleucylaminomethyl resin, Merck Biosciences; 4-[(2,4-Dimethoxyphenyl) (Fmoc-amino)methyl]phenoxy acetamido methyl resin, Agilent Technologies) were used for the synthesis of peptide amides with loadings in the range of 0.2-0.7 mmol/g.

Fmoc protected natural amino acids were purchased from Protein Technologies Inc., Senn Chemicals, Merck Biosciences, Novabiochem, Iris Biotech, Bachem, Chem-Impex International or MATRIX Innovation. The following standard amino acids were used throughout the syntheses: Fmoc-L-Ala-OH, Fmoc-Arg(Pbf)-OH, Fmoc-L-Asn(Trt)-OH, Fmoc-L-Asp(OtBu)-OH, OH, Fmoc-L-Gln(Trt)-OH, Fmoc-L-Glu(OtBu)-OH, Fmoc-L-Leu-OH, Fmoc-L-Lys(Boc)-OH, Fmoc-D-Lys(Boc)-OH, Fmoc-L-Phe-OH, Fmoc-L-Thr(tBu)-OH, Fmoc-L-Trp(Boc)-OH, Fmoc-L-Tyr(tBu)-OH, Fmoc-Cys(Trt)-OH.

In addition, the following special amino acids were purchased from the same suppliers as above: Fmoc-L-1-Nal-OH, Fmoc-L-2-Nal-OH, Fmoc-L-Phe(2-Ae-Boc)-OH (Fmoc-L-Yae(Boc)-OH), Fmoc-L-Phe(Me)-OH (Fmoc-L-Yme-OH), Fmoc-Yde-OH, Fmoc-L-Orn(Boc), Fmoc-L-Trp(Me)-OH, Fmoc-L-Agb(Pbf)-OH, Fmoc-Ahx-OH, Fmoc-Aoa-OH, Fmoc-Apt-OH, Fmoc-Bal-OH, Fmoc-Hme-OH, Fmoc-Kme(Boc)-OH, Fmoc-L-Lys(Ac)-OH, ε-Fmoc-L-Lys(αAc)—OH, Fmoc-Mkdm-OH, Fmoc-Mle-OH, Fmoc-Mep-OH, Fmoc-Mly(Boc)-OH, ε-Fmoc-Ndmk-OH, Fmoc-Nmapt-OH, Fmoc-Nmc(Trt)-OH, Fmoc-Nmd(O-tBu)-OH, Fmoc-Nme(O-tBu)-OH, Fmoc-Nmha-OH, Fmoc-Nmn(Trt)-OH, Fmoc-Pal-OH, Fmoc-Pen(Trt)-OH, Fmoc-Pyal-OH, Fmoc-Sar-OH and Fmoc-Thp-OH.

In addition, the dicarboxylic acid Dad was purchased and used from the same suppliers as mentioned for the Amino acids in form of the Di-N-hydroxysuccinimide ester: Dad (Di-O-Succ).

### Synthesis of Mkdm

470 mg (2S)-6-(tert-butoxycarbonylamino)-2-(9H-fluoren-9-ylmethoxycarbonylamino) hexanoic acid was treated with 5 mL TFA for two hours. Then, 100 mL water were added, and the solution was frozen and lyophilized. The residue was taken up with 10 mL methanol. Then, 129 mg DIPEA, 126 mg NaCNBH₃ and 50 mg formaldehyde were added, and the mixture was stirred for one hour. The product was purified by chromatography to give 230 mg yield.

### Analytical UPLC/UV/MS Methods

### Method A: Detection at 214 nm

HPLC: Waters i-class UPLC
column: Waters ACQUITY UPLC® CSH™ C18 1.7 μm (75×2.1 mm) at 50° C. solvent: H2O+0.05% TFA: ACN+0.045% TFA (flow 0.7 ml/min)
gradient: 95:5 (0 min) to 95:5 (1 min) to 20:80 (12 min) to 5:95 (12.5 min) to 5:95 (13.5 min) to 95:5 (14 min) to 95:5 (16 min)
with mass analyser: Waters Xevo G2-XS QTof, electrospray positive ion mode, mass range: 300-3200 m/z, resolution mode

### Method B: Detection at 214 nm

HPLC: Agilent 1290 Infinity II UPLC
column: Waters ACQUITY UPLC® CSH™ C18 1.7 μm (150×2.1 mm) at 50° C. solvent: H2O+0.05% TFA: ACN+0.035% TFA (flow 0.5 ml/min)
gradient: 80:20 (0 min) to 80:20 (3 min) to 25:75 (23 min) to 2:98 (23.5 min) to 2:98 (30.5 min) to 80:20 (31 min) to 80:20 (37 min)
with mass analyser: Agilent 6230 Accurate-Mass TOF, Agilent Dual Jet Stream ESI+, mass range: 300-3200 m/z, extended dynamic range mode (2 Ghz)

### Method C: Detection at 214 nm

HPLC: Agilent 1290 Infinity II UPLC
column: Waters ACQUITY UPLC® CSH™ C18 1.7 μm (150×2.1 mm) at 50° C. solvent: H2O+0.05% TFA: ACN+0.035% TFA (flow 0.5 ml/min)
gradient: 95:5 (0 min) to 95:5 (3 min) to 40:60 (23 min) to 2:98 (23.5 min) to 2:98 (30.5 min) to 95:5 (31 min) to 95:5 (37 min)
with mass analyser: Agilent 6230 Accurate-Mass TOF, Agilent Dual Jet Stream ESI, mass range: 300-3200 m/z, extended dynamic range mode (2 Ghz)

### Method D: Detection at 220 nm

HPLC: Shimadzu Nexera X2
column: Waters ACQUITY UPLC® BEH™ C18 1.7 μm (100×2.1 mm) at 40° C. solvent: H2O+0.1% TFA: ACN+0.1% TFA (flow 0.5 ml/min)
gradient: 95:5 (0 min) to 95:5 (3 min) to 40:60 (23 min) to 2:98 (23.5 min) to 2:98 (30.5 min) to 95:5 (31 min) to 95:5 (37 min)
with mass analyser: LCMS 2020, DL-temp: 250 C, Nebulization gas flow: 1.5 L/min, Detector voltage: 1.4 KV

### Method E: Detection at 214 nm

HPLC: Agilent 1290 Infinity II UPLC
column: Waters ACQUITY UPLC® CSH™ C18 1.7 μm (150×2.1 mm) at 50° C. solvent: H2O+0.05% TFA: ACN+0.035% TFA (flow 0.5 ml/min)
gradient: 99:1 (0 min) to 99:1 (3 min) to 70:30 (18 min) to 2:98 (18.5 min) to 2:98 (25.5 min) to 99:1 (26 min) to 99:1 (32 min)
with mass analyser: Agilent 6230 Accurate-Mass TOF, Agilent Dual Jet Stream ESI, mass range: 300-3200 m/z, extended dynamic range mode (2 Ghz)

### Method F: Detection at 214 nm

HPLC: Agilent 1290 Infinity II UPLC
Column: Waters ACQUITY UPLC® CSH™ C18 1.7 μm (150×2.1 mm) at 50° C. solvent: H2O+0.05% TFA: ACN+0.045% TFA (flow 0.5 ml/min)
gradient: 95:5 (0 min) to 95:5 (3 min) to 80:20 (25 min) to 2:98 (25.5 min) to 2:98 (30.5 min) to 95:5 (31 min) to 95:5 (35 min)
with mass analyser: Agilent 6230 Accurate-Mass TOF, Agilent Dual Jet Stream ESI, mass range: 300-3200 m/z, extended dynamic range mode (2 Ghz)

### Method G: Detection at 214 nm

HPLC: Agilent 1290 Infinity II UPLC
Column: Waters ACQUITY UPLC® CSH™ C18 1.7 μm (150×2.1 mm) at 50° C. solvent: H2O+0.05% TFA: ACN+0.045% TFA (flow 0.5 ml/min)
gradient: 95:5 (0 min) to 95:5 (3 min) to 80:20 (30 min) to 2:98 (30.5 min) to 2:98 (34.5 min) to 95:5 (35 min) to 95:5 (40 min)
with mass analyser: Agilent 6230 Accurate-Mass TOF, Agilent Dual Jet Stream ESI, mass range: 300-3200 m/z, extended dynamic range mode (2 Ghz)

### General Preparative HPLC Purification Procedure

The crude peptide compounds were purified either on an Äkta Purifier System, a Jasco semiprep HPLC System, Waters Autopurification System, an Agilent 1100 HPLC system or a similar HPLC system. Preparative RP-C18-HPLC columns of different sizes and with different flow rates were used depending on the amount of crude peptide to be purified, e.g., the following columns have been used: Waters XSelect CSH C18 OBD Prep 5 μm 30×250 mm, Waters SunFire C18 OBD Prep 5 μm 30×250 mm, Waters SunFire C18 OBD Prep 5 μm 50×150 mm, and Phenomenex Luna Prep C18 5 μm 21.2×250 mm. Acetonitrile (B) and water+0.1% TFA (A) or water+0.1% TFA (A) were employed as eluents. Product-containing fractions were collected and lyophilized to obtain the purified product, typically as TFA salt.

Alternatively, the peptide compounds can be isolated as acetate salts via the following procedure: The peptide compound was dissolved in water and the solution adjusted to pH 7.05 with NaHCO₃. Then, the dissolved compound was purified with a RP Kinetex 21.2×250 mm (Column Volume CV 88 ml, 5 μm, C18, 100A, Äkta avant 25): The column was equilibrated with solvent A (3×CV), the compound was injected and then washed with a mixture of solvent A (95%) and solvent B (5%) with 3 CV. Then, a gradient solvent A: B (95:5) to A: B (20:80) was run with 15 CV. The purified peptide was collected and lyophilized.

### General Synthesis of Peptide Compounds

### Solid Phase Peptide Synthesis

The solid phase peptide syntheses were performed on a Prelude Peptide Synthesizer (Protein Technologies) or a similar automated synthesizer using standard Fmoc chemistry and HBTU/DIPEA or HATU/DIPEA activation. DMF was used as the solvent.

Deprotection: 20% piperidine/DMF for 2×2.5 min.

Washes: 7×DMF.

Coupling: May 5, 2013 200 mM AA/500 mM HBTU in DMF/2M DIPEA in NMP 2× for 20 min. Washes: 5×DMF.

HBTU/DIPEA activation was used for all standard couplings.

For N-terminal acetylated peptides, the N-terminal Fmoc protecting group was removed and the peptide was treated twice with 10% solution of acetic acid anhydride and DIPEA for 20 minutes with shaking.

The peptides were cleaved from the resin with King's cleavage cocktail consisting of 82.5% TFA, 5% phenol, 5% water, 5% thioanisole, and 2.5% EDT or a modified cleavage cocktail consisting of 82.5% TFA, 5% phenol, 5% water, 5% thioanisole, and 2.5% DODT. The crude peptides were then precipitated in diethyl or diisopropyl ether, centrifuged, and lyophilized. Peptides were analysed by analytical HPLC and checked by ESI mass spectrometry. Crude peptides were purified by a conventional preparative RP-HPLC purification procedure as described in the HPLC section.

### Peptide Synthesis for PyEA in Position X13

In case of PyEA in position X13, Wang resin was used. The solid phase synthesis was started from C-terminal attachment of the X12 amino acid to the resin, following the built-up of the sequence on the resin, cleavage of the peptide from the resin and subsequent cyclisation.

The free C-terminal carboxylic acid of the peptide was treated with TSTU (1 eq), DIPEA (2 eq) in DMF for 1 hour. Then PyEA (1 eq) was added to the solution to give the desired product. The mixture was purified by chromatography as described in the UPLC section.

### Peptide Synthesis with a Dicarboxylic Acid (Dad) in Position X1

The purified peptide was dissolved in DMF. 10 eq DiPEA were added and then 0.95 eq Di-OSu-Ester in DMF added dropwise and mixed for 5 min at RT. The mixture was then purified by chromatography.

### Synthesis of Peptides According to Formula (Ia)

### Synthesis of Compound SEQ ID NO: 67

### Synthesis of Fmoc-Mly-Palm-OH

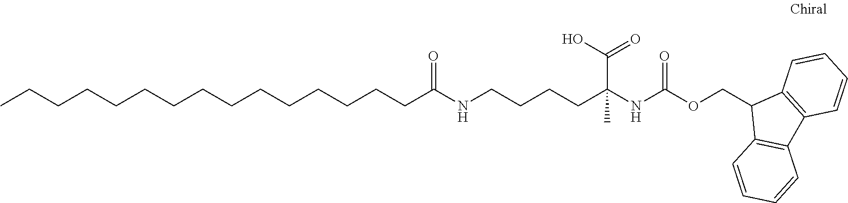 [SMILES: CCCCCCCCCCCCCCCC(=O)NCCCC[C@](C)(NC(=O)OCC1c2ccccc2-c2ccccc21)C(=O)O]

(2S)-6-(tert-butoxycarbonylamino)-2-(9H-fluoren-9-ylmethoxycarbonylamino)-2-methyl-hexanoic acid 31 g were dissolved in 50 mL TFA (90%). Then, 1000 mL water were added and the solution was lyophylisated. The residue was taken up in 150 mL DMF and 10.5 g DIPEA followed by 9.6 g (2,5-dioxopyrrolidin-1-yl) hexadecanoate were added. After one hour stirring at room temperature, the solution was concentrated in vacuo, 500 ml water was added and the mixture was lyophylisated. The residue was purified by preparative HPLC to give the desired product (16 g).

This amino acid derivative was used in position X10 the same way as a regular amino acid during SPPS and the peptide was obtained as described for the general SPPS synthesis method.

### Synthesis of Compound Seq ID No. 71

Synthesis of Sema is described in WO2011080102, which is incorporated by reference.

For the Sema side chain, Fmoc-protected lysine (1 eq) and 1000 mg (1 eq) of tert-butyl 18-oxo-18-[rac-(1S)-1-tert-butoxycarbonyl-4-[2-[2-[2-[2-[2-[2-(2,5-dioxopyrrolidin-1-yl)oxy-2-oxo-ethoxy]ethoxy]ethylamino]-2-oxo-ethoxy]ethoxylethylamino]-4-oxo-butyl]amino]octadecenoate (i.e. the succinimide ester-activated 8-oxo-18-[(1S)-1-carboxy-4-[2-[2-[2-[2-[2-(carboxymethoxy)ethoxy]ethylamino]-2-oxo-ethoxy]ethoxy]ethylamino]-4-oxo-butyl]amino]octadecanoic acid, wherein the glutamic acid carboxy group and the terminal carboxy group are protected as tert. butylester) were treated with TSTU (1.5 eq.) in DMF (30 mL) and DIPEA (4 eq.) and stirred for 15 minutes. The solution was concentrated in vacuo, water added (100 mL) and lyophylisated.

This derivatised amino acid was used in the SPPS. The cysteins in position X2 and X7 were used in form of Fmoc-Cys(Mmt)-OH. The resin was flushed with dichloromethane and then treated with a iodine solution in methanol (5 eq.) (16 mg/mL). Residues of iodine were removed with ascorbic acid after 30 minutes to give the oxidized product. After flushing with dichloromethane (100 mL) Fmoc protection group was cleaved by swelling the resin with DMF (50 mL) and addition of piperidine (8 eq., 40 mL) for 10 minutes. This procedure was repeated once.

Then, the peptide (0.1 mmol) was cleaved from the resin with 100 mL Kings Cocktail (85 mL TFA/5,355 g Phenol/5 mL H2O/5 mL Thioanisole/2.5 mL Ethandithiol) for 3 hours at room temperature. The dissolved peptide was poured into cold ether (700 mL). The precipitated peptide was centrifuged and washed with ether (800 mL). The suspension was filtered, and the solid peptide was dried in vacuo for 1 hour. Then, peptide was dissolved in 600 ml water/acetic acid 99/1 (1 hour) and lyophylisated. The residue was purified by preparative HPLC.

30 mg of the dry peptide was dissolved in 10 mL DMF and 10 eq. diisopropyl ethylamine were added. 0.95 eq. disuccinimidyl succinate were added and stirred for 1 hour at room temperature. The solution was concentrated and purified by preparative HPLC to give the desired product (16 mg).

### Peptide Cyclisation

### Disulfide Cyclisation

In case of peptides with two sulfhydryl moieties, compounds were suspended (partly dissolved) in acetonitrile/water (2 mg/mL) and the mixture was treated dropwise with a solution of acetic acid/iodine (5 mL/80 mg) until the solution has a bright yellow color. Then, ascorbic acid (1.7 g in 100 mL water) was added until the yellow color disappeared.

The solution was diluted with water and lyophilized. The crude peptide was purified by a conventional preparative RP-HPLC purification procedure as described in the HPLC method section. Fractions containing pure product were then freeze-dried on a lyophilizer.

### Lactam Cyclisation

Subsequent peptide cyclization was performed under argon atmosphere. The peptide was dissolved in DMF, then 10 eq. DIPEA, 3 eq. HOAT and 2 eq. EDC was added, and the reaction was stirred overnight at room temperature.

The peptide containing the free thiol (eg Cysteine, homo Cysteine (hCys)) and homo Serine (OTBDMS) was assembled on a Rink Amide-MBHA resin following general Fmoc-SPPS procedure.

### Cyclisation by Thioether Formation (X2 is Abu and X7 is Cys)

For the formation of a thioether mono-methoxy-trityl (Mmt) protected Cystein was used. The MMT group was cleaved by using 2% TFA in dichloromethane (DCM) and 0.5% TIPS for 15 minutes. The procedure was repeated twice. The resin was washed with DCM three times. Chlorination was carried out with Dichlorotriphenylphosphorane (6 eq) along with Pinene (6 eq) and thioanisole (4 eq) scavengers at room temperature for 4 hours. The peptide was cleaved from the resin by treatment with cleavage reagent 90% trifluoroacetic acid, 5% water, 2.5% 1,2-ethanedithiol, 2.5% tri-isopropylsilane. The cleaved peptides were precipitated in cold diethyl ether followed by two washings with ethyl ether. The filtrate was poured off and a second aliquot of cold ether was added, and the procedure repeated. The crude peptide was dissolved in a solution of acetonitrile:water (7:3 with 1% TFA) and filtered giving the wanted uncyclized crude peptide. The crude peptide possessing a free thiol (eg Cys, hCys, (D)Cys or (D)hCys) and the alkyl halide (hSer(CI)) at either the X2 and X7 position or X7 and X2 position was dissolved in 0.1M TRIS buffer pH 8.5. Cyclization was allowed to take place overnight at RT. The solvent mixture was then purified by first being diluted two-fold with water and then loaded onto a reverse phase HPLC machine Fractions containing pure product were then freeze-dried on a lyophilizer.

### Lactam Cyclisation

Alternatively, the peptide was dissolved in DMF, cooled to 0° C. before 20 eq. NaHCO₃, 3 eq. HOAT and 2.5 eq EDC·HCl was added. The reaction was stirred and allowed to slowly equilibrate to room temperature overnight. NaHCO₃ was filtered over gravity and the soluble fraction dried under rotary evaporation. The crude cyclic peptide was dissolved in ACN: Water and freeze-dried. The desired cyclic peptide product was isolated by a conventional preparative RP-HPLC purification procedure.

In case of Yae in the sequence, after cleavage from the resin with King's cleavage cocktail and, if it applies, disulfide ring closure under conditions described above, the N-terminus was kept Fmoc protected, the deprotected Yae moiety was protected by BOC-OSu (2 eq.) and diisopropyl-ethylamine in DMF. Then, the N-terminal Fmoc group was cleaved with piperidine (20 eq.). After workup and purification with RP-HPLC, the peptide cyclization was performed. Finally, the BOC protecting group was cleaved with TFA (50 eq.). The desired cyclic peptide product was isolated by a conventional preparative RP-HPLC purification procedure.

Cyclisation with Butanedial or 4-Oxo-Butanoic Acid (X1 is Buty or Butyl)

After cleavage from the resin and purification by HPLC, the freeze-dried peptide was dissolved in methanol. Sodium cyanoborohydrate (1 eq.) and DIPEA (1 eq.) was added, and the mixture was stirred for 16 hours. In case of butanedial one eq. was added together with DIPEA. The solvent was evaporated, and the residue purified by HPLC and freeze-dried to give the desired peptide.

### Peptides with Pen as X2 and Pen or Cys at X7

The following peptide compounds in which Cys in position X2, or in positions X2 and X7 is/are replaced with Pen (Pen peptide compound) where prepared as described for the compounds of the disclosure.

TABLE 4
Peptide compounds with Pen as X2 and X7

| SEQ ID | |
|---|---|
| NO : | SEQUENCE |
| 159 | Apt(§1)-Pen(@2)-N-T-W-Q-Pen(@2)-Yme-Nal-Aib-E(§1)-N—N—NH2 |
| 160 | Ahx(§1)-Pen(@2)-N-T-W-Q-Pen(@2)-Yme-Nal-Aib-E(§1)-N—N—NH2 |
| 161 | Ahx(§1)-Pen(@2)-N-T-W-Q-Pen(@2)-Yme-Nal-Thp-E(§1)-N-G-NH2 |
| 162 | Ahx(§1)-Pen(@2)-N-T-W-Q-Pen(@2)-Yme-Nal-Mle-D(§1)-N—N—NH2 |
| 163 | Apt(§1)-Pen(@2)-N-T-W-Q-Pen(@2)-Yae-Nal-Thp-E(§1)-N—N—NH2 |
| 164 | Ahx(§1)-Pen(@2)-N-T-W-Q-C(@2)-Yme-Nal-Aib-D(§1)-N—N—NH2 |

(@) indicates the formation of an ester, a thioeter, or a disulfide bridge between amino acid residues.

(§) indicates the formation of a lactam bridge between the amino acid residues.

By these methods the compounds with SEQ ID NO: 1 to 74, 101 to 106, 115, 116, 122, 126, 127, and 149 to 158 were obtained and compounds with SEQ ID NO: 75 to 101, 107 to 121, 123 to 125, 127 to 148 are obtainable. Representative structures are shown in Table 5.

TABLE 5
representative structures of peptide compounds of the disclosure

| SEQ | | Mol |
|---|---|---|
| ID | | mass |
| NO: | Formula | [g/mol] |
| 5 | 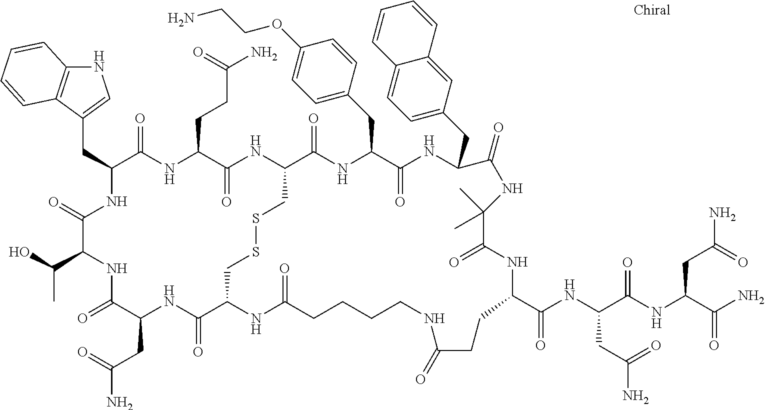 [SMILES: C[C@@H](O)[C@@H]1NC(=O)[C@H](CC(N)=O)NC(=O)[C@@H]2CSSC[C@H](NC(=O)[C@H](CCC(N)=O)NC(=O)[C@H](Cc3c[nH]c4ccccc34)NC1=O)C(=O)N[C@@H](Cc1ccc(OCCN)cc1)C(=O)N[C@H](Cc1ccc3ccccc3c1)C(=O)NC(C)(C)C(=O)N[C@H](C(=O)N[C@@H](CC(N)=O)C(=O)N[C@@H](CC(N)=O)C(N)=O)CCC(=O)NCCCCC(=O)N2] | 1677.9 |
| 8 | 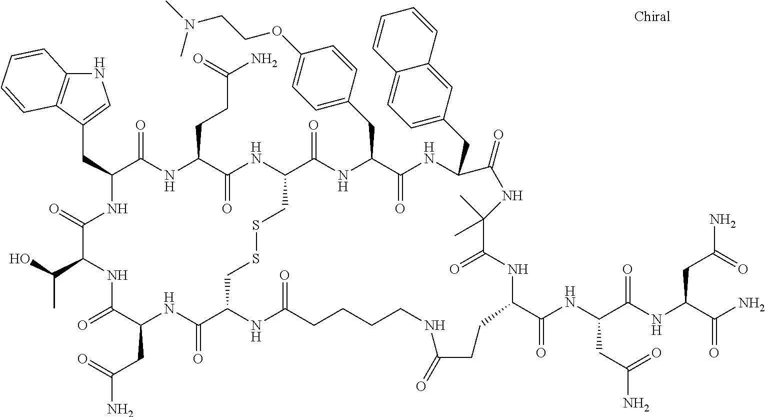 [SMILES: C[C@@H](O)[C@@H]1NC(=O)[C@H](CC(N)=O)NC(=O)[C@@H]2CSSC[C@H](NC(=O)[C@H](CCC(N)=O)NC(=O)[C@H](Cc3c[nH]c4ccccc34)NC1=O)C(=O)N[C@@H](Cc1ccc(OCCN(C)C)cc1)C(=O)N[C@H](Cc1ccc3ccccc3c1)C(=O)NC(C)(C)C(=O)N[C@H](C(=O)N[C@@H](CC(N)=O)C(=O)N[C@@H](CC(N)=O)C(N)=O)CCC(=O)NCCCCC(=O)N2] | 1705.9 |
| 14 | 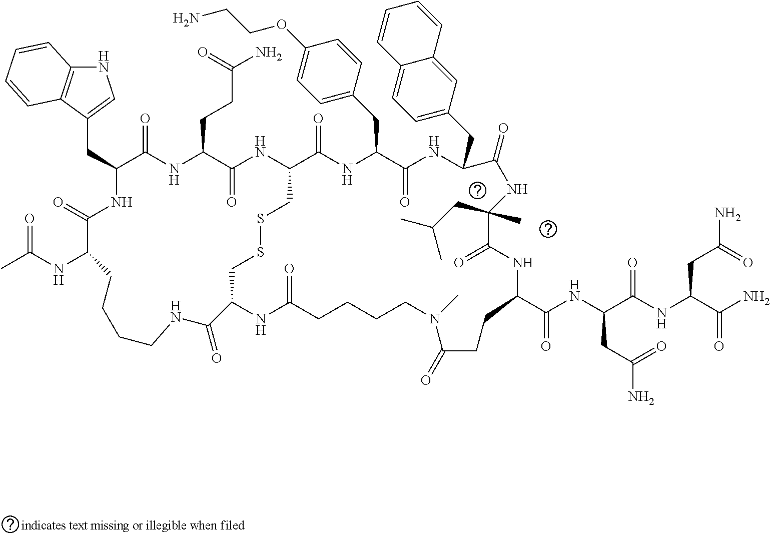 [SMILES: CC(=O)N[C@H]1CCCCNC(=O)[C@@H]2CSSC[C@H](NC(=O)[C@H](CCC(N)=O)NC(=O)[C@H](Cc3c[nH]c4ccccc34)NC1=O)C(=O)N[C@@H](Cc1ccc(OCCN)cc1)C(=O)N[C@H](Cc1ccc3ccccc3c1)C(=O)N[C@@](C)(CC(C)C)C(=O)N[C@@H](C(=O)N[C@H](CC(N)=O)C(=O)N[C@@H](CC(N)=O)C(N)=O)CCC(=O)N(C)CCCCC(=O)N2] | 1689.0 |
| 35 | 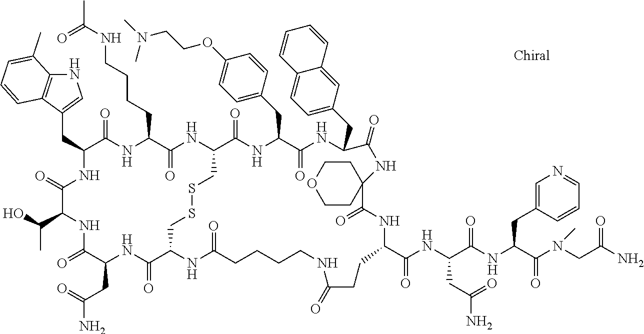 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H]2CSSC[C@H](NC1=O)C(=O)N[C@@H](Cc1ccc(OCCN(C)C)cc1)C(=O)N[C@H](Cc1ccc3ccccc3c1)C(=O)NC1(CCOCC1)C(=O)N[C@H](C(=O)N[C@@H](CC(N)=O)C(=O)N[C@@H](Cc1cccnc1)C(=O)N(C)CC(N)=O)CCC(=O)NCCCCC(=O)N2] | 1909.2 |
| 59 | 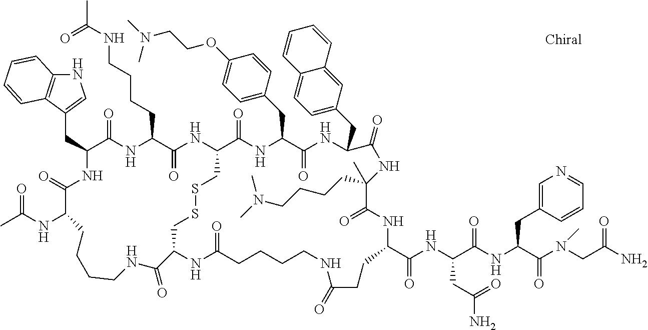 [SMILES: CC(=O)NCCCC[C@@H]1NC(=O)[C@H](Cc2c[nH]c3ccccc23)NC(=O)[C@@H](NC(C)=O)CCCCNC(=O)[C@@H]2CSSC[C@H](NC1=O)C(=O)N[C@@H](Cc1ccc(OCCN(C)C)cc1)C(=O)N[C@H](Cc1ccc3ccccc3c1)C(=O)N[C@@](C)(CCCCN(C)C)C(=O)N[C@H](C(=O)N[C@@H](CC(N)=O)C(=O)N[C@@H](Cc1cccnc1)C(=O)N(C)CC(N)=O)CCC(=O)NCCCCC(=O)N2] | 1893.3 |
| 66 | 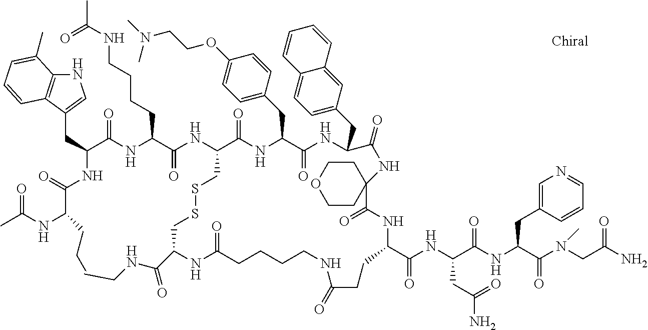 [SMILES: CCC[C@H](NC(C)=O)C(=O)N[C@@H](Cc1c[nH]c2c(C)cccc12)C(=O)N[C@@H](CCCCNC(C)=O)C(=O)N[C@H]1CSSC[C@@H](C(=O)NCC)NC(=O)CCCCNC(=O)CC[C@@H](C(=O)N[C@@H](CC(N)=O)C(=O)N[C@@H](Cc2cccnc2)C(=O)N(C)CC(N)=O)NC(=O)C2(CCOCC2)NC(=O)[C@@H](Cc2ccc3ccccc3c2)NC(=O)[C@H](Cc2ccc(OCCN(C)C)cc2)NC1=O] | 1864.2 |
| 128 | 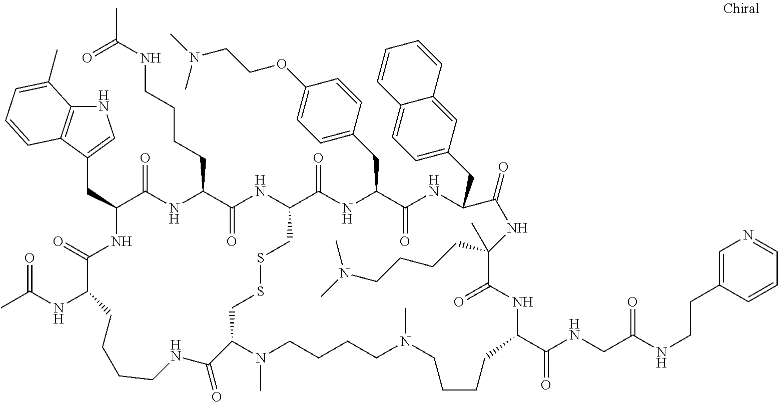 [SMILES: CC(=O)NCCCC[C@@H]1NC(=O)[C@H](Cc2c[nH]c3c(C)cccc23)NC(=O)[C@@H](NC(C)=O)CCCCNC(=O)[C@@H]2CSSC[C@H](NC1=O)C(=O)N[C@@H](Cc1ccc(OCCN(C)C)cc1)C(=O)N[C@H](Cc1ccc3ccccc3c1)C(=O)N[C@@](C)(CCCCN(C)C)C(=O)N[C@H](C(=O)NCC(=O)NCCc1cccnc1)CCCCN(C)CCCCN2C] | 1708.2 |
| 122 | 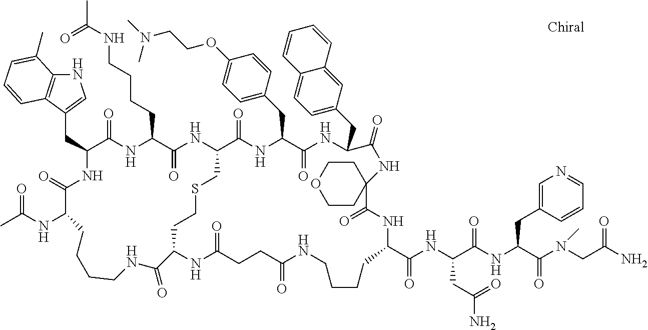 [SMILES: CC(=O)NCCCC[C@@H]1NC(=O)[C@H](Cc2c[nH]c3c(C)cccc23)NC(=O)[C@@H](NC(C)=O)CCCCNC(=O)[C@@H]2CCSC[C@H](NC1=O)C(=O)N[C@@H](Cc1ccc(OCCN(C)C)cc1)C(=O)N[C@H](Cc1ccc3ccccc3c1)C(=O)NC1(CCOCC1)C(=O)N[C@H](C(=O)N[C@@H](CC(N)=O)C(=O)N[C@@H](Cc1cccnc1)C(=O)N(C)CC(N)=O)CCCCNC(=O)CCC(=O)N2] | 1846.2 |
| 143 | 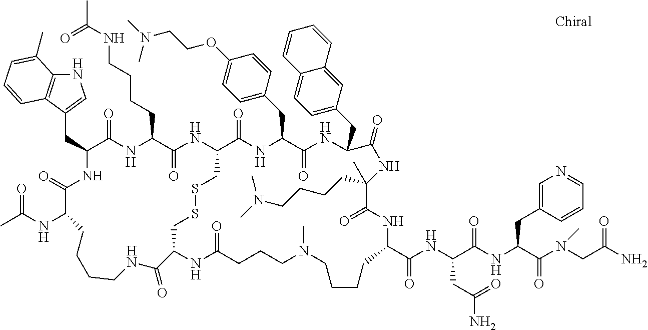 [SMILES: CC(=O)NCCCC[C@@H]1NC(=O)[C@H](Cc2c[nH]c3c(C)cccc23)NC(=O)[C@@H](NC(C)=O)CCCCNC(=O)[C@@H]2CSSC[C@H](NC1=O)C(=O)N[C@@H](Cc1ccc(OCCN(C)C)cc1)C(=O)N[C@H](Cc1ccc3ccccc3c1)C(=O)N[C@@](C)(CCCCN(C)C)C(=O)N[C@H](C(=O)N[C@@H](CC(N)=O)C(=O)N[C@@H](Cc1cccnc1)C(=O)N(C)CC(N)=O)CCCCN(C)CCCC(=O)N2] | 1907.3 |
| 145 | 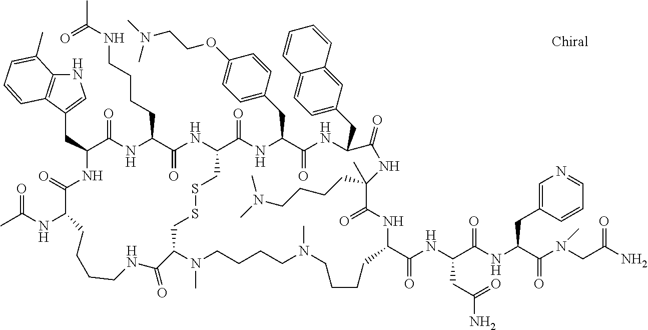 [SMILES: CC(=O)NCCCC[C@@H]1NC(=O)[C@H](Cc2c[nH]c3c(C)cccc23)NC(=O)[C@@H](NC(C)=O)CCCCNC(=O)[C@@H]2CSSC[C@H](NC1=O)C(=O)N[C@@H](Cc1ccc(OCCN(C)C)cc1)C(=O)N[C@H](Cc1ccc3ccccc3c1)C(=O)N[C@@](C)(CCCCN(C)C)C(=O)N[C@H](C(=O)N[C@@H](CC(N)=O)C(=O)N[C@@H](Cc1cccnc1)C(=O)N(C)CC(N)=O)CCCCN(C)CCCCN2C] | 1907.4 |
| 149 | 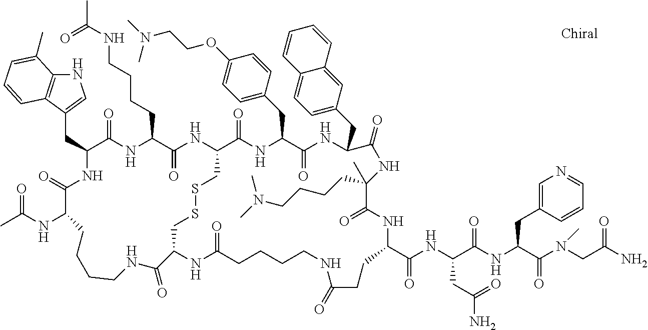 [SMILES: CC(=O)NCCCC[C@@H]1NC(=O)[C@H](Cc2c[nH]c3c(C)cccc23)NC(=O)[C@@H](NC(C)=O)CCCCNC(=O)[C@@H]2CSSC[C@H](NC1=O)C(=O)N[C@@H](Cc1ccc(OCCN(C)C)cc1)C(=O)N[C@H](Cc1ccc3ccccc3c1)C(=O)N[C@@](C)(CCCCN(C)C)C(=O)N[C@H](C(=O)N[C@@H](CC(N)=O)C(=O)N[C@@H](Cc1cccnc1)C(=O)N(C)CC(N)=O)CCC(=O)NCCCCC(=O)N2] | 1907.3 |
| 1- | See Table 3 | |
| 158 | | |

TABLE 6
List of synthesized peptides and comparison
of calculated vs. found molecular weight

| | | | Retention | |
|---|---|---|---|---|
| SEQ | Calc | Found | time | |
| ID NO | mass | mass | [min] | Method |
| 1 | 1647.66 | 1647.67 | 10.69 | B |
| 2 | 1661.67 | 1661.68 | 10.93 | B |
| 3 | 1647.66 | 1647.67 | 16.60 | C |
| 4 | 1590.63 | 1590.64 | 9.98 | B |
| 5 | 1676.68 | 1676.99 | 7.29 | D |
| 6 | 1661.67 | 1661.68 | 11.28 | B |
| 7 | 1704.68 | 1704.69 | 9.47 | B |
| 8 | 1704.71 | 1704.73 | 6.22 | B |
| 9 | 1619.66 | 1620 | 11.40 | D |
| 10 | 1745.80 | 1745.81 | 12.44 | B |
| 11 | 1647.66 | 1648 | 10.21 | D |
| 12 | 1645.71 | 1646 | 10.13 | D |
| 13 | 1687.72 | 1688 | 10.60 | D |
| 14 | 1687.76 | 1688 | 11.81 | D |
| 15 | 1588.69 | 1589 | 11.49 | D |
| 16 | 1630.70 | 1631 | 11.57 | D |
| 17 | 1587.70 | 1588 | 11.93 | D |
| 18 | 1587.73 | 1588 | 11.35 | D |
| 19 | 1601.75 | 1602 | 12.73 | D |
| 20 | 1783.79 | 1783.79 | 14.14 | C |
| 21 | 1825.80 | 1825.81 | 8.53 | B |
| 22 | 1825.84 | 1825.84 | 14.98 | C |
| 23 | 1825.84 | 1825.85 | 9.70 | B |
| 24 | 1850.82 | 1850.83 | 10.63 | B |
| 25 | 1718.73 | 1718 | 11.00 | D |
| 26 | 1746.73 | 1746.73 | 6.58 | B |
| 27 | 1746.76 | 1746.76 | 7.53 | B |
| 28 | 1701.78 | 1701.78 | 7.92 | B |
| 29 | 1602.71 | 1602.71 | 7.56 | B |
| 30 | 1644.72 | 1644.73 | 7.49 | B |
| 31 | 1644.75 | 1644.75 | 8.53 | B |
| 32 | 1559.70 | 1559.69 | 8.54 | B |
| 33 | 1657.78 | na | na | na |
| 34 | 1643.76 | 1644 | 9.51 | D |
| 35 | 1907.84 | 1907.87 | 8.58 | B |
| 36 | 1877.94 | 1877.93 | 11.70 | C |
| 37 | 1893.86 | 1893.83 | 14.82 | C |
| 38 | 1859.84 | 1859.83 | 15.71 | C |
| 39 | 1539.71 | 1539 | 9.68 | D |
| 40 | 1759.82 | 1759.82 | 10.81 | B |
| 41 | 1760.78 | 1760.78 | 7.73 | B |
| 42 | 1760.78 | 1760.78 | 7.73 | B |
| 43 | 1714.73 | 1714.75 | 16.32 | E |
| 44 | 1643.79 | 1644 | 13.78 | D |
| 45 | 1854.83 | 1854.84 | 17.24 | E |
| 46 | 1896.84 | 1896.84 | 6.48 | B |
| 47 | 1896.88 | 1896.88 | 7.50 | B |
| 48 | 1854.83 | 1854.83 | 6.53 | B |
| 49 | 1896.84 | 1896.85 | 17.04 | E |
| 50 | 1896.88 | 1896.89 | 7.36 | B |
| 51 | 1836.81 | 1836.82 | 6.81 | B |
| 52 | 1741.74 | 1741.76 | 16.32 | E |
| 53 | 1460.63 | 1461 | 8.68 | D |
| 54 | 1844.91 | 1844.92 | 8.39 | B |
| 55 | 1936.87 | 1936.88 | 6.83 | B |
| 56 | 1880.83 | 1880.84 | 6.82 | B |
| 57 | 1817.80 | 1817.81 | 13.26 | F |
| 58 | 1936.91 | 1936.92 | 15.48 | E |
| 59 | 1891.92 | 1891.92 | 11.69 | F |
| 60 | 1752.82 | 1752.83 | 13.16 | F |
| 61 | 1905.97 | 1905.98 | 12.19 | G |
| 62 | 1823.79 | na | na | na |
| 63 | 1865.83 | 1865.85 | 14.05 | G |
| 64 | 1865.84 | na | na | na |
| 65 | 1907.88 | 1907.89 | 14.90 | G |
| 66 | 1862.86 | 1862.87 | 14.40 | G |
| 67 | 2116.14 | 2116.14 | 22.67 | G |
| 68 | 1819.89 | 1819.90 | 19.08 | G |
| 69 | 1833.91 | 1833.92 | 19.04 | G |
| 70 | 1790.90 | 1790.91 | 19.10 | G |
| 71 | na | na | na | na |
| 72 | 1820.85 | 1820.86 | 7.29 | B |
| 73 | 1862.90 | 1862.90 | 8.21 | B |
| 74 | 1890.90 | 1890.91 | 14.74 | G |
| 101 | 2116.14 | 2116.15 | 22.77 | G |
| 102 | 1907.85 | 1907.68 | 6.84 | B |
| 103 | 1907.89 | 1907.88 | 7.89 | B |
| 104 | 1820.85 | 1820.86 | 7.36 | B |
| 105 | 1862.86 | 1862.87 | 7.22 | B |
| 106 | 1862.90 | 1862.91 | 8.28 | B |
| 115 | 1844.91 | 1845 | 8.21 | D |
| 116 | 1802.81 | 1803 | 8.33 | D |
| 122 | 1844.91 | 1845 | 8.23 | D |
| 126 | 1862.86 | 1862.87 | 7.36 | B |
| 127 | 1862.94 | 1863.6 | 3.07 | na |
| 149 | 1905.94 | 1905.95 | 16.67 | E |
| 150 | 1822.87 | 1822 | 9.20 | D |
| 151 | 1950.93 | 1950.93 | 16.26 | E |
| 152 | 1908.88 | 1908.89 | 15.36 | E |
| 153 | 1908.88 | na | na | na |
| 154 | 1893.91 | 1893.91 | 7.57 | B |
| 155 | 1850.90 | 1850.91 | 7.85 | B |
| 156 | 1964.94 | 1964.95 | 16.34 | E |
| 157 | 1865.84 | 1865.85 | 6.99 | B |
| 158 | 1805.88 | 1805.89 | 11.51 | B |

na: not available

### Solubility Testing of Peptides at Different PH

Prior to the solubility measurement of a synthesized peptide batch, its purity was determined through UPLC/MS (methods B-G).

For solubility testing the target concentration was 1 mg pure compound/ml. Therefore, solutions from solid samples were prepared in a buffer system at a concentration of 1 mg/mL compound. The solid samples were obtained by drying down aliquots of a dimethyl sulfoxide stock solution using a Thermo Speedvac vacuum centrifuge. The dry down time was 3 hours.
Solubility buffer system A) 50 mM HCl pH 1.2
Solubility buffer system B) 50 mM phosphate buffer pH 6.5
Solubility buffer system C) 50 mM phosphate buffer pH 7.4
Solubility buffer system D) 50 mM phosphate buffer pH 9.0

UPLC-UV (Method A) was performed after 2 hours of gentle agitation from the filtered (0.45 μm filter plates) solutions, which was obtained by 10 mins of centrifugation forced filtration at 1500 RCF (relative centrifugal acceleration).

The solubility was determined by the comparison of the UV peak area of 2 μL-injections of a buffered sample diluted 1:5 with a standard curve of a dimethyl sulfoxide reference sample of the diluted in dimethyl sulfoxide with known concentration. The different extinction coefficients were taken into account for the calculation.

Results are shown in Table 7.

TABLE 7
Solubility of peptides in buffer formulations

| | Buffer | Buffer | Buffer | Buffer |
|---|---|---|---|---|
| SEQ | pH 1.2 | pH 6.5 | pH 7.4 | pH 9.0 |
| ID NO: | [mg/ml] | [mg/ml] | [mg/ml] | [mg/ml] |
| 1 | 0.10 | <0.05 | <0.05 | na |
| 2 | 0.10 | <0.05 | <0.05 | na |
| 4 | 0.03 | 0.02 | 0.02 | 0.01 |
| 5 | >=1.11 | >=1.23 | >=1.23 | na |
| 6 | 0.04 | 0.03 | 0.02 | na |
| 7 | 0.20 | 0.16 | 0.16 | 0.15 |
| 8 | >=0.99 | >=1.00 | >=1.02 | >=1.04 |
| 9 | >=1.04 | >=1.06 | >=0.96 | 0.75 |
| 10 | <0.01 | 0.07 | 0.01 | <0.01 |
| 11 | 0.12 | 0.10 | 0.09 | 0.08 |
| 12 | 0.31 | 0.29 | 0.19 | 0.11 |
| 13 | >=1.05 | >=1.07 | 0.77 | >=0.99 |
| 14 | 0.05 | 0.03 | 0.02 | <0.01 |
| 15 | >=0.97 | >=0.96 | >=0.91 | 0.67 |
| 16 | >=1.07 | >=0.95 | >=1.00 | 0.81 |
| 17 | >=1.03 | >=1.00 | >=0.97 | >=0.88 |
| 18 | 0.01 | <0.01 | <0.01 | <0.01 |
| 19 | 0.01 | 0.02 | <0.01 | <0.01 |
| 21 | >=1.08 | >=1.01 | >=0.97 | 0.23 |
| 23 | >=0.97 | =0.98 | 0.21 | 0.06 |
| 24 | >=1.02 | >=1.10 | >=1.00 | 0.30 |
| 27 | >=0.89 | >=0.87 | 0.81 | 0.27 |
| 32 | 0.74 | >=0.88 | >=0.87 | 0.17 |
| 33 | >=1.00 | >=0.89 | 0.64 | 0.13 |
| 34 | >=0.97 | >=0.91 | 0.27 | 0.14 |
| 35 | >=1.07 | >=1.03 | >=1.06 | >=1.14 |
| 36 | >=1.01 | >=1.10 | >=1.08 | >=1.15 |
| 37 | >=0.89 | >=0.94 | 0.59 | 0.09 |
| 40 | <0.01 | <0.01 | <0.01 | <0.01 |
| 44 | <0.01 | <0.01 | <0.01 | <0.01 |
| 45 | >=1.16 | >=1.06 | >=1.18 | 0.35 |
| 46 | >=1.08 | >=1.06 | >=1.06 | >=1.03 |
| 47 | >=1.00 | >=0.95 | 0.54 | 0.11 |
| 48 | >=1.06 | >=1.04 | >=0.99 | >=1.04 |
| 49 | >=1.11 | >=1.09 | >=1.05 | >=1.04 |
| 50 | >=1.00 | >=0.98 | 0.67 | 0.44 |
| 51 | >=1.02 | >=0.93 | 0.43 | 0.10 |
| 55 | >=1.11 | >=1.04 | >=0.96 | na |
| 56 | >=1.09 | >=0.92 | >=1.03 | na |
| 59 | >=1.09 | >=1.03 | >=1.09 | na |
| 60 | >=1.02 | >=1.00 | >=0.93 | na |
| 63 | >=0.91 | >=0.98 | >=0.91 | na |
| 65 | >=0.98 | >=0.91 | >=0.96 | na |
| 66 | >=1.630 | >=1.040 | >=1.290 | >=1.130 |
| 74 | >=1.390 | >=0.980 | 0.330 | 0.180 |
| 101 | >=1.430 | 0.750 | >=0.870 | 0.810 |
| 127 | >=1.520 | >=1.130 | >=1.120 | 0.620 |

na: not available

### Chemical Stability Assessment of Peptides

Purity of a peptide batch was determined through UPLC/MS (Methods B-G) prior to chemical stability measurement. The target concentration was 1 mg/ml pure compound. Solutions from solid samples were prepared in the following buffer systems with a concentration of 1 mg/ml compound based on the previously determined % purity:
Solubility buffer system A) 50 mM HCl pH 1.2+Acetonitrile (1:1)
Solubility buffer system B) 50 mM phosphate buffer pH 6.5+Acetonitrile (1:1)
Solubility buffer system C) 50 mM phosphate buffer pH 7.4+Acetonitrile (1:1)

Prepared solutions were stored for 24 hours at 2° C. and 37° C. After this time course, the samples were centrifuged for 15 min at 2500 RCF to create a 1:5 dilution of the supernatant. Then 2 μl of the dilution were analysed with UHPLC-UV.

The chemical stability was rated through the relative loss of purity calculated by the equation:

[(purity after 0 hours at 37° C.)−(purity after 24 hours at 37° C.)]/(purity after 0 hours at 37° C.)]*100%

The purity is calculated as [(peak area peptide)/(total peak area)]*100%.

Results are shown in Table 8.

TABLE 8
Chemical stability (relative purity loss)
of peptides in buffer formulations

| SEQ | Buffer pH 1.2 | Buffer pH 6.5 | Buffer pH 7.4 |
|---|---|---|---|
| ID NO: | [%] | [%] | [%] |
| 2 | 27.5 | 0.1 | 0.8 |
| 4 | 22.4 | 0.7 | 7.0 |
| 6 | 27.4 | na | 3.1 |
| 7 | 28.9 | 1.3 | 6.0 |
| 9 | 13.8 | 5.5 | 19.8 |
| 10 | 6.7 | 0.1 | 0.4 |
| 11 | 25.7 | 0.2 | 1.0 |
| 12 | 24.5 | 1.1 | 3.6 |
| 13 | 23.3 | 0.2 | 1.6 |
| 14 | 25.8 | 0.6 | 0.1 |
| 15 | 14.3 | 2.9 | 5.1 |
| 16 | 13.2 | 1.1 | 4.2 |
| 17 | 5.6 | 0.0 | 0.0 |
| 18 | 11.4 | 0.1 | 0.0 |
| 19 | 7.9 | 0.0 | 0.0 |
| 23 | 11.3 | 0.0 | 0.0 |
| 24 | 7.2 | 0.0 | 0.4 |
| 27 | 23.8 | 0.0 | 8.7 |
| 30 | 18.7 | 2.1 | 3.7 |
| 32 | 10.5 | 0.0 | 1.0 |
| 33 | 5.9 | 0.5 | 0.1 |
| 34 | 7.9 | 0.0 | 0.3 |
| 35 | 10.1 | 1.3 | 1.7 |
| 36 | 2.6 | 1.1 | 0.9 |
| 40 | 8.3 | 1.3 | 3.0 |
| 44 | 4.1 | 0.1 | 0.2 |
| 45 | 15.7 | 0.0 | 2.3 |
| 46 | 9.9 | 1.9 | 1.8 |
| 47 | 2.9 | 0.6 | 0.9 |
| 48 | 12.5 | 1.3 | 2.8 |
| 49 | 11.6 | 1.2 | 1.4 |
| 50 | 13.0 | 0.7 | 1.5 |
| 51 | 17.1 | 0.2 | 0.7 |
| 101 | 12.0 | 10.0 | 9.0 |
| 127 | 6.0 | 9.0 | 11.0 |

### Protease Stability Assessment of Peptides

Studies were carried out in simulated intestinal fluid (SIF) and simulated gastric fluid (SGF) to evaluate gastric stability of the peptide compounds disclosed herein.

For clarification the term “Pepsin-SGF” refers to a solution of simulated gastric fluid containing pepsin whereas “SGF media” refers to a solution of simulated gastric fluid without pepsin. Pepsin-SGF was prepared by dissolving 320 mg Pepsin (Merck Millipore) in 100 mL SGF media concentrate (ProSense).

Similarly, the term “Pancreatin-SIF” refers to a solution of simulated intestinal fluid containing pancreatin whereas “SIF-media” refers to a solution of simulated intestinal fluid without pancreatin.

Pancreatin-SIF was prepared by dissolving 1 mg Pancreatin (MP Biomedicals) in 10 mL SIF media concentrate (ProSense).

Peptides were first dissolved at 1 mM stock concentration in 0.01 HCl (+/−10% of DMSO) and were than diluted further with water to a stock concentration of 20 μM. Final peptide concentration in the incubation was 1 μM.

To 76 μL of the Pepsin-SGF buffer, 4 μL of the 20 μM peptide stock solution was added and incubated at 37° C. At each timepoint (0, 60, 120 and 240 minutes) the reaction was quenched with addition of 80 μL Ethanol (0.1% HCOOH). The samples were centrifuged for 20 minutes and an aliquot of the supernatant 100 μL were transferred to a LoBind-MTP plate and analyzed by LCMS/MS. The peak area response was calculated at each time point for each test compound. As internal reference, the peak area response of the same test compound at TO was calculated. The percent remaining at each timepoint was calculated based on the peak area response ratio of the test compound at TO. Time 0 was set to 100%, and all later timepoints were calculated relative to time 0.

To 76 μL of the Pepsin-SGF buffer, 4 μL of the 20 UM peptide stock solution was added and incubated at 37° C. At each timepoint (0, 60, 120 and 240 minutes) the reaction was quenched with addition of 80 μL Ethanol (0.1% HCOOH). The samples were centrifuged for 20 minutes and an aliquot of the supernatant 100 μL were transferred to a LoBind-MTP plate and analyzed by LCMS/MS. The peak area response was calculated at each time point for each test compound. As internal reference, the peak area response of the same test compound at TO was calculated. The percent remaining at each timepoint was calculated based on the peak area response ratio of the test compound at TO. Time 0 was set to 100%, and all later timepoints were calculated relative to time 0.

TABLE 9
Protease stability in simulated gastric fluids
(FaSSGF) and simulated intestinal fluids (FaSSIF)
expressed as remaining percent of peptide

| SEQ | FaSSIF | FaSSIF | FaSSIF | FaSSIF | FaSSGF |
|---|---|---|---|---|---|
| ID NO: | 0 (min) | 60 (min) | 120 (min) | 240 (min) | 60 (min) |
| 1 | 100 | 72 | 74 | 58 | 2 |
| 2 | 100 | 71 | 71 | 47 | 1 |
| 3 | 100 | 76 | 64 | 38 | 1 |
| 4 | 100 | 91 | 82 | 73 | 1 |
| 5 | 100 | 94 | 86 | 64 | 82 |
| 6 | 100 | 78 | 77 | 51 | 1 |
| 7 | 100 | 84 | 68 | 47 | 0 |
| 8 | 100 | 97 | 91 | 82 | 92 |
| 9 | 100 | 102 | 100 | 85 | 2 |
| 10 | 100 | 84 | 81 | 80 | 77 |
| 11 | 100 | 80 | 76 | 65 | 2 |
| 12 | 100 | 74 | 59 | 41 | 53 |
| 13 | 100 | 83 | 70 | 54 | 73 |
| 14 | 100 | 83 | 68 | 49 | 72 |
| 15 | 100 | 81 | 65 | 43 | 30 |
| 16 | 100 | 83 | 67 | 50 | 62 |
| 17 | 100 | 81 | 75 | 64 | 71 |
| 18 | 100 | 81 | 76 | 76 | 60 |
| 19 | 100 | 92 | 81 | 76 | 79 |
| 20 | 100 | 85 | 81 | 72 | 95 |
| 21 | 100 | 89 | 77 | 75 | 91 |
| 22 | 100 | 82 | 79 | 79 | 60 |
| 23 | 100 | 79 | 68 | 67 | 69 |
| 24 | 100 | 80 | 79 | 76 | 84 |
| 25 | 100 | 71 | 52 | 45 | 74 |
| 26 | 100 | 95 | 95 | 94 | 95 |
| 27 | 100 | 81 | 82 | 73 | 82 |
| 28 | 100 | 80 | 68 | 65 | 83 |
| 29 | 100 | 73 | 58 | 47 | 70 |
| 30 | 100 | 93 | 92 | 83 | 87 |
| 31 | 100 | 83 | 72 | 55 | 99 |
| 32 | 100 | 89 | 77 | 55 | 78 |
| 33 | 100 | 100 | 100 | 85 | 100 |
| 34 | 100 | 100 | 99 | 96 | 84 |
| 35 | 100 | 91 | 92 | 92 | 92 |
| 36 | 100 | 97 | 93 | 92 | 95 |
| 37 | 100 | 80 | 79 | 63 | 77 |
| 38 | 100 | 100 | 96 | 84 | 86 |
| 40 | 100 | 77 | 70 | 54 | 12 |
| 41 | 100 | 83 | 71 | 57 | 90 |
| 42 | 100 | 86 | 79 | 63 | 90 |
| 43 | 100 | 83 | 72 | 67 | 100 |
| 44 | 100 | 64 | 61 | 59 | 88 |
| 45 | 100 | 82 | 83 | 74 | 100 |
| 46 | 100 | 89 | 82 | 81 | 100 |
| 47 | 100 | 87 | 85 | 82 | 96 |
| 48 | 100 | 80 | 80 | 76 | 100 |
| 49 | 100 | 85 | 75 | 74 | 100 |
| 50 | 100 | 95 | 85 | 85 | 99 |
| 51 | 100 | 92 | 86 | 78 | 94 |
| 52 | 100 | 81 | 76 | 65 | 102 |
| 53 | 100 | 95 | 89 | 75 | 0 |
| 54 | 100 | 100 | 100 | 90 | 91 |
| 55 | 100 | 89 | 80 | 82 | 91 |
| 56 | 100 | 90 | 76 | 77 | 86 |
| 57 | 100 | 95 | 80 | 52 | 61 |
| 58 | 100 | 100 | 98 | 80 | 93 |
| 59 | 100 | 98 | 97 | 97 | 77 |
| 60 | 100 | 99 | 91 | 91 | 68 |
| 61 | 100 | 90 | 74 | 62 | 96 |
| 62 | 100 | 95 | 87 | 84 | 100 |
| 63 | 100 | 90 | 86 | 81 | 95 |
| 64 | 100 | 96 | 92 | 92 | 91 |
| 65 | 100 | 96 | 96 | 92 | 86 |
| 66 | 100 | 94 | 94 | 88 | 94 |
| 67 | 100 | 99 | 98 | 98 | 102 |
| 68 | 100 | 91 | 91 | 86 | 39 |
| 69 | 100 | 97 | 89 | 83 | 79 |
| 70 | 100 | 94 | 90 | 87 | 78 |
| 71 | 100 | n.a. | n.a. | n.a. | 76 |
| 72 | 100 | 91 | 90 | 83 | 92 |
| 73 | 100 | 82 | 81 | 77 | 87 |
| 74 | 100 | 87 | 84 | 78 | 92 |
| 102 | 100 | 82 | 77 | 74 | 97 |
| 103 | 100 | 75 | 73 | 67 | 89 |
| 104 | 100 | 80 | 80 | 76 | 100 |
| 105 | 100 | 78 | 80 | 77 | 93 |
| 106 | 100 | 76 | 76 | 74 | 90 |
| 126 | 100 | 90 | 87 | 83 | 80 |
| 127 | 100 | 99 | 101 | 97 | 100 |
| 149 | 100 | 95 | 99 | 97 | 101 |
| 151 | 100 | 97 | 97 | 96 | 101 |
| 152 | 100 | 100 | 100 | 98 | 99 |
| 154 | 100 | 99 | 97 | 96 | 96 |
| 155 | 100 | 98 | 98 | 98 | 98 |
| 156 | 100 | 99 | 99 | 97 | 100 |
| 157 | 100 | 80 | 78 | 74 | 100 |
| 158 | 100 | 80 | 73 | 74 | 37 |

na: not available

### Assay for Specific Human IL23R Antagonists

Antagonism of compounds for human interleukin-23 receptor antagonists, was determined by an ELISA assay.

A 384-well plate was coated with 50 μl/well of human IL23 at a final concentration of 1 g/ml and incubated overnight at 4° C. The wells were washed 3 times with 80 μl wash buffer and blocked with 80 μl blocking buffer for 60 minutes at room temperature and washed again. 20 μl of serially diluted test peptides was added to each well and subsequently 20 μl of recombinant human IL-23R-Fc chimera at final concentration of 0.1 μg/ml. The plate was incubated for 60 minutes at room temperature. After the wells were washed, bound IL23R-Fc was detected with goat anti-hu-IgG1-HRP antibody. Signals were visualized with QuantaBlu Fluorogenic Peroxidase Substrate.

### Assays for Selectivity of Specific IL23R Antagonists

For selectivity of the IL23R antagonistic peptides binding to human IL12Rß1, recombinant human IL-12Rß1-Fc was used for the ELISA assay.

For selectivity of the IL23R antagonistic peptides binding to mouse IL23R, recombinant mouse IL-23R-Fc chimera was used, and bound mouse IL23R-FC was detected with goat anti-mouse-IgG1-HRP antibody.

### IL-23 Induced Stat3 Signaling in Hekblue™ IL23 Cells

HEK-Blue™ IL-23 cells (Invivogen) are designed for the detection of bioactive human (hIL-23) and murine IL-23 (mIL-23) by monitoring the activation of the STAT3 pathway. Binding of IL-23 to its receptor on the surface of HEK-Blue™ IL-23 cells triggers a signalling cascade leading to the activation of STAT3 and the subsequent production of secreted embryonic alkaline phosphatase (SEAP). This can be readily assessed using QUANTI-Blue™ Solution, a SEAP detection reagent.

HEK-Blue™ IL-23 cells were cultured as described by Invivogen.

On the first day of assay 10 μL of the HEK-Blue IL23 cell suspension (final concentration: 10,000 cells/well) and 10 μl of serially diluted peptides solutions were dispensed into a 384-well assay plate. After pre-incubation of the reaction mixtures at 37° C./5% CO2/95% humidity for 60 minutes, 10 μl of the human IL23 cytokine (final concentration: 0.25 ng/ml) was added to each well for the STAT3 signalling activation. The plate was incubated overnight at 37° C./5% CO2/95% humidity.

On the second day 20 μl per well of Quanti-Blue detection solution (prepared according to the manufacture instruction) was dispensed into a new 384-well plate and followed by the transfer of 5 μl supernatant of the overnight incubated cell/sample mixtures. After incubation of 45 min at room temperature signals were measured with PHERAstar (BMG) at OD 620-655 nm. Dose response data were fitted with XLfit.

TABLE 10
ELISA IC50 and pSTA3 values of compounds of the disclosure

| | Human ELISA | |
|---|---|---|
| SEQ | IL23/IL23R IC₅₀ | pSTAT3 |
| ID NO: | [nM] | (nM) |
| 1 | 0.72 | 18.52 |
| 2 | 0.6 | 54 |
| 3 | 0.7 | 17.2 |
| 4 | 0.95 | 34.8 |
| 5 | 0.61 | 9.66 |
| 6 | 0.68 | 72.9 |
| 7 | 0.6 | 25 |
| 8 | 0.24 | 5.54 |
| 9 | 0.44 | 64.99 |
| 10 | 0.58 | 25.84 |
| 11 | 0.7 | 53.49 |
| 12 | 0.59 | 27.93 |
| 13 | 0.61 | 31.98 |
| 14 | 0.36 | 18.66 |
| 15 | 0.2 | 29.25 |
| 16 | 0.33 | 32.14 |
| 17 | 0.28 | 61.95 |
| 18 | 0.71 | 69.49 |
| 19 | 0.74 | 88.34 |
| 20 | 0.5 | 3.12 |
| 21 | 0.31 | 2.67 |
| 22 | 0.49 | 1.37 |
| 23 | 0.26 | 3.32 |
| 24 | 0.4 | 0.59 |
| 25 | 0.59 | 39.23 |
| 26 | 0.29 | 3.37 |
| 27 | 0.4 | 1.71 |
| 28 | 0.39 | 4.11 |
| 29 | 0.45 | 8.16 |
| 30 | 0.54 | 5.41 |
| 31 | 0.36 | 5.23 |
| 32 | 0.48 | 20.5 |
| 33 | 0.56 | 53.79 |
| 34 | 0.57 | 13.3 |
| 35 | 0.34 | 0.34 |
| 36 | 0.5 | 13.9 |
| 37 | 0.38 | 2 |
| 38 | 0.36 | 11.9 |
| 39 | 1.1 | 26.4 |
| 40 | 1 | 50.8 |
| 41 | 0.35 | 1.6 |
| 42 | 0.32 | 1.42 |
| 43 | 0.33 | 1.31 |
| 44 | 1.16 | 42.5 |
| 45 | 0.32 | 0.67 |
| 46 | 0.36 | 0.84 |
| 47 | 0.31 | 0.57 |
| 48 | 0.28 | 0.6 |
| 49 | 0.33 | 1.79 |
| 50 | 0.29 | 0.98 |
| 51 | 0.19 | 0.05 |
| 52 | 0.5 | 1.25 |
| 53 | 0.7 | 60.8 |
| 54 | 0.51 | 2.59 |
| 55 | 0.4 | 0.95 |
| 56 | 0.57 | 0.16 |
| 57 | 0.23 | 5.5 |
| 58 | 0.16 | 0.1 |
| 59 | 0.24 | 0.07 |
| 60 | 0.21 | 0.16 |
| 61 | 0.36 | 4.4 |
| 62 | 0.35 | 0.2 |
| 63 | 0.41 | 0.2 |
| 64 | 0.27 | 0.22 |
| 65 | 0.37 | 0.23 |
| 66 | 0.52 | 0.15 |
| 67 | 2.27 | 22.62 |
| 68 | 1.48 | 2.69 |
| 69 | 2.17 | 3.07 |
| 70 | 2.07 | 3.94 |
| 71 | 0.31 | 2.35 |
| 72 | 0.59 | 0.12 |
| 73 | 0.58 | 0.1 |
| 74 | 0.54 | 0.12 |
| 101 | 3.47 | n.a. |
| 102 | 0.3 | 0.09 |
| 103 | 0.56 | 0.11 |
| 104 | 0.64 | 0.1 |
| 105 | 0.55 | 0.1 |
| 106 | 0.65 | 0.13 |
| 122 | 2.3 | 0.37 |
| 126 | 0.56 | 0.11 |
| 127 | 0.53 | n.a. |
| 149 | 0.64 | 0.04 |
| 150 | 0.91 | 0.13 |
| 151 | 0.61 | 0.06 |
| 152 | 0.38 | 0.04 |
| 153 | 0.45 | 0.06 |
| 154 | 0.52 | 0.08 |
| 155 | 0.7 | 0.1 |
| 156 | 0.61 | 0.08 |
| 157 | 0.42 | 0.06 |
| 158 | 1.93 | 0.39 |

na: not available

TABLE 11
Comparison of ELISA IC50 between compounds
of the disclosure and Pen peptide compounds

| Pen peptide | Elisa | Compounds of | Elisa |
|---|---|---|---|
| compounds | IC50 [nM] | the disclosure | IC50 [nM] |
| SEQ ID NO: | Pen | SEQ ID NO: | Cys |
| 159 | 280.45 | 1 | 0.72 |
| 160 | 72.25 | 2 | 0.6 |
| 161 | 76.64 | 4 | 0.95 |
| 162 | 33.47 | 27 | 0.4 |
| 163 | 163.1 | 26 | 0.29 |
| 164 | 23.15 | 3 | 0.7 |

As shown in Table 11, the peptide compounds of the disclosure have a lower ELISA IC₅₀ for inhibiting the binding of IL23 to its receptor, compared to the Pen peptide compounds, demonstrating a higher potency.

### Items of the Disclosure

The disclosure is further characterized by the following items:

Item 1: A compound of the formula (I):

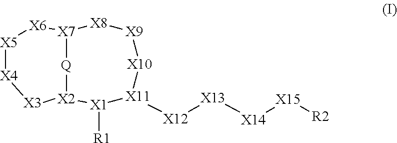

wherein
   X1 is a residue selected from a residue of Ahx, Aoa, Apt, Nmha, Nmapt, Lys, Pipa, Pipb, Pipp, Dab, Dad and Dap, and is linked via an amide bond to X11,
or is a residue selected from a residue of Buty and Butyl, and is linked via an alkyl bond to X11 and when X1 is a residue of Butyl, the residue of Butyl is linked to X2 via an alkyl bond,
   R1 is absent or, if X1 is selected from a residue of Ahx, Aoa, Apt, Lys, Dab and Dap, then R1 is H, C₁-C₄-alkyl or acetyl, and is linked to an amino function of X1,
   X2 is an amino acid residue selected from a residue of Cys, Glu, Asp, Nmc, Nmd, Nme, Nmt, Thr, and Abu, and their corresponding d-forms. In some embodiments, X2 is an amino acid residue selected from a residue of Cys, Glu, Asp, Nmc, Nmd, Nme, Nmt, and Thr, and their corresponding d-forms,
   X7 is an amino acid residue selected from a residue of Cys, Glu, Asp, Thr, and Abu, and their corresponding d-forms,
wherein X2 and X7 are linked to each other via Q, wherein Q is a disulfide bond, a thioether bond or an ester bond,
   X3 is an amino acid residue selected from a residue of Sar, His, Lys, Ala, Ahx, Apt, Arg, Asn, Gab, Gin, Trp, Thr, Ndmk, Nmn, Ntmk, and Lys(αAc),
   X4 is absent, or is an amino acid residue selected from a residue of His, Lys, Ahx, Apt, Arg, Asn, Gab, Gln, Trp, and Thr,
   X5 is an amino acid residue selected from a residue of Trp, Wme, Nak, Wcl, Wim, Wdm, and Wfl,
   X6 is an amino acid residue selected from a residue of Gin, Aib, Glu, Phe, Ile, Iva, Lys, Lys(Ac), Leu, Mle, Mly, Mva, Thr, Val, Trp, and Phe,
   X8 is an amino acid residue selected from a residue of Yme, Tyr, Trp, Yde, and Yae. In some embodiments, X8 is an amino acid residue selected from a residue of Yme, Tyr, Trp, and Yde,
   X9 is an amino acid residue selected from a residue of Nal, Nak, and Trp,
   X10 is an amino acid residue selected from a residue of Aib, Cba, Cit, d-Trp, d-Tza, Lys, Lys(Ac), Leu, Mle, Mly, Gln, Mkdm, and Thp,
   X11 is an amino acid residue selected from a residue of Asp, Bal, Gab, Glu, Kme, Lys, and Orn, and is linked via an amide bond to X1,
   X12 is absent or is an amino acid residue selected from a residue of Asn, Gly, Ser and Ala,
   X13 is absent or is an amino acid residue selected from a residue of Asn, Ala, Bal, Gly, Leu, Ile, Iva, Hol, Hph, Pal, Pyal, PyEA, His, Hme, Val, and their corresponding d-forms,
   X14 is absent or is an amino acid residue selected from a residue of Mep, Sar, Bal, Ile, Dnmy, and Hme,
   X15 is absent or is an amino acid residue of Lys,
   R2 is absent or is —NH₂, —OH or —N(C₂H₅)₂,
and
wherein amide bonds between X1 and X2, X2 and X3, X3 and X4, X4 and X5, X11 and X1 are optionally methylated wherein X2,
or a salt or solvate thereof,
and
wherein the compound of formula (I) inhibits the binding of interleukin-23 to an interleukin-23-receptor.

Item 2: A compound of item 1, wherein X2 is:
either an amino acid residue selected from an amino acid residue of Cys, Glu, Asp, Nmc, Nmd, Nme, Nmt, Thr and Abu, and their corresponding d-forms, and X8 is an amino acid residue selected from an amino acid residue of Yme, Tyr, Trp, and Yde,
or an amino acid residue selected from an amino acid residue of Cys, Glu, Asp, Nmc, Nmd, Nme, Nmt, and Thr, and their corresponding d-forms, and X8 is an amino acid residue selected from an amino acid residue of Yme, Tyr, Trp, Yde and Yae.

Item 3: A compound of item 1, wherein when X2 is Abu, or its corresponding d-forms, then X8 is not an amino acid residue of Yae.

Item 4: A compound of item 1, wherein when X8 is Yae, then X2 is not an amino acid residue of Abu, or its corresponding d-forms.

Item 5: A compound of the formula (I), wherein:
X1 is a residue of Ahx, Aoa, Apt, Nmha, Nmapt, Lys, Pipa, Pipb, Pipp, Dab, Dad or Dap, and is linked via an amide bond to X11,
or is a residue of Buty or Butyl, and is linked via an alkyl bond to X11 and when X1 is a residue of Butyl, the residue of Butyl is linked to X2 via an alkyl bond,
R1 is absent, or
if X1 is a residue of Ahx, Aoa, Apt, Lys, Dab or Dap, then R1 is H, C₁-C₄-alkyl or acetyl, and is linked to an amino function of X1,
X2 is an amino acid residue of Cys, Glu, Asp, Nmc, Nmd, Nme, Nmt, Thr or Abu, and their corresponding d-forms,
X7 is an amino acid residue of Cys, Glu, Asp, Thr or Abu, and their corresponding d-forms,
wherein X2 and X7 are linked to each other via Q, wherein Q is a disulfide bond, a thioether bond or an ester bond,
X3 is an amino acid residue of Sar, His, Lys, Ala, Ahx, Apt, Arg, Asn, Gab, Gin, Trp, Thr, Ndmk, Nmn, Ntmk or Lys(αAc),
X4 is absent, or is an amino acid residue of His, Lys, Ahx, Apt, Arg, Asn, Gab, Gln, Trp or Thr,
X5 is an amino acid residue of Trp, Wme, Nak, Wel, Wim, Wdm or Wfl,
X6 is an amino acid residue of Gln, Aib, Glu, Phe, Ile, Iva, Lys, Lys(Ac),
Leu, Mle, Mly, Mva, Thr, Val, Trp or Phe,
X8 is an amino acid residue of Yme, Tyr, Trp, or Yde,
X9 is an amino acid residue of Nal, Nak or Trp,
X10 is an amino acid residue of Aib, Cba, Cit, d-Trp, d-Tza, Lys, Lys(Ac), Leu, Mle, Mly, Gln, Mkdm or Thp,
X11 is an amino acid residue of Asp, Bal, Gab, Glu, Kme, Lys or Orn, and is linked via an amide bond to X1,
X12 is absent or is an amino acid residue of Asn, Gly, Ser or Ala,
X13 is absent or is an amino acid residue of Asn, Ala, Bal, Gly, Leu, Ile, Iva, Hol, Hph, Pal, Pyal, PyEA, His, Hme, or Val, and their corresponding d-forms,
X14 is absent or is an amino acid residue of Mep, Sar, Bal, Ile, Dnmy or Hme,
X15 is absent or is an amino acid residue of Lys,
R2 is absent or is —NH₂, —OH or —N(C₂H₅)₂,
and
wherein amide bonds between X1 and X2, X2 and X3, X3 and X4, X4 and X5, X11 and X1 are optionally methylated,
or a salt or solvate thereof, and
wherein the compound of formula (I) inhibits the binding of interleukin-23 to an interleukin-23-receptor.

Item 6: A compound of item 5, wherein:
X1 is a residue selected from a residue of Ahx, Apt, Nmha, Nmapt, Lys and Dad, and is linked via an amide bond to X11,
or is a residue selected from a residue of Buty and Butyl, and is linked via an alkyl bond to X11 and when X1 is a residue of Butyl, the residue of Butyl is linked to X2 via an alkyl bond,
R1 is absent, or
if X1 is a residue selected from a residue of Ahx, Apt and Lys, then R1 is H, C₁-C₄-alkyl or acetyl, and is linked to an amino function of X1,
X2 is an amino acid residue selected from a residue of Cys, Glu, Asp, Nmc,
Nmd, Nme, Nmt, Thr and Abu,
X7 is an amino acid residue selected from a residue of Cys, Glu, Asp, Thr and Abu,
wherein X2 and X7 are linked to each other via Q, wherein Q is a disulfide bond, a thioether bond or an ester bond,
X3 is an amino acid residue selected from a residue of Apt, Asn, Ndmk, Nmn and Lys(αAc),
X4 is absent or is an amino acid residue of Thr,
X5 is an amino acid residue selected from a residue of Trp, Wme and Wim,
X6 is an amino acid residue selected from a residue of Gin, Lys(Ac) and Leu,
X8 is an amino acid residue selected from a residue of Yme, Trp, and Yde,
X9 is an amino acid residue of Nal,
X10 is an amino acid residue selected from a residue of Aib, Lys, Lys(Ac), Mle, Mly, Mkdm and Thp,
X11 is an amino acid residue selected from a residue of Asp, Glu, Kme, Lys and Orn, and is linked via an amide bond to X1,
X12 is absent or is an amino acid residue selected from a residue of Asn, Gly, Ser and Ala,
X13 is absent or is an amino acid residue selected from a residue of Asn, Ala, Bal, Gly, d-Leu, Ile, Pal, Pyal, PyEA, His and Hme,
X14 is absent or is an amino acid residue selected from a residue of Mep, Sar, Bal, Ile and Hme,
X15 is absent or is an amino acid residue of Lys,
R2 is absent or is —NH₂, —OH or —N(C₂H₅)₂,
or a salt or solvate thereof.

Item 7: A compound of the formula (I), wherein:
X1 is a residue selected from a residue of Ahx, Aoa, Apt, Nmha, Nmapt, Lys, Pipa, Pipb, Pipp, Dab, Dad and Dap, and is linked via an amide bond to X11,

or is a residue selected from a residue of Buty and Butyl, and is linked via an alkyl bond to X11 and when X1 is a residue of Butyl, the residue of Butyl is linked to X2 via an alkyl bond,
R1 is absent, or
if X1 is a residue selected from a residue of Ahx, Aoa, Apt, Lys, Dab and Dap, then R1 is H, C₁-C₄-alkyl or acetyl, and is linked to an amino function of X1,
X2 is an amino acid residue selected from a residue of Cys, Glu, Asp, Nmc, Nmd, Nme, Nmt, and Thr, and their corresponding d-forms,
X7 is an amino acid residue selected from a residue of Cys, Glu, Asp, Thr and Abu, and their corresponding d-forms,
wherein X2 and X7 are linked to each other via Q, wherein Q is a disulfide bond, a thioether bond or an ester bond,
X3 is an amino acid residue selected from a residue of Sar, His, Lys, Ala, Ahx, Apt, Arg, Asn, Gab, Gin, Trp, Thr, Ndmk, Nmn, Ntmk and Lys(αAc),
X4 is absent, or is an amino acid residue selected from a residue of His, Lys, Ahx, Apt, Arg, Asn, Gab, Gin, Trp and Thr,
X5 is an amino acid residue selected from a residue of Trp, Wme, Nak, Wcl, Wim, Wdm and Wfl,
X6 is an amino acid residue selected from a residue of Gin, Aib, Glu, Phe, Ile, Iva, Lys, Lys(Ac), Leu, Mle, Mly, Mva, Thr, Val, Trp and Phe,
X8 is an amino acid residue selected from a residue of Yme, Tyr, Trp, Yde and Yae,
X9 is an amino acid residue selected from a residue of Nal, Nak and Trp,
X10 is an amino acid residue selected from a residue of Aib, Cba, Cit, d-Trp, d-Tza, Lys, Lys(Ac), Leu, Mle, Mly, Gin, Mkdm and Thp,
X11 is an amino acid residue selected from a residue of Asp, Bal, Gab, Glu, Kme, Lys and Orn, and is linked via an amide bond to X1,
X12 is absent or is an amino acid residue selected from a residue of Asn, Gly, Ser and Ala,
X13 is absent or is an amino acid residue selected from a residue of Asn, Ala, Bal, Gly, Leu, Ile, Iva, Hol, Hph, Pal, Pyal, PyEA, His, Hme, Val, and their corresponding d-forms,
X14 is absent or is an amino acid residue selected from a residue of Mep, Sar, Bal, Ile, Dnmy and Hme,
X15 is absent or is an amino acid residue of Lys,
R2 is absent or is —NH₂, —OH or —N(C₂H₅)₂, and
wherein amide bonds between X1 and X2, X2 and X3, X3 and X4, X4 and X5, X11 and X1 are optionally methylated, or a salt or solvate thereof, and
wherein the compound of formula (I) inhibit the binding of interleukin-23 to an interleukin-23-receptor.

Item 8: A compound of item 7, wherein;
X1 is a residue selected from a residue of Ahx, Apt, Nmha, Nmapt, Lys and Dad, and is linked via an amide bond to X11,
or is a residue selected from a residue of Buty and Butyl, and is linked via an alkyl bond to X11 and when X1 is a residue of Butyl, the residue of Butyl is linked to X2 via an alkyl bond,
R1 is absent, or

if X1 is a residue selected from a residue of Ahx, Apt and Lys, then R1 is H, C₁-C₄-alkyl or acetyl, and is linked to an amino function of X1,
X2 is an amino acid residue selected from a residue of Cys, Glu, Asp, Nmc, Nmd, Nme, Nmt, and Thr,
X7 is an amino acid residue selected from a residue of Cys, Glu, Asp, Thr and Abu,
wherein X2 and X7 are linked to each other via Q, wherein Q is a disulfide bond, a thioether bond or an ester bond,
X3 is an amino acid residue selected from a residue of Apt, Asn, Ndmk, Nmn and Lys(αAc),
X4 is absent or is an amino acid residue of Thr,
X5 is an amino acid residue selected from a residue of Trp, Wme and Wim,
X6 is an amino acid residue selected from a residue of Gin, Lys(Ac) and Leu,
X8 is an amino acid residue selected from a residue of Yme, Trp, Yde and Yae,
X9 is an amino acid residue of Nal,
X10 is an amino acid residue selected from a residue of Aib, Lys, Lys(Ac), Mle, Mly, Mkdm and Thp,
X11 is an amino acid residue selected from a residue of Asp, Glu, Kme, Lys and Orn, and is linked via an amide bond to X1,
X12 is absent or is an amino acid residue selected from a residue of Asn, Gly, Ser and Ala,
X13 is absent or is an amino acid residue selected from a residue of Asn, Ala, Bal, Gly, d-Leu, Ile, Pal, Pyal, PyEA, His and Hme,
X14 is absent or is an amino acid residue selected from a residue of Mep, Sar, Bal, Ile and Hme,
X15 is absent or is an amino acid residue Lys,
R2 is absent or is —NH₂, —OH or —N(C₂H₅)₂, or a salt or solvate thereof.

Item 9: A compound of item 1 to 8, wherein R1 is absent.

Item 10: A compound of item 1 to 8, wherein R1 is H.

Item 11: A compound of item 1 to 8, wherein R1 is C₁-C₄-alkyl.

Item 12: A compound of item 1 to 8, wherein R1 is acetyl.

Item 13: A compound of item 1 to 12, wherein R2 is absent.

Item 14: A compound of item 1 to 12, wherein R2 is —NH₂.

Item 15: A compound of item 1 to 12, wherein R2 is-OH.

Item 16: A compound of item 1 to 12, wherein R2 is-N(C₂H₅)₂.

Item 17: A compound of item 1 to 16, wherein
X1 is a residue selected from a residue of Ahx, Aoa, Apt, Nmha, Nmapt, Lys, Pipa, Pipb, Pipp, Dab and Dap, and X11 is a amino acid residue selected from a residue of Asp and Glu,
or
X1 is a residue Dad and X11 is an amino acid residue selected from a residue of Lys and Kme,
or
X1 is a residue selected from a residue of Buty or Butyl and X11 is an amino acid residue of Kme.

Item 18: A compound of item 1 to 16, wherein
X1 is a residue of Ahx, and X11 is an amino acid residue selected from a residue of Glu or Asp.

Item 19: A compound of item 1 to 16, wherein
X1 is a residue of Apt, and X11 is an amino acid residue selected from a residue of Glu or Asp.

Item 20: A compound of item 1 to 16, wherein
X1 is a residue of Apt, and X11 is an amino acid residue Glu.

Item 21: A compound of item 1 to 16, wherein
X1 is a residue of Nmapt and X11 is an amino acid residue selected from a residue of Glu or Asp.

Item 22: A compound of item 1 to 16, wherein
X1 is a residue of Nmapt and X11 is an amino acid residue Glu.

Item 23: A compound of item 1 to 16, wherein
X1 is a residue of Nmapt and X11 is an amino acid residue of Asp.

Item 24: A compound of item 1 to 16, wherein
X1 is a residue of Nmha and X11 is an amino acid residue selected from a residue of Glu or Asp.

Item 25: A compound of item 1 to 16, wherein
X1 is a residue of Nmha and X11 is an amino acid residue of Glu.

Item 26: A compound of item 1 to 16, wherein
X1 is a residue of Nmha and X11 is an amino acid residue of Asp.

Item 27: A compound of item 1 to 16, wherein
X1 is a residue of Lys and X11 is an amino acid residue selected from a residue of Glu or Asp.

Item 28: A compound of item 1 to 16, wherein
X1 is a residue of Lys and X11 is an amino acid residue of Glu.

Item 29: A compound of item 1 to 16, wherein
X1 is a residue of Lys and X11 is an amino acid residue of Asp.

Item 30: A compound of item 1 to 16, wherein
X1 is a residue of Dad and X11 is an amino acid residue selected from a residue of Lys or Kme.

Item 31: A compound of item 1 to 16, wherein
X1 is a residue of Dada and X11 is an amino acid residue of Lys.

Item 32: A compound of item 1 to 16, wherein
X1 is a residue of Dad and X11 is an amino acid residue of Kme.

Item 33: A compound of item 1 to 16, wherein
X1 is a residue of Buty and X11 is an amino acid residue selected from a residue of Lys or Kme.

Item 34: A compound of item 1 to 16, wherein
X1 is a residue of Buty and X11 is an amino acid residue of Lys.

Item 35: A compound of item 1 to 16, wherein
X1 is a residue of Buty and X11 is an amino acid residue of Kme.

Item 36: A compound of item 1 to 16, wherein
X1 is a residue of Butyl and X11 is an amino acid residue selected from a residue of Lys or Kme.

Item 37: A compound of item 1 to 16, wherein
X1 is a residue of Butyl and X11 is an amino acid residue of Lys.

Item 38: A compound of item 1 to 16, wherein
X1 is a residue of Butyl and X11 is an amino acid residue of Kme.

Item 39: A compound of item 1 to 38, wherein X2 is an amino acid residue selected from a residue of Cys, Glu, Asp, Nmc, Nmd, Nme, Nmt, Thr and Abu, and their corresponding d-forms.

Item 40: A compound of item 1 to 38, wherein X2 is an amino acid residue selected from a residue of Cys, Glu, Asp, Nmc, Nmd, Nme, Nmt, and Thr, and their corresponding d-forms.

Item 41: A compound of item 1 to 38, wherein
X2 is an amino acid residue selected from a residue of Cys and Nmc, and their corresponding d-forms,
X7 is an amino acid residue selected from a residue of Cys and its corresponding d-form,
wherein X2 and X7 are linked to each other via Q, wherein Q is a disulfide bond,
or
X2 is an amino acid residue selected from a residue of Cys, Nmc, Thr, and Abu and their corresponding d-forms,
X7 is an amino acid residue selected from a residue of Cys, Thr, and Abu and their corresponding d-forms,
wherein X2 and X7 are linked to each other via Q, wherein Q is a thioether bond,
or
X2 is an amino acid residue selected from a residue of Glu, Asp, Nmd, Nme, Nmt and Thr and their corresponding d-forms,
X7 is an amino acid residue selected from a residue of Glu, Asp, Thr and their corresponding d-forms,
wherein X2 and X7 are linked to each other via Q, wherein Q is an ester bond.

Item 42: A compound of item 1 to 38, wherein
X2 is an amino acid residue selected from a residue of Cys and its corresponding d-form,
X7 is an amino acid residue selected from a residue of Cys and its corresponding d-form,
wherein X2 and X7 are linked to each other via Q, wherein Q is a disulfide bond, or
X2 is an amino acid residue selected from a residue of Cys, Glu, Thr, and Abu, and their corresponding d-forms,
X7 is an amino acid residue selected from a residue of Cys, Thr, and Abu, and their corresponding d-form,
wherein X2 and X7 are linked to each other via Q, wherein Q is a thioether bond,
or
X2 is an amino acid residue selected from a residue of Glu and Thr and their corresponding d-forms,
X7 is an amino acid residue selected from a residue of Glu, Asp and Thr and their corresponding d-forms,
wherein X2 and X7 are linked to each other via Q, wherein Q is an ester bond.

Item 43: A compound of item 1 to 38, wherein
X2-Q-X7 is selected from a group of
Cys-Q-Cys, Nmc-Q-Cys and Cys-Q-Nmc, wherein Q is a disulfide bond, or
Abu-Q-Cys, Cys-Q-Abu, wherein Q is a thioether bond, or
Thr-Q-Asp, Thr-Q-Glu, Glu-Q-Thr, wherein Q is an ester bond.

Item 44: A compound of item 1 to 38, wherein X2-Q-X7 is selected from a group of:
Cys-Q-Cys, Nmc-Q-Cys and Cys-Q-Nmc, wherein Q is a disulfide bond, or
Abu-Q-Cys, Cys-Q-Abu, wherein Q is a thioether bond, or
Thr-Q-Asp, Thr-Q-Glu, Glu-Q-Thr, wherein Q is an ester bond.

Item 45: A compound of item 1 to 38, wherein X2-Q-X7 is selected from a group of:
Cys-Q-Cys, Nmc-Q-Cys and Cys-Q-Nmc, wherein Q is a disulfide bond, or
Cys-Q-Abu, wherein Q is a thioether bond, or
Thr-Q-Asp, Thr-Q-Glu, Glu-Q-Thr, wherein Q is an ester bond.

Item 46: A compound of item 1 to 38, wherein
X2-Q-X7 is selected from a group of Cys-Q-Cys and Nmc-Q-Cys, wherein Q is a disulfide bond, or Abu-Q-Cys wherein Q is a thioether bond, or
Glu-Q-Thr, wherein Q is an ester bond.

Item 47: A compound of item 1 to 38, wherein
X2-Q-X7 is selected from a group of Cys-Q-Cys and Nmc-Q-Cys, wherein Q is a disulfide bond.

Item 48: A compound of item 1 to 38, wherein
X2-Q-X7 is Abu-Q-Cys wherein Q is a thioether bond.

Item 49: A compound of item 1 to 38, wherein
X2-Q-X7 is Glu-Q-Thr, wherein Q is an ester bond.

Item 50: A compound of item 1 to 49, wherein X3 is an amino acid residue selected from a residue of Apt, Ndmk, Nmn and Lys(αAc).

Item 51: A compound of item 1 to 49, wherein X3 is an amino acid residue of Sar.

Item 52: A compound of item 1 to 49, wherein X3 is an amino acid residue of His.

Item 53: A compound of item 1 to 49, wherein X3 is an amino acid residue of Lys.

Item 54: A compound of item 1 to 49, wherein X3 is an amino acid residue of Ala.

Item 55: A compound of item 1 to 49, wherein X3 is an amino acid residue of Ahx.

Item 56: A compound of item 1 to 49, wherein X3 is an amino acid residue of Apt.

Item 57: A compound of item 1 to 49, wherein X3 is an amino acid residue of Arg.

Item 58: A compound of item 1 to 49, wherein X3 is an amino acid residue of Asn.

Item 59: A compound of item 1 to 49, wherein X3 is an amino acid residue of Gab.

Item 60: A compound of item 1 to 49, wherein X3 is an amino acid residue of Gln.

Item 61: A compound of item 1 to 49, wherein X3 is an amino acid residue of Trp.

Item 62: A compound of item 1 to 49, wherein X3 is an amino acid residue of Thr.

Item 63: A compound of item 1 to 49, wherein X3 is an amino acid residue of Ndmk,

Item 64: A compound of item 1 to 49, wherein X3 is an amino acid residue of Nmn.

Item 65: A compound of item 1 to 49, wherein X3 is an amino acid residue of Ntmk.

Item 66: A compound of item 1 to 49, wherein X3 is an amino acid residue of Lys(αAc).

Item 67: A compound of item 1 to 66, wherein X4 is absent.

Item 68: A compound of item 1 to 49, wherein X3 is an amino acid residue selected from a residue of Lys, Ahx, Apt, Thr, Ndmk, Nmn, Ntmk and Lys(αAc), and X4 is absent.

Item 69: A compound of item 1 to 49 wherein X3 is an amino acid residue selected from a residue of Apt, Ndmk, Nmn and Lys(αAc), and X4 is absent.

Item 70: A compound of item 1 to 49, wherein X3 is an amino acid residue selected from a residue of Apt, Ndmk and Lys(αAc), and X4 is absent.

Item 71: A compound of item 1 to 49, wherein X3 is an amino acid residue of Lys(αAc), and X4 is absent.

Item 72: A compound of item 1 to 66, wherein X4 is an amino acid residue selected from a residue of His, Lys, Arg, Asn, Gab, Gln, Trp and Thr.

Item 73: A compound of item 1 to 66, wherein X4 is an amino acid residue selected from a residue of His, Lys, Arg, Asn, Gin, Trp and Thr.

Item 74: A compound of item 1 to 66, wherein X4 is an amino acid residue of His.

Item 75: A compound of item 1 to 66, wherein X4 is an amino acid residue of Lys.

Item 76: A compound of item 1 to 66, wherein X4 is an amino acid residue of Ahx.

Item 77: A compound of item 1 to 66, wherein X4 is an amino acid residue of Apt.

Item 78: A compound of item 1 to 66, wherein X4 is an amino acid residue of Arg.

Item 79: A compound of item 1 to 66, wherein X4 is an amino acid residue of Asn.

Item 80: A compound of item 1 to 66, wherein X4 is an amino acid residue of Gab.

Item 81: A compound of item 1 to 66, wherein X4 is an amino acid residue of Gln.

Item 82: A compound of item 1 to 66, wherein X4 is an amino acid residue of Trp.

Item 83: A compound of item 1 to 66, wherein X4 is an amino acid residue of Thr.

Item 84: A compound of item 1 to 83, wherein X5 is an amino acid residue selected from a residue of Trp, Wme, Nak, Wcl, Wim, Wdm and Wfl.

Item 85: A compound of item 1 to 83, wherein X5 is an amino acid residue selected from a residue of Trp, Wme, Wcl, Wim, Wdm and Wfl.

Item 86: A compound of item 1 to 83, wherein X5 is an amino acid residue selected from a residue of Trp, and Wim.

Item 87: A compound of item 1 to 83, wherein X5 is an amino acid residue of Trp.

Item 88: A compound of item 1 to 83, wherein X5 is an amino acid residue of Wme.

Item 89: A compound of item 1 to 83, wherein X5 is an amino acid residue of Nak.

Item 90: A compound of item 1 to 83, wherein X5 is an amino acid residue of Wcl.

Item 91: A compound of item 1 to 83, wherein X5 is an amino acid residue of Wim.

Item 92: A compound of item 1 to 83, wherein X5 is an amino acid residue of Wdm.

Item 93: A compound of item 1 to 83, wherein X5 is an amino acid residue of Wfl.

Item 94: A compound of item 1 to 93, wherein X6 is an amino acid residue selected from a residue of Gin, Aib, Glu, Phe, Ile, Iva, Lys, Lys(Ac), Leu, Mle, Mly, Mva, Thr, Val, Trp and Phe.

Item 94: A compound of item 1 to 93, wherein X6 is an amino acid residue selected from a residue of Gin, Aib, Glu, Ile, Iva, Lys, Lys(Ac), Leu, Mle, Mly, Mva, and Val.

Item 95: A compound of item 1 to 93, wherein X6 is an amino acid residue selected from a residue of Gin, Lys(Ac) and Leu.

Item 96: A compound of item 1 to 93, wherein X6 is an amino acid residue of Gin.

Item 97: A compound of item 1 to 93, wherein X6 is an amino acid residue of Aib.

Item 98: A compound of item 1 to 93, wherein X6 is an amino acid residue of Glu.

Item 99: A compound of item 1 to 93, wherein X6 is an amino acid residue of Phe.

Item 100: A compound of item 1 to 93, wherein X6 is an amino acid residue of Ile.

Item 101: A compound of item 1 to 93, wherein X6 is an amino acid residue of Iva.

Item 91021: A compound of item 1 to 93, wherein X6 is an amino acid residue of Lys.

Item 103: A compound of item 1 to 93, wherein X6 is an amino acid residue of Lys(Ac).

Item 104: A compound of item 1 to 93, wherein X6 is an amino acid residue of Leu.

Item 105: A compound of item 1 to 93, wherein X6 is an amino acid residue of Mle.

Item 106: A compound of item 1 to 93, wherein X6 is an amino acid residue of Mly.

Item 107: A compound of item 1 to 93, wherein X6 is an amino acid residue of Mva.

Item 108: A compound of item 1 to 93, wherein X6 is an amino acid residue of Thr.

Item 109: A compound of item 1 to 93, wherein X6 is an amino acid residue of Val.

Item 110: A compound of item 1 to 93, wherein X6 is an amino acid residue of Trp.

Item 111: A compound of item 1 to 93, wherein X6 is an amino acid residue of Phe.

Item 112: A compound of item 1 to 111, wherein X8 is an amino acid residue selected from a residue of Yme, Tyr, Trp, Yde and Yae.

Item 113: A compound of item 1 to 111, wherein X8 is an amino acid residue selected from a residue of Yme, Tyr, Trp, and Yde

Item 114: A compound of item 1 to 111, wherein X8 is an amino acid residue selected from a residue of Yme, Tyr, Trp, Yde, and Yae.

Item 115: A compound of item 1 to 111, wherein X8 is an amino acid residue selected from a residue of Yme, Yde and Yae,

Item 116: A compound of item 1 to 111, wherein X8 is an amino acid residue of Yme.

Item 117: A compound of item 1 to 111, wherein X8 is an amino acid residue of Tyr.

Item 118: A compound of item 1 to 111, wherein X8 is an amino acid residue of Trp.

Item 119: A compound of item 1 to 111, wherein X8 is an amino acid residue of Yde.

Item 120: A compound of item 1 to 111, wherein X8 is an amino acid residue of Yae.

Item 121: A compound of item 1 to 111, wherein X9 is an amino acid residue of Nal.

Item 122: A compound of item 1 to 111, wherein X9 is an amino acid of Nak.

Item 123: A compound of item 1 to 111, wherein X9 is an amino acid residue of Trp.

Item 124: A compound of item 1 to 123, wherein X10 is an amino acid residue selected from a residue of Aib, Cba, Cit, d-Trp, d-Tza, Lys, Lys(Ac), Leu, Mie, Mly, Gin, Mkdm and Thp.

Item 125: A compound of item 1 to 123, wherein X10 is an amino acid residue selected from a residue of Aib, Lys, Lys(Ac), Mle, Mly, Mkdm and Thp.

Item 126: A compound of item 1 to 123, wherein X10 is an amino acid residue selected from a residue of Aib, Mle, Mly, Mkdm and Thp.

Item 127: A compound of item 1 to 123, wherein X10 is an amino acid residue of Aib.

Item 128: A compound of item 1 to 123, wherein X10 is an amino acid residue of Cba.

Item 129: A compound of item 1 to 123, wherein X10 is an amino acid residue of Cit.

Item 130: A compound of item 1 to 123, wherein X10 is an amino acid residue of d-Trp.

Item 131: A compound of item 1 to 123, wherein X10 is an amino acid residue of d-Tza.

Item 132: A compound of item 1 to 123, wherein X10 is an amino acid residue of Lys.

Item 133: A compound of item 1 to 123, wherein X10 is an amino acid residue of Lys(Ac).

Item 134: A compound of item 1 to 123, wherein X10 is an amino acid residue of Leu.

Item 135: A compound of item 1 to 123, wherein X10 is an amino acid residue of Mle.

Item 136: A compound of item 1 to 123, wherein X10 is an amino acid residue of Mly.

Item 137: A compound of item 1 to 123, wherein X10 is an amino acid residue of Gln.

Item 138: A compound of item 1 to 123, wherein X10 is an amino acid residue of Mkdm.

Item 139: A compound of item 1 to 123, wherein X10 is an amino acid residue of Thp.

Item 140: A compound of item 1 to 139, wherein X12 is absent or is an amino acid residue selected from a residue of Asn, Ser and Ala.

Item 141: A compound of item 1 to 139, wherein X12 is absent.

Item 142: A compound of item 1 to 139, wherein X12 is an amino acid residue of Asn.

Item 143: A compound of item 1 to 139, wherein X12 is an amino acid residue of Ser.

Item 144: A compound of item 1 to 139, wherein X12 is an amino acid residue of Ala.

Item 145: A compound of item 1 to 144, wherein X13 is absent or is an amino acid residue selected from a residue of Asn, Ala, Bal, Gly, Leu, d-Leu, Ile, Iva, Hol, Hph, Pal, Pyal, PyEA, His, Hme, Val, and their corresponding d-forms,

Item 146: A compound of item 1 to 144, wherein X13 is absent or is an amino acid residue selected from a residue of Asn, Ala, Bal, Gly, Leu, d-Leu, Ile, Iva, Hol, Hph, Pal, Pyal, PyEA, His, Hme and Val.

Item 147: A compound of item 1 to 144, wherein X13 is absent.

Item 148: A compound of item 1 to 144, wherein X13 is an amino acid residue of Asn.

Item 149: A compound of item 1 to 144, wherein X13 is an amino acid residue of Ala.

Item 150: A compound of item 1 to 144, wherein X13 is an amino acid residue of Bal.

Item 151: A compound of item 1 to 144, wherein X13 is an amino acid residue of Gly.

Item 152: A compound of item 1 to 144, wherein X13 is an amino acid residue of Leu.

Item 153: A compound of item 1 to 144, wherein X13 is an amino acid residue of d-Leu.

Item 154: A compound of item 1 to 144, wherein X13 is an amino acid residue of Ile.

Item 155: A compound of item 1 to 144, wherein X13 is an amino acid residue of Iva.

Item 156: A compound of item 1 to 144, wherein X13 is an amino acid residue of Hol.

Item 157: A compound of item 1 to 144, wherein X13 is an amino acid residue of Hph.

Item 158: A compound of item 1 to 144, wherein X13 is an amino acid residue of Pal.

Item 159: A compound of item 1 to 144, wherein X13 is an amino acid residue of Pyal.

Item 160: A compound of item 1 to 144, wherein X13 is an amino acid residue of PyEA.

Item 161: A compound of item 1 to 144, wherein X13 is an amino acid residue of His.

Item 162: A compound of item 1 to 144, wherein X13 is an amino acid residue of Hme.

Item 163: A compound of item 1 to 144, wherein X13 is an amino acid residue of Val.

Item 164: A compound of item 1 to 163, wherein X14 is absent or is an amino acid residue selected from a residue of Mep, Sar, Bal, Ile, Dnmy and Hme.

Item 165: A compound of item 1 to 163, wherein X14 is absent.

Item 166: A compound of item 1 to 163, wherein X14 is an amino acid residue of Mep,

Item 167: A compound of item 1 to 163, wherein X14 is an amino acid residue of Sar

Item 168: A compound of item 1 to 163, wherein X14 is an amino acid residue of Bal.

Item 169: A compound of item 1 to 163, wherein X14 is an amino acid residue of Ile.

Item 170: A compound of item 1 to 163, wherein X14 is an amino acid residue of Dnmy.

Item 171: A compound of item 1 to 163, wherein X14 is an amino acid residue of Hme.

Item 172: A compound of item 1 to 171, wherein X15 is absent.

Item 172: A compound of item 1 to 171, wherein X15 is Lys.

Item 173: A compound of item 1 to 172, wherein X2-X3-X4-X5-X6-X7 is Cys-Asn-Thr-Trp-Gln-Cys,

Item 174: A compound of item 1 to 172, wherein X2-X3-X4-X5-X6-X7 is Cys-Asn-Thr-Wim-Gln-Cys.

Item 175: A compound of item 1 to 172, wherein X2-X3-X4-X5-X6-X7 is Cys-Asn-Thr-Trp-Lys(Ac)-Cys.

Item 176: A compound of item 1 to 172, wherein X2-X3-X4-X5-X6-X7 is Cys-Asn-Thr-Wim-Lys(Ac)-Cys.

Item 177: A compound of item 1 to 172, wherein X2-X3-X4-X5-X6-X7 is Cys-Nmn-Thr-Trp-Gin-Cys.

Item 178: A compound of item 1 to 172, wherein X2-X3-X4-X5-X6-X7 is Abu-Asn-Thr-Trp-Lys(Ac)-Cys.

Item 179: A compound of item 1 to 172, wherein X2-X3-X4-X5-X6-X7 is Abu-Asn-Thr-Wim-Lys(Ac)-Cys.

Item 180: A compound of item 1 to 172, wherein X2-X3-X4-X5-X6-X7 is Abu-Asn-Thr-Wme-Leu-Cys.

Item 181: A compound of item 1 to 180, wherein X12-X13- is Gly-PyEA,
X14 is absent,
X15 is absent,
R2 is absent.

Item 182: A compound of item 1 to 181, wherein X8-X9-X10 is Yde-Nal-X10, wherein X10 is selected from a residue of Aib, Mle, Thp, Mkdm or Mly.

Item 183: A compound of the formula (Ia)

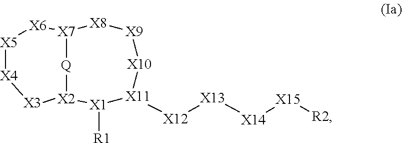

wherein
R1, R2, X1, X2, X3, X4, X5, X6, X7, X8, X9, X11, X12, X13, X14, are defined as in items 1 to 182 and
X10 is an amino acid residue selected from a residue of Aib, Cba, Cit, d-Trp, d-Tza, Lys, Lys(Ac), Leu, Mle, Mly, Gin, Mkdm and Thp,
X15 is absent or is an amino acid residue of Lys,
and wherein
amide bonds between X1 and X2, X2 and X3, X3 and X4, X4 and X5, X11 and X1 are optionally methylated,
or a salt or solvate thereof,
and
wherein at least one of X10 and X15
is an amino acid residue having a side chain with an —NH₂ group, wherein the —NH₂ side chain group is functionalized by —C(O)—R5, —C(O)O—R5, —C(O)NH—R5, —S(O)₂—R5 or R5, for example by —C(O)—R5, wherein R5 may be a moiety comprising up to 50 or up to 100 carbon atoms and optionally heteroatoms selected from halogen, N, O, S and P,
wherein the compound of formula (Ia) inhibits the binding of interleukin-23 to an interleukin-23-receptor.

Item 184: A compound of item 183, wherein compounds of formula (Ia) are such that X2 is:
either an amino acid residue selected from an amino acid residue of Cys, Glu, Asp, Nmc, Nmd, Nme, Nmt, Thr and Abu, and their corresponding d-forms, and X8 is an amino acid residue selected from an amino acid residue of Yme, Tyr, Trp, and Yde,
or an amino acid residue selected from an amino acid residue of Cys, Glu, Asp, Nmc, Nmd, Nme, Nmt, and Thr, and their corresponding d-forms, and X8 is an amino acid residue selected from an amino acid residue of Yme, Tyr, Trp, Yde and Yae.

Item 185: A compound of item 183, wherein compounds of formula (Ia) may be such that when X2 is Abu, or its corresponding d-forms, then X8 is not an amino acid residue of Yae.

Item 186: A compound of item 183, wherein compounds of formula (Ia) may be such that when X8 is Yae, then X2 is not an amino acid residue of Abu, or its corresponding d-forms.

Item 187: A compound of item 183, wherein compounds of formula (Ia) are such that:
X1 is a residue selected from a residue of Ahx, Aoa, Apt, Nmha, Nmapt, Lys, Pipa, Pipb, Pipp, Dab, Dad and Dap, and is linked via an amide bond to X11,
or is a residue selected from a residue of Buty and Butyl, and is linked via an alkyl bond to X11 and when X1 is a residue of Butyl, the residue of Butyl is linked to X2 via an alkyl bond,
R1 is absent, or
if X1 is a residue selected from a residue of Ahx, Aoa, Apt, Lys, Dab and Dap, then R1 is H, C₁-C₄-alkyl or acetyl, and is linked to an amino function of X1,
X2 is an amino acid residue selected from a residue of Cys, Glu, Asp, Nmc, Nmd, Nme, Nmt, Thr and Abu, and their corresponding d-forms,
X7 is an amino acid residue selected from a residue of Cys, Glu, Asp, Thr and Abu, and their corresponding d-forms,
wherein X2 and X7 are linked to each other via Q, wherein Q is a disulfide bond, a thioether bond or an ester bond,
X3 is an amino acid residue selected from a residue of Sar, His, Lys, Ala, Ahx, Apt, Arg, Asn, Gab, Gln, Trp, Thr, Ndmk, Nmn, Ntmk and Lys(αAc),
X4 is absent, or is an amino acid residue selected from a residue of His, Lys, Ahx, Apt, Arg, Asn, Gab, Gln, Trp and Thr,
X5 is an amino acid residue selected from a residue of Trp, Wme, Nak, Wcl, Wim, Wdm and Wfl,
X6 is an amino acid residue selected from a residue of Gln, Aib, Glu, Phe, Ile, Iva, Lys, Lys(Ac), Leu, Mle, Mly, Mva, Thr, Val, Trp and Phe,
X8 is an amino acid residue selected from a residue of Yme, Tyr, Trp, and Yde,
X9 is an amino acid residue selected from a residue of Nal, Nak and Trp,
X10 is an amino acid residue selected from a residue of Aib, Cba, Cit, d-Trp, d-Tza, Lys, Lys(Ac), Leu, Mle, Mly, Gln, Mkdm and Thp,
X11 is an amino acid residue selected from a residue of Asp, Bal, Gab, Glu, Kme, Lys and Orn and is linked via an amide bond to X1,
X12 is absent or is an amino acid residue selected from a residue of Asn, Gly, Ser and Ala,
X13 is absent or is an amino acid residue selected from a residue of Asn, Ala, Bal, Gly, Leu, Ile, Iva, Hol, Hph, Pal, Pyal, PyEA, His, Hme, Val, and their corresponding d-forms,
X14 is absent or is an amino acid residue selected from a residue of Mep, Sar, Bal, Ile, Dnmy and Hme,
X15 is absent or is an amino acid residue of Lys,
R2 is absent or is —NH₂, —OH or —N(C₂H₅)₂,
and
wherein amide bonds between X1 and X2, X2 and X3, X3 and X4, X4 and
X5, X11 and X1 are optionally methylated, and
wherein at least one of X10 and X15 is an amino acid residue having a side chain with an —NH₂ group, wherein the —NH₂ side chain group is functionalized by —C(O)—R5, —C(O)O—R5, —C(O)NH—R5, —S(O)₂—R5 or R5, wherein R5 may be a moiety comprising up to 50 or up to 100 carbon atoms and optionally heteroatoms selected from halogen, N, O, S and P,
or a salt or solvate thereof,
wherein the compound of formula (Ia) inhibits the binding of interleukin-23 to an interleukin-23-receptor.

Item 189: A compound of item 183, wherein compounds of formula (Ia) mare such that:
X1 is a residue selected from a residue of Ahx, Aoa, Apt, Nmha, Nmapt, Lys, Pipa, Pipb, Pipp, Dab, Dad and Dap, and is linked via an amide bond to X11,
or is a residue selected from a residue of Buty and Butyl, and is linked via an alkyl bond to X11 and when X1 is a residue of Butyl, the residue of Butyl is linked to X2 via an alkyl bond,
R1 is absent, or
if X1 is a residue selected from a residue of Ahx, Aoa, Apt, Lys, Dab and Dap, then R1 is H, C₁-C₄-alkyl or acetyl, and is linked to an amino function of X1,
X2 is an amino acid residue selected from a residue of Cys, Glu, Asp, Nmc, Nmd, Nme, Nmt, and Thr, and their corresponding d-forms,
X7 is an amino acid residue selected from a residue of Cys, Glu, Asp, Thr and Abu, and their corresponding d-forms,
wherein X2 and X7 are linked to each other via Q, wherein Q is a disulfide bond, a thioether bond or an ester bond,
X3 is an amino acid residue selected from a residue of Sar, His, Lys, Ala, Ahx, Apt, Arg, Asn, Gab, Gln, Trp, Thr, Ndmk, Nmn, Ntmk and Lys(αAc),
X4 is absent or is an amino acid residue selected from a residue of His, Lys, Ahx, Apt, Arg, Asn, Gab, Gin, Trp and Thr,
X5 is an amino acid residue selected from a residue of Trp, Wme, Nak, Wcl, Wim, Wdm and Wfl,
X6 is an amino acid residue selected from a residue of Gln, Aib, Glu, Phe, Ile, Iva, Lys, Lys(Ac), Leu, Mle, Mly, Mva, Thr, Val, Trp and Phe,
X8 is an amino acid residue selected from a residue of Yme, Tyr, Trp, Yde and Yae,
X9 is an amino acid residue selected from a residue of Nal, Nak and Trp,
X10 is an amino acid residue selected from a residue of Aib, Cba, Cit, d-Trp, d-Tza, Lys, Lys(Ac), Leu, Mle, Mly, Gin, Mkdm and Thp,
X11 is an amino acid residue selected from a residue of Asp, Bal, Gab, Glu, Kme, Lys and Orn and is linked via an amide bond to—X1,
X12 is absent or is an amino acid residue selected from a residue of Asn, Gly, Ser and Ala,
X13 is absent or is an amino acid residue selected from a residue of Asn, Ala, Bal, Gly, Leu,
Ile, Iva, Hol, Hph, Pal, Pyal, PyEA, His, Hme, Val, and their corresponding d-forms,
X14 is absent or is an amino acid residue selected from a residue of Mep, Sar, Bal, Ile, Dnmy and Hme,
X15 is absent or is an amino acid residue of Lys,
R2 is absent or is —NH₂, —OH or —N(C₂H₅) z,
and
wherein amide bonds between X1 and X2, X2 and X3, X3 and X4, X4 and X5, X11 and X1 are optionally methylated, and
wherein at least one of X10 and X15 is an amino acid residue having a side chain with an —NH₂ group, wherein the —NH₂ side chain group is functionalized by —C(O)—R5, —C(O)O—R5, —C(O)NH—R5, —S(O)₂—R5 or R5, wherein R5 may be a moiety comprising up to 50 or up to 100 carbon atoms and optionally heteroatoms selected from halogen, N, O, S and P,
or a salt or solvate thereof,
wherein the compound of formula (Ia) inhibits the binding of interleukin-23 to an interleukin-23-receptor.

Item 190: A compound of item 183 to 189, wherein at least one of X10 and X15 is an amino acid residue having a side chain with an —NH₂ group, wherein the —NH₂ side chain group is functionalized by —C(O)—R5, wherein R5 may be a moiety comprising up to 50 or up to 100 carbon atoms and optionally heteroatoms selected from halogen, N, O, S and P.

Item 191: A compound of item 183 to 190, wherein
X10 is an amino acid residue selected from a residue of Lys and Mly,
X15 is absent,
and wherein the —NH₂ side chain group of X10 is functionalized by —C(O)—R5, —C(O)O—R5, —C(O)NH—R5, —S(O)₂—R5 or R5, for example by —C(O)—R5, wherein R5 may be a moiety comprising up to 50 or up to 100 carbon atoms and optionally heteroatoms selected from halogen, N, O, S and P,
wherein the compound of formula (Ia) inhibits the binding of interleukin-23 to an interleukin-23-receptor.

Item 192: A compound of item 183 to 190, wherein
X10 is an amino acid residue selected from a residue of Lys and Mly, X15 is Lys,
and wherein the —NH₂ side chain group of X15 is functionalized by —C(O)—R5, —C(O)O—R5, —C(O)NH—R5, —S(O)₂—R5 or R5, for example by —C(O)—R5, wherein R5 may be a moiety comprising up to 50 or up to 100 carbon atoms and optionally heteroatoms selected from halogen, N, O, S and P,
wherein the compound of formula (Ia) inhibits the binding of interleukin-23 to an interleukin-23-receptor.

Item 193: A compound of item 191 or 192, wherein the —NH₂ side chain group of X10 or X15 is functionalized by —C(O)—R5.

Item 194: A compound of item 193, wherein-C(O)—R5 is selected from the group consisting of (S)-4-Carboxy-4-hexadecanoylamino-butyryl-, (S)-4-Carboxy-4-octadecanoylamino-butyryl-, 4-Hexadecanoylamino-butyryl-, 4-{3-[(R)-2,5,7,8-tetramethyl-2-((4R,8R)-4,8,12-trimethyl-tridecyl)-chroman-6-yloxycarbonyl]-propionylamino}-butyryl-, 4-octadecanoylamino-butyryl-, 4-((Z)-octadec-9-enoylamino)-butyryl-, 6-[(4,4-Diphenyl-cyclohexyloxy)-hydroxy-phosphoryloxy]-hexanoyl-, Hexadecanoyl-, (S)-4-Carboxy-4-(15-carboxy-pentadecanoylamino)-butyryl-, (S)-4-Carboxy-4-{3-[3-((2S,3R,4S,5R)-5-carboxy-2,3,4,5-tetrahydroxy-pentanoylamino)-propionylamino]-propionylamino}-butyryl-, (S)-4-Carboxy-4-{3-[(R)-2,5,7,8-tetramethyl-2-((4R,8R)-4,8,12-trimethyl-tridecyl)-chroman-6-yloxycarbonyl]-propionylamino}-butyryl-, (S)-4-Carboxy-4-((9Z,12Z)-octadeca-9,12-dienoylamino)-butyryl-, (S)-4-Carboxy-4-[6-((2S,3R,4S,5R)-5-carboxy-2,3,4,5-tetrahydroxy-pentanoylamino)-hexanoylamino]-butyryl-, (S)-4-Carboxy-4-((2S,3R,4S,5R)-5-carboxy-2,3,4,5-tetrahydroxy-pentanoylamino)-butyryl-, (S)-4-Carboxy-4-tetradecanoylamino-butyryl-, (S)-4-(11-Benzyloxycarbonyl-undecanoylamino)-4-carboxy-butyryl-, (S)-4-Carboxy-4-[11-((2S,3R,4R,5R)-2,3,4,5,6-pentahydroxy-hexylcarbamoyl)-undecanoylamino]-butyryl-, (S)-4-Carboxy-4-((Z)-octadec-9-enoylamino)-butyryl-, (S)-4-Carboxy-4-(4-dodecyloxy-benzoylamino)-butyryl-, (S)-4-Carboxy-4-henicosanoylamino-butyryl-, (S)-4-Carboxy-4-docosanoylamino-butyryl-, (S)-4-Carboxy-4-((Z)-nonadec-10-enoylamino)-butyryl-, (S)-4-Carboxy-4-(4-decyloxy-benzoylamino)-butyryl-, (S)-4-Carboxy-4-[(4′-octyloxy-biphenyl-4-carbonyl)-amino]-butyryl-, (S)-4-Carboxy-4-(12-phenyl-dodecanoylamino)-butyryl-, (S)-4-Carboxy-4-icosanoylamino-butyryl-, (S)-4-Carboxy-4 —((S)-4-carboxy-4-hexadecanoylamino-butyrylamino)-butyryl-, (S)-4-Carboxy-4-((S)-4-carboxy-4-octadecanoylamino-butyrylamino)-butyryl-, 3-(3-Octadecanoylamino-propionylamino)-propionyl-, 3-(3-Hexadecanoylamino-propionylamino)-propionyl-, 3-Hexadecanoylamino-propionyl-, (S)-4-Carboxy-4-[(R)-4-((3R,5S,7R,8R,9R,10S,12S,13R,14R,17R)-3,7,12-trihydroxy-8,10,13-trimethyl-hexadecahydro-cyclopenta[a]phenanthren-17-yl)-pentanoylamino]-butyryl-, (S)-4-Carboxy-4-[(R)-4-((3R,5R,8R,9S,10S,13R,14S,17R)-3-hydroxy-10,13-dimethyl-hexadecahydro-cyclopenta[a]phenanthren-17-yl)-pentanoylamino]-butyryl-, (S)-4-Carboxy-4-((9S,10R)-9,10,16-trihydroxy-hexadecanoylamino)-butyryl-, Tetradecanoyl-, 11-Carboxy-undecanoyl-, 11-Benzyloxycarbonyl-undecanoyl-, (S)-4-Carboxy-4-((S)-4-carboxy-4-tetradecanoylamino-butyrylamino)-butyryl-, 6-[Hydroxy-(naphthalene-2-yloxy)-phosphoryloxy]-hexanoyl-, 6-[Hydroxy-(5-phenyl-pentyloxy)-phosphoryloxy]-hexanoyl-, 4-(Naphthalene-2-sulfonylamino)-4-oxo-butyryl-, 4-(Biphenyl-4-sulfonylamino)-4-oxo-butyryl-, (S)-4-Carboxy-4-{(S)-4-carboxy-4-[2-(2-{2-[2-(2-{2-[(S)-4-carboxy-4-(17-carboxy-heptadecanoylamino)-butyrylamino]-ethoxy}-ethoxy)-acetylamino]-ethoxy}-ethoxy)-acetylamino]-butyrylamino}-butyryl-, (S)-4-Carboxy-4-[2-(2-{2-12-(2-{2-[(S)-4-carboxy-4-(17-carboxy-heptadecanoylamino)-butyrylamino]-ethoxy)-ethoxy)-acetylamino]-ethoxy}-ethoxy)-acetylamino]-butyryl-, (S)-4-Carboxy-2-{(S)-4-carboxy-2-[2-(2-{2-[2-(2-{2-[(S)-4-carboxy-4-(17-carboxy-heptadecanoylamino)-butyrylamino]-ethoxy]-ethoxy)-acetylamino]-(S)-4-Carboxy-2-[2-(2-{2-[2-(2-{2-ethoxy}-ethoxy)-acetylamino]-butyrylamino}-butyryl-, [(S)-4-carboxy-4-(17-carboxy-heptadecanoylamino)-butyrylamino]-ethoxy]-ethoxy)-acetylamino]-ethoxy}-ethoxy)-acetylamino]-butyryl-, (S)-4-Carboxy-4-{(S)-4-carboxy-4-[2-(2-{2-[(S)-4-carboxy-4-(17-carboxy-heptadecanoylamino)-butyrylamino]-ethoxy}-ethoxy)-acetylamino]-butyrylamino}-butyryl-, (S)-4-Carboxy-4-[2-(2-{2-[(S)-4-carboxy-4-(17-carboxy-heptadecanoylamino)-butyrylamino]-ethoxy}-ethoxy)-acetylamino]-butyryl-, (S)-4-Carboxy-2-{(S)-4-carboxy-2-[2-(2-{2-[(S)-4-carboxy-4-(17-carboxy-heptadecanoylamino)-butyrylamino]-ethoxy}-ethoxy)-acetylamino]-butyrylamino}-butyryl-, (S)-4-Carboxy-2-[2-(2-{2-[(S)-4-carboxy-4-(17-carboxy-heptadecanoylamino)-butyrylamino]-ethoxy}-ethoxy)-acetylamino]-butyryl-, 2-(2-{2-[2-(2-{2-[(S)-4-Carboxy-4-(17-carboxy-hepta-decanoylamino)-butyrylamino]-ethoxy}-ethoxy)-acetylamino]-ethoxy}-ethoxy)-acetyl-, 2-(2-{2-[(S)-4-Carboxy-4-(17-carboxy-heptadecanoylamino)-butyrylamino]-ethoxy}-ethoxy)-acetyl, (S)-4-Carboxy-4-((S)-4-carboxy-4-{(S)-4-carboxy-4-[(S)-4-carboxy-4-(19-carboxy-nonadecanoylamino)-butyrylamino]-butyrylamino}-butyrylamino)-butyryl, 2-(2-{2-[2-(2-{2-[(S)-4-Carboxy-4-(16-1H-tetrazol-5-yl-hexadecanoylamino)-butyrylamino]-ethoxy}-ethoxy)-acetylamino]-ethoxy}-ethoxy)-acetyl-, 2-(2-{2-[2-(2-{2-[(S)-4-Carboxy-4-(16-carboxy-hexadecanoylamino)-butyrylamino] -ethoxy}-ethoxy)-acetylamino]-ethoxy}-ethoxy)-acetyl-, (S)-4-Carboxy-4-{(S)-4-carboxy-4-[(S)-4-carboxy-4-(17-carboxy-heptadecanoylamino)-butyrylamino]-butyrylamino}-butyryl-, (S)-4-Carboxy-4-((S)-4-carboxy-4-{2-[2-(2-{2-[2-(2-{(S)-4-carboxy-4-[10-(4-carboxy-phenoxy)-decanoylamino]-butyrylamino}-ethoxy)-ethoxy]-acetylamino]-ethoxy)-ethoxy]-acetylamino}-butyryl-, (S)-4-Carboxy-4-{(S)-4-carboxy-4-[2-(2-{2-[2-(2-{2-[(S)-4-carboxy-4-(7-carboxy-heptanoylamino)-butyrylamino]-ethoxy}-ethoxy)-acetylamino]-ethoxy}-ethoxy)-acetylamino]-butyrylamino}-butyryl-, (S)-4-Carboxy-4-{(S)-4-carboxy-4-[2-(2-{2-[2-(2-{2-[(S)-4-carboxy-4-(11-carboxy-undecanoylamino)-butyrylamino]-ethoxy}-ethoxy)-acetylamino]-ethoxy)-ethoxy)-acetylamino]-butyrylamino]-butyryl-, (S)-4-Carboxy-4-{(S)-4-carboxy-4-[2-(2-{2-[2-(2-{2-[(S)-4-carboxy-4-(13-carboxy-tridecanoylamino)-butyrylamino]-ethoxy}-ethoxy)-acetylamino]-ethoxy]-ethoxy)-acetylamino]-butyrylamino]-butyryl-, (S)-4-Carboxy-4-{(S)-4-carboxy-4-[2-(2-{2-[2-(2-{2-[(S)-4-carboxy-4-(15-carboxy-pentadecanoylamino)-butyrylamino]-ethoxy}-ethoxy)-acetylamino]-ethoxy}-ethoxy)-acetylamino]-butyrylamino}-butyryl-, and (S)-4-Carboxy-4-{(S)-4-carboxy-4-[2-(2-{2-[2-(2-{2-[(S)-4-carboxy-4-(19-carboxy-nonadecanoylamino)-butyrylamino]-ethoxy}-ethoxy)-acetylamino]-ethoxy}-ethoxy)-acetylamino]-butyrylamino}-butyryl-.

Item 195. A compound of items 1 to 194, having a binding affinity to the interleukin 23 receptor of 100 nM or below (i.e., IC₅₀≤100 nM).

Item 196. A compound of items 1 to 194, having a binding affinity to the interleukin 23 receptor of 50 nM or below (i.e., IC₆₀≤50 nM).

Item 197. A compound of items 1 to 194, having a binding affinity to the interleukin 23 receptor of 10 nM or below (i.e., IC₅₀≤10 nM).

Item 198. A compound of items 1 to 194, having a binding affinity to the interleukin 23 receptor of 5 nM or below (i.e., IC₅₀≤5 nM).

Item 199. A compound of items 1 to 194 having a chemical stability in solution at pH 1.2 of less than 30% relative purity loss over 24 h at 37° C.

Item 200. A compound of items 1 to 194 having a chemical stability in solution at pH 1.2 of less than 25% relative purity loss over 24 h at 37° C.

Item 201. A compound of items 1 to 194 having a chemical stability in solution at pH 6.5 of less than 5% relative purity loss over 24 h at 37° C.

Item 202. A compound of items 1 to 194 having a chemical stability in solution at pH 6.5 of less than 2% relative purity loss over 24 h at 37° C.

Item 203. A compound of items 1 to 194 having a chemical stability in solution at pH 7.4 of less than 5% relative purity loss over 24 h at 37° C.

Item 204. A compound of items 1 to 194 having a chemical stability in solution at pH 7.4 of less than 3% relative purity loss over 24 h at 37° C.

1. A compound of formula (I): 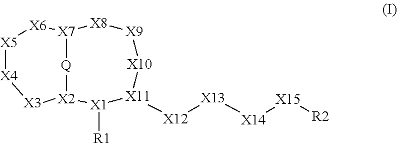
wherein
X1 is a residue selected from a residue of Ahx, Aoa, Apt, Nmha, Nmapt, Lys, Pipa, Pipb, Pipp, Dab, Dad and Dap, and is linked via an amide bond to X11,
or is a residue selected from a residue of Buty and Butyl, and is linked via an alkyl bond to X11 and when X1 is a residue selected from a residue of Butyl, the residue selected from a residue of Butyl is linked to X2 via an alkyl bond,
R1 is absent, or
if X1 is a residue selected from a residue of Ahx, Aoa, Apt, Lys, Dab and Dap, then R1 is H, C₁-C₄-alkyl or acetyl, and is linked to an amino function of X1,
X2 is an amino acid residue selected from a residue of Cys, Glu, Asp, Nmc, Nmd, Nme, Nmt, Thr and Abu, and their corresponding d-forms,
X7 is an amino acid residue selected from a residue of Cys, Glu, Asp, Thr and Abu, and their corresponding d-forms,
wherein X2 and X7 are linked to each other via Q, wherein Q is a disulfide bond, a thioether bond or an ester bond,
X3 is an amino acid residue selected from a residue of Sar, His, Lys, Ala, Ahx, Apt, Arg, Asn, Gab, Gln, Trp, Thr, Ndmk, Nmn, Ntmk and Lys(αAc),
X4 is absent, or is an amino acid residue selected from a residue of His, Lys, Ahx, Apt, Arg, Asn, Gab, Gln, Trp and Thr,
X5 is an amino acid residue selected from a residue of Trp, Wme, Nak, Wcl, Wim, Wdm and Wfl,
X6 is an amino acid residue selected from a residue of Gln, Aib, Glu, Phe, Ile, Iva, Lys, Lys(Ac), Leu, Mle, Mly, Mva, Thr, Val, Trp and Phe,
X8 is an amino acid residue selected from a residue of Yme, Tyr, Trp, and Yde,
X9 is an amino acid residue selected from a residue of Nal, Nak and Trp,
X10 is an amino acid residue selected from a residue of Aib, Cba, Cit, d-Trp, d-Tza, Lys, Lys(Ac), Leu, Mle, Mly, Gln, Mkdm and Thp,
X11 is an amino acid residue selected from a residue of Asp, Bal, Gab, Glu, Kme, Lys and Orn, and is linked via an amide bond to X1,
X12 is absent or is an amino acid residue selected from a residue of Asn, Gly, Ser and Ala,
X13 is absent or is an amino acid residue selected from a residue of Asn, Ala, Bal, Gly, Leu, Ile, Iva, Hol, Hph, Pal, Pyal, PyEA, His, Hme, and Val, and their corresponding d-forms,
X14 is absent or is an amino acid residue selected from a residue of Mep, Sar, Bal, Ile, Dnmy and Hme,
X15 is absent or is an amino acid residue selected from a residue of Lys,
R2 is absent or is —NH₂, —OH or —N(C₂H₅)₂,
and
wherein amide bonds between X1 and X2, X2 and X3, X3 and X4, X4 and X5, X11 and X1 are optionally methylated,
or a salt or solvate thereof,
and
wherein the compound of formula (I) inhibits the binding of interleukin-23 to an interleukin-23-receptor.

2. The compound of formula (I) according to claim 1 in which:
X1 is a residue selected from a residue of Ahx, Apt, Nmha, Nmapt, Lys and Dad, and is linked via an amide bond to X11,
or is a residue selected from a residue of Buty and Butyl, and is linked via an alkyl bond to X11 and when X1 is a residue of Butyl, the residue of Butyl is linked to X2 via an alkyl bond,
R1 is absent, or
if X1 is a residue selected from a residue of Ahx, Apt and Lys, then R1 is H, C₁-C₄-alkyl or acetyl, and is linked to an amino function of X1,
X2 is an amino acid residue selected from a residue of Cys, Glu, Asp, Nmc, Nmd, Nme, Nmt, Thr and Abu,
X7 is an amino acid residue selected from a residue of Cys, Glu, Asp, Thr and Abu, wherein X2 and X7 are linked to each other via Q, wherein Q is a disulfide bond, a thioether bond or an ester bond,
X3 is an amino acid residue selected from a residue of Apt, Asn, Ndmk, Nmn and Lys(αAc),
X4 is absent or is an amino acid residue of Thr,
X5 is an amino acid residue selected from a residue of Trp, Wme and Wim,
X6 is an amino acid residue selected from a residue of Gln, Lys(Ac) and Leu,
X8 is an amino acid residue selected from a residue of Yme, Trp, and Yde,
X9 is an amino acid residue of Nal,
X10 is an amino acid residue selected from a residue of Aib, Lys, Lys(Ac), Mle, Mly, Mkdm and Thp,
X11 is an amino acid residue selected from a residue of Asp, Glu, Kme, Lys and Orn, and is linked via an amide bond to X1,
X12 is absent or is an amino acid residue selected from a residue of Asn, Gly, Ser and Ala,
X13 is absent or is an amino acid residue selected from a residue of Asn, Ala, Bal, Gly, d-Leu, Ile, Pal, Pyal, PyEA, His and Hme,
X14 is absent or is an amino acid residue selected from a residue of Mep, Sar, Bal, Ile and Hme,
X15 is absent or is an amino acid residue of Lys,
R2 is absent or is —NH₂, —OH or —N(C₂H₅)₂.

3. A compound of formula (I): 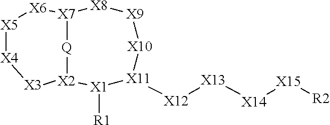
wherein
X1 is a residue selected from a residue of Ahx, Aoa, Apt, Nmha, Nmapt, Lys, Pipa, Pipb, Pipp, Dab, Dad and Dap, and is linked via an amide bond to X11,
or is a residue selected from a residue of Buty and Butyl, and is linked via an alkyl bond to X11 and when X1 is a residue of Butyl, the residue of Butyl is linked to X2 via an alkyl bond,
R1 is absent, or
if X1 is a residue selected from a residue of Ahx, Aoa, Apt, Lys, Dab and Dap, then R1 is H, C₁-C₄-alkyl or acetyl, and is linked to an amino function of X1,
X2 is an amino acid residue selected from a residue of Cys, Glu, Asp, Nmc, Nmd, Nme, Nmt, and Thr, and their corresponding d-forms,
X7 is an amino acid residue selected from a residue of Cys, Glu, Asp, Thr and Abu, and their corresponding d-forms,
wherein X2 and X7 are linked to each other via Q, wherein Q is a disulfide bond, a thioether bond or an ester bond,
X3 is an amino acid residue selected from a residue of Sar, His, Lys, Ala, Ahx, Apt, Arg, Asn, Gab, Gln, Trp, Thr, Ndmk, Nmn, Ntmk and Lys(αAc),
X4 is absent, or is an amino acid residue selected from a residue of His, Lys, Ahx, Apt, Arg, Asn, Gab, Gln, Trp and Thr,
X5 is an amino acid residue selected from a residue of Trp, Wme, Nak, Wcl, Wim, Wdm and Wfl,
X6 is an amino acid residue selected from a residue of Gln, Aib, Glu, Phe, Ile, Iva, Lys, Lys(Ac), Leu, Mle, Mly, Mva, Thr, Val, Trp and Phe,
X8 is an amino acid residue selected from a residue of Yme, Tyr, Trp, Yde and Yae,
X9 is an amino acid residue selected from a residue of Nal, Nak and Trp,
X10 is an amino acid residue selected from a residue of Aib, Cba, Cit, d-Trp, d-Tza, Lys, Lys(Ac), Leu, Mle, Mly, Gln, Mkdm and Thp,
X11 is an amino acid residue selected from a residue of Asp, Bal, Gab, Glu, Kme, Lys and Orn, and is linked via an amide bond to X1,
X12 is absent or is an amino acid residue selected from a residue of Asn, Gly, Ser and Ala,
X13 is absent or is an amino acid residue selected from a residue of Asn, Ala, Bal, Gly, Leu, Ile, Iva, Hol, Hph, Pal, Pyal, PyEA, His, Hme, Val, and their corresponding d-forms,
X14 is absent or is an amino acid residue selected from a residue of Mep, Sar, Bal, Ile, Dnmy and Hme,
X15 is absent or is an amino acid residue of Lys,
R2 is absent or is —NH₂, —OH or —N(C₂H₅)₂,
and
wherein amide bonds between X1 and X2, X2 and X3, X3 and X4, X4 and X5, X11 and X1 are optionally methylated,
or a salt or solvate thereof,
and
wherein the compound of formula (I) inhibits the binding of interleukin-23 to an interleukin-23-receptor.

4. The compound of formula (I) according to claim 3 in which:
X1 is a residue selected from a residue of Ahx, Apt, Nmha, Nmapt, Lys and Dad, and is linked via an amide bond to X11,
or is a residue selected from a residue of Buty and Butyl, and is linked via an alkyl bond to X11 and when X1 is a residue of Butyl, the residue of Butyl is linked to X2 via an alkyl bond,
R1 is absent, or
if X1 is a residue selected from a residue of Ahx, Apt and Lys, then R1 is H, C₁-C₄-alkyl or acetyl, and is linked to an amino function of X1,
X2 is an amino acid residue selected from a residue of Cys, Glu, Asp, Nmc, Nmd, Nme, Nmt, and Thr,
X7 is an amino acid residue selected from a residue of Cys, Glu, Asp, Thr and Abu, wherein X2 and X7 are linked to each other via Q, wherein Q is a disulfide bond, a thioether bond or an ester bond,
X3 is an amino acid residue selected from a residue of Apt, Asn, Ndmk, Nmn and Lys(αAc),
X4 is absent or is an amino acid residue of Thr,
X5 is an amino acid residue selected from a residue of Trp, Wme and Wim,
X6 is an amino acid residue selected from a residue of Gln, Lys(Ac) and Leu,
X8 is an amino acid residue selected from a residue of Yme, Trp, Yde and Yae,
X9 is an amino acid residue of Nal,
X10 is an amino acid residue selected from a residue of Aib, Lys, Lys(Ac), Mle, Mly, Mkdm and Thp,
X11 is an amino acid residue selected from a residue of Asp, Glu, Kme, Lys and Orn, and is linked via an amide bond to X1,
X12 is absent or is an amino acid residue selected from a residue of Asn, Gly, Ser and Ala,
X13 is absent or is an amino acid residue selected from a residue of Asn, Ala, Bal, Gly, d-Leu, Ile, Pal, Pyal, PyEA, His and Hme,
X14 is absent or is an amino acid residue selected from a residue of Mep, Sar, Bal, Ile and Hme,
X15 is absent or is an amino acid residue Lys,
R2 is absent or is —NH₂, —OH or —N(C₂H₅)₂.

5. The compound of formula (I) according to claim 1, in which:
(a) —X3 is an amino acid residue selected from a residue of Asn and Nmn, and
X4 is an amino acid residue of Thr;
or
(b) —X3 is an amino acid residue selected from a residue of Ndmk, and Lys(αAc), and
X4 is absent;
or
(c) —X1 is a residue selected from a residue of Ahx, Aoa, Apt, Nmha, Nmapt, Lys, Pipa, Pipb, Pipp, Dab and Dap, and X11 is an amino acid residue selected from a residue of Asp and Glu,
or
(d) —X1 is a residue of Dad and X11 is an amino acid residue selected from a residue of Lys and Kme,
or
(e) —X1 is a residue selected from a residue of Buty and Butyl and X11 is an amino acid residue of Kme.

6. (canceled)

7. (canceled)

8. The compound of formula (I) according to claim 1, in which X2-Q-X7 is of a group of:
Cys-Q-Cys, Nmc-Q-Cys and Cys-Q-Nmc, wherein Q is a disulfide bond, or
Abu-Q-Cys, Cys-Q-Abu, wherein Q is a thioether bond, or
Thr-Q-Asp, Thr-Q-Glu, Glu-Q-Thr, wherein Q is an ester bond.

9. The compound of formula (I) according to claim 3, in which X2-Q-X7 is of a group of:
Cys-Q-Cys, Nmc-Q-Cys and Cys-Q-Nmc, wherein Q is a disulfide bond, or
Cys-Q-Abu, wherein Q is a thioether bond, or
Thr-Q-Asp, Thr-Q-Glu, Glu-Q-Thr, wherein Q is an ester bond.

10. The compound of formula (I) according to claim 1, in which:
R1 is absent or is H, C₁-C₄-alkyl or acetyl, and is linked to an amino function of X1,
X1 is a residue of Apt, and is linked via an amide bond to X11,
X2 is an amino acid residue of Cys,
X7 is an amino acid residue of Cys,
wherein X2 and X7 are linked to each other via Q, wherein Q is a disulfide bond,
X3 is an amino acid residue selected from a residue of Asn and Lys(αAc),
X4 is absent or is an amino acid residue of Thr,
X5 is an amino acid residue of Wim,
X6 is an amino acid residue of Lys(Ac),
X8 is an amino acid residue of Yde,
X9 is an amino acid residue of Nal,
X10 is an amino acid residue selected from a residue of Mkdm and Thp,
X11 is an amino acid residue of Glu, and is linked via an amide bond to—X1,
X12 is an amino acid residue of Asn,
X13 is an amino acid residue of Pal,
X14 is an amino acid residue of Sar,
X15 is absent,
R2 is —NH₂,
and
wherein the compound of formula (I) inhibits the binding of interleukin-23 to an interleukin-23-receptor.

11. The compound of formula (I) according to claim 1 in which:
X1 is a residue selected from a residue of Ahx, Apt, Nmha, Nmapt, Lys and Dad, and is linked via an amide bond to—X11,
or is a residue selected from a residue of Buty and Butyl, and is linked via an alkyl bond to X11 and when X1 is a residue of Butyl, the residue of Butyl is linked to X2 via an alkyl bond,
R1 is absent or
if—X1 is a residue selected from a residue of Ahx, Apt and Lys, then R1 is H, C₁-C₄-alkyl or acetyl, and is linked to an amino function of—X1,
X2 is an amino acid residue selected from a residue of Cys, Nmc and Abu,
X7 is an amino acid residue selected from a residue of Cys, Glu, Asp and Thr,
wherein X2 and X7 are linked to each other via Q, wherein Q is a disulfide bond, a thioether bond or an ester bond,
X3 is an amino acid residue of Lys(αAc),
X4 is absent,
X5 is an amino acid residue selected from a residue of Trp and Wim,
X6 is an amino acid residue of Lys(Ac),
X8 is an amino acid residue of Yde,
X9 is an amino acid residue of Nal,
X10 is an amino acid residue selected from a residue of Mkdm and Thp,
X11 is an amino acid residue selected from a residue of Asp, Glu, Kme and Lys, and is linked via an amide bond to X1,
X12 is an amino acid residue of Gly,
X13 is an amino acid residue of PyEA,
X14 is absent,
X15 is absent,
R2 is absent.

12. The compound of formula (I) according to claim 1, wherein the compound selected from the compounds of SEQ ID NO: 1-66, 68-70, 72-74, 76-82, 84-95, 98, 102-106, 108-158.

13. A compound of formula (Ia): 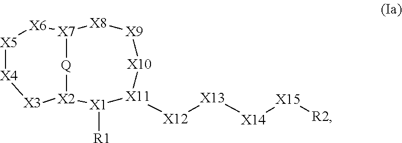
wherein
X1 is a residue selected from a residue of Ahx, Aoa, Apt, Nmha, Nmapt, Lys, Pipa, Pipb, Pipp, Dab, Dad and Dap, and is linked via an amide bond to X11,
or is a residue selected from a residue of Buty and Butyl, and is linked via an alkyl bond to X11 and when X1 is a residue of Butyl, the residue of Butyl is linked to X2 via an alkyl bond,
R1 is absent, or
if X1 is a residue selected from a residue of Ahx, Aoa, Apt, Lys, Dab and Dap, then R1 is H, C₁-C₄-alkyl or acetyl, and is linked to an amino function of X1,
X2 is an amino acid residue selected from a residue of Cys, Glu, Asp, Nmc, Nmd, Nme, Nmt, Thr and Abu, and their corresponding d-forms,
X7 is an amino acid residue selected from a residue of Cys, Glu, Asp, Thr and Abu, and their corresponding d-forms,
wherein X2 and X7 are linked to each other via Q, wherein Q is a disulfide bond, a thioether bond or an ester bond,
X3 is an amino acid residue selected from a residue of Sar, His, Lys, Ala, Ahx, Apt, Arg, Asn, Gab, Gln, Trp, Thr, Ndmk, Nmn, Ntmk and Lys(αAc),
X4 is absent, or is an amino acid residue selected from a residue of His, Lys, Ahx, Apt, Arg, Asn, Gab, Gln, Trp and Thr,
X5 is an amino acid residue selected from a residue of Trp, Wme, Nak, Wcl, Wim, Wdm and Wfl,
X6 is an amino acid residue selected from a residue of Gln, Aib, Glu, Phe, Ile, Iva, Lys, Lys(Ac), Leu, Mle, Mly, Mva, Thr, Val, Trp and Phe,
X8 is an amino acid residue selected from a residue of Yme, Tyr, Trp, and Yde,
X9 is an amino acid residue selected from a residue of Nal, Nak and Trp,
X10 is an amino acid residue selected from a residue of Aib, Cba, Cit, d-Trp, d-Tza, Lys, Lys(Ac), Leu, Mle, Mly, Gln, Mkdm and Thp,
X11 is an amino acid residue selected from a residue of Asp, Bal, Gab, Glu, Kme, Lys and Orn and is linked via an amide bond to X1,
X12 is absent or is an amino acid residue selected from a residue of Asn, Gly, Ser and Ala,
X13 is absent or is an amino acid residue selected from a residue of Asn, Ala, Bal, Gly, Leu, Ile, Iva, Hol, Hph, Pal, Pyal, PyEA, His, Hme, Val, and their corresponding d-forms,
X14 is absent or is an amino acid residue selected from a residue of Mep, Sar, Bal, Ile, Dnmy and Hme,
X15 is absent or is an amino acid residue of Lys,
R2 is absent or is —NH₂, —OH or —N(C₂H₅)₂,
and
wherein amide bonds between X1 and X2, X2 and X3, X3 and X4, X4 and X5, X11 and X1 are optionally methylated,
and
wherein at least one of X10 and X15 is an amino acid residue having a side chain with an —NH₂ group, wherein the —NH₂ side chain group is functionalized by —C(O)—R5, —C(O)O—R5, —C(O)NH—R5, —S(O)₂—R5 or R5, wherein R5 may be a moiety comprising up to 50 or up to 100 carbon atoms and optionally heteroatoms selected from halogen, N, O, S and P,
or a salt or solvate thereof,
wherein the compound of formula (Ia) inhibits the binding of interleukin-23 to an interleukin-23-receptor.

14. A compound of formula (Ia): 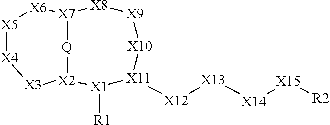
wherein
X1 is a residue selected from a residue of Ahx, Aoa, Apt, Nmha, Nmapt, Lys, Pipa, Pipb, Pipp, Dab, Dad and Dap, and is linked via an amide bond to X11,
or is a residue selected from a residue of Buty and Butyl, and is linked via an alkyl bond to X11 and when X1 is a residue of Butyl, the residue of Butyl is linked to X2 via an alkyl bond,
R1 is absent, or
if X1 is a residue selected from a residue of Ahx, Aoa, Apt, Lys, Dab and Dap, then R1 is H, C₁-C₄-alkyl or acetyl, and is linked to an amino function of X1,
X2 is an amino acid residue selected from a residue of Cys, Glu, Asp, Nmc, Nmd, Nme, Nmt, and Thr, and their corresponding d-forms,
X7 is an amino acid residue selected from a residue of Cys, Glu, Asp, Thr and Abu, and their corresponding d-forms,
wherein X2 and X7 are linked to each other via Q, wherein Q is a disulfide bond, a thioether bond or an ester bond,
X3 is an amino acid residue selected from a residue of Sar, His, Lys, Ala, Ahx, Apt, Arg, Asn, Gab, Gln, Trp, Thr, Ndmk, Nmn, Ntmk and Lys(αAc),
X4 is absent or is an amino acid residue selected from a residue of His, Lys, Ahx, Apt, Arg, Asn, Gab, Gln, Trp and Thr,
X5 is an amino acid residue selected from a residue of Trp, Wme, Nak, Wcl, Wim, Wdm and Wfl,
X6 is an amino acid residue selected from a residue of Gln, Aib, Glu, Phe, Ile, Iva, Lys, Lys(Ac), Leu, Mle, Mly, Mva, Thr, Val, Trp and Phe,
X8 is an amino acid residue selected from a residue of Yme, Tyr, Trp, Yde and Yae,
X9 is an amino acid residue selected from a residue of Nal, Nak and Trp,
X10 is an amino acid residue selected from a residue of Aib, Cba, Cit, d-Trp, d-Tza, Lys, Lys(Ac), Leu, Mle, Mly, Gln, Mkdm and Thp,
X11 is an amino acid residue selected from a residue of Asp, Bal, Gab, Glu, Kme, Lys and Orn and is linked via an amide bond to—X1,
X12 is absent or is an amino acid residue selected from a residue of Asn, Gly, Ser and Ala,
X13 is absent or is an amino acid residue selected from a residue of Asn, Ala, Bal, Gly, Leu, Ile, Iva, Hol, Hph, Pal, Pyal, PyEA, His, Hme, Val, and their corresponding d-forms,
X14 is absent or is an amino acid residue selected from a residue of Mep, Sar, Bal, Ile, Dnmy and Hme,
X15 is absent or is an amino acid residue of Lys,
R2 is absent or is —NH₂, —OH or —N(C₂H₅)₂,
and
wherein amide bonds between X1 and X2, X2 and X3, X3 and X4, X4 and X5, X11 and X1 are optionally methylated,
and
wherein at least one of X10 and X15 is an amino acid residue having a side chain with an —NH₂ group, wherein the —NH₂ side chain group is functionalized by —C(O)—R5, —C(O)O—R5, —C(O)NH—R5, —S(O)₂—R5 or R5, wherein R5 may be a moiety comprising up to 50 or up to 100 carbon atoms and optionally heteroatoms selected from halogen, N, O, S and P,
or a salt or solvate thereof,
wherein the compound of formula (Ia) inhibits the binding of interleukin-23 to an interleukin-23-receptor.

15. The compound of formula (Ia) according to claim 13, in which:
X10 is a residue selected from a residue of Lys and Mly, wherein the —NH₂ side chain group is functionalized by —C(O)—R5, and—X15 is absent, or
X15 is Lys, wherein the —NH₂ side chain group is functionalized by —C(O)—R5,
and
—C(O)—R5 is selected from the group consisting of (S)-4-Carboxy-4-hexadecanoylamino-butyryl-, (S)-4-Carboxy-4-octadecanoylamino-butyryl-, 4-Hexadecanoylamino-butyryl-, 4-{3-[(R)-2,5,7,8-tetramethyl-2-((4R,8R)-4,8,12-trimethyl-tridecyl)-chroman-6-yloxycarbonyl]-propionylamino}-butyryl-, 4-octadecanoylamino-butyryl-, 4-((Z)-octadec-9-enoylamino)-butyryl-, 6-[(4,4-Diphenyl-cyclohexyloxy)-hydroxy-phosphoryloxy]-hexanoyl-, Hexadecanoyl-, (S)-4-Carboxy-4-(15-carboxy-pentadecanoylamino)-butyryl-, (S)-4-Carboxy-4-{3-[3-((2S,3R,4S,5R)-5-carboxy-2,3,4,5-tetrahydroxy-pentanoylamino)-propionylamino]-propionylamino}-butyryl-, (S)-4-Carboxy-4-{3-[(R)-2,5,7,8-tetramethyl-2-((4R,8R)-4,8,12-trimethyl-tridecyl)-chroman-6-yloxycarbonyl]-propionylamino}-butyryl-, (S)-4-Carboxy-4-((9Z,12Z)-octadeca-9,12-dienoylamino)-butyryl-, (S)-4-Carboxy-4-[6-((2S,3R,4S,5R)-5-carboxy-2,3,4,5-tetrahydroxy-pentanoylamino)-hexanoylamino]-butyryl-, (S)-4-Carboxy-4-((2S,3R,4S,5R)-5-carboxy-2,3,4,5-tetrahydroxy-pentanoylamino)-butyryl-, (S)-4-Carboxy-4-tetradecanoylamino-butyryl-, (S)-4-(11-Benzyloxycarbonyl-undecanoylamino)-4-carboxy-butyryl-, (S)-4-Carboxy-4-[11-((2S,3R,4R,5R)-2,3,4,5,6-pentahydroxy-hexylcarbamoyl)-undecanoylamino]-butyryl-, (S)-4-Carboxy-4-((Z)-octadec-9-enoylamino)-butyryl-, (S)-4-Carboxy-4-(4-dodecyloxy-benzoylamino)-butyryl-, (S)-4-Carboxy-4-henicosanoylamino-butyryl-, (S)-4-Carboxy-4-docosanoylamino-butyryl-, (S)-4-Carboxy-4-((Z)-nonadec-10-enoylamino)-butyryl-, (S)-4-Carboxy-4-(4-decyloxy-benzoylamino)-butyryl-, (S)-4-Carboxy-4-[(4′-octyloxy-biphenyl-4-carbonyl)-amino]-butyryl-, (S)-4-Carboxy-4-(12-phenyl-dodecanoylamino)-butyryl-, (S)-4-Carboxy-4-icosanoylamino-butyryl-, (S)-4-Carboxy-4-((S)-4-carboxy-4-hexadecanoylamino-butyrylamino)-butyryl-, (S)-4-Carboxy-4-((S)-4-carboxy-4-octadecanoylamino-butyrylamino)-butyryl-, 3-(3-Octadecanoylamino-propionylamino)-propionyl-, 3-(3-Hexadecanoylamino-propionylamino)-propionyl-, 3-Hexadecanoylamino-propionyl-, (S)-4-Carboxy-4-[(R)-4-((3R,5S,7R,8R,9R,10S,12S,13R,14R,17R)-3,7,12-trihydroxy-8,10,13-trimethyl-hexadecahydro-cyclopenta[a]phenanthren-17-yl)-pentanoylamino]-butyryl-, (S)-4-Carboxy-4-[(R)-4-((3R,5R,8R,9S,10S,13R,14S,17R)-3-hydroxy-10,13-dimethyl-hexadecahydro-cyclopenta[a]phenanthren-17-yl)-pentanoylamino]-butyryl-, (S)-4-Carboxy-4-((9S,10R)-9,10,16-trihydroxy-hexadecanoylamino)-butyryl-, Tetradecanoyl-, 11-Carboxy-undecanoyl-, 11-Benzyloxycarbonyl-undecanoyl-, (S)-4-Carboxy-4-((S)-4-carboxy-4-tetradecanoylamino-butyrylamino)-butyryl-, 6-[Hydroxy-(naphthalene-2-yloxy)-phosphoryloxy]-hexanoyl-, 6-[Hydroxy-(5-phenyl-pentyloxy)-phosphoryloxy]-hexanoyl-, 4-(Naphthalene-2-sulfonylamino)-4-oxo-butyryl-, 4-(Biphenyl-4-sulfonylamino)-4-oxo-butyryl-, (S)-4-Carboxy-4-{(S)-4-carboxy-4-[2-(2-{2-[2-(2-{2-[(S)-4-carboxy-4-(17-carboxy-heptadecanoylamino)-butyrylamino]-ethoxy}-ethoxy)-acetylamino]-ethoxy}-ethoxy)-acetylamino]-butyrylamino}-butyryl-, (S)-4-Carboxy-4-[2-(2-{2-[2-(2-{2-[(S)-4-carboxy-4-(17-carboxy-heptadecanoylamino)-butyrylamino]-ethoxy}-ethoxy)-acetylamino]-ethoxy}-ethoxy)-acetylamino]-butyryl-, (S)-4-Carboxy-2-{(S)-4-carboxy-2-[2-(2-{2-[2-(2-{2-[(S)-4-carboxy-4-(17-carboxy-heptadecanoylamino)-butyrylamino]-ethoxy}-ethoxy)-acetylamino]-ethoxy}-ethoxy)-acetylamino]-butyrylamino}-butyryl-, (S)-4-Carboxy-2-[2-(2-{2-[2-(2-{2-[(S)-4-carboxy-4-(17-carboxy-heptadecanoylamino)-butyrylamino]-ethoxy}-ethoxy)-acetylamino]-ethoxy}-ethoxy)-acetylamino]-butyryl-, (S)-4-Carboxy-4-{(S)-4-carboxy-4-[2-(2-{2-[(S)-4-carboxy-4-(17-carboxy-heptadecanoylamino)-butyrylamino]-ethoxy}-ethoxy)-acetylamino]-butyrylamino}-butyryl-, (S)-4-Carboxy-4-[2-(2-{2-[(S)-4-carboxy-4-(17-carboxy-heptadecanoylamino)-butyrylamino]-ethoxy}-ethoxy)-acetylamino]-butyryl-, (S)-4-Carboxy-2-{(S)-4-carboxy-2-[2-(2-{2-[(S)-4-carboxy-4-(17-carboxy-heptadecanoylamino)-butyrylamino]-ethoxy}-ethoxy)-acetylamino]-butyrylamino}-butyryl-, (S)-4-Carboxy-2-[2-(2-{2-[(S)-4-carboxy-4-(17-carboxy-heptadecanoylamino)-butyrylamino]-ethoxy}-ethoxy)-acetylamino]-butyryl-, 2-(2-{2-[2-(2-{2-[(S)-4-Carboxy-4-(17-carboxy-heptadecanoylamino)-butyrylamino]-ethoxy}-ethoxy)-acetylamino]-ethoxy}-ethoxy)-acetyl-, 2-(2-{2-[(S)-4-Carboxy-4-(17-carboxy-heptadecanoylamino)-butyrylamino]-ethoxy}-ethoxy)-acetyl, (S)-4-Carboxy-4-((S)-4-carboxy-4-{(S)-4-carboxy-4-[(S)-4-carboxy-4-(19-carboxy-nonadecanoylamino)-butyrylamino]-butyrylamino}-butyrylamino)-butyryl, 2-(2-{2-[2-(2-{2-[(S)-4-Carboxy-4-(16-1H-tetrazol-5-yl-hexadecanoylamino)-butyrylamino]-ethoxy}-ethoxy)-acetylamino]-ethoxy}-ethoxy)-acetyl-, 2-(2-{2-[2-(2-{2-[(S)-4-Carboxy-4-(16-carboxy-hexadecanoylamino)-butyrylamino]-ethoxy}-ethoxy)-acetylamino]-ethoxy}-ethoxy)-acetyl-, (S)-4-Carboxy-4-{(S)-4-carboxy-4-[(S)-4-carboxy-4-(17-carboxy-heptadecanoylamino)-butyrylamino]-butyrylamino}-butyryl-, (S)-4-Carboxy-4-((S)-4-carboxy-4-{2-[2-(2-{2-[2-(2-{(S)-4-carboxy-4-[10-(4-carboxy-phenoxy)-decanoylamino]-butyrylamino}-ethoxy)-ethoxy]-acetylamino}-ethoxy)-ethoxy]-acetylamino}-butyryl-, (S)-4-Carboxy-4-{(S)-4-carboxy-4-[2-(2-{2-[2-(2-{2-[(S)-4-carboxy-4-(7-carboxy-heptanoylamino)-butyrylamino]-ethoxy}-ethoxy)-acetylamino]-ethoxy}-ethoxy)-acetylamino]-butyrylamino}-butyryl-, (S)-4-Carboxy-4-{(S)-4-carboxy-4-[2-(2-{2-[2-(2-{2-[(S)-4-carboxy-4-(11-carboxy-undecanoylamino)-butyrylamino]-ethoxy}-ethoxy)-acetylamino]-ethoxy}-ethoxy)-acetylamino]-butyrylamino}-butyryl-, (S)-4-Carboxy-4-{(S)-4-carboxy-4-[2-(2-{2-[2-(2-{2-[(S)-4-carboxy-4-(13-carboxy-tridecanoylamino)-butyrylamino]-ethoxy}-ethoxy)-acetylamino]-ethoxy}-ethoxy)-acetylamino]-butyrylamino}-butyryl-, (S)-4-Carboxy-4-{(S)-4-carboxy-4-[2-(2-{2-[2-(2-{2-[(S)-4-carboxy-4-(15-carboxy-pentadecanoylamino)-butyrylamino]-ethoxy}-ethoxy)-acetylamino]-ethoxy}-ethoxy)-acetylamino]-butyrylamino}-butyryl-, and (S)-4-Carboxy-4-{(S)-4-carboxy-4-[2-(2-{2-[2-(2-{2-[(S)-4-carboxy-4-(19-carboxy-nonadecanoylamino)-butyrylamino]-ethoxy}-ethoxy)-acetylamino]-ethoxy}-ethoxy)-acetylamino]-butyrylamino}-butyryl-.

16. (canceled)

17. The compound of formula (I) according to claim 1, wherein the compound is represented by any one of SEQ ID NO:1 to 158; or wherein the compound is represented by SEQ ID NO: 35, 66, 113, 128, 144, 146, 147, 148, or 149.

18. The compound of formula (Ia) according to claim 13, wherein the compound is represented by SEQ ID NO: 67, 71, 75, 83, 96, 97, 99, 100, 101 or 108.

19. (canceled)

20. A method of treating an autoimmune or an inflammatory disease, comprising administering the compound of formula (I) according to claim 1 to a subject in need thereof.

21. The method according to claim 20, wherein the autoimmune or inflammatory disease is selected from inflammatory bowel disease, such as Crohn's disease and ulcerative colitis, psoriasis, psoriatic arthritis and hidradenitis suppurativa.

22. The method according to claim 20, which is present as an active agent in a pharmaceutical composition together with at least one pharmaceutically acceptable carrier.

23. A pharmaceutical composition comprising at least one compound of formula (I) according to claim 1, or a physiological acceptable salt or solvate thereof, and at least a pharmaceutically acceptable carrier.

24. The compound of formula (I) according to claim 3, in which:
(a) —X3 is an amino acid residue selected from a residue of Asn and Nmn, and
X4 is an amino acid residue of Thr;
or
(b) —X3 is an amino acid residue selected from a residue of Ndmk, and Lys(αAc), and
X4 is absent;
or
(c) —X1 is a residue selected from a residue of Ahx, Aoa, Apt, Nmha, Nmapt, Lys, Pipa, Pipb, Pipp, Dab and Dap, and X11 is an amino acid residue selected from a residue of Asp and Glu,
or
X1 is a residue of Dad and X11 is an amino acid residue selected from a residue of Lys and Kme,
or
X1 is a residue selected from a residue of Buty and Butyl and X11 is an amino acid residue of Kme;
or
(d) —R1 is absent or is H, C₁-C₄-alkyl or acetyl, and is linked to an amino function of X1,
X1 is a residue of Apt, and is linked via an amide bond to X11,
X2 is an amino acid residue of Cys,
X7 is an amino acid residue of Cys,
wherein X2 and X7 are linked to each other via Q, wherein Q is a disulfide bond,
X3 is an amino acid residue selected from a residue of Asn and Lys(αAc),
X4 is absent or is an amino acid residue of Thr,
X5 is an amino acid residue of Wim,
X6 is an amino acid residue of Lys(Ac),
X8 is an amino acid residue of Yde,
X9 is an amino acid residue of Nal,
X10 is an amino acid residue selected from a residue of Mkdm and Thp,
X11 is an amino acid residue of Glu, and is linked via an amide bond to—X1,
X12 is an amino acid residue of Asn,
X13 is an amino acid residue of Pal,
X14 is an amino acid residue of Sar,
X15 is absent,
R2 is —NH₂,
and
wherein the compound of formula (I) inhibits the binding of interleukin-23 to an interleukin-23-receptor;
or
(e) wherein the compound is represented by SEQ ID NO: 35, 66, 113, 128, 144, 146, 147, 148, and 149.

25. The compound of formula (Ia) according to claim 12, in which:
X10 is a residue selected from a residue of Lys and Mly, wherein the —NH₂ side chain group is functionalized by —C(O)—R5, and—X15 is absent, or
X15 is Lys, wherein the —NH2 side chain group is functionalized by —C(O)—R5,
and
—C(O)—R5 is selected from the group consisting of (S)-4-Carboxy-4-hexadecanoylamino-butyryl-, (S)-4-Carboxy-4-octadecanoylamino-butyryl-, 4-Hexadecanoylamino-butyryl-, 4-{3-[(R)-2,5,7,8-tetramethyl-2-((4R,8R)-4,8,12-trimethyl-tridecyl)-chroman-6-yloxycarbonyl]-propionylamino}-butyryl-, 4-octadecanoylamino-butyryl-, 4-((Z)-octadec-9-enoylamino)-butyryl-, 6-[(4,4-Diphenyl-cyclohexyloxy)-hydroxy-phosphoryloxy]-hexanoyl-, Hexadecanoyl-, (S)-4-Carboxy-4-(15-carboxy-pentadecanoylamino)-butyryl-, (S)-4-Carboxy-4-{3-[3-((2S,3R,4S,5R)-5-carboxy-2,3,4,5-tetrahydroxy-pentanoylamino)-propionylamino]-propionylamino}-butyryl-, (S)-4-Carboxy-4-{3-[(R)-2,5,7,8-tetramethyl-2-((4R,8R)-4,8,12-trimethyl-tridecyl)-chroman-6-yloxycarbonyl]-propionylamino}-butyryl-, (S)-4-Carboxy-4-((9Z,12Z)-octadeca-9,12-dienoylamino)-butyryl-, (S)-4-Carboxy-4-[6-((2S,3R,4S,5R)-5-carboxy-2,3,4,5-tetrahydroxy-pentanoylamino)-hexanoylamino]-butyryl-, (S)-4-Carboxy-4-((2S,3R,4S,5R)-5-carboxy-2,3,4,5-tetrahydroxy-pentanoylamino)-butyryl-, (S)-4-Carboxy-4-tetradecanoylamino-butyryl-, (S)-4-(11-Benzyloxycarbonyl-undecanoylamino)-4-carboxy-butyryl-, (S)-4-Carboxy-4-[11-((2S,3R,4R,5R)-2,3,4,5,6-pentahydroxy-hexylcarbamoyl)-undecanoylamino]-butyryl-, (S)-4-Carboxy-4-((Z)-octadec-9-enoylamino)-butyryl-, (S)-4-Carboxy-4-(4-dodecyloxy-benzoylamino)-butyryl-, (S)-4-Carboxy-4-henicosanoylamino-butyryl-, (S)-4-Carboxy-4-docosanoylamino-butyryl-, (S)-4-Carboxy-4-((Z)-nonadec-10-enoylamino)-butyryl-, (S)-4-Carboxy-4-(4-decyloxy-benzoylamino)-butyryl-, (S)-4-Carboxy-4-[(4′-octyloxy-biphenyl-4-carbonyl)-amino]-butyryl-, (S)-4-Carboxy-4-(12-phenyl-dodecanoylamino)-butyryl-, (S)-4-Carboxy-4-icosanoylamino-butyryl-, (S)-4-Carboxy-4-((S)-4-carboxy-4-hexadecanoylamino-butyrylamino)-butyryl-, (S)-4-Carboxy-4-((S)-4-carboxy-4-octadecanoylamino-butyrylamino)-butyryl-, 3-(3-Octadecanoylamino-propionylamino)-propionyl-, 3-(3-Hexadecanoylamino-propionylamino)-propionyl-, 3-Hexadecanoylamino-propionyl-, (S)-4-Carboxy-4-[(R)-4-((3R,5S,7R,8R,9R,10S,12S,13R,14R,17R)-3,7,12-trihydroxy-8,10,13-trimethyl-hexadecahydro-cyclopenta[a]phenanthren-17-yl)-pentanoylamino]-butyryl-, (S)-4-Carboxy-4-[(R)-4-((3R,5R,8R,9S,10S,13R,14S,17R)-3-hydroxy-10,13-dimethyl-hexadecahydro-cyclopenta[a]phenanthren-17-yl)-pentanoylamino]-butyryl-, (S)-4-Carboxy-4-((9S,10R)-9,10,16-trihydroxy-hexadecanoylamino)-butyryl-, Tetradecanoyl-, 11-Carboxy-undecanoyl-, 11-Benzyloxycarbonyl-undecanoyl-, (S)-4-Carboxy-4-((S)-4-carboxy-4-tetradecanoylamino-butyrylamino)-butyryl-, 6-[Hydroxy-(naphthalene-2-yloxy)-phosphoryloxy]-hexanoyl-, 6-[Hydroxy-(5-phenyl-pentyloxy)-phosphoryloxy]-hexanoyl-, 4-(Naphthalene-2-sulfonylamino)-4-oxo-butyryl-, 4-(Biphenyl-4-sulfonylamino)-4-oxo-butyryl-, (S)-4-Carboxy-4-{(S)-4-carboxy-4-[2-(2-{2-[2-(2-{2-[(S)-4-carboxy-4-(17-carboxy-heptadecanoylamino)-butyrylamino]-ethoxy}-ethoxy)-acetylamino]-ethoxy}-ethoxy)-acetylamino]-butyrylamino}-butyryl-, (S)-4-Carboxy-4-[2-(2-{2-[2-(2-{2-[(S)-4-carboxy-4-(17-carboxy-heptadecanoylamino)-butyrylamino]-ethoxy}-ethoxy)-acetylamino]-ethoxy}-ethoxy)-acetylamino]-butyryl-, (S)-4-Carboxy-2-{(S)-4-carboxy-2-[2-(2-{2-[2-(2-{2-[(S)-4-carboxy-4-(17-carboxy-heptadecanoylamino)-butyrylamino]-ethoxy}-ethoxy)-acetylamino]-ethoxy}-ethoxy)-acetylamino]-butyrylamino}-butyryl-, (S)-4-Carboxy-2-[2-(2-{2-[2-(2-{2-[(S)-4-carboxy-4-(17-carboxy-heptadecanoylamino)-butyrylamino]-ethoxy}-ethoxy)-acetylamino]-ethoxy}-ethoxy)-acetylamino]-butyryl-, (S)-4-Carboxy-4-{(S)-4-carboxy-4-[2-(2-{2-[(S)-4-carboxy-4-(17-carboxy-heptadecanoylamino)-butyrylamino]-ethoxy}-ethoxy)-acetylamino]-butyrylamino}-butyryl-, (S)-4-Carboxy-4-[2-(2-{2-[(S)-4-carboxy-4-(17-carboxy-heptadecanoylamino)-butyrylamino]-ethoxy}-ethoxy)-acetylamino]-butyryl-, (S)-4-Carboxy-2-{(S)-4-carboxy-2-[2-(2-{2-[(S)-4-carboxy-4-(17-carboxy-heptadecanoylamino)-butyrylamino]-ethoxy}-ethoxy)-acetylamino]-butyrylamino}-butyryl-, (S)-4-Carboxy-2-[2-(2-{2-[(S)-4-carboxy-4-(17-carboxy-heptadecanoylamino)-butyrylamino]-ethoxy}-ethoxy)-acetylamino]-butyryl-, 2-(2-{2-[2-(2-{2-[(S)-4-Carboxy-4-(17-carboxy-hepta-decanoylamino)-butyrylamino]-ethoxy}-ethoxy)-acetylamino]-ethoxy}-ethoxy)-acetyl-, 2-(2-{2-[(S)-4-Carboxy-4-(17-carboxy-heptadecanoylamino)-butyrylamino]-ethoxy}-ethoxy)-acetyl, (S)-4-Carboxy-4-((S)-4-carboxy-4-{(S)-4-carboxy-4-[(S)-4-carboxy-4-(19-carboxy-nonadecanoylamino)-butyrylamino]-butyrylamino}-butyrylamino)-butyryl, 2-(2-{2-[2-(2-{2-[(S)-4-Carboxy-4-(16-1H-tetrazol-5-yl-hexadecanoylamino)-butyrylamino]-ethoxy}-ethoxy)-acetylamino]-ethoxy}-ethoxy)-acetyl-, 2-(2-{2-[2-(2-{2-[(S)-4-Carboxy-4-(16-carboxy-hexadecanoylamino)-butyrylamino]-ethoxy}-ethoxy)-acetylamino]-ethoxy}-ethoxy)-acetyl-, (S)-4-Carboxy-4-{(S)-4-carboxy-4-[(S)-4-carboxy-4-(17-carboxy-heptadecanoylamino)-butyrylamino]-butyrylamino}-butyryl-, (S)-4-Carboxy-4-((S)-4-carboxy-4-{2-[2-(2-{2-[2-(2-{(S)-4-carboxy-4-[10-(4-carboxy-phenoxy)-decanoylamino]-butyrylamino}-ethoxy)-ethoxy]-acetylamino}-ethoxy)-ethoxy]-acetylamino}-butyryl-, (S)-4-Carboxy-4-{(S)-4-carboxy-4-[2-(2-{2-[2-(2-{2-[(S)-4-carboxy-4-(7-carboxy-heptanoylamino)-butyrylamino]-ethoxy}-ethoxy)-acetylamino]-ethoxy}-ethoxy)-acetylamino]-butyrylamino}-butyryl-, (S)-4-Carboxy-4-{(S)-4-carboxy-4-[2-(2-{2-[2-(2-{2-[(S)-4-carboxy-4-(11-carboxy-undecanoylamino)-butyrylamino]-ethoxy}-ethoxy)-acetylamino]-ethoxy}-ethoxy)-acetylamino]-butyrylamino}-butyryl-, (S)-4-Carboxy-4-{(S)-4-carboxy-4-[2-(2-{2-[2-(2-{2-[(S)-4-carboxy-4-(13-carboxy-tridecanoylamino)-butyrylamino]-ethoxy}-ethoxy)-acetylamino]-ethoxy}-ethoxy)-acetylamino]-butyrylamino}-butyryl-, (S)-4-Carboxy-4-{(S)-4-carboxy-4-[2-(2-{2-[2-(2-{2-[(S)-4-carboxy-4-(15-carboxy-pentadecanoylamino)-butyrylamino]-ethoxy}-ethoxy)-acetylamino]-ethoxy}-ethoxy)-acetylamino]-butyrylamino}-butyryl-, and (S)-4-Carboxy-4-{(S)-4-carboxy-4-[2-(2-{2-[2-(2-{2-[(S)-4-carboxy-4-(19-carboxy-nonadecanoylamino)-butyrylamino]-ethoxy}-ethoxy)-acetylamino]-ethoxy}-ethoxy)-acetylamino]-butyrylamino}-butyryl-.

26. A method of treating an autoimmune or an inflammatory disease, comprising administering the compound of formula (I) according to claim 3 to a subject in need thereof.

27. A pharmaceutical composition comprising at least one compound of formula (I) according to claim 3, or a physiological acceptable salt or solvate thereof, and at least a pharmaceutically acceptable carrier.
